(19) United States
(12) Patent Application Publication
(10) Pub. No.: US 2026/0250737 A1
(43) Pub. Date: Aug. 27, 2026

(54) Engineered Luciferases and Luciferin Substrates

(71) Applicant: Monod Bio, Inc., Seattle, WA (US)

(72) Inventors: Alfredo Quijano Rubio, Seattle, WA (US); Daniel Adriano Silva Manzano, Seattle, WA (US); Jesus Renan Vergara Gutierrez, Seattle, WA (US); Joseph Leslie Harman Jr., Seattle, WA (US); Jonathan Richard Wagner, Seattle, WA (US); Paursa Kamalian, Seattle, WA (US); Gonçalo Bernardes, Seattle, WA (US); Hsien-Wei Yeh, Seattle, WA (US); Nicholas Lara, Seattle, WA (US); Elyse Fischer, Seattle, WA (US)

(21) Appl. No.: 19/478,669

(22) PCT Filed: Apr. 24, 2024
(86) PCT No.: PCT/US2024/026098

§ 371 Date: Oct. 24, 2025

Related U.S. Application Data
(60) Provisional application No. 63/498,236, filed on Apr. 25, 2023.
(60) Provisional application No. 63/505,939, filed on Jun. 2, 2023.
(60) Provisional application No. 63/625,901, filed on Jan. 26, 2024.

(51) Int. Cl. C12Q 1/66; C12N 9/02

(52) U.S. Cl. CPC C12Q 1/66; C12N 9/0069; C12Y 113/12007

(57) ABSTRACT

The present disclosure provides a protein having luciferase activity, comprising the secondary structure arrangement H1-L1-H2-L2-B1-L3-B2-L4-H3-L5-B3-L6-B4-L7-B5-L8-B6, wherein (i) “H” is a helical domain, “L” is a loop domain, and “B” is a beta strand domain, wherein the B5 domain is at least 11, 12, 13, or 14 amino acids in length and residue 11 of the B5 domain is F, Y, or L; and the B4 domain is at least 12 amino acids in length and residue 10 of the B4 domain is F, Y, L, I, K or M; and/or residue 12 of the B4 domain is F, L, R, D, M, Q or V; (ii) the H1 domain is at least 18 or 19 amino acids in length; residue 9 of the H1 domain is T, S, H, R, C, L, D, V, A, Q, G, E, K, I, N or M; (iii) the B3 domain is at least 6, 7, 8, 9, or 10 amino acids in length and residue 1 of the B3 domain is W or H; (iv) the B4 domain is at least 12 amino acids in length and residue 10 of the B4 domain is F, Y, L, I, K or M; or (v) the B4 domain is at least 12 amino acids in length and residue 12 of the B4 domain is L, R, D, M, Q or V. Additional proteins disclosed herein are exemplified in the claims. Also provided herein are luciferase substrates, assay buffers, and kits comprising one or more of a protein having luciferase activity or a nucleic acid encoding the protein, an assay buffer, and a luciferase substrate.

### CROSS-REFERENCE

This application claims the benefit of U.S. Provisional Patent Application No. 63/625,901 filed on Jan. 26, 2024, U.S. Provisional Patent Application No. 63/505,939 filed on Jun. 2, 2023, and U.S. Provisional Patent Application No. 63/498,236 filed on Apr. 25, 2023, which applications are herein incorporated by reference in their entirety.

### INCORPORATION BY REFERENCE OF XML SEQUENCE LISTING

A Sequence Listing is provided herewith as a Sequence Listing XML, “MOBI-010WO_SEQLIST_4-23-24.XML,” created on Apr. 23, 2024 and having a size of 2,879,287 bytes. The contents of the text file are incorporated by reference herein in their entirety.

### INTRODUCTION

Bioluminescence produced upon oxidation of a luciferin substrate by enzymatic activity of luciferases has been utilized in biological assays in cell free systems, in vitro, and in vivo. Since no excitation is needed to induce emission, luminescence occurs in the dark, providing significant advantages over fluorescence, including lower background signals and not requiring excitation which can cause phototoxicity in tissues.

Work has been performed to engineer native luciferases to improve their use as molecular probes. However, satisfactory luciferases based on native luciferases have not been generated. A synthetic luciferase, named LuxSit, has been developed by the Baker lab at University of Washington (Nature 614, 774-780 (2023)).

D-luciferin and coelenterazine, and their respective luciferases are widely known luciferin/luciferase pairs and are routinely used in majority of applications of bioluminescence such as gene assays, the detection of protein-protein interactions, high-throughput screening (HTS) in drug discovery, hygiene control, analysis of pollution in ecosystems and in vivo imaging in small mammals (Syed et al., Chem. Soc. Rev., 2021, 50, 5668).

Significant work has been done in the field of synthetic chemistry to develop both luciferins with beneficial properties. Synthetic luciferin analogues are known to have a longer-lasting and sustained bioluminescence signal compared to that of D-luciferin. Synthetic coelenterazine analogues are reported to have higher brightness and better solubility than coelenterazine (Syed et al., Chem. Soc. Rev., 2021, 50, 5668). It is still desired to develop new luciferins having improved properties.

### SUMMARY

The present disclosure provides a protein having luciferase activity, comprising the secondary structure arrangement H1-L1-H2-L2-B1-L3-B2-L4-H3-L5-B3-L6-B4-L7-B5-L8-B6, wherein (i) “H” is a helical domain, “L” is a loop domain, and “B” is a beta strand domain, wherein the B5 domain is at least 11, 12, 13, or 14 amino acids in length and residue 11 of the B5 domain is F, Y, or L; and the B4 domain is at least 12 amino acids in length and residue 10 of the B4 domain is F, Y, L, I, K or M; and/or residue 12 of the B4 domain is F, L, R, D, M, Q or V; (ii) the H1 domain is at least 18 or 19 amino acids in length; residue 9 of the H1 domain is T, S, H, R, C, L, D, V, A, Q, G, E, K, I, N or M; (iii) the B3 domain is at least 6, 7, 8, 9, or 10 amino acids in length and residue 1 of the B3 domain is W, L or H; (iv) the B4 domain is at least 12 amino acids in length and residue 10 of the B4 domain is F, Y, L, I, K or M; or (v) the B4 domain is at least 12 amino acids in length and residue 12 of the B4 domain is L, R, D, M, Q or V. Also provided are split versions of these proteins where the protein is split into two or three components which are self-complementing and have luciferase activity when associated non-covalently. Circularly permuted polypeptides having luciferase activity are also disclosed. Also provided is an assay solution for measuring luciferase activity of a protein, which assay solution includes 1 mM-1000 mM imidazole.

Also provided are luciferin substrates. These luciferin substrates can be used to measure activity of a luciferase, e.g., the luciferase activity of proteins disclosed herein.

### BRIEF DESCRIPTION OF THE DRAWINGS

FIG. 1. The secondary structure arrangement H1-L1-H2-L2-B1-L3-B2-L4-H3-L5-B3-L6-B4-L7-B5-L8-B6 mapped on the amino acid sequence (SEQ ID NO:1) of LuxSit-i.

FIG. 2. Single mutants of LuxSit-i protein enriched based on luciferase activity.

FIG. 3. Graphical representation of the mutation frequency at the positions found in the single saturation mutagenesis (SSM) as improving protein luciferase activity.

FIG. 4A. Comparison of luciferase activity of MBIO-148, MBIO-158 and LuxSit-i.

FIG. 4B. Comparison of luciferase activity of MBIO-148, MBIO-301, MBIO-302, and LuxSit-i.

FIG. 5A. Single mutants of LuxSit-i protein enriched based on stability.

FIG. 5B. Comparison of stability of LuxSit-i protein and LuxSit-i protein variants MBIO-301, MBIO-3073, and MBIO-4039.

FIG. 5C. Static light scattering (SLS) at 266 nm was used to detect the formation of small aggregates early in thermal denaturation.

FIG. 6A. Kinetic profile over one hour of circularly permuted LuxSit-i variants.

FIG. 6B. Initial Relative Light Unit (RLU) values of circularly permuted LuxSit variants.

FIG. 7. Luminescent activity of the high-affinity two-component luciferase variants fused to the rapamycin inducible FRB:FKBP system.

FIG. 8. Luminescent activity of the low-affinity two-component luciferase variants fused to the rapamycin inducible FRB:FKBP system.

FIGS. 9A and 9B. Improvement of luciferase activity in buffer with high imidazole concentration.

FIG. 10 shows bioluminescence emission spectra of synthetic luciferin substrates 1a, 1b, 1c, 1d, 1k, in, and 1p incubated with MBIO-301 enzyme.

FIG. 11 shows bioluminescence emission spectra of synthetic luciferlin substrates 2a, 2b, 2c, 2d, 2f, 2h, and 2p, incubated with MBIO-301 enzyme.

FIG. 12 shows bioluminescence emission spectra of synthetic luciferin substrates 3b and 3i incubated with MBIO-301 enzyme.

FIG. 13A shows luminescence obtained upon incubation of synthetic luciferin substrates with MBIO-301 enzyme. FIG. 13B shows luminescence obtained upon incubation of synthetic luciferin substrates with MBIO-4039 enzyme. FIG. 13C shows luminescence obtained upon incubation of synthetic luciferin substrates with MBIO-4040 enzyme.

FIG. 14 compares bioluminescence emission of synthetic luciferin substrate 1c and DTZ incubated with MBIO-301 in two assay conditions: 100% assay buffer, and 20% human serum+80% assay buffer.

FIG. 15 shows luminescence obtained upon incubation of synthetic luciferin substrates with MBIO-301-derived split enzyme.

FIG. 16A, FIG. 16B and FIG. 16C show mass spectroscopy of compound 1p_2p according to embodiments of the present disclosure.

FIG. 17A, FIG. 17B and FIG. 17C show mass spectroscopy of compound 1p according to embodiments of the present disclosure.

FIG. 18A, FIG. 18B and FIG. 18C show mass spectroscopy of compound 2a according to embodiments of the present disclosure.

FIG. 19A, FIG. 19B and FIG. 19C show mass spectroscopy of compound 2a1u according to embodiments of the present disclosure.

FIG. 20A, FIG. 20B and FIG. 20C show mass spectroscopy of compound 2a1v according to embodiments of the present disclosure.

FIG. 21A, FIG. 21B and FIG. 21C show mass spectroscopy of compound 2a1w according to embodiments of the present disclosure.

FIG. 22A, FIG. 22B and FIG. 22C show mass spectroscopy of compound 2a1x according to embodiments of the present disclosure.

FIG. 23A, FIG. 23B and FIG. 23C show mass spectroscopy of compound 2k according to embodiments of the present disclosure.

FIG. 24A, FIG. 24B and FIG. 24C show mass spectroscopy of compound 20 according to embodiments of the present disclosure.

FIG. 25A, FIG. 25B and FIG. 25C show mass spectroscopy of compound 2p according to embodiments of the present disclosure.

FIG. 26A, FIG. 26B and FIG. 26C show mass spectroscopy of compound 2p1u according to embodiments of the present disclosure.

FIG. 27A, FIG. 27B and FIG. 27C show mass spectroscopy of compound 2p1v according to embodiments of the present disclosure.

FIG. 28A, FIG. 28B and FIG. 28C show mass spectroscopy of compound 2p1w according to embodiments of the present disclosure.

FIG. 29A, FIG. 29B and FIG. 29C show mass spectroscopy of compound 2p1x according to embodiments of the present disclosure.

FIG. 30A, FIG. 30B and FIG. 30C show mass spectroscopy of compound 1n2a according to embodiments of the present disclosure.

FIG. 31A, FIG. 31B and FIG. 31C show mass spectroscopy of compound 1n2p according to embodiments of the present disclosure.

FIG. 32A, FIG. 32B and FIG. 32C show mass spectroscopy of compound 1p2a according to embodiments of the present disclosure.

FIG. 33A, FIG. 33B and FIG. 33C show mass spectroscopy of compound 1p2p according to embodiments of the present disclosure.

FIG. 34A, FIG. 34B and FIG. 34C show mass spectroscopy of compound 1w according to embodiments of the present disclosure.

FIG. 35A, FIG. 35B and FIG. 35C show mass spectroscopy of compound 1x according to embodiments of the present disclosure.

FIGS. 36A-36F show enzymatic activity of LuxSit-i variants in a function-complementation assay.

FIG. 37A-37H show enzymatic activity of LuxSit-i variants.

FIG. 38 shows comparison of enzymatic activity of LuxSit-i variant MBIO-4039 to enzymatic activity of LuxSit-i.

FIG. 39 shows enzymatic activity of LuxSit-I variant Mbio-3073 measured in different buffers, tris-buffered saline (TBS), OB2.0, and OB3.0.

FIG. 40. LuxSit-i variant, MBIO-4039, has improved yield as compared to LuxSit-i.

FIG. 41. Schematic of constructs for testing LuxSit Splits.

FIGS. 42A-42G show enzymatic activity of LuxSit-I variants measured in different buffers: phosphate-buffered saline (PBS), OPT1.0, OPT2.0, and OPT3.0.

FIGS. 43A-43D show enzymatic activity of LuxSit-i variants measured using substrate 1c and the buffers PBS, OPT1.0, OPT2.0, and OPT3.0.

FIG. 44. Schematic of constructs for measuring stability of LgLux generated by error-prone PCR and identifying LgLux with improved protease resistance.

FIG. 45. Schematic of constructs for measuring stability of LgLux generated computationally and identifying LgLux with improved protease resistance.

FIGS. 46A-46C. MBIO-4517 and MBIO-4039 were split into large and small fragments and conjugated to the proteins that bind the molecule Rapamycin (FKBP and FRB). Luciferase activity before and after rapamycin addition is shown in FIG. 46A. A plot comparing luciferase activity of the split luciferase version to full length version for MBIO-4517 and for MBIO-4039 is shown in FIG. 46B and FIG. 46C, respectively.

### DETAILED DESCRIPTION

The present disclosure provides a protein having luciferase activity, comprising the secondary structure arrangement H1-L1-H2-L2-B1-L3-B2-L4-H3-L5-B3-L6-B4-L7-B5-L8-B6, wherein (i) “H” is a helical domain, “L” is a loop domain, and “B” is a beta strand domain, wherein the B5 domain is at least 11, 12, 13, or 14 amino acids in length and residue 11 of the B5 domain is F, Y, or L; and the B4 domain is at least 12 amino acids in length and residue 10 of the B4 domain is F, Y, L, I, K or M; and/or residue 12 of the B4 domain is F, L, R, D, M, Q or V; (ii) the H1 domain is at least 18 or 19 amino acids in length; residue 9 of the H1 domain is T, S, H, R, C, L, D, V, A, Q, G, E, K, I, N or M; (iii) the B3 domain is at least 6, 7, 8, 9, or 10 amino acids in length and residue 1 of the B3 domain is W, L or H; (iv) the B4 domain is at least 12 amino acids in length and residue 10 of the B4 domain is F, Y, L, I, K or M; or (v) the B4 domain is at least 12 amino acids in length and residue 12 of the B4 domain is L, R, D, M, Q or V. Also provided are split versions of these proteins where the protein is split into two or three components which are self-complementing. These components, when not physically associated with each other, either lack or have substantially reduced luciferase activity and have luciferase activity when physically associated. The physical association may be non-covalent or covalent association. Examples of non-covalent association includes association mediates via one or more moieties, e.g., a protein or a small molecule to which the individual components bind and are brought into sufficient physical proximity to achieve functional complementation. Examples of covalent association include a linker (e.g., a peptide or a polypeptide) linking the two components (or more components) to form a polypeptide having luciferase activity. The linker may be cleavable, e.g., includes a cleavage site. Upon cleavage of the linker, the two components (or more components) are physically separated resulting in loss or substantial decrease in luciferase activity.

Circularly permuted polypeptides having luciferase activity are also disclosed.

Also provided is an assay solution, e.g., a buffer, for measuring luciferase activity of a protein or a protein complex, which assay solution includes 1 mM-1000 mM imidazole. In certain experiments, the assay solution may have an alkaline pH.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, an and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Definitions

“Derived from” in the context of an amino acid sequence or polynucleotide sequence is meant to indicate that the polypeptide or nucleic acid has a sequence that is based on that of a reference polypeptide or nucleic acid, and is not meant to be limiting as to the source or method in which the protein or nucleic acid is made.

The terms “polypeptide”, and “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids, which include, but are not limited to those listed in 37 CFR (§ 1.822(b)(4)). Furthermore, it should be noted that a dash at the beginning or end of an amino acid residue sequence indicates either a peptide bond to a further sequence of one or more amino acid residues or a covalent bond to a carboxyl or hydroxyl end group. However, the absence of a dash should not be taken to mean that such peptide bonds or covalent bond to a carboxyl or hydroxyl end group is not present, as it is conventional in representation of amino acid sequences to omit such. The term “peptide” also refers to a linear series of amino acid residues connected one to the other by peptide bonds between the alpha-amino and carboxy groups of adjacent residues but is generally shorter than a protein or a polypeptide, e.g., less than 50 amino acids long, e.g., 2-50 amino acids in length. The terms protein, polypeptide, and peptide may be used interchangeably.

As used herein, the term “binding” refers to the non-covalent interactions of the type which occur between two molecules. The strength or affinity of binding interactions can be expressed in terms of the dissociation constant (K_{D}) of the interaction, wherein a smaller K_{D} represents a greater affinity. Binding properties of selected polypeptides can be quantified using methods well known in the art.

“Isolated” refers to an entity of interest that is in an environment different from that in which the entity may naturally occur or is initially produced in. An “isolated” compound (e.g., an “isolated” polypeptide) is separated from all or some of the components that accompany it and may be substantially enriched, e.g., may be purified so that the compound is at least about 70% pure, at least about 80% pure, at least about 90% pure, at least about 95% pure, at least about 98% pure, at least about 99%, or greater than 99% pure, or free of impurities, contaminants, and/or components other than the compound. “Isolated” also refers to the state of a compound separated from all or some of the components that accompany it during manufacture (e.g., chemical synthesis, recombinant expression, culture medium, and the like).

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (lie; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e., the N-terminal methionine residue may be present or absent). In all embodiments of polypeptides disclosed herein, any C-terminal glycine residues are optional (i.e., the C-terminal glycine residue may be present or absent).

The term “conservative substitution” is used in reference to proteins to reflect amino acid substitutions that do not substantially alter the activity (specificity or binding affinity) of the molecule. Typically, conservative amino acid substitutions involve substituting one amino acid for another amino acid with similar chemical properties (e.g., charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); and 6) Phenylalanine (F), Tyrosine (Y), Tryptophan (W). The polypeptides encompassed by the present disclosure include those that have one or more conservative substitutions relative to the amino acid sequences provided here.

Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends. Percent Identity=(Matches x 100)/Length of aligned region (with gaps). “Alkyl” refers to a monoradical, branched or linear, non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases, the alkyl group has 1 to 24 carbon atoms, e.g., 1 to 12, 1 to 6, or 1 to 3.

“Alkenyl” refers to a monoradical, branched or linear, non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl.

“Alkynyl” refers to a monoradical, branched or linear, non-cyclic hydrocarbonyl group that comprises a carbon-carbon triple bond. Exemplary alkynyl groups include ethynyl and n-propynyl.

“Cycloalkyl” refers to a monoradical, cyclic, saturated hydrocarbon group. Similarly, “cycloalkenyl” refers to a monoradical and cyclic group having carbon-carbon double bond whereas “cycloalkynyl” refers to a monoradical and cyclic group having carbon-carbon triple bond.

“Heterocyclyl” refers to a monoradical, cyclic group that contains a heteroatom (e.g., 0, S, N) as a ring atom and that is not aromatic (i.e., distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring, wherein each of the atoms in the ring are carbon atoms, i.e., none of the ring atoms are heteroatoms (e.g., O, S, N). In some cases, the aryl group has a second aromatic ring, e.g. that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring, wherein at least one of the atoms in the aromatic ring is a heteroatom (e.g., O, S, N). Exemplary heteroaryl groups include those obtained from removing a hydrogen atom from pyridine, pyrimidine, furan, thiophene, or benzothiophene.

The term “substituted” refers to the removal of one or more hydrogens from an atom (e.g., from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g. giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no longer be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

Exemplary substituents include alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, sulfonate, and substituted versions thereof.

In some cases, the substitutions can themselves be further substituted with one or more groups. For example, the group —C₆H4CH₂CH₃ can be considered as substituted aryl, i.e., an aryl group substituted with the ethyl, which is an alkyl group. Furthermore, the ethyl group can itself be substituted with a pyridyl group to form —C_{G}H4CH₂CH₂C₅H₅N, wherein —C₆H₄CH₂CH₂C₅H₅N can also be considered as a substituted aryl group as the term is used herein. In some cases, the substituents are not substituted with any other groups.

Diradical groups are also described herein, i.e., in contrast to the monoradical groups such as alkyl and aryl described above. The term “alkylene” refers to the diradical version of an alkyl group, i.e., an alkylene group is a diradical, branched or linear, cyclic or non-cyclic, saturated hydrocarbon group. Exemplary alkylene groups include diylmethane (—CH₂—, which is also known as a methylene group), 1,2-diylethane (—CH₂CH₂—), and 1,1-diylethane (i.e., a CHCH₃ fragment where the first atom has two single bonds to other two different groups). The term “arylene” refers to the diradical version of an aryl group, e.g., 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein.

“Alkoxy” refers to a group of formula —O(alkyl). Similar groups can be derived from alkenyl, alkynyl, aryl, heteroaryl, and other groups.

“Amino” refers to the group —NR^{X}R^{Y} wherein R^{X} and R^{Y} are each independently H or a non-hydrogen substituent. Exemplary non-hydrogen substituents include alkyl groups (e.g., methyl, ethyl, and isopropyl).

“Hydroxyl” refers to the group of formula —OH.

“Halo” and “halogen” refer to the chloro, bromo, fluoro, and iodo groups.

“Haloalkyl” refers to an alkyl group in which hydrogen atoms are replaced by a halogen.

“Nitro” refers to the group of formula —NO₂.

Unless otherwise specified, reference to an atom is meant to include all isotopes of that atom. For example, reference to H includes ¹H, ²H (i.e., D or deuterium) and ³H (i.e., tritium), and reference to C includes both ¹²C and all other isotopes of carbon (e.g., ¹³C). Unless specified otherwise, groups include all possible stereoisomers.

Numeric ranges are inclusive of the numbers defining the range.

### Polypeptides

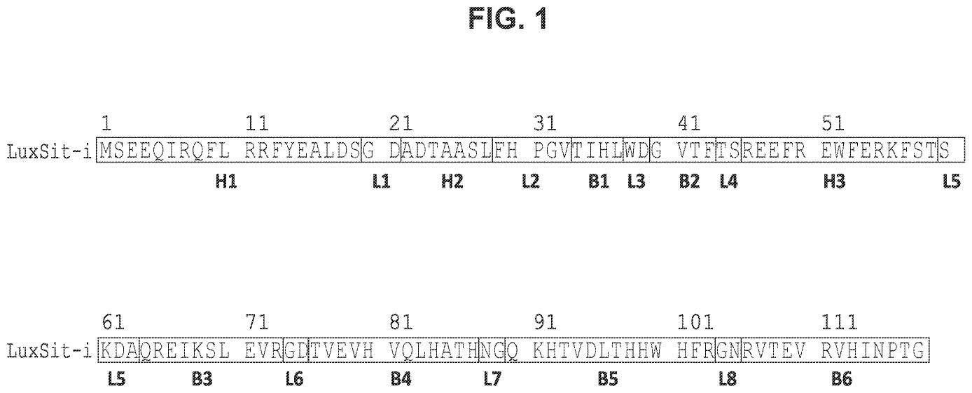

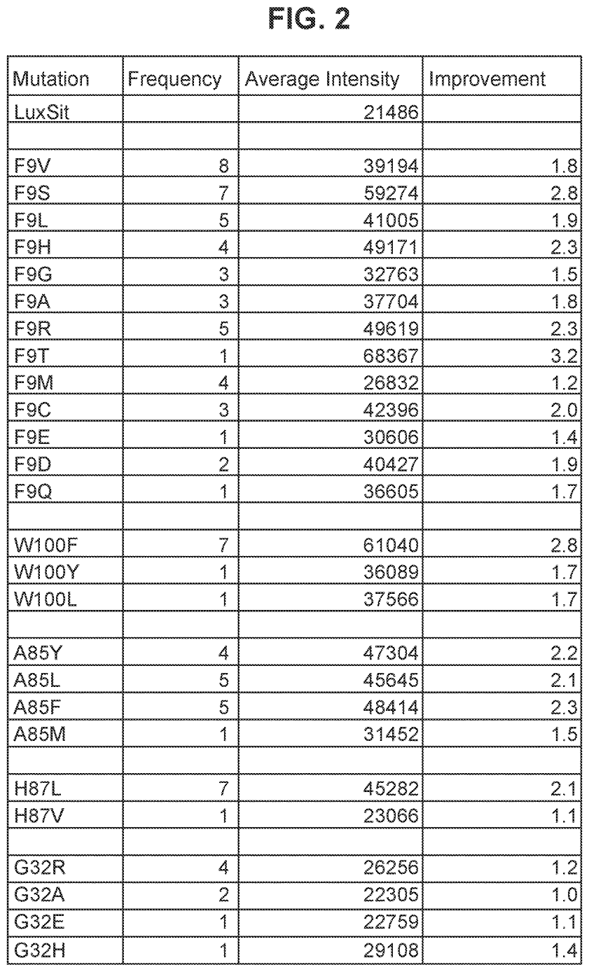

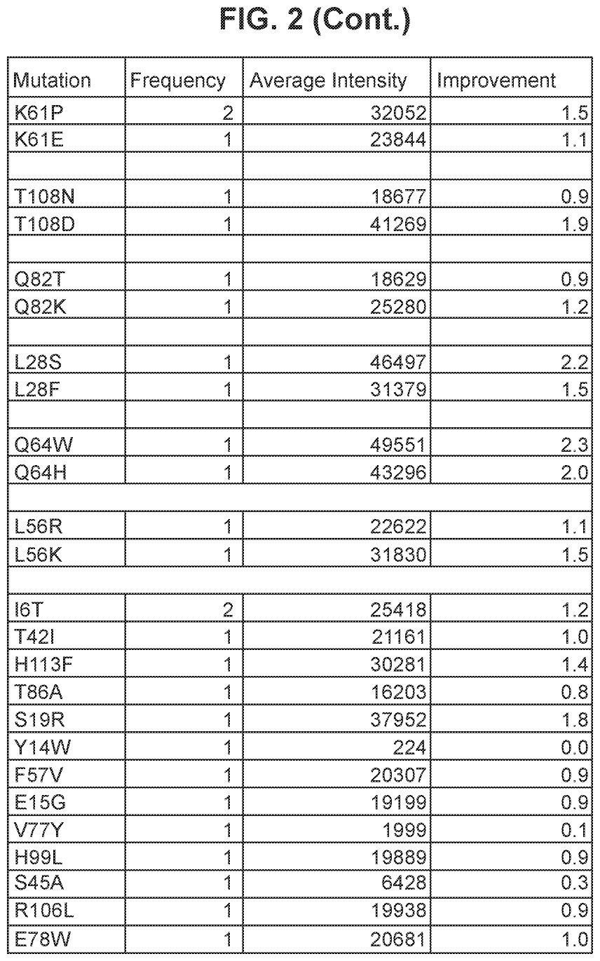

The polypeptides disclosed herein are based on a polypeptide referred to as LuxSit-i (SEQ ID NO:1, FIG. 1). LuxSit-i is an optimized version of LuxSit (Latin: let light exist). LuxSit is a de novo designed synthetic luciferase having no significant sequence similarity to naturally occurring luciferases. LuxSit is based on the toplogy of NTF2 (nuclear transport factor 2)-like suprfamily of proteins which do not have luciferase activity but contains multiple pockets having size and structure compatible for binding to luciferase substrates such as Diphenylterazine (DTZ). LuxSit was generated by (i) optimizing the core regions of the binding pocket, while allowing changes, such as substitutions and/or deletions, in more flexible regions of the proteins to identify an optimal scaffold compatible with the binding pocket; followed by (ii) screening for active sites for DTZ while keeping the scaffold stable. See Nature 614, 774-780 (2023). LuxSit has the following secondary structure that define the protein scaffold:

H1-L1-H2-L2-B1-L3-B2-L4-H3-L5-B3-L6-B4-L7-B5-L8-B6, wherein “H” is a helical domain, “L” is a loop domain, and “B” is a beta strand domain.

LuxSit includes catalytic dyads of (i) D residue at positon 18 in the H1 domain and R residue at position 2 in the B3 domain (also referred to as Asp18-Arg65) which form Dyad 1; and (ii) Y residue at position 14 in the H1 domain and H residue at position 9 in the B5 domain (also referred to as Tyr14-His98) which form Dyad 2.

The amino acid sequence of LuxSit is set forth in SEQ ID NO:92:

(SEQ ID NO: 92)
(M)SEEQIRQFL RRFYEALDSG DADTAASLFH PGVTIHLWDG
VTFTSREEFR EWFERLFSTR KDAQREIKSL EVRGDTVEVH
VQLHATHNGQ KHTVDATHHW HFRGNRVTEM RVHINPT(G)

LuxSit-i offers many advantages as compared to naturally occurring luciferases, such as, small size, stability, robust folding, and high activity. An optimized version of LuxSit having the following substitutions R60S/A96L/M110V relative to SEQ ID NO:92 was created and is referred to as LuxSit-i. LuxSit and LuxSit-i are described in Nature 614, 774-780 (2023).

The amino acid sequence of LuxSit-i is set forth in SEQ ID NO:1:

(SEQ ID NO: 1)
(M)SEEQIRQFL RRFYEALDSG DADTAASLFH PGVTIHLWDG
VTFTSREEFR EWFERLFSTS KDAQREIKSL EVRGDTVEVH
VQLHATHNGQ KHTVDLTHHW HFRGNRVTEV RVHINPT(G)

In each of the annotated sequences shown for SEQ ID NO:1 and 92:
(a) Bold and underlined residues are Dyad 1 (catalytic residues) Y14 (H1 domain residue 14)+H98 (B5 domain residue 9);
(b) Bold residues are Dyad 2 (catalytic residues) D18 (H1 domain residue 9)+R65 (B3 domain residue 2);
(c) Italicized residues are core packing (recognition residues) F13 (residue 13 of domain H1), I35 (residue 2 of domain B1), W38 (residue 1 of domain L3), F49 (residue 4 of domain H3), V81 (residue 6 of domain B4), L83 (residue 8 of domain B4), V94 (residue 5 of domain B5), A/L 97 (residue 8 of domain B5), W100 (residue 11 of domain B5), M/V110 (residue 5 of domain B6), V112 (residue 7 of domain B6); and
(d) Underlined and not bolded positions are loop domains or immediately adjacent residues that facilitate splitting the enzyme or inserting other functional domains.

The amino acids in parenthesis may be present or absent. FIG. 1 shows the secondary structure arrangement H1-L1-H2-L2-B1-L3-B2-L4-H3-L5-B3-L6-B4-L7-B5-L8-B6 mapped on the amino acid sequence of LuxSit-i.

### Polypeptides Having Luciferase Activity

### LuxSit-i Variants

The polypeptides described herein include one or more changes in the amino acid sequence of LuxSit-i which changes result in improvement in one or more properties of the protein compared to LuxSit-i.

In certain aspects, these polypeptides have improved activity compared to LuxSit-i. For example, these polypeptides have a luciferase activity that is at least 10% higher than LuxSit-i luciferase activity, e.g., at least 20% higher, at least 30% higher, at least 40% higher, at least 50% higher, at least 60% higher, at least 70% higher, at least 80% higher, at least 90% higher, at least 100% higher, at least 150% higher, or up to 150% higher, or up to 180% higher, or up to 200% higher than LuxSit-i luciferase activity. The luciferase activity may be measured using any suitable assay, including assays provided herein. The luciferase activity may be measured using a luciferin substrate, e.g., DTZ, coelenterazine, furimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine, coelenterazine-i, coelenterazine-icp, coelenterazine-v, and 2-methyl coelenterazine, or another luciferin substrate, or an analog thereof. The luciferase activity may be measured using a compound disclosed herein.

In certain aspects, these polypeptides have improved stability at high temperatures as compared to LuxSit-i. For example, these polypeptides are stable at higher temperatures as compared to LuxSit-i. Stability may be measured by enzymatic activity and/or protein misfolding measured over a period of time. In certain embodiments, stability may be measured using static light scattering (SLS). In certain embodiments, the polypeptides disclosed herein are more stable than LuxSit-i at a temperature higher than 37.C. In certain embodiments, the polypeptides disclosed herein are more stable than LuxSit-i at a temperature higher than 37.C, as measured by SLS.

In certain aspects, these polypeptides have improved specificity as compared to LuxSit-i. For example, it may have 2×, 3, ×, 5×, 10× higher specificity for a luciferin substrate as compared to LuxSit-i.

In certain aspects, these polypeptides have improved yield compared to LuxSit-i. For example, these polypeptides are expressed at higher levels and/or with lower levels of aggregated or misfolded proteins as compared to LuxSit-i when expressed in standard expression systems such as E. coli, yeast, mammalian cell lines, and the like. FIG. 40 shows improvement in yield of LuxSit-i variant compared to LuxSit-i expressed in of E. coli (BL21) culture. A 1 L culture was used for the expression of LuxSit-i variant and LuxSit-i.

In certain aspects, the polypeptides provided herein have the same secondary structure as LuxSit and LuxSit-i: H1-L1-H2-L2-B1-L3-B2-L4-H3-L5-B3-L6-B4-L7-B5-L8-B6. “H” is a helical domain, “L” is a loop domain, and “B” is a beta strand domain. In the polypeptides provided herein, the H1 domain is at least 18 or 19 amino acids in length; residue 14 of the H1 domain is Y, D, or E, and residue 9 of the H1 domain is D or E; the B3 domain is at least 6, 7, 8, 9, or 10 amino acids in length and residue 2 of the B3 domain is R; and the B5 domain is at least 10, 11, 12, 13, or 14 amino acids in length and residue 9 of the B5 domain is H or N. Accordingly, the catalytic Dyad 1 and Dyad 2 are not altered.

In the polypeptides provided herein, in some aspects, one or more of the core packing may not be altered. In some aspects, residue 13 of domain H1 is F; residue 1 of domain L3 is W; residue 5 of domain B5 is V or another hydrophobic residue; residue 8 of domain B5 is A or L or another hydrophobic residue; and/or residue 11 of domain B5 is W. In further aspects, residue 2 of domain B1 is I or another hydrophobic residue; residue 4 of domain H3 is F; residue 6 of domain B4 is V or another hydrophobic residue; residue 8 of domain B4 is L or another hydrophobic residue; residue 5 of domain B6 is M or V or another hydrophobic residue; and/or residue 7 of domain B6 is V or another hydrophobic residue.

In certain aspects, the H1 domain is 19 amino acids in length; the H2 domain is 7 amino acids in length; the B1 domain is 4 amino acids in length; the B2 domain is 4 amino acids in length; the H3 domain is 14 amino acids in length; the B3 domain is 10 amino acids in length; the B4 domain is 12 amino acids in length; the B5 domain is 14 amino acids in length; and the B6 domain is 12 or 13 amino acids in length. The loop domains may be of any length and may include insertions, relative to the sequences exemplified herein, of any residues or functional domains as deemed appropriate, including but not limited to metal binding domains, drug binding domains, GPCR receptors, protein switches, and small molecule binding domains.

In certain aspects, the H1 domain present in the polypeptides disclosed herein has an amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the amino acid sequence: MSEEQIRQFLRRFYEALDS (SEQ ID NO:2738) or SISEEQIRQFLRRFYEALDS (SEQ ID NO:2739) or IPEEQIRQFLRRFYEALDS (SEQ ID NO:2740) or EISEEQIRQFLRRFYEALDS (SEQ ID NO:2741).

In certain aspects, the H2 domain present in the polypeptides disclosed herein has an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: ADTAASL (SEQ ID NO:2742).

In certain aspects, the B1 domain present in the polypeptides disclosed herein has an amino acid sequence having at least 50%, 75%, or 100% identity to the amino acid sequence: TIHL (SEQ ID NO:2743).

In certain aspects, the B2 domain present in the polypeptides disclosed herein has an amino acid sequence having at least 50%, 75%, or 100% identity to the amino acid sequence: GVTF (SEQ ID NO:2744).

In certain aspects, the H3 domain present in the polypeptides disclosed herein has an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: REEFREWFERLFST (SEQ ID NO:2745).

In certain aspects, the B3 domain present in the polypeptides disclosed herein has an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: WREIKSLEVR (SEQ ID NO:2746).

In certain aspects, the B4 domain present in the polypeptides disclosed herein has an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: TVEVHVQLHFTL (SEQ ID NO:2747) or TVVVVVRLDFTL (SEQ ID NO:2748).

In certain aspects, the B5 domain present in the polypeptides disclosed herein has an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: QKHTVDLTHHFHFR (SEQ ID NO:2749) or QKHTVILTHVFRFR (SEQ ID NO:2750).

In certain aspects, the B6 domain present in the polypeptides disclosed herein has an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: RVTEVRVHINPTG (SEQ ID NO:2751) or RVTEVRVEIVPV (SEQ ID NO:2752).

In certain aspects, the L1, L2, L3, L4, L5, L6, L7, and L8 domains are at least 1, 2, 3, 4, or 5 amino acids in length and comprise any amino acid and optionally are up to 5 amino acids in length. In certain aspects, the L1, L2, L3, L4, L5, L6, L7, and L8 domains include insertions that do not change the overall protein conformation.

In certain aspects, some of the LuxSit-i variants provided herein that have the same secondary structure arrangement as LuxSit-i have an amino acid sequence having at least 50%, 60%, 70%, 75%, 80%, 85%, 90%, 91%, 92%, 93%, 94%, 95%, 96%, 97%, 98%, or 99% identity to the amino acid sequence:

(SEQ ID NO: 1)
MSEEQIRQFLRRFYEALDSGDADTAASLFHPGVTIHLWDGVTFTSREEFR
EWFERLFSTSKDAQREIKSLEVRGDTVEVHVQLHATHNGQKHTVDLTHHW
HFRGNRVTEVRVHINPTG.

LuxSit-i Variant with Substitutions in B4 Domain

In certain aspects, a protein having luciferase activity may include the secondary structure arrangement H1-L1-H2-L2-B1-L3-B2-L4-H3-L5-B3-L6-B4-L7-B5-L8-B6, as described herein, where the B4 domain is at least 12 amino acids in length. In certain aspects, residue 10 of the B4 domain is F, L, Y, I, K or M. In certain aspects, residue 10 of the B4 domain is F. In contrast, residue 10 of the B4 domain of LuxSit-i is A. Residue 10 of the B4 may also be referred to by the position of the amino acid this residue corresponds to in the B4 domain in SEQ ID NO:1, where the residue 10 in B4 domain is position 85 in SEQ ID NO:1.

A protein having luciferase activity may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:1, where residue 85 is F, Y, L, I, K or M, when numbered relative to SEQ ID NO:1.

In all aspects, a relative position with reference to SEQ ID NO:1 may be determined by aligning an amino acid sequence to the amino acid sequence of SEQ ID NO:1.

In another aspect, residue 12 of the B4 domain is F, L, R, D, M, Q or V. In certain aspects, residue 12 of the B4 domain is F. In contrast, residue 12 of the B4 domain of LuxSit-i is H. Residue 12 of the B4 may also be referred to by the position of the amino acid this residue corresponds to in the B4 domain in SEQ ID NO:1, where the residue 12 in B4 domain is position 87 in SEQ ID NO:1.

In certain aspects, a protein having luciferase activity may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99%, identical to the amino acid sequence of SEQ ID NO:1, where residue 87 is F, L, R, D, M, Q or V, when numbered relative to SEQ ID NO:1.

In another aspect, residue 12 of the B4 domain is F, L, R, D, M, Q or V and residue 10 of the B4 domain is F, L, Y, I, K or M. In certain aspects, residue 10 of the B4 domain is F and residue 12 of the B4 domain is F.

In certain aspects, a protein having luciferase activity may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, or 98% identical to the amino acid sequence of SEQ ID NO:1, where residue 87 is F, L, R, D, M, Q or V and residue 85 is F, Y, L, I, K or M, when numbered relative to SEQ ID NO:1.

In certain aspects, a protein having luciferase activity comprises an amino acid sequence having at least 30%, 40%, 50%, 60%, 70%, 75%, 80%, 85%, 90%, 91%, 92%, 93%, 94%, 95%, 96%, 97%, 98%, or 99% identity to the amino acid sequence of SEQ ID NO:1 and
(i) comprises an amino acid substitution at one or more of the following positions relative to SEQ ID NO:1:
E3, I6, Y14, E15, S19, L28, G32, T42, F43, S45, L56, F57, T59, K61, Q64, V77, E78 Q82, A85, T86, H92, L96, H99, W100, R106, T108, and H113, relative to SEQ ID NO:1; and/or
(ii) lacks one or more lysine residues, relative to SEQ ID NO:1 or lacks lysine residues. In certain embodiments, the protein lacks lysine residues present in SEQ ID NO:1, where the lysine residues are replaced with another amino acid, such as R, Q, T, S, L, Y, etc.

In certain embodiments, a protein having luciferase activity comprises an amino acid sequence having at least 30%, 40%, 50%, 60%, 70%, 75%, 80%, 85%, 90%, 91%, 92%, 93%, 94%, 95%, 96%, 97%, 98%, or 99% identity to the amino acid sequence of SEQ ID NO:1 and comprises:
(i) one or more of the substitutions E3D, I6T/K, Y14W, E15G, S19R, L28S/F, G32R/D/A/E/H, T421, F43L/G, S45A, L56R/K/Q, L56R/K/Q, F57V, T59K, Q64W/H, K61P/E, V77Y, E78W, Q82T/K, A85F/Y/L/I/M, T86A, H87L/V, H99L, W100F/Y/L, R106L, V1071, T108N/D, and H113F, relative to SEQ ID NO:1; and/or
(ii) lacks lysine residues and comprises one or more of the substitutions: F9, D23, H30, H36, V41, R46, R55, L56, Q64, K68, H80, Q82, H84, A85, H87, H92, T97, H98, H99, W100, H101, R103, T108, E109, H113, and I114, relative to SEQ ID NO:1.

In certain embodiments, the protein lacks lysine residues which are replaced with another amino acid, e.g., R, Q, T, S, L, Y, etc.

In certain embodiments, the amino acid sequence of the protein comprises all of the following substitutions: F9V/S/N, D231, H30D, H36T, V41T, R46V, R55S, L56Q, Q64L, K68S, H80T, Q82R, H84S, A85F, H87R, H92S, T97L, H98Q, H99L, W100F, H101K/R, R103V, T108D/V, E109A, H113Y, and I114V.

LuxSit-i Variant with Substitutions in B4 and B5 Domains

In certain aspects, a protein having luciferase activity may include the secondary structure arrangement H1-L1-H2-L2-B1-L3-B2-L4-H3-L5-B3-L6-B4-L7-B5-L8-B6 as described herein, where residue 12 of the B4 domain is F, L, R, D, M, Q or V and/or residue 10 of the B4 domain is F, L, Y, I, K or M, as described in the preceding section, and the B5 domain is at least 11, 12, 13, or 14 amino acids in length and residue 11 of the B5 domain is F, Y, or L. In contrast, residue 11 of the B5 domain in LuxSit-i is W. Residue 11 of the B5 may also be referred to by the position of the amino acid this residue corresponds to in the B5 domain in SEQ ID NO:1, where the residue 11 in B5 domain is position 100 in SEQ ID NO:1.

In certain aspects, the residue 11 of the B5 domain is F, Y, or L; residue 10 of the B4 domain is F, L, Y, I, K or M; and residue 12 of the B4 domain is F, L, R, D, M, Q or V. In certain aspects, residue 11 of the B5 domain is F or Y; residue 10 of the B4 domain is F or L, and residue 12 of the B4 domain is D, F or L; and/or residue 1 of the B3 domain is W/L/H.

In certain aspects:
(i) residue 11 of the B5 domain is F, Y, or L;
(ii) residue 10 of the B4 domain is F, L, Y, I, K or M;
(iii) residue 12 of the B4 domain is F, L, R, D, M, Q or V;
(vi) residue 9 of the H1 domain is D, K, L, N, R, S, T, Q, V, or Y;
(v) residue 2 of the L2 domain is not H and further optionally wherein the residue 2 of the L2 domain is D, F, K, L, N, R, S, T, Q, V, or Y;
(vi) residue 3 of the B1 domain is not H and further optionally wherein the residue 3 of the B1 domain is D, F, K, L, N, R, S, T, Q, V, or Y;
(vii) residue 2 of the B2 domain is D, F, K, L, N, R, S, T, Q, or Y;
(viii) residue 1 of the H3 domain is D, F, K, L, N, S, T, Q, V, or Y
(ix) residue 5 of the B4 domain is not H and further optionally wherein the residue 5 of the B4 domain is D, F, K, L, N, R, S, T, Q, V, or Y;
(x) residue 9 of the B4 domain is not H and further optionally wherein the residue 9 of the B4 domain is D, F, K, L, N, R, S, T, Q, V, or Y;
(xi) residue 8 of the B5 domain is D, F, K, L, N, R, S, Q, V, or Y;
(xii) residue 9 of the B5 domain is not H and further optionally wherein the residue 9 of the B5 domain is D, F, K, L, N, R, S, T, Q, V, or Y;
(xiii) residue 10 of the B5 domain is not H and further optionally wherein the residue 10 of the B5 domain is D, F, K, L, N, R, S, T, Q, V, or Y;
(xiv) residue 12 of the B5 domain is not H and further optionally wherein the residue 12 of the B5 domain is D, F, K, L, N, R, S, T, Q, V, or Y;
(xv) residue 14 of the B5 domain is D, F, K, L, N, S, T, Q, V, or Y;
(xvi) residue 3 of the B6 domain is D, F, K, L, N, R, S, Q, V, or Y;
(xvii) residue 4 of the B6 domain is D, F, K, L, N, R, S, T, Q, V, or Y;
(xviii) residue 8 of the B6 domain is not H and further optionally wherein the residue 8 of the B6 domain is D, F, K, L, N, R, S, T, Q, V, or Y; and/or
(xix) residue 9 of the B6 domain is D, F, K, L, N, R, S, T, Q, V, or Y.

In certain aspects:
(i) residue 11 of the B5 domain is F;
(ii) residue 10 of the B4 domain is F, L, Y, I, K or M;
(iii) residue 12 of the B4 domain is R;
(vi) residue 9 of the H1 domain is N;
(v) residue 2 of the L2 domain is not H and further optionally wherein the residue 2 of the L2 domain is D;
(vi) residue 3 of the B1 domain is not H and further optionally wherein the residue 3 of the B1 domain is T;
(vii) residue 2 of the B2 domain is T;
(viii) residue 1 of the H3 domain is V;
(ix) residue 5 of the B4 domain is not H and further optionally wherein the residue 5 of the B4 domain is T;
(x) residue 9 of the B4 domain is not H and further optionally wherein the residue 9 of the B4 domain is S;
(xi) residue 8 of the B5 domain is L;
(xii) residue 9 of the B5 domain is not H and further optionally wherein the residue 9 of the B5 domain is Q;
(xiii) residue 10 of the B5 domain is not H and further optionally wherein the residue 10 of the B5 domain is L;
(xiv) residue 12 of the B5 domain is not H and further optionally wherein the residue 12 of the B5 domain is K;
(xv) residue 14 of the B5 domain is V;
(xvi) residue 3 of the B6 domain is V;
(xvii) residue 4 of the B6 domain is D;
(xviii) residue 8 of the B6 domain is not H and further optionally wherein the residue 8 of the B6 domain is Y; and/or
(xix) residue 9 of the B6 domain is T.

In certain aspects:
(i) the H1 domain is at least 18 or 19 amino acids in length; residue 9 of the H1 domain is N, T, S, H, R, C, L, D, V, A, Q, G, E, K, I, N or M, residue 9 of the H1 domain corresponds to position 9 of SEQ ID NO:1, in contrast, residue 9 in SEQ ID NO:1 is F;
(ii) residue 1 of the B3 domain is L, W, or H, residue 1 of the B3 domain corresponds to position 64 of SEQ ID NO:1, in contrast, the amino acid at position 64 in SEQ ID NO:1 is Q; (iii) residue 10 of the B4 domain is F, Y, L, I, K or M, which corresponds to positon 85 of SEQ ID NO:1, in contrast, the amino acid at position 85 in SEQ ID NO:1 is A;
(iv) residue 12 of the B4 domain is F, L, R, D, M, Q or V;
(v) residue 10 of the B5 domain is L;
(vi) residue 3 of B6 domain is D or N;
(vii) residue 8 of B6 domain is Y, F, or L; and/or
(viii) residue 11 of the B5 domain is F or Y.

In certain aspects, in addition to the amino acids specified in (i)-(viii) above: (i) residue 9 of the Hi domain is N, T, S, H, R, C, L, D, V, A, Q, G, E, K, I, N or M; (ii) residue 1 of the B3 domain is L, W, or H;
(iii) residue 10 of the B4 domain is F, Y, L, I, K or M;
(iv) residue 12 of the B4 domain is F, D, Y, L, I, K or M;
(v) residue 10 of the B5 domain is L;
(vi) residue 3 of B6 domain is D or N;
(vii) residue 8 of B6 domain is Y, F, or L; and
(viii) residue 11 of the B5 domain is F or Y.

In certain aspects,
(i) residue 9 of the Hi domain is N, T, S, H, R, C, L, D, V, A, Q, G, E, K, I, N or M;
(ii) residue 1 of the B3 domain is L, W, or H;
(iii) residue 10 of the B4 domain is F, Y, L, I, K or M;
(iv) residue 12 of the B4 domain is F, L, R, D, M, Q or V;
(v) residue 10 of the B5 domain is L;
(vi) residue 3 of B6 domain is D or N;
(vii) residue 8 of B6 domain is Y, F, or L;
(viii) residue 11 of the B5 domain is F or Y; and/or
(ix) residue 2 of the L2 domain is not H and further optionally wherein the residue 2 of the L2 domain is D, F, L, Q, R, S, T, W or Y. Additionally, in certain embodiments, the protein comprising the amino acids specified in (i) to (ix) above is further mutated to replace one or more Histidine with other amino acid, for example:
(x) residue 3 of the B1 domain is not H and further optionally wherein the residue 3 of the B1 domain is D, F, L, Q, R, S, T, W or Y;
(xi) residue 5 of the B4 domain is not H and further optionally wherein the residue 5 of the B4 domain is D, F, L, Q, R, S, T, W or Y;
(xii) residue 9 of the B4 domain is not H and further optionally wherein the residue 9 of the B4 domain is D, F, L, Q, R, S, T, W or Y;
(xiii) residue 3 of the B5 domain is not H and further optionally wherein the residue 3 of the B5 domain is D, F, L, Q, R, S, T, W or Y;
(xiv) residue 9 of the B5 domain is not H and further optionally wherein the residue 9 of the B5 domain is D, F, L, Q, R, S, T, W or Y;
(xiv) residue 10 of the B5 domain is not H and further optionally wherein the residue 10 of the B5 domain is D, F, L, Q, R, S, T, W or Y;
(xvi) residue 12 of the B5 domain is not H and further optionally wherein the residue 12 of the B5 domain is D, F, L, Q, R, S, T, W or Y; and/or
(xvii) residue 8 of the B6 domain is not H and further optionally wherein the 8 residue of the B6 domain is D, F, L, Q, R, S, T, W or Y.

In certain aspects,
(i) residue 9 of the H1 domain is N;
(ii) residue 1 of the B3 domain is L;
(iii) residue 10 of the B4 domain is F;
(iv) residue 12 of the B4 domain is R;
(v) residue 10 of the B5 domain is L;
(vi) residue 3 of B6 domain is D;
(vii) residue 8 of B6 domain is Y;
(viii) residue 11 of the B5 domain is F;
(ix) residue 2 of the L2 domain is not H and further optionally wherein the residue 2 of the L2 domain is D;
(x) residue 3 of the B1 domain is not H and further optionally wherein the residue 3 of the B1 domain is T;
(xi) residue 5 of the B4 domain is not H and further optionally wherein the residue 5 of the B4 domain is T;
(xii) residue 9 of the B4 domain is not H and further optionally wherein the residue 9 of the B4 domain is S;
(xiii) residue 3 of the B5 domain is not H and further optionally wherein the residue 3 of the B5 domain is S;
(xiv) residue 9 of the B5 domain is not H and further optionally wherein the residue 9 of the B5 domain is Q;
(xiv) residue 10 of the B5 domain is not H and further optionally wherein the residue 10 of the B5 domain is L;
(xvi) residue 12 of the B5 domain is not H and further optionally wherein the residue 12 of the B5 domain is R; and/or
(xvii) residue 8 of the B6 domain is not H and further optionally wherein the 8 residue of the B6 domain is Y.

In certain aspects,
(i) residue 9 of the HI domain is N, T, S, H, R, C, L, D, V, A, Q, G, E, K, I, N or M; (ii) residue 1 of the B3 domain is L, W, or H;
(iii) residue 10 of the B4 domain is F, Y, L, I, K or M;
(iv) residue 12 of the B4 domain is F, L, R, D, M, Q or V;
(v) residue 10 of the B5 domain is L;
(vi) residue 8 of B6 domain is Y, F, or L; and/or
(vii) residue 11 of the B5 domain is F or Y;
(viii) residue 2 of the H2 domain is A, F, I, K, L, N, R, S, T, Q, V, or Y; (ix) residue 2 of the L2 domain is not H and further optionally wherein the 2 residue of the L2 domain is A, D, F, I, K, L, N, R, S, T, Q, V, or Y;
(x) residue 3 of the B1 domain is not H and further optionally wherein the 3 residue of the B1 domain is A, D, F, I, K, L, N, R, S, T, Q, V, or Y;
(xi) residue 2 of the B2 domain is A, D, F, I, K, L, N, R, S, T, Q, or Y;
(xii) residue 1 of the H3 domain is A, D, F, I, K, L, N, S, T, Q, V, or Y;
(xiii) residue 10 of the H3 domain is A, D, F, I, K, L, N, S, T, Q, V, or Y; (xiv) residue 11 of the H3 domain is A, D, F, I, K, N, R, S, T, Q, V, or Y; (xv) residue 5 of the B3 domain is A, D, F, I, L, N, R, S, T, Q, V, or Y;
(xvi) residue 5 of the B4 domain is is not H and further optionally wherein the residue 5 of B4 is A, D, F, I, K, L, N, R, S, T, Q, V, or Y;
(xvii) residue 7 of the B4 domain is A, D, F, I, L, N, R, S, T, V, or Y;
(xviii) residue 9 of the B4 domain is not H and further optionally wherein the residue 9 of B4 is A, D, F, I, L, K, N, R, S, T, Q, V, or Y;
(xix) residue 8 of the B5 domain is A, D, F, I, L, K, N, R, S, Q, or Y;
(xx) residue 9 of the B5 domain is not H and further optionally wherein the residue 9 of B5 is A, D, F, I, L, K, N, R, S, Q, V, or Y;
(xxi) residue 12 of the B5 domain is not H and further optionally wherein the residue 12 of B5 is A, D, F, I, L, K, N, R, S, Q, V, or Y;
(xxii) residue 14 of the B5 domain is A, D, F, I, L, K, N, S, Q, V, or Y;
(xxiii) residue 3 of the B6 domain is A, D, F, I, L, K, N, R, S, Q, V, or Y; (xxiv) residue 4 of the B6 domain is A, D, F, I, L, K, N, R, S, Q, V, or Y; and/or (xxv) residue 9 of the B6 domain is A, D, F, L, K, N, R, S, Q, V, or Y.

In certain aspects,
(i) residue 9 of the H1 domain is N;
(ii) residue 1 of the B3 domain is L;
(iii) residue 10 of the B4 domain is F;
(iv) residue 12 of the B4 domain is R;
(v) residue 10 of the B5 domain is L;
(vi) residue 8 of B6 domain is Y;
(vii) residue 11 of the B5 domain is F;
(viii) residue 2 of the H2 domain is I;
(ix) residue 2 of the L2 domain is not H and further optionally wherein the 2 residue of the L2 domain is D;
(x) residue 3 of the B1 domain is not H and further optionally wherein the 3 residue of the B1 domain is T;
(xi) residue 2 of the B2 domain is T;
(xii) residue 1 of the H3 domain is V;
(xiii) residue 10 of the H3 domain is S;
(xiv) residue 11 of the H3 domain is Q;
(xv) residue 5 of the B3 domain is S;
(xvi) residue 5 of the B4 domain is is not H and further optionally wherein the residue 5 of B4 is T;
(xvii) residue 7 of the B4 domain is R;
(xviii) residue 9 of the B4 domain is not H and further optionally wherein the residue 9 of B4 is S; (xix) residue 8 of the B5 domain is L;
(xx) residue 9 of the B5 domain is not H and further optionally wherein the residue 9 of B5 is Q; (xxi) residue 12 of the B5 domain is not H and further optionally wherein the residue 12 of B5 is K;
(xxii) residue 14 of the B5 domain is V;
(xxiii) residue 3 of the B6 domain is V;
(xxiv) residue 4 of the B6 domain is A; and/or
(xxv) residue 9 of the B6 domain is V.

In other aspects, the polypeptide may have an amino acid sequence where:
(i) residue 7 of the H2 domain is S;
(ii) residue 4 of L2 domain is H;
(iii) residue 10 of B3 domain is R;
(iv) residue 1 of the B3 domain is L, W, or H;
(v) residue 7 of B4 domain is K;
(vi) residue 10 of the B4 domain is F, Y, L, I, K or M;
(vii) residue 12 of the B4 domain is F, D, Y, L, I, K or M;
(viii) residue 3 of B6 domain is D or N;
(ix) residue 8 of B6 domain is Y, F, or L; and
(x) residue 11 of the B5 domain is W, Y or F.

The amino acid(s) that may be present at a particular position in a domain of the polypeptide of the present disclosure and having luciferase activity; its position relative to SEQ ID NO:1; and the amino acid at that positon in SEQ ID NO:1 are listed below:

| | Position | Amino |
|---|---|---|
| | Relative to | acid in |
| | SEQ ID | SEQ ID |
| Amino acid in Polypeptide Domain | NO: 1 | NO: 1 |
| residue 9 of the H1 domain is N, T, S, H, R, C, L, | 9 | F |
| D, V, A, Q, G, E, K, I, N or M | | |
| residue 7 of the H2 domain is S | 28 | L |
| residue 2 of L2 domain is D | 30 | H |
| residue 4 of L2 domain is H | 32 | G |
| residue 3 of B1 domain is T | 36 | H |
| residue 2 of B2 domain is T | 41 | V |
| residue 1 of H3 domain is V | 46 | R |
| residue 10 of H3 domain is S | 55 | R |
| residue 10 of B3 domain is R, Q | 56 | L |
| residue 1 of the B3 domain is L, W, or H | 64 | Q |
| residue 5 of the B3 domain is S | 68 | K |
| residue 5 of the B4 domain is T | 80 | H |
| residue 7 of B4 domain is K, R | 82 | Q |
| residue 9 of the B4 domain is S | 84 | H |
| residue 10 of the B4 domain is F, Y, L, I, K or M | 85 | A |
| residue 12 of the B4 domain is F, L, R, D, M, Q | 87 | H |
| or V | | |
| residue 8 of the B5 domain is L | 97 | T |
| residue 9 of the B5 domain is Q | 98 | H |
| residue 10 of the B5 domain is L | 99 | H |
| residue 11 of the B5 domain is F or Y | 100 | W |
| residue 12 of the B5 domain is K | 101 | H |
| residue 14 of the B5 domain is V | 103 | R |
| residue 3 of B6 domain is V, D or N | 108 | T |
| residue 4 of B6 domain is A | 109 | E |
| residue 8 of B6 domain is Y, F, or L. | 113 | H |
| residue 9 of B6 domain is V | 114 | I |

In certain aspects, a protein having luciferase activity may have an amino acid sequence at least 30%, 35%, 40%, 45%, 50%, 55%, 60%, 65%, 70%, 75%, 80%, 85%, 90%, 91%, 92%, 93%, 94%, or 95% identical to the amino acid sequence of SEQ ID NO:1, where residue 87 is F, L, R, D, M, Q or V; residue 85 is F, Y, L, I, K or M, and residue 100 is F, Y, or L, when numbered relative to SEQ ID NO:1.

### LuxSit-i Variant With Substitutions in H1 Domain

In certain aspects, a protein having luciferase activity may have the secondary structure arrangement H1-L1-H2-L2-B1-L3-B2-L4-H3-L5-B3-L6-B4-L7-B5-L8-B6 as described herein, wherein the H1 domain is at least 18 or 19 amino acids in length; residue 9 of the H1 domain is T, S, H, R, C, L, V, A, Q, G, E, K, I, N or M. In contrast, residue 9 of the H1 domain in LuxSit-i is F. Residue 9 of the H1 may also be referred to by the position of the amino acid this residue corresponds to in the H1 domain in SEQ ID NO:1, where the residue 9 in H1 domain is position 9 in SEQ ID NO:1. In certain aspects, a protein having luciferase activity may have an amino acid sequence at least 30%, 35%, 40%, 45%, 50%, 55%, 60%, 65%, 70%, 75%, 80%, 85%, 90%, 91%, 92%, 93%, 94%, or 95% identical to the amino acid sequence of SEQ ID NO:1, where residue 9 is T, S, H, R, C, L, V, A, Q, G, E, K, I, N or M, when numbered relative to SEQ ID NO:1.

In certain aspects, a protein having luciferase activity comprises an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:1, wherein the amino acid at position 9 is any amino acid other than F, wherein the position 9 is numbered based on SEQ ID NO:1. In some embodiments, the amino acid at position 9 is D, E, Q, R, S, T, H, I, L, V, A, G, C, N, K, or M.

In certain aspects, the protein may further include a B4 domain that is at least 12 amino acids in length and residue 10 of the B4 domain is F, Y, L, I, K or M; and/or residue 12 of the B4 domain is L, R, D, M, Q, or V.

In certain aspects, residue 9 of the H1 domain is V, residue 10 of the B4 domain is F, Y, L, I, K or M and residue 12 of the B4 domain is L, R, D, M, Q or V. In certain aspects, residue 9 of the Hi domain is V, residue 10 of the B4 domain is F, and residue 12 of the B4 domain is L. the B3 domain is at least 6, 7, 8, 9, or 10 amino acids in length and residue 1 of the B3 domain is W or H. residue 1 of the B3 domain is W.

In certain aspects, the polypeptide may include one or more of the amino acids in the domains listed below. Its position relative to SEQ ID NO:1 and the amino acid at that positon in SEQ ID NO:1 are also listed.

| Amino acid in Polypeptide Domain | Position | Amino |
|---|---|---|
| residue 9 of the H1 domain is N, T, S, H, R, C, L, D, | 9 | F |
| V, A, Q, G, E, K, I, N or M | | |
| residue 10 of the B4 domain is F, Y, L, I, K or M | 85 | A |
| residue 12 of the B4 domain is F, L, R, D, M, Q or V | 87 | H |
| residue 1 of the B3 domain is L, W, or H | 64 | Q |
| residue 10 of the B5 domain is L | 99 | H |
| residue 19 of the H1 domain is R | 19 | S |
| residue 4 of L2 domain is H | 32 | G |
| residue 3 of B6 domain is D or N | 108 | T |
| residue 8 of B6 domain is Y, F, or L | 113 | H |
| residue 11 of the B5 domain is F or Y. | 100 | W |
| residue 7 of the H2 domain is S or F | 28 | L |
| residue 2 of L5 is Q | 61 | K |
| residue 10 of B3 domain is R | 56 | L |
| residue 7 of B4 domain is K | 82 | Q |

In certain embodiments, the protein may further comprise a substitution at position H30, H36, H80, H84, H87, H92, H98, H99, or H101 with another amino acid, e.g., R, Q, T, S, L, Y, etc. In some embodiments, the protein further comprises one or more of the substitutions H30D, H36T, H80T, H84S, H87R, H92S, H98Q, H99L, and H101R. In other embodiments, the protein further comprises a substitution at one or more of position V41, R46, T97, R103, E109, and I114. In still other embodiments, the protein further comprises one or more of the substitutions V41T, R46V, T97L, R103V, E109D, and I114T LuxSit-i Variant With Substitutions in B3 Domain

In certain aspects, a protein having luciferase activity may have the secondary structure arrangement H1-L1-H2-L2-B1-L3-B2-L4-H3-L5-B3-L6-B4-L7-B5-L8-B6 as described herein, the B3 domain is at least 6, 7, 8, 9, or 10 amino acids in length and residue 1 of the B3 domain is W or H. In contrast, residue 1 of the B3 domain in LuxSit-i is Q. Residue residue 1 of the B3 domain may also be referred to by the position of the amino acid this residue corresponds to in the B3 domain in SEQ ID NO:1, where the residue 1 of the B3 domain is position 64 in SEQ ID NO:1.

### Proteins Having Amino Acid Sequence Identity to LuxSit-i Variants

In certain aspects, a protein having luciferase activity may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:1, where residue 64 is W or H, when numbered relative to SEQ ID NO:1.

In certain embodiments, a protein of the present disclosure having luciferase activity may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of a LuxSit-i variant disclosed herein, e.g., a LuxSit-i variant listed in Table 5.

In certain embodiments, a protein of the present disclosure having luciferase activity may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence set forth in any one of SEQ ID NOs: 2-91, 93-143, 2601-2604, 2663, 2665, 2682-2732, and 2753-2769.

In certain embodiments, a protein of the present disclosure may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence set forth in any one of SEQ ID NOs:2668-2671, wherein the protein does not have significant luciferase activity. This protein may have luciferase activity when associated with a complementing polypeptide, e.g., a fragment having an amino acid sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identity to the amino acid sequence of any one of SEQ ID NOs: 2605, 2607, 2615-2617, 2619, 2621, 2623, 2625, 2627, 2629, 2631, 2633, 2635, 2637, 2638, 2640-2655, 2672-2673, 2676, 2678, and 2734.

In certain embodiments, a protein of the present disclosure may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of any LuxSit-i variant or a fragment thereof disclosed herein. In certain embodiments, a protein of the present disclosure may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of any LuxSit-i variant or a fragment thereof disclosed herein and may comprise one or more substitutions relative to the amino acid sequence set forth in SEQ ID NO:1, where the one or more substitutions are conservative amino acid substitutions.

In certain embodiments, a protein of the present disclosure may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of any LuxSit-i variant or a fragment thereof disclosed herein and lacks lysine residues, where the lysine residues present in SEQ ID NO:1 are replaced with another amino acid such as R, Q, T, S, L, Y, etc. In certain embodiments, a protein of the present disclosure may have an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:1 or a fragment thereof disclosed herein, lacks lysine residues and contains an arginine or histidine in place of lysine relative to SEQ ID NO:1.

In certain embodiments, a protein of the present disclosure comprises the substitution F9V/S/N, and optionally comprises one or more of the substitutions Q64W, A85F, and H87L.

In certain embodiments, a protein of the present disclosure comprises the substitution F9N, and optionally comprises one or more of the substitutions Q64L, A85F, H87R, H99L, W100F, T108D, and H113Y.

In certain embodiments, a protein of the present disclosure comprises the substitution F9N, and optionally comprises one or more of the substitutions H30D, H36T, Q64L, H80T, H84S, A85F, H87R, H92S, H98Q, H99L, W100F, H101R, T108D, and H113Y.

In certain embodiments, a protein of the present disclosure comprises the substitution F9N, and optionally comprises one or more of the substitutions H30D, H36T, L56Q, Q64L, H80T, H84S, A85F, H87R, H92S, H98Q, H99L, W100F, H101K/R, T108D, and H113Y.

In certain embodiments, a protein of the present disclosure comprises the substitution F9N, and optionally comprises one or more of the substitutions D231, H30D, H36T, V41T, R46V, R55S, L56Q, Q64L, K68S, H80T, Q82R, H84S, A85F, H87R, H92S, T97L, H98Q, H99L, W100F, H101K/R, R103V, T108D/V, E109A, H113Y, and I114V, further optionally, wherein the amino acid sequence does not include lysine.

Sequences of LuxSit-i variants are set forth in SEQ ID NOs: 2-91, 93-143, 2601-2604, 2663, or 2665 in Table 5.

TABLE 5

| | SEQ |
|---|---|
| | ID |
| LuxSit-i Variant | NO |
| MSEEQIRQFLRRFYEALDSGDADTAASLFHPGVTIHLWDGVTFTSREEFREWFERLFSTSKDAWREIKSLEVRGDTVEVHVQLHFTLNGQKHTVDLTHHFHFRGNRVTEVRVHINPTG | 2 |
| MSEEQIRQFLRRFYEALDSGDADTAASLFHPGVTIHLWDGVTFTSREEFREWFERLFSTSKDAQREIKSLEVRGDTVEVHVQLHFTLNGQKHTVDLTHHFHFRGNRVTEVRVHINPTG | 3 |
| MSEEQIRQVLRRFYEALDSGDADTAASLFHPGVTIHLWDGVTFTSREEFREWFERLFSTSKDAWREIKSLEVRGDTVEVHVQLHFTLNGQKHTVDLTHHWHFRGNRVTEVRVHINPTG | 4 |
| MSEEQIRQVLRRFYEALDSGDADTAASLFHPGVTIHLWDGVTFTSREEFREWFERLFSTSKDAQREIKSLEVRGDTVEVHVQLHFTLNGQKHTVDLTHHWHFRGNRVTEVRVHINPTG | 5 |
| MSEEQIRQSLRRFYEALDSGDADTAASLFHPGVTIHLWDGVTFTSREEFREWFERLFSTSKDAWREIKSLEVRGDTVEVHVQLHFTLNGQKHTVDLTHHWHFRGNRVTEVRVHINPTG | 6 |
| MSEEQIRQSLRRFYEALDSGDADTAASLFHPGVTIHLWDGVTFTSREEFREWFERLFSTSKDAQREIKSLEVRGDTVEVHVQLHFTLNGQKHTVDLTHHWHFRGNRVTEVRVHINPTG | 7 |
| TSEEQIRQFLRRFYEALDSGDADTAASLFHPGVTIHLWDGVTFTSREEFREWFERLFSTSKDAQREIKSLEVRGDTVEVHVQLHATHNGQKYTVDLTHHWHFRGNRVTEVRVHINPTG | 8 |
| MSEEQIRQLLRRFYEALDSGDADTAASLFHPGVTIHLWDGVTFTSREEFREWFERLFSTSKDAQREIKSLEVRDDTVEVHVQLHATHNGQKHTVDLTHHWHFRGNRVTEVRVHINPTG | 9 |
| MSEEQIRQSLRRFYEALDSGDADTAASLFHPGVTIHLWDGVTFTSREEFREWFERLFTTSKDAQREIKSLEVRGDTVEVHVQLHATHNGQKHTVDLTHHWHFRGNRVTEVRVHINPTG | 10 |
| MSEEQIRQFLRRFYEALDSGDADTAASLFHPGVTIHLWDGVTFTSRAEFREWFERLFSTSKDAQREIKSLEVRGDTVEVHVQLHATHNGQKHTVDLTHHWHFRGNRVTEVCVHINPTG | 11 |
| MSEEQIRQFLRRFYEALDSGDADTAASLFHPGVTIHLWDGVTFTSREEFIEWFERLFSTSKDAHREIKSLEVRGDTVEVHVQLHATQNGQKHTVDLTHHWHFRGNRVTEVRVHINPTG | 12 |
| MSEEQIRQSLRRFYEALDSGDADTAASLFHPGVTVHLWDGVTFTSREEFREWFERLFSTSKDAQREIKSLEVRGDTVEVHVQLHATHNGQKHTVDLTHHWHFRGNRVTEVRVHINPTG | 13 |
| MSEEQIRQFLRRFYEALDSGDADTAASLFHPGVTIHLWDGVTFTSREEFREWFERQFSTSKDAQREIKSLEVRGDTVEVHVQLHATHNGQKHTVDLTHHWHFRGNRVTEVRVQINPTG | 14 |
| MSEEQIRQFLRRFYEALDSGDADTAASLFHPGVTIHLWDGVTLTSREEFREWFERLFSTSKDVQREIKSLEVRGDTVEVHVQLHATHNGQKHTVDLTHHWHFRGNRVTEVRVHINPTG | 15 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWQFRGNRATEVRVHISPTG | 16 |
| MSEEQIRQFLRRFYEALDSGDADTAASLFHPGVTIHLWDGVTLTSREEFREWFERLFSTSKDAQREIKSLEVRGDTVEVHVQLHATHNGQKHTVDLTHHWHFRGNRVTEVRVHINPTG | 17 |
| MSEEQIRQFLRRFYEALDSGDADTAASLFHPGVTIHLWDGVTFTSREEFREWFERQFSTSKDAQREIKSLEVRGDTVEVHVQLHATHNGQKHTVDLTHHWHFRGNRVTEVRVHINPTG | 18 |
| MSEEQIRQLLRRFYEALDSGDADTAASLFHPGVTIHLWDGVTFTSREEFREWFERLFSTSKDAQREIKSLEVRGDTVEVHVQLHATHNGQKHTVDLTHHWYFRGNRVTEVRVHINPTG | 19 |
| MSEEQIRQFLRRFYEALDSGDADTAASLFHPGVTIHLWDGVTFTSREEFREWFERLFSTSKDAQREIKSLEVRGDTVEVHVQLHATHNGQKYTVDLTHHWHFRGNRVTEVRVHINPTG | 20 |
| MSEEQIRQFLRRFYEALDSGDADTAASLFHPGVTIHLWDGVTFTSREEFKEWFERLFSTSKDAQREIKSLEVRGDTVEVHVQLHATHNGQKHTVDLTHHWHFRGNRVTEVRVHINPTD | 21 |
| MSEEQIRQILRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 22 |
| MSEEQIRQFLRRFYEALDSGDADTAASLFHPGVTFHLWDGVTFTSREESREWFERLFSTSKDAQREIKSLEVRGDTVEVHVQLHATHNGQKHTVDLTHHWHFRGNRVTEVRVHINPTG | 23 |
| MSEEQIRQFLRRFYEALDRGDADTAASLFHPRVTIHLWDGVTFTSREEFREWFERLFSTSKDAQREIKSLEVRGDTVEVHVQLHATHNGQKHTVDLTHHWHFRGNRVTEVRVHINPTG | 24 |
| MSEEQIRQFLRRFYEALDSGDADTAASLFHPDVTIHLWDGVTFTSREEFREWFERLFSTSKDAQREIKSLEVRGDTVEVHVQLHATHNGQKHTVDLTHHWHFRGNRVTEVRVHINPTG | 25 |
| MSEEKIRQFLCRFYEALDSGDADTAASLFHPGATIHLWDGVTFTSREEFREWFERLFSTSKDAQREIKSLEVRGDTVEVHVQLHATHNGQKHTVDLTHHWHFRGNRVTEVRVHINPTG | 26 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRITEVRVHINPTG | 27 |
| MSEEQIRQFLRRFYEALDSGDADTAASLFHPGVTIHQWDGVTFTSREEFREWFERLFSTSKDAQREIKSLEVRGDTVEVHVQLHATHNGQKHTVDLTHHWHFRGSRVTEVRVHINPTG | 28 |
| MSEELIRQFLRRFYEALDSGDADTAASLFHPDVTIHLWDGVTFTSREEFREWFERLFSTSKDAHREIKSLEVRGDTVEVHVQLHATHNGQKHTVDLTHHWHFRGNRVTEVRVHINPTG | 29 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNLVTEVRVHINPTG | 30 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHLWHFRGNRVTEVRVHINPTG | 31 |
| MSEEQIRQFLRRFYEALDSGDADTAASLFHPGVTIHLWDGVTFTSREEFREWFERLFSTSEDAQREIKSLEVRGDTVEVHVQLHATHNGQKHTVDLTHHWHFRGNRVTEVRVHINPTG | 32 |
| MSEEQIRQFLRRFYEALDSGDADTAASLFHPGVTIHLWDGVTFTSREEFREWFERLFSTSKDAHREIKSLEVRGDTVEVHVQLHATHNGQKHTVDLTHHWHFRGNRVTEVRVHINPTG | 33 |
| MSEEQIRQFLRRFYEALDSGDADTAASIFHPGVTIHLWDGVTFTSREEFKEWFERLFSTSKDAQREIKSLEVRGDTVEVHVQLHATHNGQKHTVDLTHHWHFRGNRVTEVRVHINPTG | 34 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVNEVRVHINPTG | 35 |
| MSEEQIRQFLRRFYEALDSGDADTAASLFHPGVTIHLWDGVTFTSREEFREWFERLFSTSKDAQREIKSLEVRGDTVETHVQLHATHNGQKHTVDLTHHWHFRGNRVTEVRVHINSTG | 36 |
| MSEEQIRQFLRRFYEALDSGDADTAASLFHPGVTIHLWDGVTFTSREEFREWFERLFSTSKDAQREIKSLEVRGDTVWVHVQLHATHNGQKHTVDLTHHWHFRGNRVTEVRVHINPTG | 37 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDRTHHWHFRGNRVTEVRVHINPTG | 38 |
| MSEEQIRQHLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 39 |
| MSEEQKRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 40 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHLHFRGNRVTEVRVHINPTG | 41 |
| MSEEQIRQFLRRFYEALDSGDADTAASLFHPGVTIHLWDGVTFTAREEFREWFERLFSTSKDAQREIKSLEVRGDTVEVHVQLHATHNGQKHTVDLTHHWHFRGNRVTEVRVHINPTG | 42 |
| MSEEQTRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 43 |
| MSEEQIRQTLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 44 |
| MSEEQIRQFLRRFYEALDSGDADTAASLFHPGVTIHLWDGVTFTSREEFREWFERLFSTSPDAQREIKSLEVRGDTVEVHVQLHATHNGQKHTVDLTHHWHFRGNRVTEVRVHINPTG | 45 |
| MSEEQIRQFLRRFYEALDSGDADTAASLFHPGVTIHLWDGVTFTSREEFREWFERLVSTSKDAQREIKSLEVRGDTVEVHVQLHATHNGQKHTVDLTHHWHFRGNRVTEVRVHINPTG | 46 |
| MSEEQIRQFLRRFYEALDSGDADTAASLFHPGVTIHLWDGVTFTSREEFREWFERLFSTSKDAWREIKSLEVRGDTVEVHVQLHATHNGQKHTVDLTHHWHFRGNRVTEVRVHINPTG | 47 |
| MSEEQIRQFLRRFYEALDSGDADTAASFFHPGVTIHLWDGVTFTSREEFREWFERLFSTSKDAQREIKSLEVRGDTVEVHVQLHATHNGQKHTVDLTHHWHFRGNRVTEVRVHINPTG | 48 |
| MSEEQIRQFLRRFYEALDSGDADTAASLFHPGVTIHLWDGVTGTSREEFREWFERLFSTSKDAQREIKSLEVRGDTVEVHVQLHATHNGQKHTVDLTHHWHFRGNRVTEVRVHINPTG | 49 |
| MSEEQIRQVLRRFYEALDSGDADTAASLFHPGVTIHLWDGVTFTSREEFREWFERLFSESKDAQREIKSLEVRGDTVEVHVQLHATHNGQKHTVDLTHHWHFRGNRVTEVRVHINPTG | 50 |
| MSEEQIRQFLRRFYEALDSGDADTAASLFHPHVTIHLWDGVTFTSREEFREWFERLFSTSKDAQREIKSLEVRGDTVEVHVQLHATHNGQKHTVDLTHHWHFRGNRVTEVRVHINPTG | 51 |
| MSEEQIRQFLRRFYEALDSGDADTAASLFHPRVTIHLWDGVTFTSREEFREWFERLFSTSKDAQREIKSLEVRGDTVEVHVQLHATHNGQKHTVDLTHHWHFRGNRVTEVRVHINPTG | 52 |
| MSEEQIRQFLRRFYEALDSGDADTAASLFHPGVTIHLWDGVTFTSREEFREWFERLFSTSKDAQREIKSLEVRGDTVEVHVQLHAAHNGQKHTVDLTHHWHFRGNRVTEVRVHINPTG | 53 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHYHFRGNRVTEVRVHINPTG | 54 |
| MSEEQIRQFLRRFYEALDSGDADTAASLFHPGVTIHLWDGVIFTSREEFREWFERLFSTSKDAQREIKSLEVRGDTVEVHVQLHATHNGQKHTVDLTHHWHFRGNRVTEVRVHINPTG | 55 |
| MSEEQIRQFLRRFYEALDSGDAVTAASLFHPGVTIHLWDGVTFTSREEFREWFERLFSTSKDAQREIMSLEVRGDTVEVHVQLHATHNGQKHTVDLTHHWHFRGNRVTEVRVHINPTG | 56 |
| MSEEQIRQFLRRFYEALDSGDADTAASLFHPGVTIHLWDGVTFTSREEFREWFERLFSQSKDAQREIKSLEVRGDTVEVHVQLHATMNGQKHTVDLTHHWHFRGNRVTEVRVHINPTG | 57 |
| MSEEQIRQFLRRFYEALDSGDADTAASSFHPGVTIHLWDGVTFTSREEFREWFERLFSTSKDAQREIKSLEVRGDTVEVHVQLHATHNGQKHTVDLTHHWHFRGNRVTEVRVHINPTG | 58 |
| MSEEQIRQFLRRFYEALDSGDADTAASLFHPGVTIHLWDGVTFTSREEFREWFERLFSTSKDAQREIKSLEVRGDTVEVHVQLHITHNGQKHTVDLTHHWHFRGNRVTEVRVHINPTG | 59 |
| MSEEQIRQFLRRFYEALDSGDADTAASLFHPGVTIHLWDGVTFTSREEFREWFERLFSTSKDAQREIKSLEVRGDTVEVHVKLHATHNGQKHTVDLTHHWHFRGNRVTEVRVHINPTG | 60 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVDEVRVHINPTG | 61 |
| MSEEQIRQFLRRFYEALDSGDADTAASLFHPGVTIHLWDGVTFTSREEFREWFERLFSTSKDAQREIKSLEVRGDTYEVHVQLHATHNGQKHTVDLTHHWHFRGNRVTEVRVHINPTG | 62 |
| MSEEQIRQFLRRFWEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 63 |
| MSEEQIRQELRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 64 |
| MSEEQIRQFLRRFYEALDSGDADTAASLFHPGVTIHLWDGVTFTSREEFREWFERLFSKSKDAQREIKSLEVRGDTVEVHVQLHATHNGQKHTVDLTHHWHFRGNRVTEVRVHINPTG | 65 |
| MSDEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 66 |
| MSEEQIRQFLRRFYEALDSGDADTAASLFHPGVTIHLWDGVTFTSREEFREWFERLFSTSKDAQREIKSLEVRGDTVEVHVTLHATHNGQKHTVDLTHHWHFRGNRVTEVRVHINPTG | 67 |
| MSEEQIRQFLRRFYEALDSGDADTAASLFHPAVTIHLWDGVTFTSREEFREWFERLFSTSKDAQREIKSLEVRGDTVEVHVQLHATHNGQKHTVDLTHHWHFRGNRVTEVRVHINPTG | 68 |
| MSEEQIRQFLRRFYEALDSGDADTAASLFHPGVTIHLWDGVTFTSREEFREWFERRFSTSKDAQREIKSLEVRGDTVEVHVQLHATHNGQKHTVDLTHHWHFRGNRVTEVRVHINPTG | 69 |
| MSEEQIRQLLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 70 |
| MSEEQIRQFLRRFYEALDRGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 71 |
| MSEEQIRQALRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVWINPTG | 72 |
| MSEEQIRQSLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 73 |
| MSEEQIRQFLRRFYEALDSGDADTAASLFHPGVTIHLWDGVTFTSREEFREWFERLFSTSKDAQREIKSLEVRGDTVEVHVQLHMTHNGQKHTVDLTHHWHFRGNRVTEVRVHINPTG | 74 |
| MSEEQIRQNLRRFYEALDSGDADTAASLFHPGVTIHLWDGVTFTSREEFREWFERLFSTSKDAQREIKSLEVRGDTVEVHVQLHATHNGQKHTVDLTHHWRFRGNRVTEVRVHINPTG | 75 |
| MSEEQIRQFLRRFYEALDSGDADTAASLFHPEVTIHLWDGVTFTSREEFREWFERLFSTSKDAQREIKSLEVRGDTVEVHVQLHATHNGQKHTVDLTHHWHFRGNRVTEVRVHINPTG | 76 |
| MSEEQIRQFLRRFYEALDSGDADTAASLFHPMVTIHLWDGVTFTSREEFREWFERLFSTSKDAQREIKSLEVRGDTVEVHVTLHATHNGQKHTVDLTHHWHFRGNRVTEVRVHINPTG | 77 |
| MSEEQIRQDLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 78 |
| MSEEQIRQFLRRFYEALDSGDADTAASLFHPGVTIHLWDGVTFTSREEFREWFERLFSTSKDAQREIKSLEVRGDTVEVHVQLHATVNGQKHTVDLTHHWHFRGNRVTEVRVHINPTG | 79 |
| MSEEQIRQMLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 80 |
| MSEEQIRQQLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 81 |
| MSEEQIRQFLRRFYEALDSGDADTAASLFHPGVTIHLWDGVTFTSREEFREWFERLFSTSKDAQREIKSLEVRGDTVEVHVQLHFTHNGQKHTVDLTHHWHFRGNRVTEVRVHINPTG | 82 |
| MSEEQIRQFLRRFYEALDSGDADTAASLFHPGVTIHLWDGVTFTSREEFREWFERKFSTSKDAQREIKSLEVRGDTVEVHVQLHATHNGQKHTVDLTHHWHFRGNRVTEVRVHINPTG | 83 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVFINPTG | 84 |
| MSEEQIRQFLRRFYEALDSGDADTAASLFHPGVTIHLWDGVTFTSREEFREWFERLFSTSKDAQREIKSLEVRGDTVEVHVQLHLTHNGQKHTVDLTHHWHFRGNRVTEVRVHINPTG | 85 |
| MSEEQIRQVLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 86 |
| MSEEQIRQFLRRFYEALDSGDADTAASLFHPGVTIHLWDGVTFTSREEFREWFERLFSTSKDAQREIKSLEVRGDTVEVHVQLHYTHNGQKHTVDLTHHWHFRGNRVTEVRVHINPTG | 87 |
| MSEEQIRQFLRRFYEALDSGDADTAASLFHPGVTIHLWDGVTFTSREEFREWFERLFSTSKDAQREIKSLEVRGDTVEVHVQLHATLNGQKHTVDLTHHWHFRGNRVTEVRVHINPTG | 88 |
| MSEEQIRQFLRRFYGALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 89 |
| MSEEQIRQRLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 90 |
| MSEEQIRQCLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 91 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVXEVRVHINPTG | 93 |
| MSEEQIRQALRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 94 |
| MSEEQIRQFLRRFYEALDSGDADTAASLFHPGVTIHLWDGVTFTSREEFREWFERLFSTSKDAQREIKSLEVRGDTVEVHVQLHATHNGQKHTVDLTHHFHFRGNRVTEVRVHINPTG | 95 |
| MSEEQIRQGLRRFYEALDSGDADTAASLFHPGVTIHLWDGVTFTSREEFREWFERLFSTSKDAQREIKSLEVRGDTVEVHVQLHATHNGQKHTVDLTHHWHFRGNRVTEVRVHINPTG | 96 |
| MSEEQIRQSLRRFYEALDRGDADTAASLFHPDVTIHLWDGVTFTSREEFREWFERQFSTSKDAQREIKSLEVRGDTVEVHVKLHATHNGQKHTVDLTHLFHFRGNRVNEVRVYINPTG | 97 |
| MSEEQIRQSLRRFYEALDSGDADTAASLFHPHVTIHLWDGVTFTSREEFREWFERRFSTSKDAQREIKSLEVRGDTVEVHVQLHATHNGQKHTVDLTHLFHFRGNRVTEVRVYINPTG | 98 |
| MSEEQIRQGLRRFYEALDSGDADTAASFFHPQVTIHLWDGVTFTSREEFREWFERRFSTSPDAQREIKSLEVRGDTVEVHVKLHATHNGQKHTVDLTHLFHFRGNRVDEVRVYINPTG | 99 |
| MSEEQIRQHLRRFYEALDSGDADTAASLFHPDVTIHLWDGVTFTSREEFREWFERRFSTSKDAQREIKSLEVRGDTVEVHVTLHATHNGQKHTVDLTHHFHFRGNRVAEVRVFINPTG | 100 |
| MSEEQIRQVLRRFYEALDSGDADTAASFFHPDVTIHLWDGVTFTSREEFREWFERRFSTSKDAQREIKSLEVRGDTVEVHVKLHATHNGQKHTVDLTHHFHFRGNRVNEVRVLINPTG | 101 |
| MSEEQIRQGLRRFYEALDRGDADTAASLFHPEVTIHLWDGVTFTSREEFREWFERMFSTSKDASREIKSLEVRGDTVEVHVQLHLTHNGQKHTVDLTHLFHFRGNRVDEVRVHINPTG | 102 |
| MSEEQIRQLLRRFYEALDSGDADTAASLFHPGVTIHLWDGVTFTSREEFREWFERLFSTSKDAFREIKSLEVRGDTVEVHVQLHFTRNGQKHTVDLTHLFHFRGNRVDEVRVYINPTG | 103 |
| MSEEQIRQNLRRFYEALDSGDADTAASLFHPGVTIHLWDGVTFTSREEFREWFERLFSTSKDALREIKSLEVRGDTVEVHVQLHFTRNGQKHTVDLTHLFHFRGNRVDEVRVYINPTG | 104 |
| MSEEQIRQALRRFYEALDRGDADTAASLFHPGVTIHLWDGVTFTSREEFREWFERLFSTSKDANREIKSLEVRGDTVEVHVQLHYTRNGQKHTVDLTHHFHFRGNRVNEVRVYINPTG | 105 |
| MSEEQIRQLLRRFYEALDRGDADTAASFFHPQVTIHLWDGVTFTSREEFREWFERRFSTSTDAQREIKSLEVRGDTVEVHVTLHATHNGQKHTVDLTHLFHFRGNRVDEVRVLINPTG | 106 |
| MSEEQIRQDLRRFYEALDRGDADTAASLFHPHVTIHLWDGVTFTSREEFREWFERRFSTSQDAQREIKSLEVRGDTVEVHVKLHATHNGQKHTVDLTHLFHFRGNRVDEVRVYINPTG | 107 |
| MSEEQIRQGLRRFYEALDSGDADTAASFFHPQVTIHLWDGVTFTSREEFREWFERRFSTSPDAQREIKSLEVRGDTVEVHVKLHATHNGQKHTVDLTHLWHFRGNRVDEVRVYINPTG | 108 |
| MSEEQIRQVLRRFYEALDSGDADTAASFFHPDVTIHLWDGVTFTSREEFREWFERRFSTSKDAQREIKSLEVRGDTVEVHVKLHATHNGQKHTVDLTHHWHFRGNRVNEVRVLINPTG | 109 |
| MSEEQIRQSLRRFYEALDSGDADTAASLFHPHVTIHLWDGVTFTSREEFREWFERRFSTSKDAQREIKSLEVRGDTVEVHVQLHATHNGQKHTVDLTHLWHFRGNRVTEVRVYINPTG | 110 |
| MSEEQIRQSLRRFYEALDSGDADTAASLFHPDVTIHLWDGVTFTSREEFREWFERRFSTSTDAQREIKSLEVRGDTVEVHVTLHATHNGQKHTVDLTHHWHFRGNRVDEVRVLINPTG | 111 |
| MSEEQIRQRLRRFYEALDRGDADTAASLFHPEVTIHLWDGVTFTSREEFREWFERRFSTSKDAQREIKSLEVRGDTVEVHVQLHATHNGQKHTVDLTHLWHFRGNRVAEVRVLINPTG | 112 |
| MSEEQIRQHLRRFYEALDSGDADTAASLFHPDVTIHLWDGVTFTSREEFREWFERRFSTSKDAQREIKSLEVRGDTVEVHVTLHATHNGQKHTVDLTHHWHFRGNRVAEVRVFINPTG | 113 |
| MSEEQIRQGLRRFYEALDRGDADTAASLFHPEVTIHLWDGVTFTSREEFREWFERMFSTSKDASREIKSLEVRGDTVEVHVQLHLTHNGQKHTVDLTHLWHFRGNRVDEVRVHINPTG | 114 |
| MSEEEIRQKLRRFYEALDSGDADTAASLFHPGVTIHLWDGVTFTSREEFREWFERRFSTSKDAQREIKSLEVRGDTVEVHVTLHATHNGQKHTVDLTHLWHFRGNRVNEVRVYINPTG | 115 |
| MSEEQIRQKLRRFYEALDSGDADTAASLFHPGVTIHLWDGVTFTSREEFREWFERLFSTSKDAVREIKSLEVRGDTVEVHVQLHFTRNGQKHTVDLTHHWHFRGNRVTEVRVHINPTG | 116 |
| MSEEQIRQLLRRFYEALDSGDADTAASLFHPGVTIHLWDGVTFTSREEFREWFERLFSTSKDAFREIKSLEVRGDTVEVHVQLHFTRNGQKHTVDLTHLWHFRGNRVDEVRVYINPTG | 117 |
| MSEEQIRQSLRRFYEALDSGDADTAASLFHPGVTIHLWDGVTFTSREEFREWFERRFSTSPDAQREIKSLEVRGDTVEVHVTLHATHNGQKHTVDLTHHWHFRGNRVDEVRVYINPTG | 118 |
| MSEEQIRQKLRRFYEALDRGDADTAASLFHPEVTIHLWDGVTFTSREEFREWFERKFSTSKDAQREIKSLEVRGDTVEVHVKLHATHNGQKHTVDLTHHWHFRGNRVNEVRVYINPTG | 119 |
| MSEEQIRQKLRRFYEALDSGDADTAASLFHPGVTIHLWDGVTFTSREEFREWFERRFSTSPDAQREIKSLEVRGDTVEVHVTLHATHNGQKHTVDLTHLWHFRGNRVDEVRVFINPTG | 120 |
| MSEEQIRQDLRRFYEALDRGDADTAASFFHPAVTIHLWDGVTFTSREEFREWFERRFSTSKDAQREIKSLEVRGDTVEVHVKLHATHNGQKHTVDLTHLWHFRGNRVDEVRVYINPTG | 121 |
| MSEEQIRQLLRRFYEALDSGDADTAASLFHPDVTIHLWDGVTFTSREEFREWFERRFSTSEDAQREIKSLEVRGDTVEVHVTLHATHNGQKHTVDLTHLWHFRGNRVTEVRVFINPTG | 122 |
| MSEEQIRQCLRRFYEALDRGDADTAASFFHPDVTIHLWDGVTFTSREEFREWFERRFSTSKDAQREIKSLEVRGDTVEVHVTLHATHNGQKHTVDLTHLWHFRGNRVNEVRVLINPTG | 123 |
| MSEEQIRQSLRRFYEALDRGDADTAASLFHPDVTIHLWDGVTFTSREEFREWFERQFSTSKDAQREIKSLEVRGDTVEVHVKLHATHNGQKHTVDLTHLWHFRGNRVNEVRVYINPTG | 124 |
| MSEEQIRQELRRFYEALDRGDADTAASLFHPDVTIHLWDGVTFTSREEFREWFERRFSTSQDAQREIKSLEVRGDTVEVHVKLHATHNGQKHTVDLTHLWHFRGNRVNEVRVFINPTG | 125 |
| MSEEQIRQLLRRFYEALDSGDADTAASLFHPGVTIHLWDGVTFTSREEFREWFERLFSTSKDAYREIKSLEVRGDTVEVHVQLHFTRNGQKHTVDLTHHWHFRGNRVTEVRVHINPTG | 126 |
| MSEEQIRQTLRRFYEALDSGDADTAASLFHPGVTIHLWDGVTFTSREEFREWFERRFSTSQDAQREIKSLEVRGDTVEVHVKLHATHNGQKHTVDLTHHWHFRGNRVNEVRVFINPTG | 127 |
| MSEEQIRQNLRRFYEALDSGDADTAASLFHPGVTIHLWDGVTFTSREEFREWFERLFSTSKDALREIKSLEVRGDTVEVHVQLHFTRNGQKHTVDLTHLWHFRGNRVDEVRVYINPTG | 128 |
| MSEEQIRQYLRRFYEALDSGDADTAASLFHPGVTIHLWDGVTFTSREEFREWFERRFSTSPDAQREIKSLEVRGDTVEVHVQLHFTVNGQKHTVDLTHHWHFRGNRVDEVRVLINPTG | 129 |
| MSEEQIRQGLRRFYEALDSGDADTAASLFHPGVTIHLWDGVTFTSREEFREWFERLFSTSKDAYREIKSLEVRGDTVEVHVQLHVTVNGQKHTVDLTHHWHFRGNRVDEVRVYINPTG | 130 |
| MSEEQIRQILRRFYEALDSGDADTAASLFHPGVTIHLWDGVTFTSREEFREWFERMFSTSKDASREIKSLEVRGDTVEVHVQLHLTRNGQKHTVDLTHHWHFRGNRVTEVRVYINPTG | 131 |
| MSEEQIRQILRRFYEALDRGDADTAASLFHPPVTIHLWDGVTFTSREEFREWFERRFSTSKDAYREIKSLEVRGDTVEVHVQLHLTHNGQKHTVDLTHHWHFRGNRVTEVRVFINPTG | 132 |
| MSEEQIRQSLRRFYEALDSGDADTAASLFHPGVTIHLWDGVTFTSREEFREWFERLFSTSKDAKREIKSLEVRGDTVEVHVQLHFTVNGQKHTVDLTHHWHFRGNRVTEVRVHINPTG | 133 |
| MSEEQIRQKLRRFYEALESGDADTAASLFHPGVTIHLWDGVTFTSREEFREWFERLFSTSKDAHREIKSLEVRGDTVEVHVQLHYTRNGQKHTVDLTHHWHFRGNRVDEVRVFINPTG | 134 |
| MSEEQIRQELRRFYEALDRGDADTAASLFHPQVTIHLWDGVTFTSREEFREWFERRFSTSKDANREIKSLEVRGDTVEVHVQLHFTHNGQKHTVDLTHLWHFRGNRVNEVRVYINPTG | 135 |
| MSEEQIRQALRRFYEALDRGDADTAASLFHPGVTIHLWDGVTFTSREEFREWFERLFSTSKDANREIKSLEVRGDTVEVHVQLHYTRNGQKHTVDLTHHWHFRGNRVNEVRVYINPTG | 136 |
| MSEEQIRQSLRRFYEALDRGDADTAASLFHPGVTIHLWDGVTFTSREEFREWFERRFSTSQDAQREIKSLEVRGDTVEVHVKLHFTLNGQKHTVDLTHLFHFRGNRVNEVRVLINPTG | 137 |
| MSEEQIRQLLRRFYEALDRGDADTAASSFHPAVTIHLWDGVTFTSREEFREWFERQFSTSKDAWREIKSLEVRGDTVEVHVKLHKTDNGQKHTVDLTHLFHFRGNRVNEVRVYINPTG | 138 |
| MSEEQIRQWLRRFYEALDRGDADTAASLFHPEVTIHLWDGVTFTSREEFREWFERRFSTSKDAQREIKSLEVRGDTVEVHVKLHATLNGQKHTVDLTHLFHFRGNRVDEVRVLINPTG | 139 |
| MSEEQIRQALRRFYEALDRGDADTAASFFHPQVTIHLWDGVTFTSREEFREWFERRFSTSKDAWREIKSLEVRGDTVEVHVKLHYTHNGQKHTVDLTHLYHFRGNRVNEVRVLINPTG | 140 |
| MSEEQIRQILRRFYEALDRGDADTAASLFHPAVTIHLWDGVTFTSREEFREWFERRFSTSQDAWREIKSLEVRGDTVEVHVKLHYTDNGQKHTVDLTHLFHFRGNRVNEVRVLINPTG | 141 |
| MSEEQIRQNLRRFYEALDRGDADTAASFFHPEVTIHLWDGVTFTSREEFREWFERRFSTSKDARREIKSLEVRGDTVEVHVKLHFTLNGQKHTVDLTHHFHFRGNRVAEVRVLINPTG | 142 |
| MSEEQIRQMLRRFYEALDSGDADTAASSFHPEVTIHLWDGVTFTSREEFREWFERRFSTSPDAWREIKSLEVRGDTVEVHVKLHYTHNGQKHTVDLTHLLHFRGNRVDEVRVFINPTG | 143 |
| MSEEQIRQFLRRFYEALDSGDADTAASSFHPHVTIHLWDGVTFTSREEFREWFERRFSTSKDAWREIKSLEVRGDTVEVHVKLHLTDNGQKHTVDLTHHYHFRGNRVDEVRVYINPTG | 2663 |
| MSEEQIRQELRRFYEALDRGDADTAASLFHPQVTIHLWDGVTFTSREEFREWFERRFSTSKDANREIKSLEVRGDTVEVHVQLHFTHNGQKHTVDLTHLFHFRGNRVNEVRVYINPTG | 2665 |

### Conjugated Proteins

The LuxSit-i variants disclosed herein may be conjugated to another moiety. The moiety may be a small molecule, peptide, polypeptide, nucleic acid, or lipid. The LuxSit-i variants may also be tagged with a sequence for localization of the variants to a cellular compartment, cell membrane, or for secretion. The LuxSit-i variants disclosed herein can be used as biosensors by conjugating a moiety to the N-terminus, the C-terminus, or in between the N- and the C-terminus. The moiety may be conjugated directly to the LuxSit-i variant, e.g., via a peptide bond to the N-terminus and/or the C-terminus and/or to an amino acid side chain or may be conjugated to the LuxSit-i variant via a linker. The linker may be a polymer, e.g., an amino acid linker or a sugar linker.

A variety of linkers may be used and may include alkyl groups, methylene carbon chains, ether, polyether, alkyl amide linker, a peptide linker, a modified peptide linker, a Poly(ethylene glycol) (PEG) linker, a streptavidin-biotin or avidin-biotin linker, polyaminoacids (e.g., polylysine), functionalised PEG, polysaccharides, glycosaminoglycans, oligonucleotide linker, phospholipid derivatives, alkenyl chains, alkynyl chains, disulfide, or a combination thereof. In some embodiments, the linker is cleavable (e.g., enzymatically (e.g., TEV protease site), chemically, photoinduced cleavage, etc.).

In certain aspects, the moiety may be a heterologous amino acid sequence. In certain aspects, the moiety is conjugated to the LuxSit-i variant post-translationally. In certain aspects, the moiety is conjugated to the LuxSit-i variant during translation, e. g., a nucleic acid may encode a fusion protein comprising the Lux-Sit-i variant and the moiety.

In certain aspects, the heterologous amino acid sequence includes a protein binding domain, such as one that binds IL-17RA, e.g., IL-17A, or the IL-17A binding domain of IL-17RA, Jun binding domain of Erg, or the EG binding domain of Jun; a potassium channel voltage sensing domain, e.g., one useful to detect protein conformational changes, the GTPase binding domain of a Cdc42 or rac target, or other GTPase binding domains, domains associated with kinase or phosphotase activity, e.g., regulatory myosin light chain, PKC6, pleckstrin containing PH and DEP domains, other phosphorylation recognition domains and substrates; glucose binding protein domains, glutamate/aspartate binding protein domains, PKA or a cAMP-dependent binding substrate, InsP3 receptors, GKI, PDE, estrogen receptor ligand binding domains, apoK1-er, or calmodulin binding domains.

In certain aspects, a fusion protein comprising a LuxSit-i variant fused to a heterologous amino acid sequence may be a biosensor. The biosensor is useful to detect a GTPase, e.g., binding of Cdc42 or Rac to a EBFP, EGFP PAK fragment, Raichu-Rac, Raichu-Cdc42, integrin alphavbeta3, IBB of importin-a, DMCA or NBD-Ras of CRaf1 (for Ras activation), binding domain of Ras/Rap Ral RBD with Ras prenylation sequence. In one embodiment, the biosensor detects PI(4,5)P2 (e.g., using PH-PCLdelta1, PH-GRP1), PI(4,5)P2 or PI(4)P (e.g., PH-OSBP), PI(3,4,5)P3 (e.g., using PH-ARNO, or PH-BTK, or PH-Cytohesin1), PI(3,4,5)P3 or PI(3,4)P2 (e.g., using PH Akt), PI(3)P (e.g., using FYVE-EEA1), or Ca2+ (cytosolic) (e.g., using calmodulin, or C2 domain of PKC.

In one aspect, a fusion protein comprising a LuxSit-i variant is fused to a protein domain. In one embodiment, the domain is one with a phosphorylated tyrosine (e.g., in Src, Ab1 and EGFR), that detects phosphorylation of ErbB2, phosphorylation of tyrosine in Src, Ab1 and EGFR, activation of MKA2 (e.g., using MK2), cAMP induced phosphorylation, activation of PKA, e.g., using KID of CREG, phosphorylation of CrkII, e.g., using SH2 domain pTyr peptide, binding of bZIP transcription factors and REL proteins, e.g., bFos and bJun ATF2 and Jun, or p65 NFkappaB, or microtubule binding, e.g., using kinesin.

The LuxSit-i variants disclosed herein as well as the circularly permuted versions of LuxSit-i and LuxSit-i variants and the self-complementing components of LuxSit-1, LuxSit-i variants, and circularly permuted versions of LuxSit-i and LuxSit-i variants may be conjugated to an antibody or an antigen binding fragment thereof.

The LuxSit-i variants disclosed herein as well as the circularly permuted versions of LuxSit-i and LuxSit-i variants may include deletions of residues at the original (e.g., prior to being circularly permuted)N- or C-termini, or both, e.g., deletion of 1 to 3 or more residues at the N-terminus and 1 to 6 or more residues at the C-terminus, as well as inclusion of sequences that directly or indirectly interact with a molecule of interest, such as, the molecules described herein.

### Self-Complementing Mutipartite Protein Having Luciferase Activity

A self-complementing multipartite protein having luciferase activity is provided. In certain aspects, the multipartite protein may have two self-complementing components or three self-complementing components.

Self-complementing refers to the characteristic of two or more polypeptides of being able to form a complex with each other to regain enzymatic activity absent or substantially absent when the two or more polypeptides are not associated. Complementary polypeptides may require assistance to form a stable complex (e.g., from interaction elements), for example, to place the polypeptides in the proper conformation for complementarity, to co-localize complementary polypeptides, to lower interaction energy for polypeptides, etc.

Multipartite protein refers to a protein complex in which the polypeptide components of the multipartite protein are in direct and/or indirect contact with one another. In one aspect, direct contact means two or more molecules are close enough so that attractive noncovalent interactions between the molecules, such as Van der Waal forces, hydrogen bonding, ionic and hydrophobic interactions, and the like, influence the interaction of the molecules. An example of direct contact can include a multipartite protein comprising from N-terminus to C-terminus, a first polypeptide component, a linker, and a second polypeptide component, where the first polypeptide component and the second polypeptide component associate and have luciferase activity and upon cleavage of the linker are separated and lack or have substantially reduced cleavage activity. In one aspect, indirect contact means two or more molecules interact when bridging moieties conjugated to the two or more molecules bring the two or more molecules close together in a stable comples so that attractive noncovalent interactions between the molecules, such as Van der Waal forces, hydrogen bonding, ionic and hydrophobic interactions, and the like, influence the interaction of the molecules.

### Self-Complementing Multipartite Protein Having Two or More Components

In certain aspects, the self-complementing multipartite protein includes at least a first polypeptide component and a second polypeptide component, where the at least first polypeptide component and the second polypeptide component are not covalently linked or are covalently linked via a linker (e.g., a cleavable linker), where in total the first polypeptide component and the second polypeptide component comprise the secondary structure arrangement H1-L1-H2-L2-B1-L3-B2-L4-H3-L5-B3-L6-B4-L7-B5-L8-B6, where each domain is as described herein and (a) each H and B domain is fully present within one polypeptide component of either the first polypeptide component or the second polypeptide component, (b) the first polypeptide component and the second polypeptide component do not include all of the H and B domains, (c) the relative order of the H, L, and B domains when present in either of the first polypeptide component or the second polypeptide component is unchanged with reference to the order set forth in the secondary structure arrangement H1-L1-H2-L2-B1-L3-B2-L4-H3-L5-B3-L6-B4-L7-B5-L8-B6, and (d) the first component and the second component when not present in the self-complementing multipartite protein do not possess detectable luciferase activity or have luciferase activity lower than the luciferase activity of the self-complementing multipartite protein.

In certain aspects, the first polypeptide component and the second polypeptide component comprise the secondary structure arrangement as set forth in Table 1:

TABLE 1

| first polypeptide component | second polypeptide component |
|---|---|
| H1-(L1) | (L1)-H2-L2-B1-L3-B2-L4-H3-L5- |
| | B3-L6-B4-L7-B5-L8-B6 |
| H1-L1-H2-(L2) | (L2)-B1-L3-B2-L4-H3-L5-B3-L6- |
| | B4-L7-B5-L8-B6 |
| H1-L1-H2-L2-B1-(L3) | (L3)-B2-L4-H3-L5-B3-L6-B4-L7- |
| | B5-L8-B6 |
| H1-L1-H2-L2-B1-L3-B2-(L4) | (L4)-H3-L5-B3-L6-B4-L7-B5-L8- |
| | B6 |
| H1-L1-H2-L2-B1-L3-B2-L4-H3-(L5) | (L5)-B3-L6-B4-L7-B5-L8-B6 |
| H1-L1-H2-L2-B1-L3-B2-L4-H3-L5- | (L6)-B4-L7-B5-L8-B6 |
| B3-(L6) | |
| H1-L1-H2-L2-B1-L3-B2-L4-H3-L5- | (L7)-B5-L8-B6 |
| B3-L6-B4-(L7) | |
| H1-L1-H2-L2-B1-L3-B2-L4-H3-L5- | (L8)-B6 |
| B3-L6-B4-L7-B5-(L8) | |
| (L1)-H2-L2-B1-L3-B2-L4-H3-L5- | H1-(L1) |
| B3-L6-B4-L7-B5-L8-B6 | |
| (L2)-B1-L3-B2-L4-H3-L5-B3-L6- | H1-L1-H2-(L2) |
| B4-L7-B5-L8-B6 | |
| (L3)-B2-L4-H3-L5-B3-L6-B4-L7- | H1-L1-H2-L2-B1-(L3) |
| B5-L8-B6 | |
| (L4)-H3-L5-B3-L6-B4-L7-B5-L8-B6 | H1-L1-H2-L2-B1-L3-B2-(L4) |
| (L5)-B3-L6-B4-L7-B5-L8-B6 | H1-L1-H2-L2-B1-L3-B2-L4-H3- |
| | (L5) |
| (L6)-B4-L7-B5-L8-B6 | H1-L1-H2-L2-B1-L3-B2-L4-H3- |
| | L5-B3-(L6) |
| (L7)-B5-L8-B6 | H1-L1-H2-L2-B1-L3-B2-L4-H3- |
| | L5-B3-L6-B4-(L7) |
| (L8)-B6 | H1-L1-H2-L2-B1-L3-B2-L4-H3- |
| | L5-B3-L6-B4-L7-B5-(L8) |

The L domain in parenthesis is (i) present in one but not both of the first and second components, (ii) is split between the first and second components, or (iii) absent.

In certain aspects, one or both of the first component and the second component includes an additional domain. The additional domain may be covalently linked to one or both of the first component and the second component. The domain may be a small molecule, a peptide, a polypeptide, nucleic acid, lipid, an aptamer, etc.

In certain aspects, the first component is a fusion protein that includes a first domain and the second component is a fusion protein that includes a second domain. The H and B domains present in the first and second components may be as described herein, such as, those having substitutions with respect the H and B domains of LuxSit-i.

In some aspects, the first component and the second component have high affinity for each other and form a high-affinity two-component protein having luciferase activity by direct interaction. In other words, the two components form a stable complex having luciferase activity when present in close vicinity, e.g., in a polypeptide, in a cell, in a cell lysate, in a cell free solution, etc.

In some aspects, the first component and the second component have low affinity for each other and form a two-component protein having luciferase activity by indirect interaction mediated by a binding pair. In other words, the two components form a stable complex having luciferase activity when each is conjugated to a member of a binding pair and the interaction between the binding pair members allow formation of a two-component protein having luciferase activity.

Binding pairs can be a ligand and a receptor; an antigen and an antibody; self-complementing enzyme fragments, such as, beta-galactosidase; biotin-avidin; two complementary nucleic acids; two polypeptides capable of dimerization (e.g., homodimer, heterodimer, etc.); and the like.

In some embodiments, the self-complementing multipartite protein comprises from N-terminus to C-terminus: a first polypeptide component, a linker, and a second polypeptide component or a second polypeptide component, a linker, and a first polypeptide component and has luciferase activity. The linker may be cleavable linker. For example, the linker may include a cleavage site for a protease. In the presence of the protease, the linker is cleaved resulting in separation of the first and second polypeptide components and loss or significant reduction of the luciferase activity as compared to the luciferase activity of the self-complementing multipartite protein. In certain embodiments, the protease may be a neurotoxin and the self-complementing multipartite protein may be used to detect presence of the protease. In certain embodiments, the self-complementing multipartite protein may include spacer regions between the linker and the first and/or the second component. In certain embodiments, the neurotoxin cleavage site comprises a Clostridium botulinum neurotoxin (BoNT) or a Tetanus neurotoxin cleavage site.

### Self-Complementing Multipartite Protein Having Three or More Components

In certain aspects, a self-complementing multipartite protein having luciferase activity as provided herein includes at least a first polypeptide component, a second polypeptide component, and a third polypeptide component, wherein the at least first polypeptide component, the second polypeptide component, and the third polypeptide component are not covalently linked, or are covalently linked via one ore more linkers (e.g., one or more cleavable linkers) wherein in total the first polypeptide component, the second polypeptide component, and the third polypeptide component comprise the secondary structure arrangement H1-L1-H2-L2-B1-L3-B2-L4-H3-L5-B3-L6-B4-L7-B5-L8-B6, wherein each domain is as described herein and (a) each H and B domain is fully present within one polypeptide component of the first polypeptide component, the second polypeptide component, or the third polypeptide component, (b) the first polypeptide component, the second polypeptide component, and the third polypeptide component do not include all of the H and B domains, (c) the relative order of the H, L, and B domains when present in the first polypeptide component, the second polypeptide component, or the third polypeptide component is unchanged with reference to the order in the secondary structure arrangement H1-L1-H2-L2-B1-L3-B2-L4-H3-L5-B3-L6-B4-L7-B5-L8-B6 and (d) the first component, the second polypeptide component, and the third polypeptide component when not present in the self-complementing multipartite protein do not possess detectable luciferase activity or have luciferase activity lower than the luciferase activity of the self-complementing multipartite protein.

In certain aspects, the H and B domains of the protein are separated into the first polypeptide component, the second polypeptide component, and the third polypeptide component at an L domain, wherein the point of separation is the N-terminus of the L domain, the C-terminus of the L domain or in the L domain. Accordingly, a first component may include the H1 domain, the second component may include the H2 domain, and the third component may include the remainder of the domains, wherein the L1 domain may be in the first component, the second component, split between the two components, or absent from both components and the L2 domain may be in the second component, the third component, split between the two components, or absent from both components.

A non-limiting list of three component systems is provided below:

| first polypeptide | second polypeptide | third polypeptide |
|---|---|---|
| component | component | component |
| H1-(L1) | (L1)-H2-(L2) | (L2)-B1-L3-B2-L4-H3-L5-B3- |
| | | L6-B4-L7-B5-L8-B6 |
| H1-L1-H2-(L2) | (L2)-B1-(L3) | (L3)-B2-L4-H3-L5-B3-L6-B4- |
| | | L7-B5-L8-B6 |
| H1-L1-H2-L2-B1-(L3) | (L3)-B2-(L4) | (L4)-H3-L5-B3-L6-B4-L7-B5- |
| | | L8-B6 |
| H1-L1-H2-L2-B1-L3-B2-(L4) | (L4)-H3-(L5) | (L5)-B3-L6-B4-L7-B5-L8-B6 |
| H1-L1-H2-L2-B1-L3-B2-L4-H3-(L5) | (L5)-B3-(L6) | (L6)-B4-L7-B5-L8-B6 |
| H1-L1-H2-L2-B1-L3-B2-L4-H3-L5- | (L6)-B4-L7-B5-(L8) | (L8)-B6 |
| B3-(L6) | | |

In certain aspects, at least one of the first component, the second component, and the third component comprises an additional domain. In certain aspects, the additional domain is covalently linked to at least one of the first component, the second component, and the third component.

In certain aspects, the first component is a fusion protein comprising a first domain, the second component is a fusion protein comprising a second domain, and the third component is a fusion protein comprising a third domain. The H and B domains present in the first, second, and third components may be as described herein, such as, those having substitutions with respect the H and B domains of LuxSit-i.

### Circularly Permuted Polypeptide Having Luciferase Activity

In certain embodiments, a circularly permuted polypeptide having luciferase activity is disclosed. The N-terminus and the C-terminus of the circularly permuted polypeptide are different from the N-terminus and C-terminus, respectively, of a protein having luciferase activity and comprising the secondary structure arrangement H1-L1-H2-L2-B1-L3-B2-L4-H3-L5-B3-L6-B4-L7-B5-L8-B6, where the H, B, and L domains are as set forth for LuxSit-i or variants of LuxSit-i described herein. The N-terminus and C-terminus of the protein having luciferase activity are joined by a linker sequence and the circularly permuted polypeptide comprises the secondary structure arrangement:
H2-L2-B1-L3-B2-L4-H3-L5-B3-L6-B4-L7-B5-L8-B6-LINKER-H1-(L1) (I),
B1-L3-B2-L4-H3-L5-B3-L6-B4-L7-B5-L8-B6-LINKER-H1-L1-H2-(L2) (II),
B2-L4-H3-L5-B3-L6-B4-L7-B5-L8-B6-LINKER-H1-L1-H2-L2-B1-(L3) (Ill),
H3-L5-B3-L6-B4-L7-B5-L8-B6-LINKER-H1-L1-H2-L2-B1-L3-B2-(L4) (IV),
B3-L6-B4-L7-B5-L8-B6-LINKER-H1-L1-H2-L2-B1-L3-B2-L4-H3-(L5) (V),
B4-L7-B5-L8-B6-LINKER-H1-L1-H2-L2-B1-L3-B2-L4-H3-L5-B3-(L6) (VI),
B5-L8-B6-LINKER-H1-L1-H2-L2-B1-L3-B2-L4-H3-L5-B3-L6-B4-(L7) (VII), or
B6-LINKER-H1-L1-H2-L2-B1-L3-B2-L4-H3-L5-B3-L6-B4-L7-B5-(L8) (VIII),
   wherein the L domain in parenthesis is present at the C-terminus, or the N-terminus, or is split between the C-terminus and the N-terminus or is absent.

In some embodiments, the B5 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: QKHTVDLTHHWHFR or QKHTVILTHVFRFR. In some embodiments, B4 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence:

TVEVHVQLHATH
or
TVVVVVRLDFTL.

In some aspects, the linker has a length of 10-100 amino acids, e.g., 10-90, 10-80, 10-70, 10-60, or 10-50 amino acids in length. In some aspects, the linker comprises the secondary structure H4-L9. In some aspects, the linker comprises the secondary structure H4-L9-H5-L10. In some aspects, the linker comprises the secondary structure H4-L9-H5-L10-H6-L11. H4, H5, and H6 can be helical domains of any amino acid sequence that provide a helical tracuture. L9, L10, and L11 can be linker sequences and can range in length from 1, 2, 3, 4, 5, or more amino acids.

In some aspects, the linker comprises an amino acid sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the amino acid VDDVEEVLARVLEEGERLVERLRAERPEA; TGEEPEKPEFKETFGPS; VESEEELPAALARAEELGRELLERTLAEEGAGGPP; APSLDEESIEARVAEARRLAEERLAELGDPPP; TGEEPEPPEFRERFGPSA; DLSPEAIEAAIAKALARADALLAELGAPPP; TGEEPERPEFVERFGPSS; SLDEAAIEAAIARARARADELLAELGAPPA; CPSLDEASIAAAIAEAEALAAERLAELGAPPP; TGEEPEPPEFRERFGPSS; or DPDEETRLAAAREALERAGVPEEMRRAALELLERGERELFRPSA.

Also encompassed by the present disclosure are split-component multipartite proteins comprising at least two components or at least three components derived from splitting the circularly permuted polypeptides described here. The B, H, and L domains may be as specified herein.

In some aspects, a circularly permuted protein is derived from the LuxSit-i variant having an amino acid sequence set forth in SEQ ID NO:2600 and has the amino acid sequence set forth in SEQ ID NOs: 2227 or 2236 and has luciferase activity similar to SEQ ID NO:2600.

The circularly permuted proteins provided herein may be used in a method similar to those described herein for the LuxSit-i variants and in methods known in the art for using luciferases. The split versions of a circularly permuted protein may be used in methods as is known in the art and those described herein for the self-complementing multipartite proteins.

In certain aspects, a polypeptide encompassed by the present disclosure includes one having an amino acid sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the amino acid of any polypeptide provided here.

In certain aspects, a circularly permuted protein comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence set forth in any one of SEQ ID NOs: 144-2599.

In certain aspects, a first component encompassed by the present disclosure includes one having an amino acid sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the amino acid of any first component provided here.

In certain aspects, a second component encompassed by the present disclosure includes one having an amino acid sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the amino acid of any second component provided here.

In certain aspects, a third component encompassed by the present disclosure includes one having an amino acid sequence having at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identity to the amino acid of any third component provided here.

### Nucleic Acids

In some aspects, where the polypeptide is relatively short, e.g., include one or a few H or B domains, such polypeptides may be synthesized using synthetic chemistry. In other aspects, the present disclosure provides nucleic acids comprising nucleotide sequences encoding the polypeptides described herein. These nucleic acids may be used for a cell-free transcription and translation. A nucleotide sequence encoding a subject polypeptide can be operably linked to one or more regulatory elements, such as a promoter and enhancer, that allow expression of the nucleotide sequence in a recombinant cell that is genetically modified to produce the polypeptide.

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, cytomegalovirus immediate early promoter; herpes simplex virus thymidine kinase promoter; early and late SV40 promoters; promoter present in long terminal repeats from a retrovirus; mouse metallothionein-I promoter; and the like.

A nucleotide sequence encoding a subject polypeptide can be present in an expression vector and/or a cloning vector. An expression vector can include a selectable marker, an origin of replication, and other features that provide for replication and/or maintenance of the vector. Large numbers of suitable vectors and promoters are known to those of skill in the art; many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia). Expression vectors generally have convenient restriction sites located near the promoter sequence to provide for the insertion of nucleic acid sequences encoding polpeptides. A selectable marker operative in the expression host cell may be present.

Nucleic acids, e.g., as described herein, may, in some instances, be introduced into a cell, e.g., by contacting the cell with the nucleic acid. Cells with introduced nucleic acids will generally be referred to herein as genetically modified cells. Various methods of nucleic acid delivery may be employed including but not limited to e.g., naked nucleic acid delivery, viral delivery, chemical transfection, biolistics, and the like.

The nucleic acids of the present disclosure may be provided in a kit. The kit may include additional components such as resconstitution buffer for resuspending the nucleic acid provided in the kit in a lyophilized form.

### Host Cells

The present disclosure provides isolated genetically modified cells (e.g., in vitro cells, ex vivo cells, cultured cells, etc.) that are genetically modified with a subject nucleic acid. In some aspects, a subject isolated genetically modified cell can produce a subject polypeptide. In some instances, a genetically modified cell may be used in the screening, and/or discovery of protein-protein interaction; protein-drug interactions; protein-nucleic acid interaction, etc.

Suitable cells include eukaryotic cells, such as a mammalian cell, an insect cell, a yeast cell; and prokaryotic cells, such as a bacterial cell. Introduction of a subject nucleic acid into the host cell can be affected, for example by calcium phosphate precipitation, DEAE dextran mediated transfection, liposome-mediated transfection, electroporation, or other known methods.

### Kits

Aspects of the present disclosure include kits for measuring luciferase activity of a luciferase. The kit may include components for measuring activity of a luciferase. The components may be present in separate compartments, e.g., in separate vials. Aspects of the present disclosure include kits for measuring luciferase activity of a polypeptide having luciferase activity and/or a self-complementing multipartite protein having luciferase activity. In certain aspects, the kit may include one or more of the polypeptides, the first component, the second component, and/or the third component as disclosed herein.

Aspects of the present disclosure include kits comprising one or more nucleic acids encoding the polypeptides, the first component, the second component, and/or the third component.

In certain aspects, the kit may include an assay buffer suitable for measuring luciferase activity. The kit may include one or more container means such as vials, tubes, and the like, each of the container means comprising the different polypeptides, substrates, assay buffer, etc., to be used in a method for measuring luciferase activity. For example, one of the containers may include a polypeptide having luciferase activity or a polynucleotide (e.g., in the form of a vector) encoding the polypeptide. A second container may contain a substrate for the polypeptide. The assay buffer may be any suitable buffer such as a solution described in the present disclosure.

The kit may include a luciferin substrate, such as, DTZ, coelenterazine, furimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine, coelenterazine-i, coelenterazine-icp, coelenterazine-v, and 2-methyl coelenterazine, or another luciferin substrate, or an analog thereof.

In certain aspects, the compounds of Formula (I) disclosed herein may be provided as part of the kit. In some embodiments, the kit may include one or more luciferases (in the form of a polypeptide, a polynucleotide, or both, as disclosed herein) and a bioluminescent luciferin substrate of Formula (I).

The kit may also include one or more buffers, such as the solution or assay buffer disclosed herein. The kit may include instructions to enable a user to perform assays such as those disclosed herein. In certain aspects, the kit includes instructions for a method for detecting luminescence in a cell comprises contacting a cell with a luciferin analog, wherein the luciferin analog is a compound of Formula (I), or a stereoisomer, a tautomer or a salt thereof; and detecting luminescence. In certain aspects, the cell contains a live cell. In certain aspects, the cell is in vivo, ex vivo, or in vitro.

In certain aspects, a kit comprises a luciferin analog, wherein the luciferin analog is a compound of Formula (I), or a stereoisomer, a tautomer or a salt thereof. In certain aspects, a kit further comprises a polypeptide having luciferase activity as disclosed herein. In certain aspects, a kit further comprises a buffer reagent.

In certain aspects, the kit may include a luciferin substrate. In certain aspects, the kit may include a luciferin substrate of formula (I):

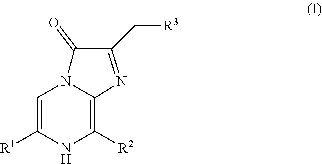

wherein R¹, R², and R³ are independently selected from: a C₃₋₆ cycloalkyl; an aryl; an aryl substituted with at least one of C₁₋₃ alkyl, halogen, C₁₋₃ haloalkyl, hydroxyl, alkoxy, nitro or amino alcohol; 5-10 membered heteroaryl having 1-3 ring heteroatoms independently selected from O, S, Se and N, and a heterocycle;
wherein:
if R¹ is an aryl, then R² and R³ are independently selected from: a C₃₋₆ cycloalkyl; an aryl; an aryl substituted with at least one of C₁₋₃ alkyl, halogen, C₁₋₃ haloalkyl, hydroxy, alkoxy or nitro; and 5-10 membered heteroaryl having 1-3 ring heteroatoms independently selected from O, S, Se and N;
if R³ is an aryl, then R¹ and R² are independently selected from: a C₃₋₆ cycloalkyl; an aryl; an aryl substituted with at least one of C₁₋₃ alkyl, halogen, C₁₋₃ haloalkyl, hydroxy, alkoxy, nitro or amino alcohol; 5 membered heteroaryl having 1-3 ring heteroatoms independently selected from O, S, Se and N; and 10 membered heteroaryl having 1-3 ring heteroatoms independently selected from O, S, and Se; 6 membered heteroaryl having 1-3 ring heteroatoms independently selected from O, S, Se, and N; a heterocycle and
if R² s an aryl, then R¹ and R³ are independently selected from: a C₃₋₆ cycloalkyl; an aryl; an aryl substituted with at least one of C₁₋₃ alkyl, halogen, C₁₋₃ haloalkyl, hydroxy, alkoxy, nitro or amino alcohol; 5 membered heteroaryl having 1-3 ring heteroatoms independently selected from O, S, Se and N; 10 membered heteroaryl having 1-3 ring heteroatoms independently selected from O, S, Se and N, and a heterocycle.

In certain aspects, the, R¹ and R² are aryl. In certain aspects, R² and R³ are aryl. In certain aspects, R¹ and R³ are aryl.

In certain aspects, any one of R¹, R², and R³ is selected from C₃₋ₛ cycloalkyl. The C₃-s cycloalkyl group includes cycloalkyl groups having 3 to 6 carbon atoms, e.g., cyclopropyl, cyclobutyl, cyclopentyl and cyclohexyl. In certain aspects, any one of R¹, R², and R³ is selected from cyclopropyl.

In certain aspects, any one of R¹, R², and R³ is selected from an aryl substituted with at least one of C₁₋₃ alkyl, halogen, C₁₋₃ haloalkyl, hydroxyl, alkoxy, nitro or amino alcohol.

In certain aspects, any one of R¹, R², and R³ is selected from an aryl substituted with C₁₋₃ alkyl. The C₁₃ alkyl group includes straight or branched alkyl groups having 1 to 3 carbon atoms, e.g., methyl, ethyl, n-propyl and isopropyl. In certain aspects, any one of R¹, R², and R³ is selected from an aryl substituted with methyl.

In certain aspects, any one of R¹, R², and R³ is selected from an aryl substituted with halogen. The halogen group includes halogen atoms, e.g., fluorine (F), chlorine (CI), bromine (Br) and iodine (I). In certain aspects, any one of R¹, R², and R³ is selected from an aryl substituted with fluorine.

In certain aspects, any one of R¹, R², and R³ is selected from an aryl substituted with C₁₋₃ haloalkyl. The C₁₋₃ haloalkyl group includes straight or branched haloalkyl groups having 1 to 3 carbon atoms obtained by substituting one or more hydrogen atoms with halogen atoms, e.g., fluoromethyl, difluoromethyl, trifluoromethyl, chloromethyl, and dichloromethyl. In certain aspects, any one of R¹, R², and R³ is selected from an aryl substituted with trifluoromethyl.

In certain aspects, any one of R¹, R², and R³ is selected from an aryl substituted with hydroxyl. In certain aspects, any one of R¹, R², and R³ is selected from an aryl substituted with alkoxy. The alkoxy group includes straight or branched alkyl groups having 1 to 3 carbon atoms, e.g., methoxy, ethoxy, n-propoxy and isopropoxy. In certain aspects, any one of R¹, R², and R³ is selected from an aryl substituted with methoxy.

In certain aspects, any one of R¹, R², and R³ is selected from an aryl substituted with nitro.

In certain aspects, any one of R¹, R², and R³ is selected from an aryl substituted with halogen and hydroxyl. In certain aspects, any one of R¹, R², and R³ is selected from an aryl substituted with fluorine and hydroxyl.

In certain aspects, any one of R¹, R², and R³ is selected from 5-10 membered heteroaryl having 1-3 ring heteroatoms independently selected from O, S, Se and N. The 5-10 membered heteroaryl group includes pyrrole, furan, thiophene, selenophene, pyridine, imidazole, thiazole, isothiazole, oxazole, isoxazole, quinoline and isoquinoline. In certain aspects, R³ is selected from 6-membered heteroaryl having a N heteroatom, e.g., pyridine. In certain aspects, R¹ and R² are not a 6-membered heteroaryl having a N heteroatom, e.g., pyridine.

In certain aspects, R¹ is an aryl, R² is an aryl and R³ is cyclopropyl. In certain aspects, R¹ is an aryl, R² is an aryl and R³ is an aryl substituted with methyl. In certain aspects, R¹ is an aryl, R² is an aryl and R³ is an aryl substituted with fluorine. In certain aspects, R¹ is an aryl, R² is an aryl and R³ is an aryl substituted with trifluoromethyl. In certain aspects, R¹ is an aryl, R² is an aryl and R³ is an aryl substituted with hydroxyl. In certain aspects, R¹ is an aryl, R² is an aryl and R³ is an aryl substituted with methoxy. In certain aspects, R¹ is an aryl, R² is an aryl and R³ is an aryl substituted with nitro. In certain aspects, R¹ is an aryl, R² is an aryl and R³ is selected from 5 membered heteroaryl having O, S, Se or N heteroatom.

In certain aspects, R¹ is an aryl, R² is an aryl and R³ is 6-membered heteroaryl having a N heteroatom. In certain aspects, R² is an aryl, R³ is an aryl and R¹ is cyclopropyl. In certain aspects, R² is an aryl, R³ is an aryl and R¹ is an aryl substituted with methyl. In certain aspects, R² is an aryl, R³ is an aryl and R¹ is an aryl substituted with fluorine. In certain aspects, R² is an aryl, R³ is an aryl and R¹ is an aryl substituted with trifluoromethyl. In certain aspects, R² is an aryl, R³ is an aryl and R¹ is an aryl substituted with hydroxyl. In certain aspects, R² is an aryl, R³ is an aryl and R¹ is an aryl substituted with methoxy. In certain aspects, R² is an aryl, R³ is an aryl and R¹ is an aryl substituted with nitro. In certain aspects, R² is an aryl, R³ is an aryl and R¹ is selected from 5 membered heteroaryl having O, S, Se or N heteroatom.

In certain aspects, R¹ is an aryl, R³ is an aryl and R² is cyclopropyl. In certain aspects, R¹ is an aryl, R³ is an aryl and R² is an aryl substituted with methyl. In certain aspects, R¹ is an aryl, R³ is an aryl and R² is an aryl substituted with fluorine. In certain aspects, R¹ is an aryl, R³ is an aryl and R² is an aryl substituted with trifluoromethyl. In certain aspects, R¹ is an aryl, R³ is an aryl and R² is an aryl substituted with hydroxyl. In certain aspects, R¹ is an aryl, R³ is an aryl and R² is an aryl substituted with methoxy. In certain aspects, R¹ is an aryl, R³ is an aryl and R² is an aryl substituted with nitro. In certain aspects, R¹ is an aryl, R³ is an aryl and R² is selected from 5 membered heteroaryl having O, S, Se or N heteroatom.

In certain aspects, R¹ is an aryl substituted with hydroxyl, R² is an aryl substituted with methoxy and R³ is an aryl. In certain aspects, R¹ is an aryl substituted with hydroxyl, R² is an aryl substituted with hydroxyl and R³ is an aryl. In certain aspects, R¹ is an aryl substituted with hydroxyl, R² is an aryl substituted with fluorine and R³ is an aryl. In certain aspects, R¹ is an aryl substituted with hydroxyl, R² is selected from 5 membered heteroaryl having O, S, Se or N heteroatom and R³ is an aryl. In certain aspects, R¹ is an aryl substituted with hydroxyl, R² is an imidazole and R³ is an aryl. In certain aspects, R¹ is an aryl substituted with hydroxyl, R² is selected from 10 membered heteroaryl having O, S, Se or N heteroatom and R³ is an aryl. In certain aspects, R¹ is an aryl substituted with hydroxyl, R² is a quinoline and R³ is an aryl. In certain aspects, R¹ is an aryl substituted with hydroxyl, R² is 6-membered heteroaryl having a N heteroatom and R³ is an aryl. In certain aspects, R¹ is an aryl substituted with hydroxyl, R² is a pyridine and R³ is an aryl.

Representative compounds of Formula (I) include, but are not limited to:

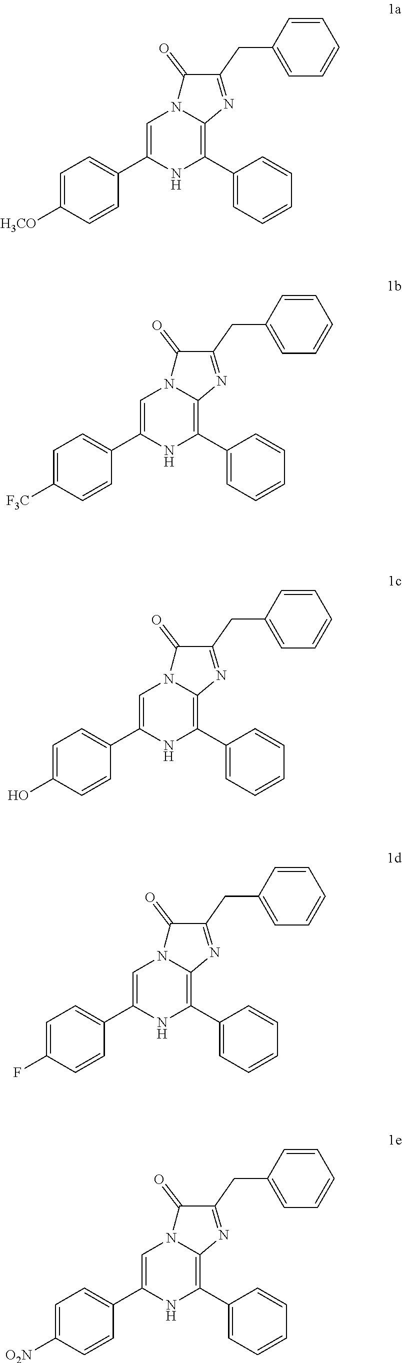 [SMILES: COc1ccc(-c2cn3c(=O)c(Cc4ccccc4)nc-3c(-c3ccccc3)[nH]2)cc1 ; O=c1c(Cc2ccccc2)nc2c(-c3ccccc3)[nH]c(-c3ccc(C(F)(F)F)cc3)cn1-2 ; O=c1c(Cc2ccccc2)nc2c(-c3ccccc3)[nH]c(-c3ccc(O)cc3)cn1-2 ; O=c1c(Cc2ccccc2)nc2c(-c3ccccc3)[nH]c(-c3ccc(F)cc3)cn1-2 ; O=c1c(Cc2ccccc2)nc2c(-c3ccccc3)[nH]c(-c3ccc([N+](=O)[O-])cc3)cn1-2]

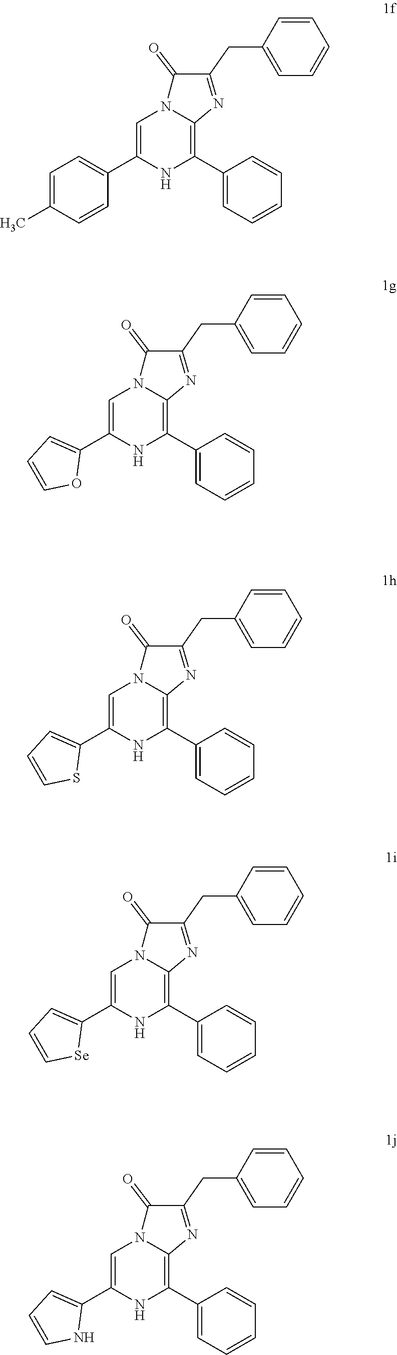 [SMILES: Cc1ccc(-c2cn3c(=O)c(Cc4ccccc4)nc-3c(-c3ccccc3)[nH]2)cc1 ; O=c1c(Cc2ccccc2)nc2c(-c3ccccc3)[nH]c(-c3ccco3)cn1-2 ; O=c1c(Cc2ccccc2)nc2c(-c3ccccc3)[nH]c(-c3cccs3)cn1-2 ; O=c1c(Cc2ccccc2)nc2c(-c3ccccc3)[nH]c(-c3ccc[se]3)cn1-2 ; O=c1c(Cc2ccccc2)nc2c(-c3ccccc3)[nH]c(-c3ccc[nH]3)cn1-2]

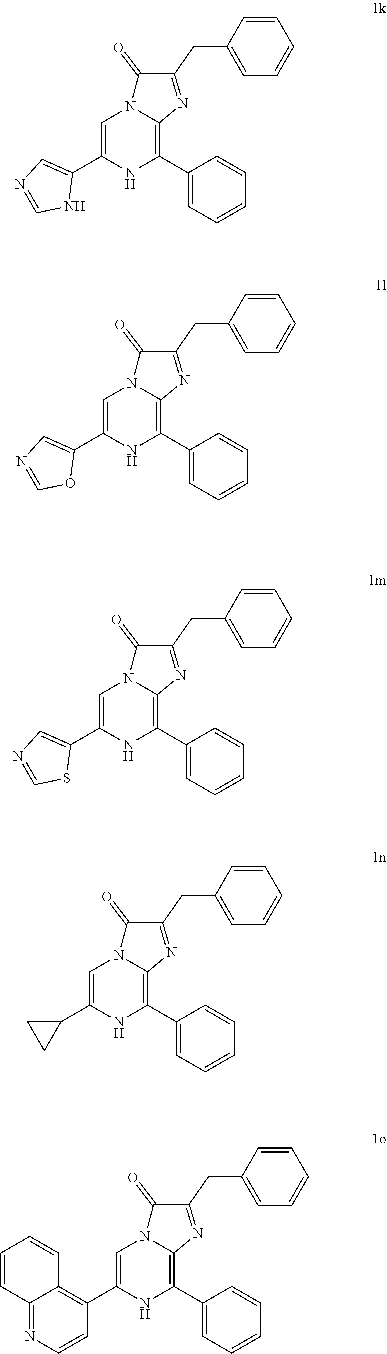 [SMILES: O=c1c(Cc2ccccc2)nc2c(-c3ccccc3)[nH]c(-c3cnc[nH]3)cn1-2 ; O=c1c(Cc2ccccc2)nc2c(-c3ccccc3)[nH]c(-c3cnco3)cn1-2 ; O=c1c(Cc2ccccc2)nc2c(-c3ccccc3)[nH]c(-c3cncs3)cn1-2 ; O=c1c(Cc2ccccc2)nc2c(-c3ccccc3)[nH]c(C3CC3)cn1-2 ; O=c1c(Cc2ccccc2)nc2c(-c3ccccc3)[nH]c(-c3ccnc4ccccc34)cn1-2]

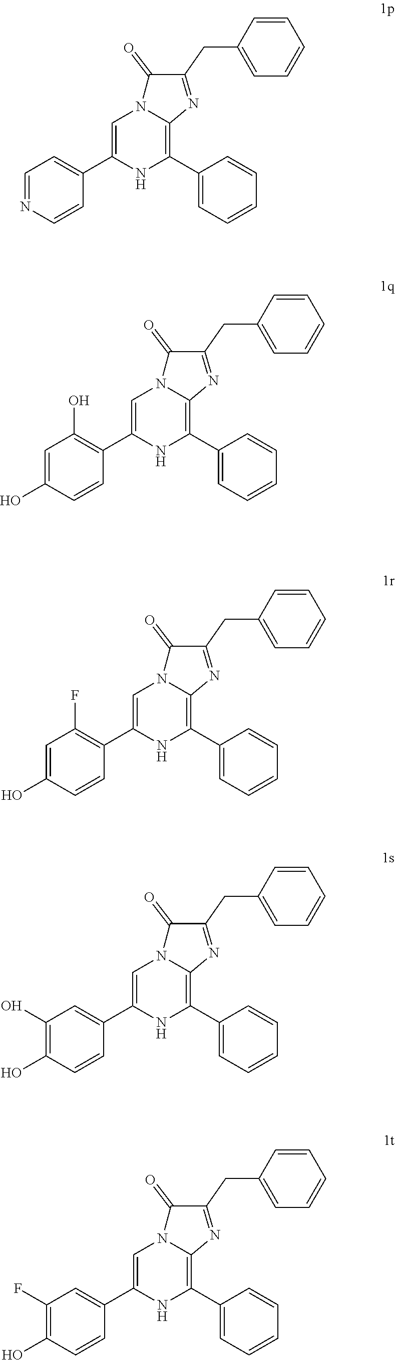 [SMILES: O=c1c(Cc2ccccc2)nc2c(-c3ccccc3)[nH]c(-c3ccncc3)cn1-2 ; O=c1c(Cc2ccccc2)nc2c(-c3ccccc3)[nH]c(-c3ccc(O)cc3O)cn1-2 ; O=c1c(Cc2ccccc2)nc2c(-c3ccccc3)[nH]c(-c3ccc(O)cc3F)cn1-2 ; O=c1c(Cc2ccccc2)nc2c(-c3ccccc3)[nH]c(-c3ccc(O)c(O)c3)cn1-2 ; O=c1c(Cc2ccccc2)nc2c(-c3ccccc3)[nH]c(-c3ccc(O)c(F)c3)cn1-2]

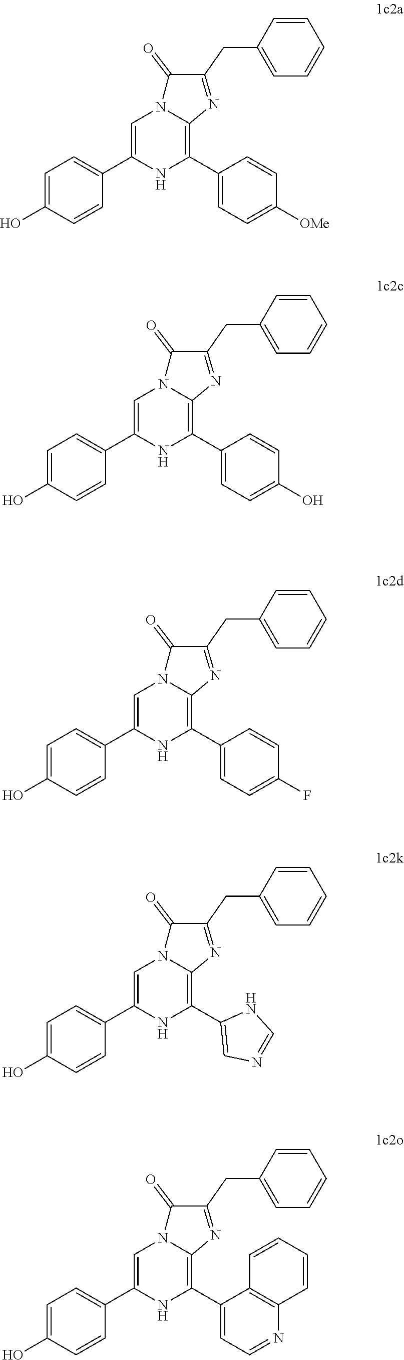 [SMILES: COc1ccc(-c2[nH]c(-c3ccc(O)cc3)cn3c(=O)c(Cc4ccccc4)nc2-3)cc1 ; O=c1c(Cc2ccccc2)nc2c(-c3ccc(O)cc3)[nH]c(-c3ccc(O)cc3)cn1-2 ; O=c1c(Cc2ccccc2)nc2c(-c3ccc(F)cc3)[nH]c(-c3ccc(O)cc3)cn1-2 ; O=c1c(Cc2ccccc2)nc2c(-c3cnc[nH]3)[nH]c(-c3ccc(O)cc3)cn1-2 ; O=c1c(Cc2ccccc2)nc2c(-c3ccnc4ccccc34)[nH]c(-c3ccc(O)cc3)cn1-2]

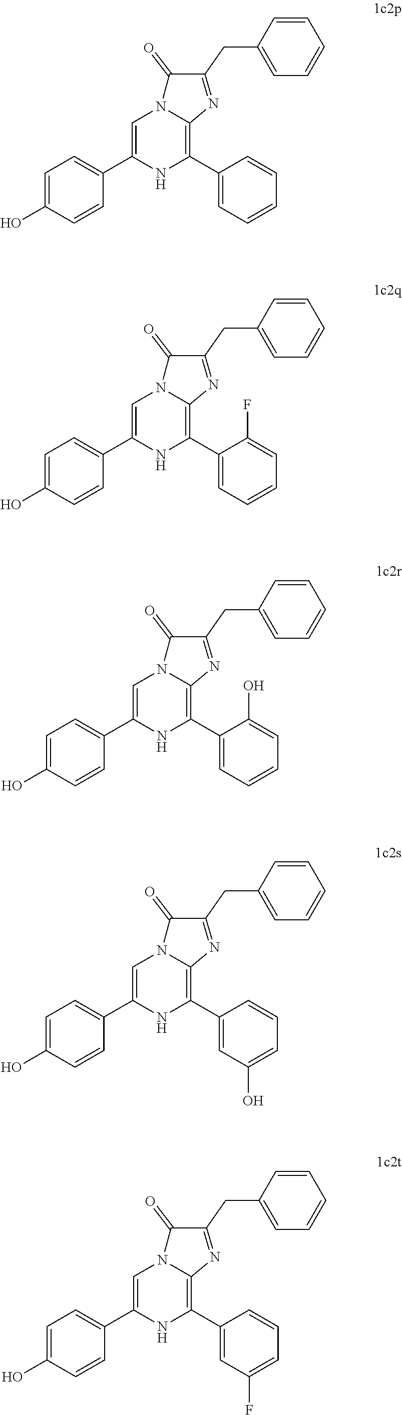 [SMILES: O=c1c(Cc2ccccc2)nc2c(-c3ccccc3)[nH]c(-c3ccc(O)cc3)cn1-2 ; O=c1c(Cc2ccccc2)nc2c(-c3ccccc3F)[nH]c(-c3ccc(O)cc3)cn1-2 ; O=c1c(Cc2ccccc2)nc2c(-c3ccccc3O)[nH]c(-c3ccc(O)cc3)cn1-2 ; O=c1c(Cc2ccccc2)nc2c(-c3cccc(O)c3)[nH]c(-c3ccc(O)cc3)cn1-2 ; O=c1c(Cc2ccccc2)nc2c(-c3cccc(F)c3)[nH]c(-c3ccc(O)cc3)cn1-2]

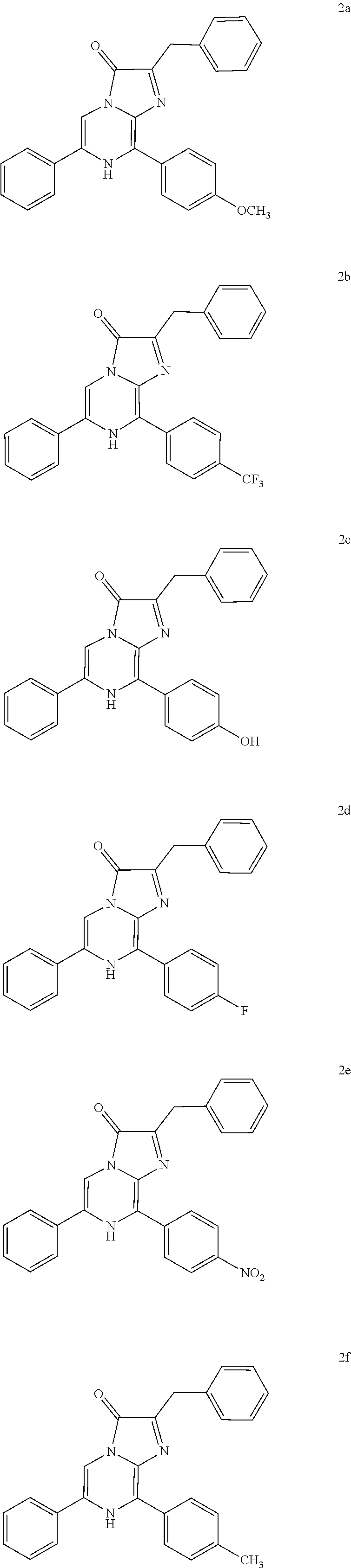 [SMILES: COc1ccc(-c2[nH]c(-c3ccccc3)cn3c(=O)c(Cc4ccccc4)nc2-3)cc1 ; O=c1c(Cc2ccccc2)nc2c(-c3ccc(C(F)(F)F)cc3)[nH]c(-c3ccccc3)cn1-2 ; O=c1c(Cc2ccccc2)nc2c(-c3ccc(O)cc3)[nH]c(-c3ccccc3)cn1-2 ; O=c1c(Cc2ccccc2)nc2c(-c3ccc(F)cc3)[nH]c(-c3ccccc3)cn1-2 ; O=c1c(Cc2ccccc2)nc2c(-c3ccc([N+](=O)[O-])cc3)[nH]c(-c3ccccc3)cn1-2 ; Cc1ccc(-c2[nH]c(-c3ccccc3)cn3c(=O)c(Cc4ccccc4)nc2-3)cc1]

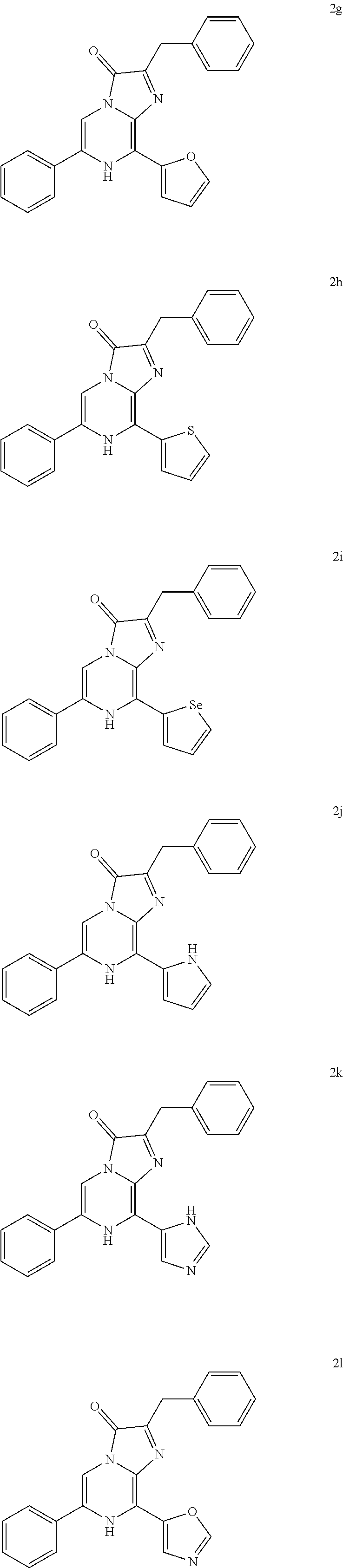 [SMILES: O=c1c(Cc2ccccc2)nc2c(-c3ccco3)[nH]c(-c3ccccc3)cn1-2 ; O=c1c(Cc2ccccc2)nc2c(-c3cccs3)[nH]c(-c3ccccc3)cn1-2 ; O=c1c(Cc2ccccc2)nc2c(-c3ccc[se]3)[nH]c(-c3ccccc3)cn1-2 ; O=c1c(Cc2ccccc2)nc2c(-c3ccc[nH]3)[nH]c(-c3ccccc3)cn1-2 ; O=c1c(Cc2ccccc2)nc2c(-c3cnc[nH]3)[nH]c(-c3ccccc3)cn1-2 ; O=c1c(Cc2ccccc2)nc2c(-c3cnco3)[nH]c(-c3ccccc3)cn1-2]

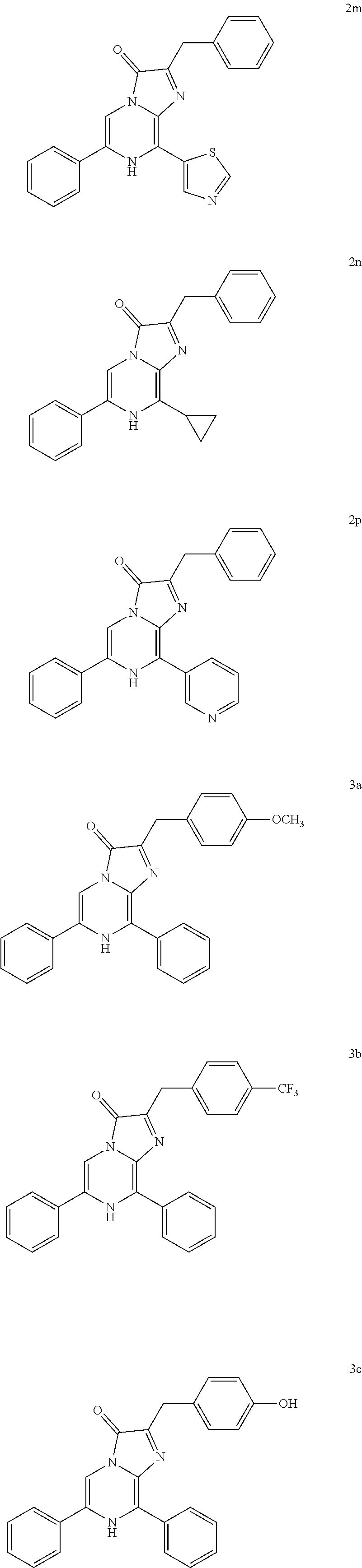 [SMILES: O=c1c(Cc2ccccc2)nc2c(-c3cncs3)[nH]c(-c3ccccc3)cn1-2 ; O=c1c(Cc2ccccc2)nc2c(C3CC3)[nH]c(-c3ccccc3)cn1-2 ; O=c1c(Cc2ccccc2)nc2c(-c3cccnc3)[nH]c(-c3ccccc3)cn1-2 ; COc1ccc(Cc2nc3c(-c4ccccc4)[nH]c(-c4ccccc4)cn-3c2=O)cc1 ; O=c1c(Cc2ccc(C(F)(F)F)cc2)nc2c(-c3ccccc3)[nH]c(-c3ccccc3)cn1-2 ; O=c1c(Cc2ccc(O)cc2)nc2c(-c3ccccc3)[nH]c(-c3ccccc3)cn1-2]

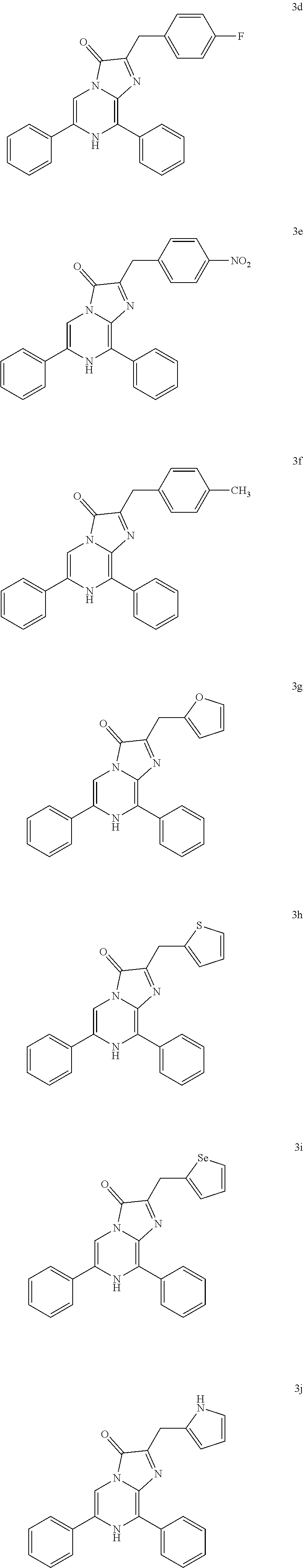

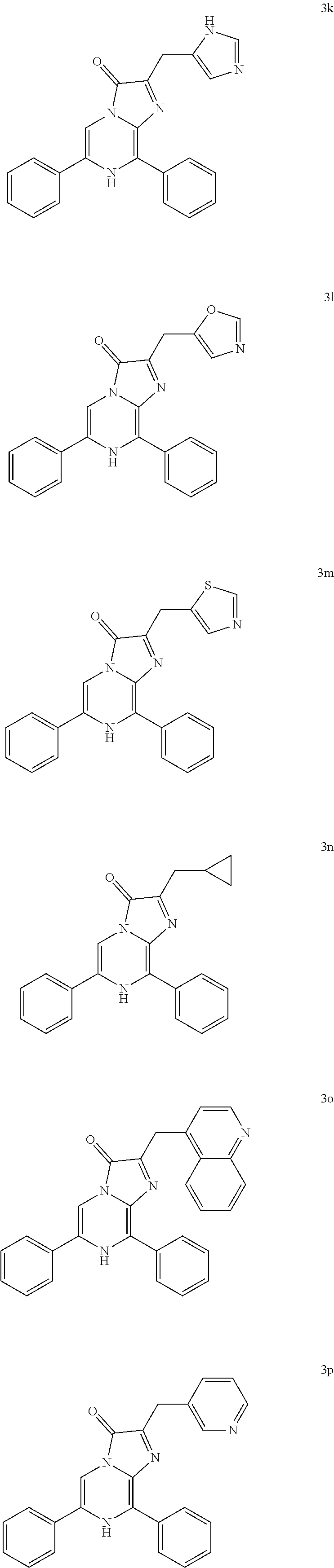 [SMILES: O=c1c(Cc2cnc[nH]2)nc2c(-c3ccccc3)[nH]c(-c3ccccc3)cn1-2 ; O=c1c(Cc2cnco2)nc2c(-c3ccccc3)[nH]c(-c3ccccc3)cn1-2 ; O=c1c(Cc2cncs2)nc2c(-c3ccccc3)[nH]c(-c3ccccc3)cn1-2 ; O=c1c(CC2CC2)nc2c(-c3ccccc3)[nH]c(-c3ccccc3)cn1-2 ; O=c1c(Cc2ccnc3ccccc23)nc2c(-c3ccccc3)[nH]c(-c3ccccc3)cn1-2 ; O=c1c(Cc2cccnc2)nc2c(-c3ccccc3)[nH]c(-c3ccccc3)cn1-2]

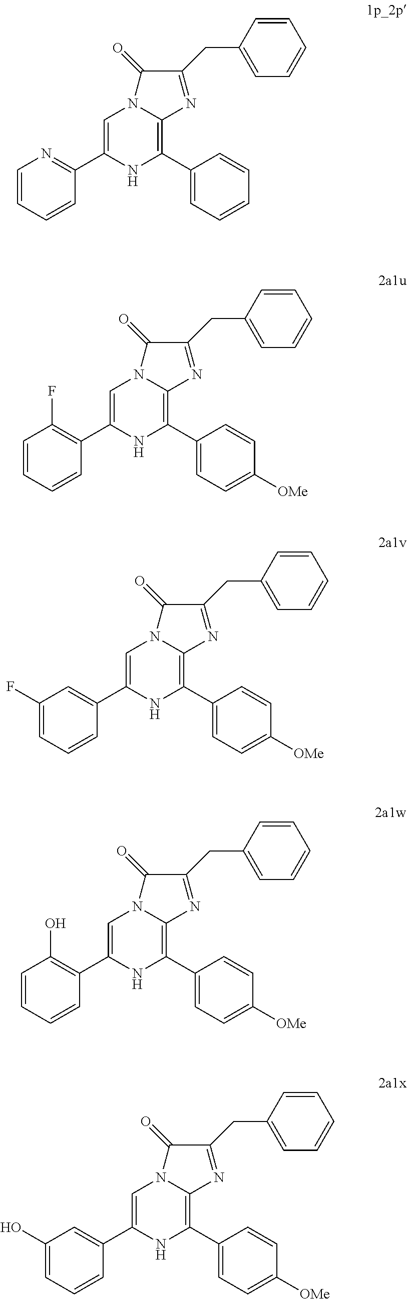 [SMILES: O=c1c(Cc2ccccc2)nc2c(-c3ccccc3)[nH]c(-c3ccccn3)cn1-2 ; COc1ccc(-c2[nH]c(-c3ccccc3F)cn3c(=O)c(Cc4ccccc4)nc2-3)cc1 ; COc1ccc(-c2[nH]c(-c3cccc(F)c3)cn3c(=O)c(Cc4ccccc4)nc2-3)cc1 ; COc1ccc(-c2[nH]c(-c3ccccc3O)cn3c(=O)c(Cc4ccccc4)nc2-3)cc1 ; COc1ccc(-c2[nH]c(-c3cccc(O)c3)cn3c(=O)c(Cc4ccccc4)nc2-3)cc1]

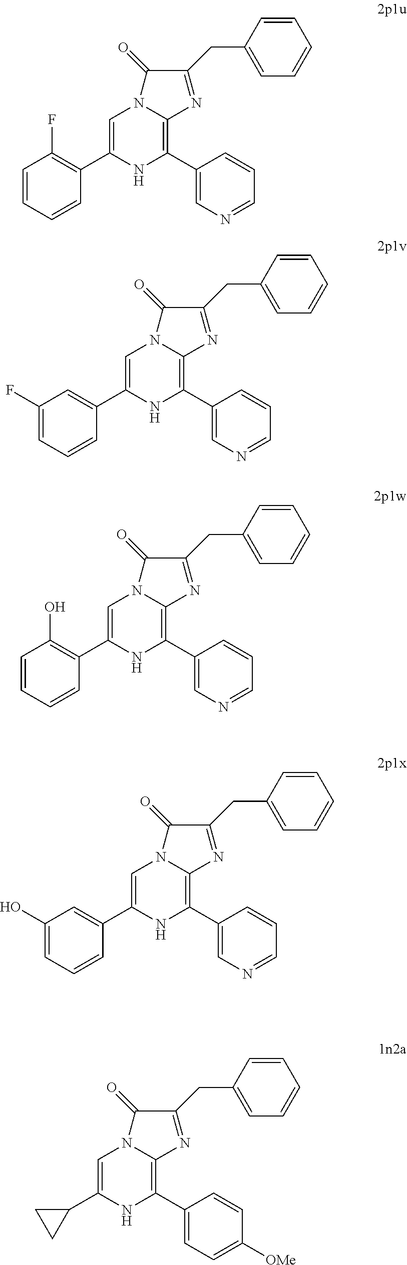 [SMILES: O=c1c(Cc2ccccc2)nc2c(-c3cccnc3)[nH]c(-c3ccccc3F)cn1-2 ; O=c1c(Cc2ccccc2)nc2c(-c3cccnc3)[nH]c(-c3cccc(F)c3)cn1-2 ; O=c1c(Cc2ccccc2)nc2c(-c3cccnc3)[nH]c(-c3ccccc3O)cn1-2 ; O=c1c(Cc2ccccc2)nc2c(-c3cccnc3)[nH]c(-c3cccc(O)c3)cn1-2 ; COc1ccc(-c2[nH]c(C3CC3)cn3c(=O)c(Cc4ccccc4)nc2-3)cc1]

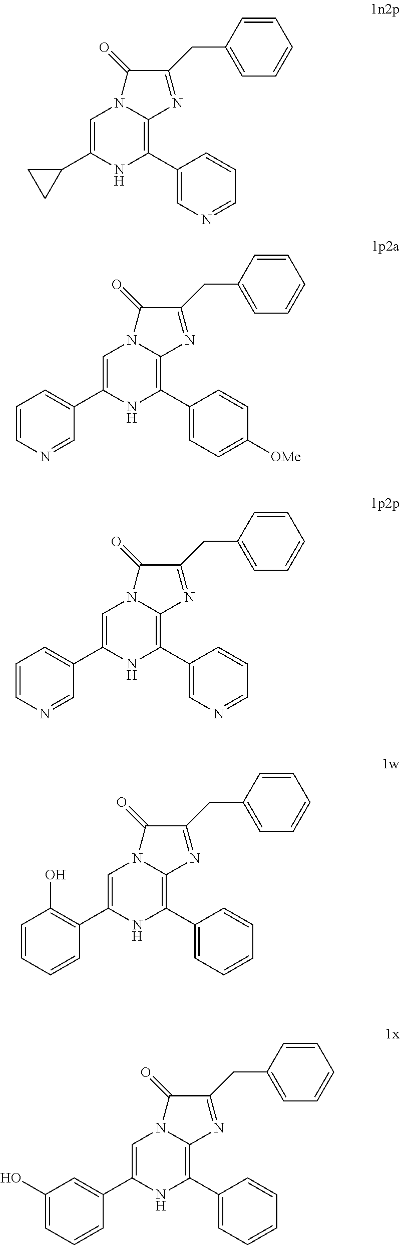 [SMILES: O=c1c(Cc2ccccc2)nc2c(-c3cccnc3)[nH]c(C3CC3)cn1-2 ; COc1ccc(-c2[nH]c(-c3cccnc3)cn3c(=O)c(Cc4ccccc4)nc2-3)cc1 ; O=c1c(Cc2ccccc2)nc2c(-c3cccnc3)[nH]c(-c3cccnc3)cn1-2 ; O=c1c(Cc2ccccc2)nc2c(-c3ccccc3)[nH]c(-c3ccccc3O)cn1-2 ; O=c1c(Cc2ccccc2)nc2c(-c3ccccc3)[nH]c(-c3cccc(O)c3)cn1-2]

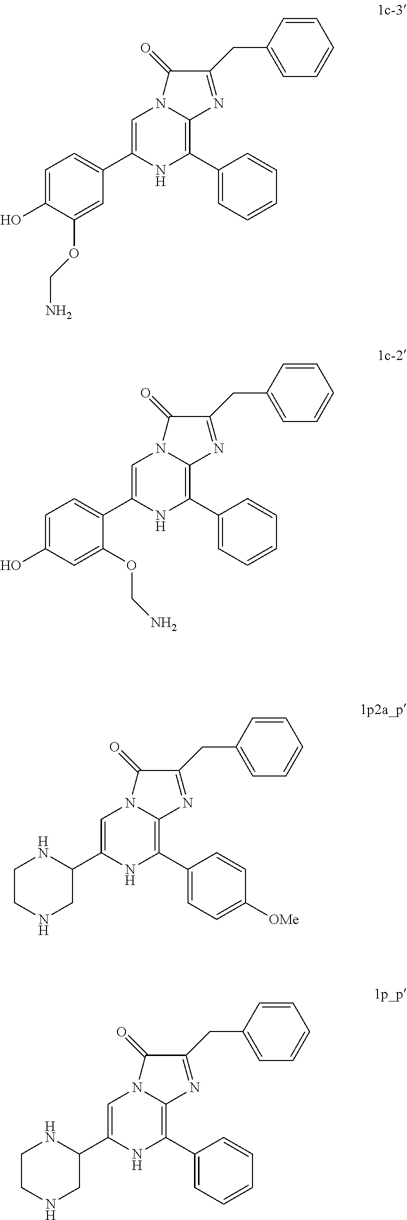 [SMILES: NCOc1cc(-c2cn3c(=O)c(Cc4ccccc4)nc-3c(-c3ccccc3)[nH]2)ccc1O ; NCOc1cc(O)ccc1-c1cn2c(=O)c(Cc3ccccc3)nc-2c(-c2ccccc2)[nH]1 ; COc1ccc(-c2[nH]c(C3CNCCN3)cn3c(=O)c(Cc4ccccc4)nc2-3)cc1 ; O=c1c(Cc2ccccc2)nc2c(-c3ccccc3)[nH]c(C3CNCCN3)cn1-2]

The compounds may exist as stereoisomers wherein asymmetric or chiral centers are present. The stereoisomers are “R “or “S “depending on the configuration of substituents around the chiral carbon atom. The terms “R” and “S” used herein are configurations as defined in IUPAC 1974 Recommendations for Section E, Fundamental Stereo chemistry, in Pure Appl. Chem., 1976, 45: 13-30. The disclosure contemplates various stereoisomers and mixtures thereof, and these are specifically included within the scope of this invention. Stereoisomers include enantiomers and diastereomers and mixtures of enantiomers or diastereomers. Individual stereoisomers of the compounds may be prepared synthetically from commercially available starting materials, which contain asymmetric or chiral centers or by preparation of racemic mixtures followed by methods of resolution well—known to those of ordinary skill in the art. These methods of resolution are exemplified by (1) attachment of a mixture of enantiomers to a chiral auxiliary, separation of the resulting mixture of diastereomers by recrystallization or chromatography, and optional liberation of the optically pure product from the auxiliary as described in Furniss, Hannaford, Smith, and Tatchell, “Vogels Text book of Practical Organic Chemistry”, 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compounds may possess tautomeric forms, as well as geometric isomers, and that these also constitute an aspect of the invention.

### Properties of the Compounds of Formula (I)

The compounds of Formula (I) are bioluminescent luciferin substrates, which can be used by luciferases or photoproteins to produce luminescence. The bioluminescent luciferin substrates as described herein may have improved properties such as better luminescence and better serum stability than diphenylterazine (DTZ). The bioluminescent luciferin substrates as described herein may also exhibit better solubility than DTZ.

As used herein, “luminescence” refers to the detectable electromagnetic radiation, generally, UV, IR or visible light radiation that is produced when the excited product of an exergic chemical process reverts to its ground state with the emission of light. Chemiluminescence is luminescence that results from a chemical reaction. Bioluminescence is chemiluminescence that results from a chemical reaction using biological molecules or synthetic versions or analogs thereof as substrates and/or enzymes.

As used herein, “bioluminescence,” which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme (luciferase) that is an oxygenase that acts on a substrate luciferin (a bioluminescence luciferin substrate) in the presence of molecular oxygen and transforms the substrate to an excited state, which upon return to a lower energy level releases the energy in the form of detectable electromagnetic radiation.

Luminescence is the light output of a luciferase under appropriate conditions, e.g., in the presence of a suitable substrate such as a diphenylterazine analog. The light output may be measured as an instantaneous or near-instantaneous measure of light output (which is sometimes referred to as “T=0” luminescence or “flash”) at the start of the luminescence reaction, which may be initiated upon addition of the luciferin substrate. The luminescence reaction in various embodiments is carried out in a solution. In other embodiments, the luminescence reaction is carried out on a solid support. The solution may contain a lysate, for example from the cells in a prokaryotic or eukaryotic expression system. In other embodiments, expression occurs in a cell-free system, or the luciferase protein is secreted into an extracellular medium, such that, in the latter case, it is not necessary to produce a lysate. In some embodiments, the reaction is started by injecting appropriate materials, e.g., diphenylterazine analog, buffer, etc., into a reaction chamber (e.g., a well of a multiwell plate such as a 96-well plate) containing the luminescent protein. In still other embodiments, the luciferase and/or diphenylterazine analogs (e.g., compounds of Formula (I)) are introduced into a host and measurements of luminescence are made on the host or a portion thereof, which can include a whole organism or cells, tissues, explants, or extracts thereof. The reaction chamber may be situated in a reading device which can measure the light output, e.g., using a luminometer or photomultiplier. The light output or luminescence may also be measured over time, for example in the same reaction chamber for a period of seconds, minutes, hours, etc. The light output or luminescence may be reported as the average over time, the half-life of decay of signal, the sum of the signal over a period of time, or the peak output. Luminescence may be measured in Relative Light Units (RLUs).

The terms “luminescence” and “bioluminescence” are used herein interchangeably.

### Synthesis of compounds of Formula (I)

Disclosed is a method of preparing bioluminescent luciferin substrates, where the luciferin substrate includes an imidazopyrazine backbone. In general, the method includes modifying positions C2, C6 or C8 of the imidazopyrazine backbone.

In certain embodiments, the method is carried out according to the following Scheme I:

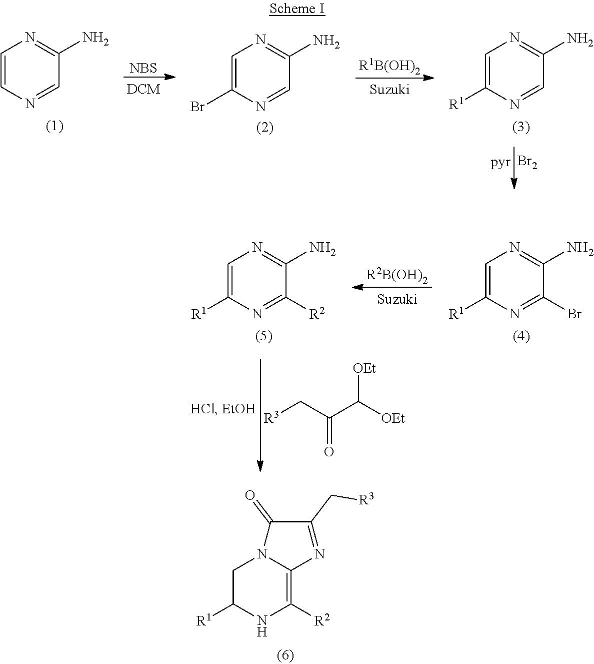

In Scheme I, R¹, R², and R³ are same as defined above; NBS is N-Bromosuccinimide; DCM is Dichloromethane; Br₂ is Bromine; Pyr is pyridine; EtOH is Ethyl alcohol or Ethanol; and R¹B(OH)₂ and R²B(OH)₂ are boronic acids.

The method of preparing compound of Formula (I) uses Suzuki coupling reaction as the key reaction.

### Utility

The compounds of the disclosure may be used in any way that luciferin substrates have been used. For example, they may be used in a bioluminogenic method which employs a luciferin substrate to detect one or more molecules in a sample, e.g., an enzyme, a cofactor for an enzymatic reaction, an enzyme substrate, an enzyme inhibitor, an enzyme activator, or OH radicals, or one or more conditions, e.g., redox conditions. The sample may include an animal (e.g., a vertebrate), a plant, a fungus, physiological fluid (e.g., blood, plasma, urine, mucous secretions), a cell, a cell lysate, a cell supernatant, or a purified fraction of a cell (e.g., a subcellular fraction). The presence, amount, spectral distribution, emission kinetics, or specific activity of such a molecule may be detected or quantified. The molecule may be detected or quantified in solution, including multiphasic solutions (e.g., emulsions or suspensions), or on solid supports (e.g., particles, capillaries, or assay vessels).

In certain aspects, the compounds of Formula (I) can be used for detecting luminescence in live cells. In some aspects, a luciferase can be expressed in cells (as a reporter or otherwise), and the cells treated with a bioluminescent luciferin substrate (e.g., a compound of Formula (I)), which will permeate cells in culture, react with the luciferase and generate luminescence. In some embodiments, the compounds of Formula (I) containing chemical modifications known to increase the stability of native diphenylterazine in media can be synthesized and used for more robust, live cell luciferase-based reporter assays. In still other aspects, a sample (including cells, tissues, animals, etc.) containing a luciferase and a compound of Formula (I) may be assayed using various microscopy and imaging techniques.

### Luciferin Substrates

The present disclosure provides luciferin substrates. The luciferin substrates may be compounds of formula (Ia):

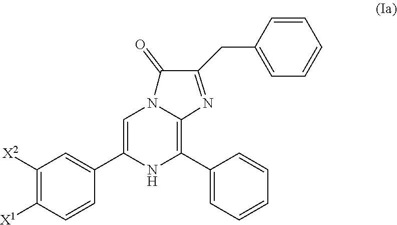

or a stereoisomer, a tautomer or a salt thereof, wherein:
X¹-X² are independently selected from a group consisting of: halogen, hydroxyl, haloalkyl, alkyl or nitro;
With proviso that:
when X² is hydrogen, then X¹ is selected from a group consisting of: haloalkyl, alkyl or nitro;
when X¹ is hydroxyl, then X² is selected from halogen.

In certain aspects, X² is hydrogen and X¹ is haloalkyl. The haloalkyl group includes straight or branched haloalkyl groups obtained by substituting one or more hydrogen atoms with halogen atoms, e.g., fluoromethyl, difluoromethyl, trifluoromethyl, chloromethyl, and dichloromethyl. In certain aspects, X¹ is trifluoromethyl.

In certain aspects, X² is hydrogen and X¹ is alkyl. The alkyl group includes straight or branched alkyl groups, e.g., methyl, ethyl, n-propyl and isopropyl. In certain aspects X¹ is methyl.

In certain aspects, X² is hydrogen and X¹ is nitro. In certain aspects, X² is hydrogen and X¹ is halogen. In certain aspects, X² is selected from fluorine, chlorine, bromine or iodine. In certain aspects, X¹ is hydroxyl and X² is fluorine.

In certain aspects, the luciferin substrates may be compounds of Formula (Ib) is:

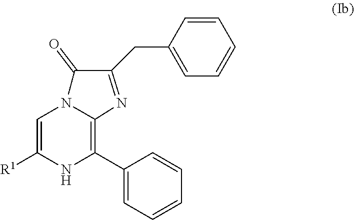

or a stereoisomer, a tautomer or a salt thereof, wherein:
   R¹ is selected from: a cycloalkyl, 5 membered heteroaryl having 1-3 ring heteroatoms independently selected from O, S, Se and N; and 10 membered heteroaryl having 1-3 ring heteroatoms independently selected from O, S, Se and N;
or R¹ is selected from:

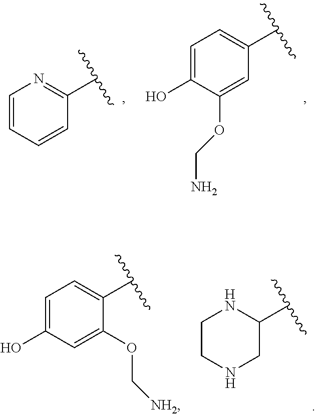 [SMILES: *c1ccccn1 ; *c1ccc(O)c(OCN)c1 ; *c1ccc(O)cc1OCN ; *C1CNCCN1]

In certain aspects, R¹ is a cycloalkyl. The cycloalkyl group includes cycloalkyl groups, e.g., cyclopropyl, cyclobutyl, cyclopentyl and cyclohexyl. In certain aspects, R¹ is a cyclopropyl.

In certain aspects, R¹ is a 5 membered heteroaryl having 1-3 ring heteroatoms independently selected from O, S, Se and N. In certain aspects, R¹ is selected from pyrrole, furan, thiophene, selenophene, imidazole, thiazole and oxazole. In certain aspects, R¹ is a 10 membered heteroaryl having 1-3 ring heteroatoms independently selected from O, S, Se and N. In certain aspects, R¹ is quinoline.

In certain aspects, R¹ is

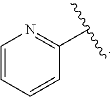 [SMILES: *c1ccccn1]

In certain aspects, R¹ is

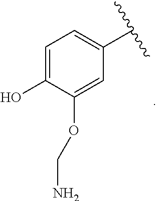 [SMILES: *c1ccc(O)c(OCN)c1]

In certain aspects, R¹ is

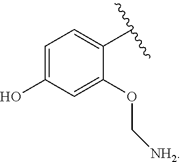 [SMILES: *c1ccc(O)cc1OCN]

In certain aspects, R¹ is H

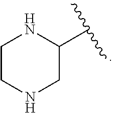 [SMILES: *C1CNCCN1]

In certain aspects, the luciferin substrates may be compounds of Formula (Ic):

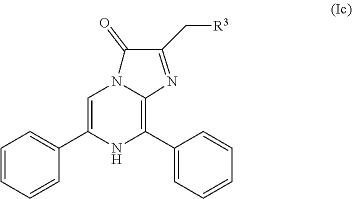

or a stereoisomer, a tautomer or a salt thereof, wherein:

R³ is selected from: a cycloalkyl, 5 membered heteroaryl having 1-3 ring heteroatoms independently selected from O, S, Se and N; and 10 membered heteroaryl having 1-3 ring heteroatoms independently selected from O, S, Se and N.

In certain aspects, R³ is a cycloalkyl. The cycloalkyl group includes cycloalkyl groups, e.g., cyclopropyl, cyclobutyl, cyclopentyl and cyclohexyl. In certain aspects, R³ is a cyclopropyl.

In certain aspects, R³ is a 5 membered heteroaryl having 1-3 ring heteroatoms independently selected from O, S, Se and N. In certain aspects, R³ is selected from pyrrole, furan, thiophene, selenophene, imidazole, thiazole and oxazole. In certain aspects, R³ is a 10 membered heteroaryl having 1-3 ring heteroatoms independently selected from O, S, Se and N. In certain aspects, R³ is quinoline.

In certain aspects, the luciferin substrates may be compounds of Formula (Id):

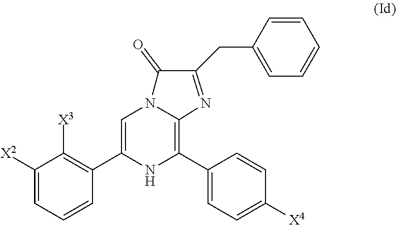

or a stereoisomer, a tautomer or a salt thereof, wherein:
X²-X³ are independently selected from: hydrogen, halogen, or hydroxy,
X⁴ is alkoxy;
with proviso that either one of X²-X³ is hydrogen.

In certain aspects, X² is hydrogen and X³ is a halogen. In certain aspects, X² is hydrogen and X³ is selected from fluorine, chlorine, bromine or iodine. In certain aspects, X² is hydrogen and X³ is fluorine. In certain aspects, X² is hydrogen and X³ is hydroxy. In certain aspects, X³ is hydrogen and X² is a halogen. In certain aspects, X³ is hydrogen and X² is selected from fluorine, chlorine, bromine or iodine. In certain aspects, X³ is hydrogen and X² is fluorine. In certain aspects, X³ is hydrogen and X² is hydroxy.

In certain aspects, the luciferin substrates may be compounds of Formula (Ie) is:

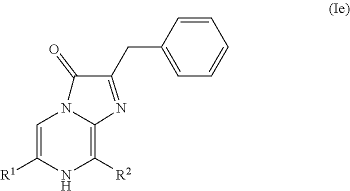

or a stereoisomer, a tautomer or a salt thereof, wherein:
   R¹ is selected from:

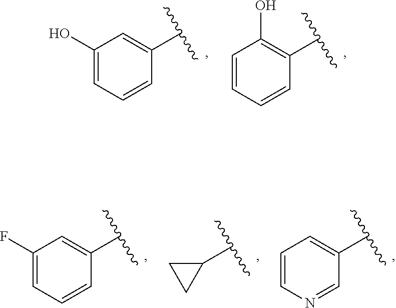

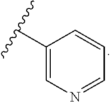 [SMILES: *c1cccnc1]

or R² is selected from:

In certain aspects, R¹ is

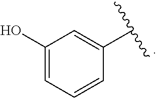 [SMILES: *c1cccc(O)c1]

In certain aspects, R¹ is

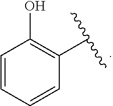 [SMILES: *c1ccccc1O]

In certain aspects, R¹ is

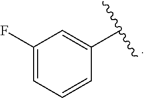 [SMILES: *c1cccc(F)c1]

In certain aspects, R¹ is

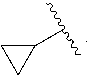 [SMILES: *C1CC1]

In certain aspects, R¹ is

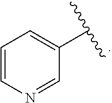 [SMILES: *c1cccnc1]

In certain aspects, the luciferin substrates may be compounds selected from:

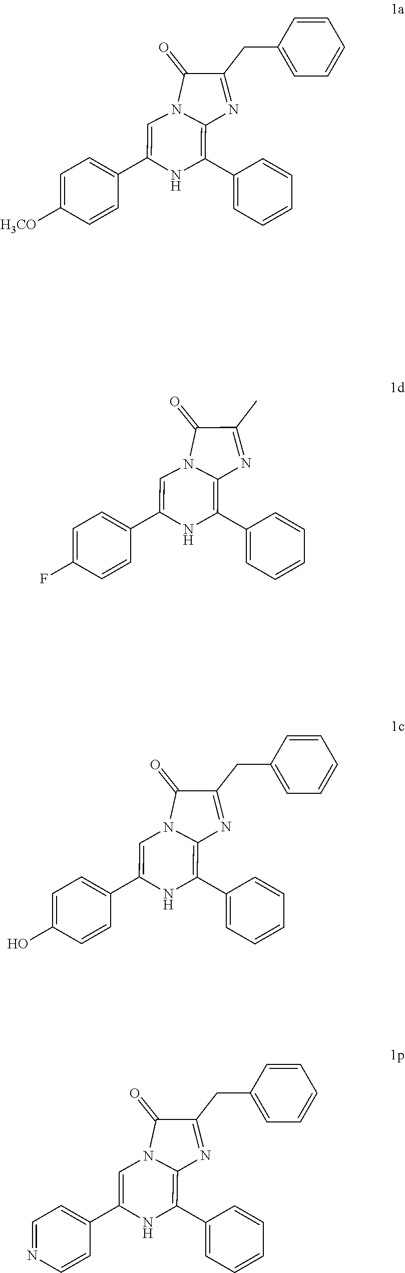 [SMILES: COc1ccc(-c2cn3c(=O)c(Cc4ccccc4)nc-3c(-c3ccccc3)[nH]2)cc1 ; Cc1nc2c(-c3ccccc3)[nH]c(-c3ccc(F)cc3)cn-2c1=O ; O=c1c(Cc2ccccc2)nc2c(-c3ccccc3)[nH]c(-c3ccc(O)cc3)cn1-2 ; O=c1c(Cc2ccccc2)nc2c(-c3ccccc3)[nH]c(-c3ccncc3)cn1-2]

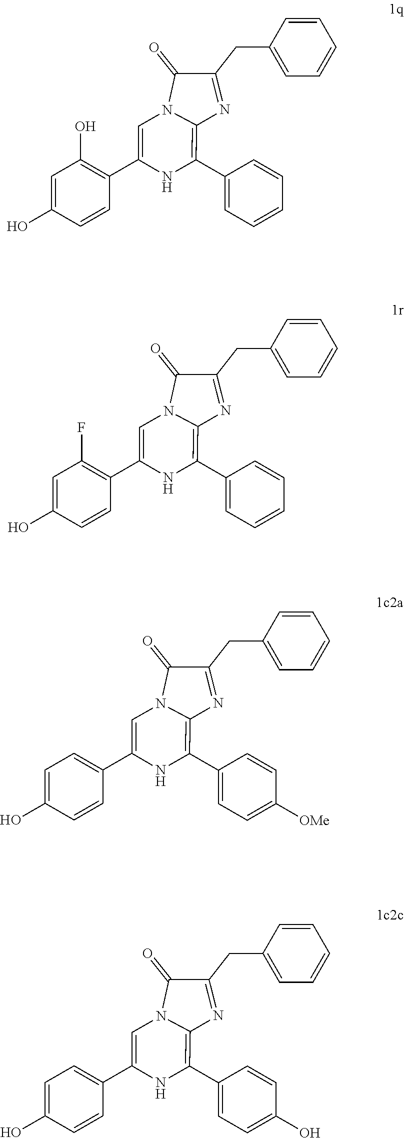 [SMILES: O=c1c(Cc2ccccc2)nc2c(-c3ccccc3)[nH]c(-c3ccc(O)cc3O)cn1-2 ; O=c1c(Cc2ccccc2)nc2c(-c3ccccc3)[nH]c(-c3ccc(O)cc3F)cn1-2 ; COc1ccc(-c2[nH]c(-c3ccc(O)cc3)cn3c(=O)c(Cc4ccccc4)nc2-3)cc1 ; O=c1c(Cc2ccccc2)nc2c(-c3ccc(O)cc3)[nH]c(-c3ccc(O)cc3)cn1-2]

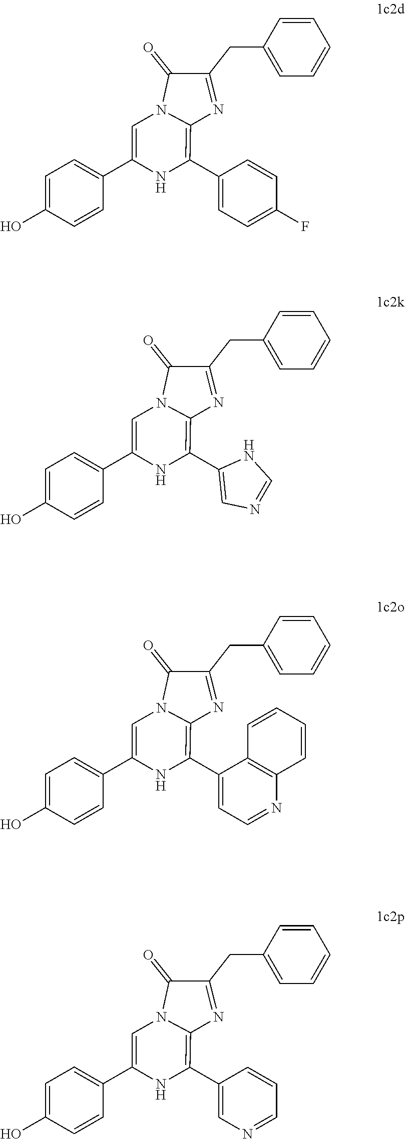 [SMILES: O=c1c(Cc2ccccc2)nc2c(-c3ccc(F)cc3)[nH]c(-c3ccc(O)cc3)cn1-2 ; O=c1c(Cc2ccccc2)nc2c(-c3cnc[nH]3)[nH]c(-c3ccc(O)cc3)cn1-2 ; O=c1c(Cc2ccccc2)nc2c(-c3ccnc4ccccc34)[nH]c(-c3ccc(O)cc3)cn1-2 ; O=c1c(Cc2ccccc2)nc2c(-c3cccnc3)[nH]c(-c3ccc(O)cc3)cn1-2]

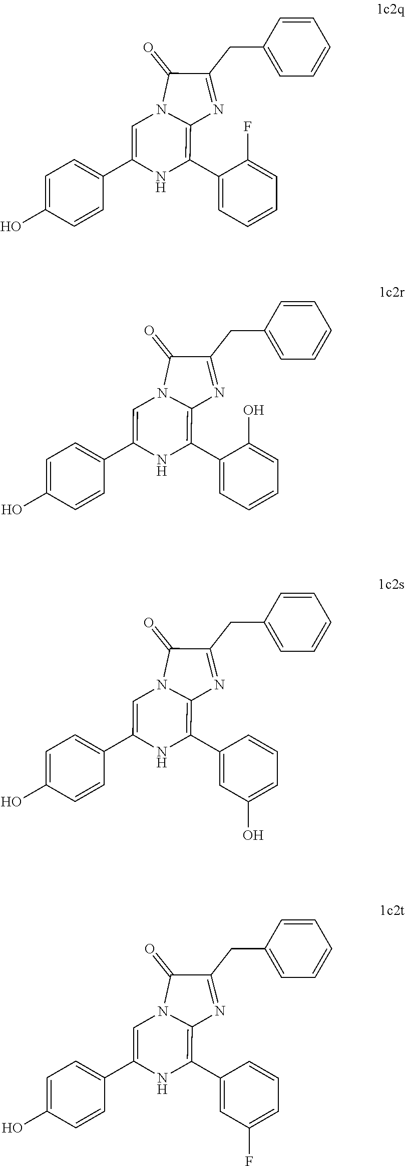 [SMILES: O=c1c(Cc2ccccc2)nc2c(-c3ccccc3F)[nH]c(-c3ccc(O)cc3)cn1-2 ; O=c1c(Cc2ccccc2)nc2c(-c3ccccc3O)[nH]c(-c3ccc(O)cc3)cn1-2 ; O=c1c(Cc2ccccc2)nc2c(-c3cccc(O)c3)[nH]c(-c3ccc(O)cc3)cn1-2 ; O=c1c(Cc2ccccc2)nc2c(-c3cccc(F)c3)[nH]c(-c3ccc(O)cc3)cn1-2]

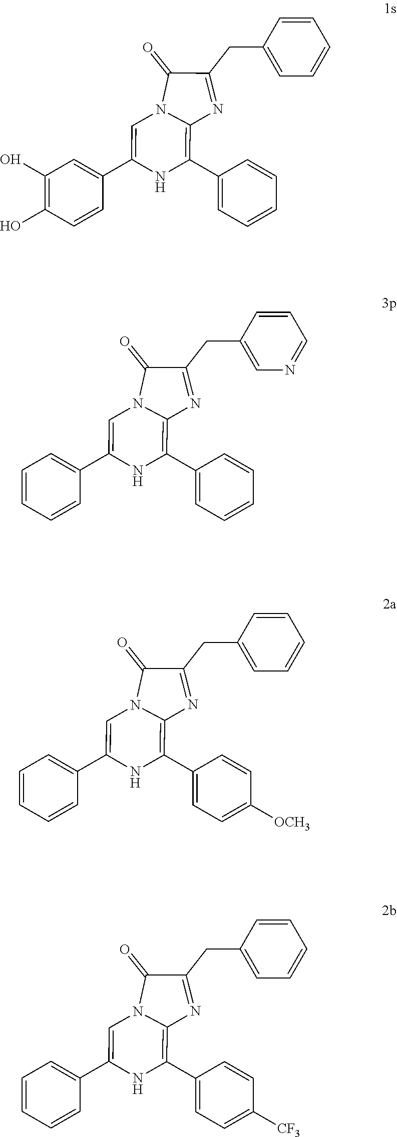 [SMILES: O=c1c(Cc2ccccc2)nc2c(-c3ccccc3)[nH]c(-c3ccc(O)c(O)c3)cn1-2 ; O=c1c(Cc2cccnc2)nc2c(-c3ccccc3)[nH]c(-c3ccccc3)cn1-2 ; COc1ccc(-c2[nH]c(-c3ccccc3)cn3c(=O)c(Cc4ccccc4)nc2-3)cc1 ; O=c1c(Cc2ccccc2)nc2c(-c3ccc(C(F)(F)F)cc3)[nH]c(-c3ccccc3)cn1-2]

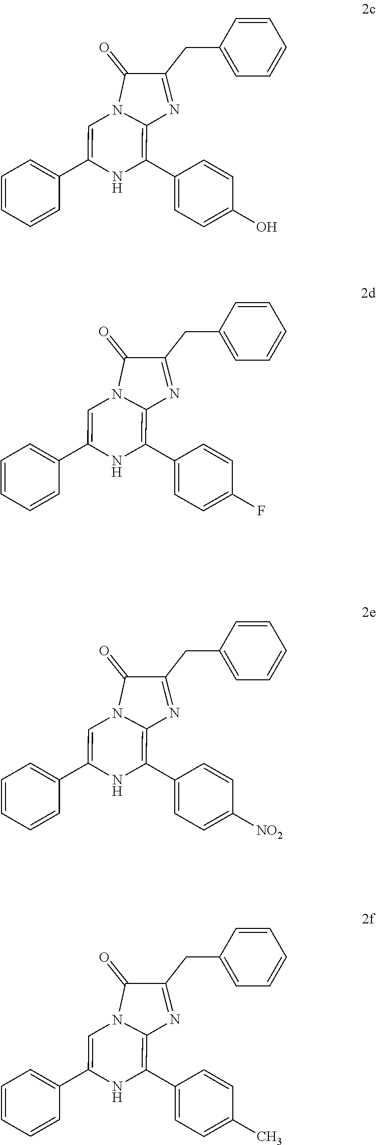 [SMILES: O=c1c(Cc2ccccc2)nc2c(-c3ccc(O)cc3)[nH]c(-c3ccccc3)cn1-2 ; O=c1c(Cc2ccccc2)nc2c(-c3ccc(F)cc3)[nH]c(-c3ccccc3)cn1-2 ; O=c1c(Cc2ccccc2)nc2c(-c3ccc([N+](=O)[O-])cc3)[nH]c(-c3ccccc3)cn1-2 ; Cc1ccc(-c2[nH]c(-c3ccccc3)cn3c(=O)c(Cc4ccccc4)nc2-3)cc1]

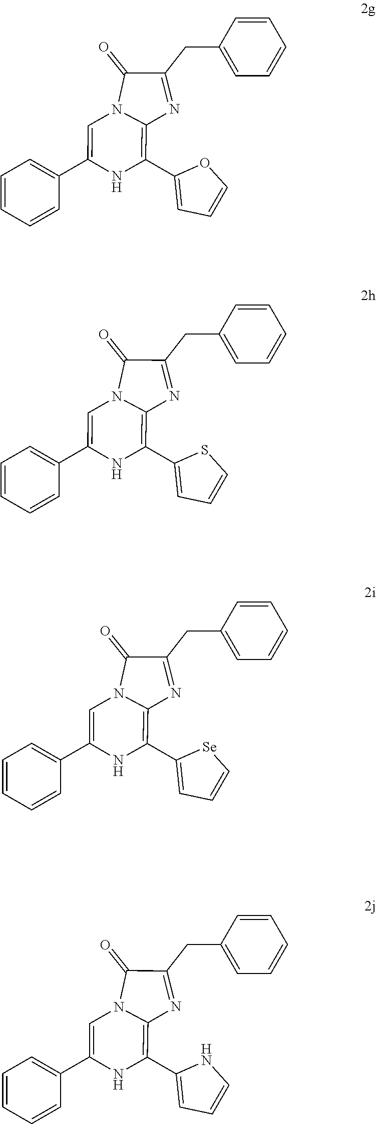 [SMILES: O=c1c(Cc2ccccc2)nc2c(-c3ccco3)[nH]c(-c3ccccc3)cn1-2 ; O=c1c(Cc2ccccc2)nc2c(-c3cccs3)[nH]c(-c3ccccc3)cn1-2 ; O=c1c(Cc2ccccc2)nc2c(-c3ccc[se]3)[nH]c(-c3ccccc3)cn1-2 ; O=c1c(Cc2ccccc2)nc2c(-c3ccc[nH]3)[nH]c(-c3ccccc3)cn1-2]

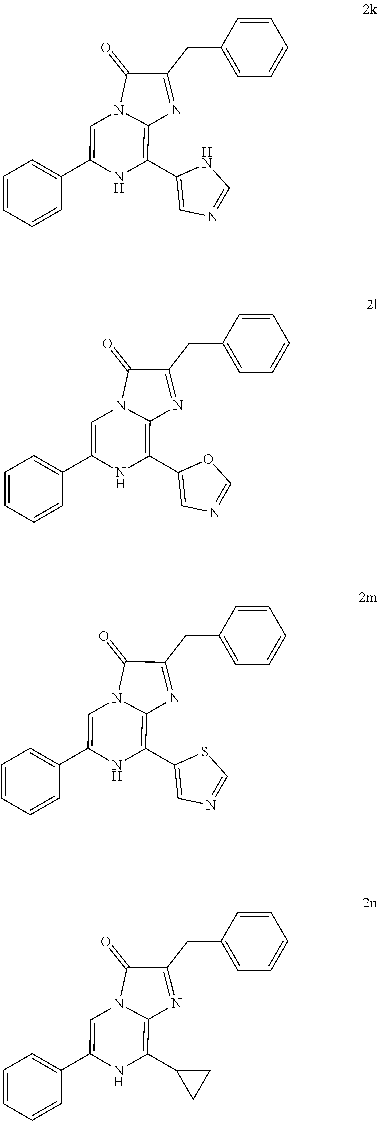 [SMILES: O=c1c(Cc2ccccc2)nc2c(-c3cnc[nH]3)[nH]c(-c3ccccc3)cn1-2 ; O=c1c(Cc2ccccc2)nc2c(-c3cnco3)[nH]c(-c3ccccc3)cn1-2 ; O=c1c(Cc2ccccc2)nc2c(-c3cncs3)[nH]c(-c3ccccc3)cn1-2 ; O=c1c(Cc2ccccc2)nc2c(C3CC3)[nH]c(-c3ccccc3)cn1-2]

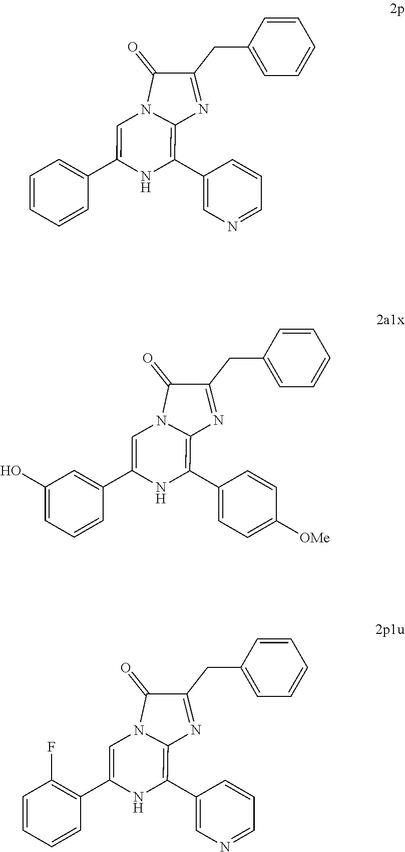 [SMILES: O=c1c(Cc2ccccc2)nc2c(-c3cccnc3)[nH]c(-c3ccccc3)cn1-2 ; COc1ccc(-c2[nH]c(-c3cccc(O)c3)cn3c(=O)c(Cc4ccccc4)nc2-3)cc1 ; O=c1c(Cc2ccccc2)nc2c(-c3cccnc3)[nH]c(-c3ccccc3F)cn1-2]

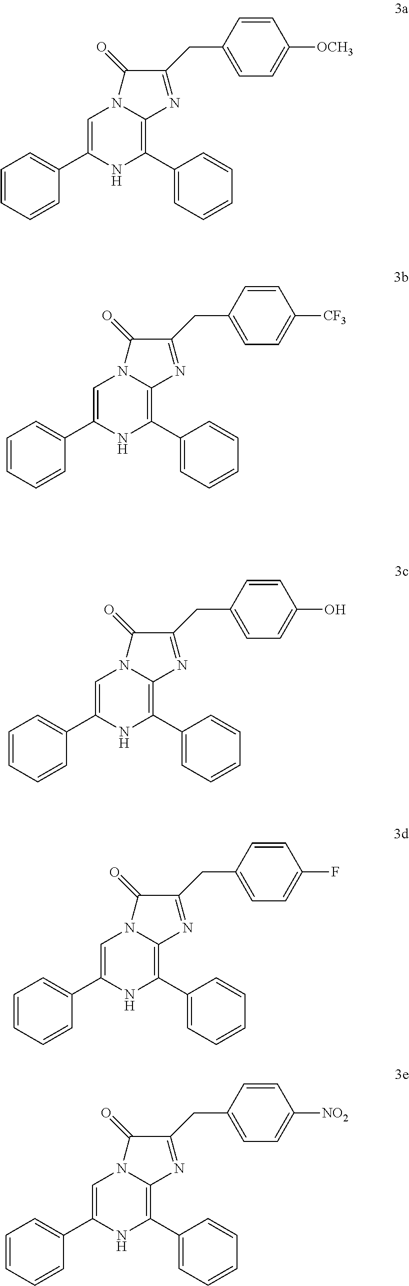 [SMILES: COc1ccc(Cc2nc3c(-c4ccccc4)[nH]c(-c4ccccc4)cn-3c2=O)cc1 ; O=c1c(Cc2ccc(C(F)(F)F)cc2)nc2c(-c3ccccc3)[nH]c(-c3ccccc3)cn1-2 ; O=c1c(Cc2ccc(O)cc2)nc2c(-c3ccccc3)[nH]c(-c3ccccc3)cn1-2 ; O=c1c(Cc2ccc(F)cc2)nc2c(-c3ccccc3)[nH]c(-c3ccccc3)cn1-2 ; O=c1c(Cc2ccc([N+](=O)[O-])cc2)nc2c(-c3ccccc3)[nH]c(-c3ccccc3)cn1-2]

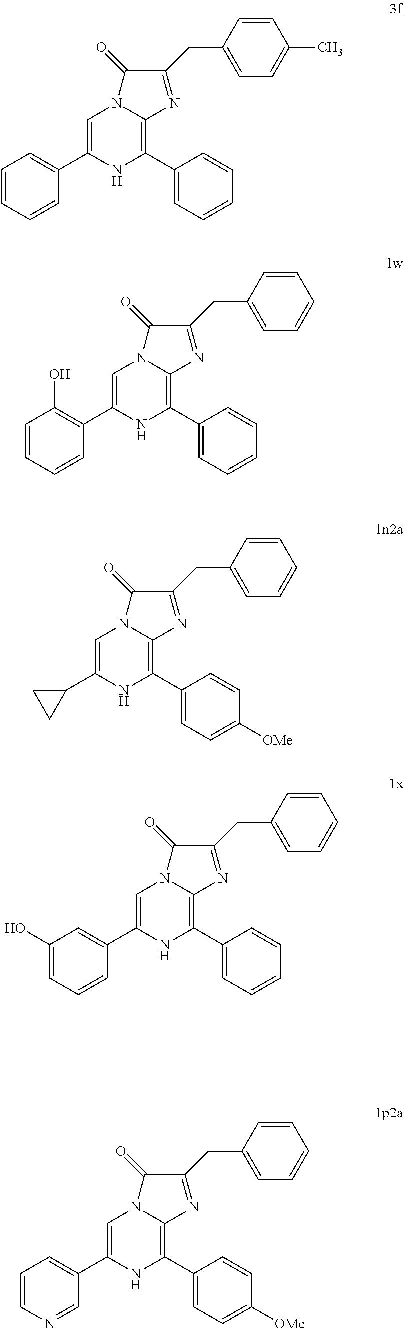 [SMILES: Cc1ccc(Cc2nc3c(-c4ccccc4)[nH]c(-c4ccccc4)cn-3c2=O)cc1 ; O=c1c(Cc2ccccc2)nc2c(-c3ccccc3)[nH]c(-c3ccccc3O)cn1-2 ; COc1ccc(-c2[nH]c(C3CC3)cn3c(=O)c(Cc4ccccc4)nc2-3)cc1 ; O=c1c(Cc2ccccc2)nc2c(-c3ccccc3)[nH]c(-c3cccc(O)c3)cn1-2 ; COc1ccc(-c2[nH]c(-c3cccnc3)cn3c(=O)c(Cc4ccccc4)nc2-3)cc1]

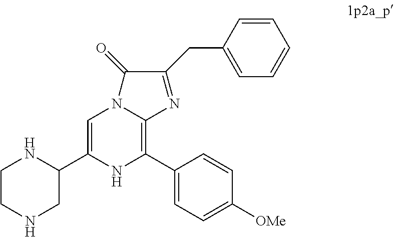 [SMILES: COc1ccc(-c2[nH]c(C3CNCCN3)cn3c(=O)c(Cc4ccccc4)nc2-3)cc1]

### Methods

Methods disclosed herein include use of a polypeptide having luciferase activity, as disclosed herein, for imaging cells expressing the polypeptide. In certain aspects, the polypeptide having luciferase activity may be expressed as a fusion protein for imaging cells expressing a protein of interest fused to the polypeptide. The polypeptides having luciferase activity, as disclosed herein, may be used for imaging live mammalian cells, e.g., a mammal.

Methods disclosed herein include use of the self-complementing multipartite protein having luciferase activity to assay for the detection of molecular interactions (e.g., transient association, stable association, complex formation, etc.) between a first moiety and a second moiety. The first and second moieties can be a peptide, a protein, a nucleic acid, a small molecule, etc. The first moiety may be conjugated to a first polypeptide component of the self-complementing multipartite and the second moiety conjugating the second polypeptide component to the other moiety, where the two components do not stably associate and produce no signal (e.g., substantially no signal) in the absence of the molecular interaction between the first and second moieties, but stably associate to form the self-complementing multipartite protein and produce a detectable (e.g., bioluminescent) signal upon interaction of the first and second moieties. In such embodiments, assembly of the self-complementing multipartite protein is operated by the molecular interaction of the first and second moieties. If the first and second moieties engage in a sufficiently stable interaction, the self-complementing multipartite protein having luciferase activity forms, and a bioluminescent signal is generated. If the first and second moieties fail to engage in a sufficiently stable interaction, the self-complementing multipartite protein having luciferase activity does not form, or only weakly forms, and a bioluminescent signal is not generated or is substantially reduced (e.g., substantially undetectable, essentially not detectable, differentially detectable as compared to a stable control signal, etc.). In some embodiments, the magnitude of the detectable bioluminescent signal is proportional (e.g., directly proportional) to the amount, strength, favorability, and/or stability of the molecular interactions between the first and second moieties. In certain aspects, the first moiety may be a protein and the second moiety may be a small molecule or vice versa. In certain aspects, the first moiety is a protein and is conjugated to the first component where the first component is larger than the second component and the second component is conjugated to a second moiety that is a small molecule or vice versa.

Methods disclosed herein include use of the self-complementing multipartite protein having luciferase activity to assay for the detection of molecular interactions (e.g., transient association, stable association, complex formation, etc.) between a first moiety and a second moiety. The method may involve use of a first polypeptide component and a second polypeptide component that can associate to form the self-complementing multipartite protein having luciferase activity, when either the first or the second or both components are conjugated to a moiety and do not associate when the moity(ies) are bound to another moiety. For example, the first polypeptide component may be fused to a first moiety and may associate with the second polypeptide component to form the self-complementing multipartite protein having luciferase activity. However, when a moiety, e.g., a ligand interacts with the first moiety, the first and second components can no longer associate to form the self-complementing multipartite protein having luciferase activity.

In some aspects, the interaction is detected in living cells, in vivo or in vitro, by detecting the bioluminescence signal emitted by the cells. In some embodiments, the interaction is detected outside a living cell, where the first and second components are secreted by the cell. In some embodiments, the interaction is detected in living organism, either inside the cells or inside tissues of the living organism.

In some aspects, an alteration in the interaction resulting from an alteration of the environment of the cells is detected by detecting a difference in the emitted bioluminescent signal relative to control cells absent the altered environment. In some embodiments, the altered environment is the result of adding or removing a molecule from the culture medium (e.g., a drug).

The polypeptides having luciferase activity as described herein, e.g., LuxSit-i variants, LuxSit-i variant derived self complementing multipaptite proteins, circularly permuted LuxSit-i and LuxSit-i variants are useful for many purposes including, but not limited to, detecting the amount or presence of a particular molecule (a biosensor), isolating a particular molecule, detecting conformational changes in a particular molecule, e.g., due to binding, phosphorylation or ionization, facilitating high or low throughput screening, detecting protein-protein, protein-DNA or other protein-based interactions, or selecting or evolving biosensors. For instance, a polypeptides having luciferase activity or a fusion thereof, is useful to detect, e.g., in an in vitro or cell-based assay, the amount, presence or activity of a particular kinase (for example, by inserting a kinase site into the protein), RNAi (e.g., by inserting a sequence suspected of being recognized by RNAi into a coding sequence for the protein, then monitoring reporter activity after addition of RNAi), or protease, such as one to detect the presence of a particular viral protease, which in turn is indicator of the presence of the virus, or an antibody; to screen for inhibitors, e.g., protease inhibitors; to identify recognition sites or to detect substrate specificity, e.g., using a luciferase with a selected recognition sequence or a library of polypeptides having luciferase activity having a plurality of different sequences with a single molecule of interest or a plurality (for instance, a library) of molecules; to select or evolve biosensors or molecules of interest, e.g., proteases; or to detect protein-protein interactions via complementation or binding, e.g., in an in vitro or cell-based approach. In one aspect, a polypeptide having luciferase activity which includes an inserted amino acid sequence is contacted with a random library or mutated library of molecules, and molecules identified which interact with the inserted amino acid sequence. In another aspect, a library of polypeptides having luciferase activity having a plurality insertions is contacted with a molecule, and polypeptides having luciferase activity which interact with the molecule identified. In one embodiment, a polypeptide having luciferase activity or fusion thereof, is useful to detect, e.g., in an in vitro or cell-based assay, the amount or presence of cAMP or cGMP (for example, by inserting a cAMP or cGMP binding site into the polypeptide having luciferase activity), to screen for inhibitors or activators of, e.g., cAMP or cGMP, inhibitors or activators of cAMP binding to a cAMP binding site or inhibitors or activators of G protein coupled receptors (GPCR), to identify recognition sites or to detect substrate specificity, e.g., using a polypeptide having luciferase activity with a selected recognition sequence or a library of polypeptides having luciferase activity having a plurality of different sequences with a single molecule of interest or a plurality (for instance, a library) of molecules, to select or evolve cAMP or cGMP binding sites, or in whole animal imaging.

Also encompassed herein are methods to monitor the expression, location and/or trafficking of molecules in a cell, as well as to monitor changes in microenvironments within a cell, using a polypeptide having luciferase activity or a fusion protein thereof. In one aspect, a polypeptide having luciferase activity comprises a recognition site for a molecule, and when the molecule interacts with the recognition site, that results in an increase in activity, and thus can be employed to detect or determine the presence or amount of the molecule. For example, in one aspect, a polypeptide having luciferase activity comprises an internal insertion containing two domains which interact with each other under certain conditions. In one embodiment, one domain in the insertion contains an amino acid which can be phosphorylated and the other domain is a phosphoamino acid binding domain. In the presence of the appropriate kinase or phosphatase, the two domains in the insertion interact and change the conformation of the polypeptide having luciferase activity resulting in an alteration in the detectable activity of the modified luciferase. In another embodiment, a modified luciferase comprises a recognition site for a molecule, and when the molecule interacts with the recognition site, results in an increase in activity, and so can be employed to detect or determine the presence of amount or the other molecule.

In certain aspects, a method for detecting luminescence in a cell further comprises contacting the cell with a polypeptide having luciferase activity. In certain aspects, the polypeptide is fused to a targeting moiety that specifically binds to the cell. In certain aspects, the targeting moiety is a peptide, lipid, protein, or a small molecule. In certain aspects, the targeting moiety is an antibody or an antigen binding fragment thereof, a receptor, a ligand, or a substrate.

In certain aspects, the cell is contacted with a luciferin analog after contacting the cell with the polypeptide having luciferase activity, wherein the luciferin analog is a compound described herein or a stereoisomer, a tautomer or a salt thereof. In certain aspects, the cell is in a tissue sample. In certain aspects, the cell is in vivo in a subject.

In certain aspects, the method comprises contacting the tissue with the polypeptide having luciferase activity and fused to a targeting moiety and contacting the tissue with a luciferin analog, wherein the luciferin analog is a compound of Formula (I), or a stereoisomer, a tautomer or a salt thereof, or any compound described herein; and detecting localization of the polypeptide in the tissue.

In certain aspects, the method comprises administering to the subject the polypeptide having luciferase activity and fused to a targeting moiety for localizing the polypeptide to the cell, administering to the subject a luciferin analog, wherein the luciferin analog is a compound of Formula (I), or a stereoisomer, a tautomer or a salt thereof or any compound described herein; and detecting luminescence to determine localization of the polypeptide to the cell. In certain aspects, the subject is a mammal, a primate, or a human.

In certain aspects, a method for detecting luminescence in a transgenic animal comprises administering a luciferin analog to a transgenic animal, wherein the luciferin analog is a compound of Formula (I), or a stereoisomer, a tautomer or a salt thereof or any compound described herein; and detecting luminescence. In certain aspects, the transgenic animal expresses a polypeptide having luciferase activity.

### Assay Solutions

Assay buffers that increase and stabilize the signal output of luciferase assay are described. In certain aspects, an assay buffer may include imidazole, e.g., about 10 mM-1000 mM imidazole, about 50 mM-1000 mM imidazole, about 10 mM-500 mM imidazole, about 50 mM-500 mM imidazole, about 75 mM-250 mM imidazole, about 75 mM-150 mM imidazole, or about 100 mM imidazole. In certain aspects, the assay buffer may have a pH of about 8, e.g., about pH6-pH9, about pH7-pH9, or about pH7.5-8.5, such as pH7.6, 7.8, 8.0, 8.2, or 8.4.

In certain aspects, the assay buffer results in a signal from luciferase activity that is at least 10% higher than the signal obtained using an assay buffer not containing imidazole, e.g., at least 20% higher, at least 30% higher, at least 40% higher, at least 50% higher, at least 60% higher, at least 70% higher, at least 80% higher, at least 90% higher, at least 100% higher, at least 150% higher, or up to 150% higher, or up to 180% higher, or up to 200% higher.

In certain aspects, the assay buffer may include a buffering agent, e.g., phosphate buffered saline, Tris, a histidine buffer, N-(2-Hydroxyethyl)piperazine-N-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc. In certain aspects, the assay buffer may include one or more of a stabilizing agent; an anti-foaming agent; an anti-oxidant; and a reducing agent.

In certain aspects, the stabilizing agent may be an alcohol, e.g., propylene Glycol; an anti-foaming agent, such as, alcohols (cetostearyl alcohol), insoluble oils (castor oil), stearates, polydimethylsiloxanes and other silicones derivatives, ether and glycols; an anti-oxidant, e.g., ascorbic acid, glutathione, cysteine, methionine or citric acid; a reducing agent, e.g., thiourea.

In certain aspects, a kit may comprise an assay buffer of the present disclosure and a luciferin substrate. The luciferin substrate may be any luciferin substrate, such as, a luciferin substrate of the present disclosure.

In certain aspects, the kits provided herein may include one or more of the polypeptides, the substrates, and the assay buffers provided herein.

### EXAMPLES

The following examples are offered to illustrate, but not to limit any embodiments provided by the present disclosure.

### Example 1: LuxSit-i Variants Having Improved Stability

LuxSit-i (SEQ ID NO:1) sequence is provided in FIG. 1. Secondary structure, H1-L1-H2-L2-B1-L3-B2-L4-H3-L5-B3-L6-B4-L7-B5-L8-B6, is mapped onto the primary structure. As described in Yeh, A. H W., et al. De novo design of luciferases using deep learning. Nature 614, 774-780 (2023), LuxSit-i (SEQ ID NO:1) sequence is derived from LuxSit. The amino acid sequence of LuxSit is set forth in SEQ ID NO:92.

A single saturation mutagenesis (SSM) was performed to evaluate by yeast display the effect of single mutations in the stability of LuxSit-i (SEQ ID NO:1). The SSM library was transformed into electrocompetent cells of the strain EBY100. Twenty million cells were labeled with anti-myc FITC antibody in order to detect protein expression on the yeast surface by measuring fluorescence emission. Cells were sorted using a Sony SH800 cell sorter and those showing protein expression (FITC signal) were collected and grown. DNA was extracted from the sorted cells using conventional DNA extraction conventional methods. The DNA library was sequenced using Nanopore technology. Single amino acid substitutions that increase protein stability are enriched after sorting.

Single saturation mutagenesis (SSM) was performed to evaluate (by yeast display) the effect of single mutations in the stability of LuxSit-i-i. The SSM library was transformed into electrocompetent cells of the strain EBY100. Twenty million cells were labeled with an anti-myc FITC antibody to detect protein expression on the yeast surface by measuring the emission of fluorescence by FITC. Yeast were incubated with a mixture of 3.5 uM trypsin and 1.4 uM chymotrypsin (1/729 dilution of the stock solution) at 25° C. for 15 min. Cells were sorted using a Sony SH800 cell sorter and those showing protein expression (FITC signal) after protease incubation were collected and grown. DNA was extracted from the sorted cells using conventional DNA extraction conventional methods. The DNA library was sequenced using Nanopore technology. Single amino acid substitutions that increase protein stability are enriched after sorting.

Single saturation mutagenesis (SSM) was performed to evaluate (by yeast display) the effect of single mutations in the stability of LuxSit-i. The SSM library was transformed into electrocompetent cells of the strain EBY100. Twenty million cells were labeled with an anti-myc FITC antibody in order to detect protein expression on the yeast surface by measuring the emission of fluorescence by FITC. Yeast were incubated with a mixture of 10 uM trypsin and 4 uM chymotrypsin (1/243 dilution of the stock solution) at 25° C. for 15 min. Cells were sorted using a Sony SH800 cell sorter and those showing protein expression (FITC signal) after protease incubation were collected and grown. DNA was extracted from the sorted cells using conventional DNA extraction conventional methods. The DNA library was sequenced using Nanopore technology. Single amino acid substitutions that increase protein stability are enriched after sorting.

Single saturation mutagenesis (SSM) was performed to evaluate (by yeast display) the effect of single mutations in the stability of LuxSit-i. The SSM library was transformed into electrocompetent cells of the strain EBY100. Twenty million cells were labeled with an anti-myc FITC antibody in order to detect protein expression on the yeast surface by measuring the emission of fluorescence by FITC. Yeast were incubated with a mixture of 15 uM trypsin and 6 uM chymotrypsin (1/162 dilution of the stock solution) at 25° C. for 15 min. Cells were sorted using a Sony SH800 cell sorter and those showing protein expression (FITC signal) after protease incubation were collected and grown. DNA was extracted from the sorted cells using conventional DNA extraction conventional methods. The DNA library was sequenced using Nanopore technology. Single amino acid substitutions that increase protein stability are enriched after sorting.

Single saturation mutagenesis (SSM) was performed to evaluate (by yeast display) the effect of single mutations in the stability of LuxSit-i. The SSM library was transformed into electrocompetent cells of the strain EBY100. Twenty million cells were labeled with an anti-myc FITC antibody in order to detect protein expression on the yeast surface by measuring the emission of fluorescence by FITC. Yeast were incubated with a mixture of 30 uM trypsin and 12 uM chymotrypsin (1/81 dilution of the stock solution) at 25° C. for 15 min. Cells were sorted using a Sony SH800 cell sorter and those showing protein expression (FITC signal) after protease incubation were collected and grown. DNA was extracted from the sorted cells using conventional DNA extraction conventional methods. The DNA library was sequenced using Nanopore technology. Single amino acid substitutions that increase protein stability are enriched after sorting.

### Example 2: LuxSit-i Variants Having Improved Activity

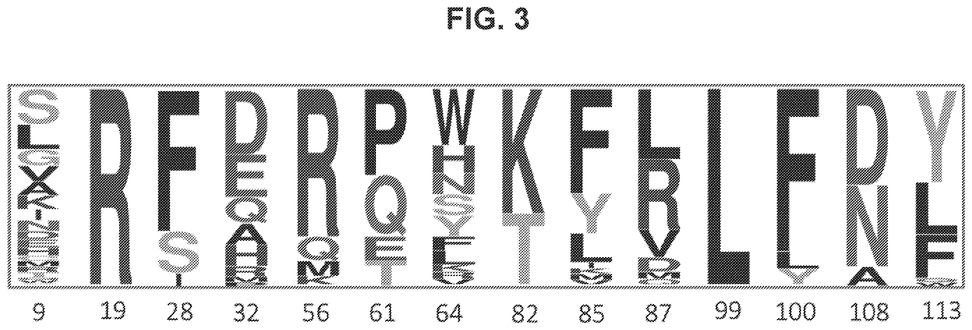

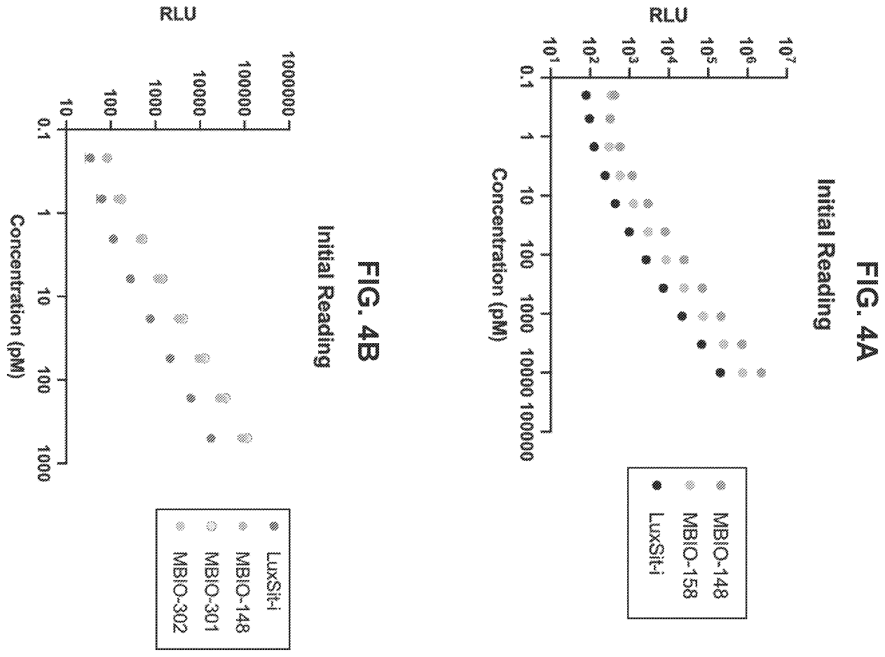

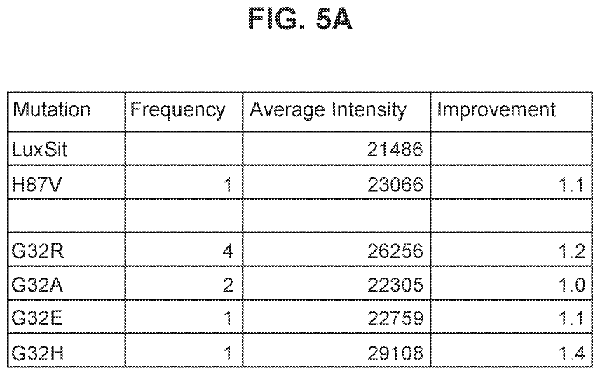

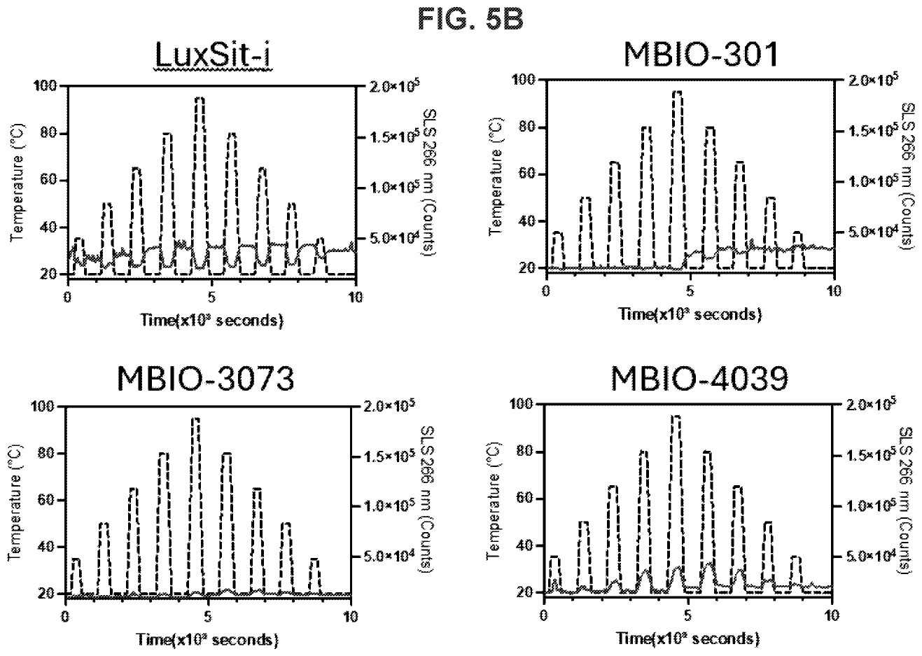

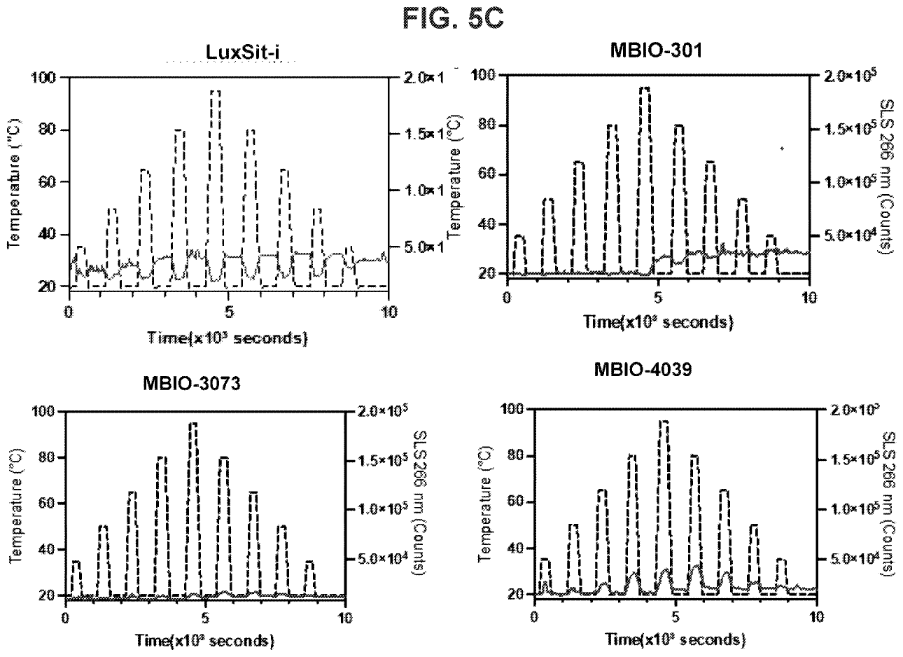

SSM was performed to evaluate the effect of single mutations on the luciferase activity of LuxSit-i. FIG. 3 graphically presents the mutation frequency at the positions found in the SSM as beneficial for increasing luciferase activity.

Table 2 summarizes properties of exemplary LuxSit-i variants. The amino acid substitutions are relative to SEQ ID NO:1 (LuxSit-i). Stability and brightness are relative to LuxSit-i.

TABLE 2

| | | Brightness | Stability |
|---|---|---|---|
| ID | Substitution | improvement | improvement |
| MBIO-149 | A85F | Slight increase | No change |
| MBIO-150 | F9S | No change | Monomeric |
| MBIO-153 | F9V | No change | Monomeric |
| MBIO-156 | H87L | Slight increase | No change |
| MBIO-157 | Q64W | Slight increase | No change |
| MBIO-158 | W100F | Slight increase | Monomeric |

MBIO-158 includes a single amino acid substitution W100F relative to SEQ ID NO:1.

Single amino acid substitutions that improved luciferase activity and/or stability were combined and assayed for luciferase activity and stability. Exemplary variants are listed in Table 3.

TABLE 3
Stability and brightness are relative to LuxSit-i:

| | | Brightness | |
|---|---|---|---|
| ID | Substitutions | improvement | Stability improvement |
| MBIO-147 | W100F_A85F_H87L | Increase | Monomeric |
| (SEQ ID NO: 3) | | | |
| MBIO-148 | W100F_A85F_H87L_Q64W | Increase | Monomeric |
| (SEQ ID NO: 2600) | | | |
| MBIO-151 | F9S_A85F_H87L | No change | No change |
| (SEQ ID NO: 7) | | | |
| MBIO-152 | F9S_A85F_H87L_Q64W | Slight increase | Slight improvement |
| (SEQ ID NO: 6) | | | |
| MBIO-154 | F9V_A85F_H87L | Slight increase | Slight improvement |
| (SEQ ID NO: 5) | | | |
| MBIO-155 | F9V_A85F_H87L_Q64W | Slight increase | Slight improvement |
| (SEQ ID NO: 4) | | | |

FIG. 4A provides data for luciferase activity for MBIO-148, MBIO-158 and Luxsit-i. MBIO-148, MBIO-158, and Luxsit-i were serially diluted starting from a final in-well concentration of 10,000 pM. Each dilution was combined with Diphenylterazine (DTZ) to a final in-well concentration of 10 M. Points shown are from the initial reading once the plate read was started.

FIG. 4B provides data for luciferase activity for MBIO-148, MBIO-301, MBIO-302, and LuxSit-i. MBIO-148, MBIO-301, MBIO-302, and Luxsit-i were serially diluted starting from a final in-well concentration of 500 pM. Each dilution was combined with DTZ to a final in-well concentration of 10 μM. Points shown are from the initial reading once the plate read was started.

Table 4 provides the substitutions present in these and additional variants relative to SEQ ID NO:1.

TABLE 4

| Variant | SEQ ID NO | Substitutions |
|---|---|---|
| MBIO-148 | 2 | Q64W, A85F, H87L, W100F |
| MBIO-301 | 104 | F9N, Q64L, A85F, H87R, H99L, T108D, H113Y, W100F |
| MBIO-304 | 2665 | F9E, S19R, G32Q, L56R, Q64N, A85F, H99L, T108N, H113Y, W100F |
| MBIO-302 | 128 | F9N, Q64L, A85F, H87F, H99L, T108D, H113Y |

Table 6 provides the substitutions present in variants of LuxSit-i relative to the sequence of LuxSit-i set forth in SEQ ID NO:1. The LuxSit-i variants exhibit increased luciferase activity as compared to LuxSit-i.

TABLE 6

| Variant | SEQ ID NO | Substitutions |
|---|---|---|
| MBIO-156 | 88 | H87L |
| MBIO-157 | 47 | Q64W |
| MBIO-149 | 82 | A85F |
| MBIO-150 | 73 | F9S |
| MBIO-152 | 6 | F9S, Q64W, A85F, H87L |
| | 95 | W100F |
| MBIO-155 | 4 | F9V, Q64W, A85F, H87L |
| MBIO-147 | 3 | A85F, H87L, W100F |
| MBIO-148 | 2 | Q64W, A85F, H87L, W100F |
| MBIO-301 | 104 | F9N, Q64L, A85F, H87R, H99L, W100F, T108D, H113Y |
| MBIO-2466 | 2703 | F9N, H30D, H36T, Q64L, H80T, H84S, A85F, H87R, H92S, H98Q, H99L, |
| | | W100F, H101R, T108D, H113Y |
| MBIO-3075 | 2665 | F9N, H30D, H36T, L56Q, Q64L, H80T, H84S, A85F, H87R, H92S, H98Q, H99L, |
| | | W100F, H101R, T108D, H113Y |
| MBIO-4039 | 2730 | F9N, D23I, H30D, H36T, V41T, R46V, R55S, L56Q, Q64L, K68S, H80T, Q82R, |
| | | H84S, A85F, H87R, T97L, H98Q, H99L, W100F, H101K, R103V, T108V, |
| | | E109A, H113Y, I114V |

LuxSit-i variants showing increased luciferase activity and/or increase stability relative to LuxSit-I are listed in Table 7. The substitutions are shown relative to the sequence of LuxSit-i set forth in SEQ ID NO:1. Table 7 lists single mutants. Mutants were obtained from an Error Prone PCR.

TABLE 7

| SEQ ID | | SEQ ID | | SEQ ID | | SEQ ID | |
|---|---|---|---|---|---|---|---|
| NO | Mutation | NO | Mutation | NO | Mutation | NO | Mutation |
| 66 | E3D | 58 | L28S | 62 | V77Y | 61 | T108D |
| 40 | I6K | 48 | L28F | 37 | E78W | 35 | T108N |
| 43 | I6T | 52 | G32R | 60 | Q82K | 84 | H113F |
| 78 | F9D | 25 | G32D | 67 | Q82T | | |
| 64 | F9E | 76 | G32E | 82 | A85F | | |
| 81 | F9Q | 51 | G32H | 87 | A85Y | | |
| 90 | F9R | 68 | G32A | 85 | A85L | | |
| 73 | F9S | 55 | T42I | 59 | A85I | | |
| 44 | F9T | 17 | F43L | 74 | A85M | | |
| 39 | F9H | 49 | F43G | 53 | T86A | | |
| 22 | F9I | 42 | S45A | 88 | H87L | | |
| 70 | F9L | 69 | L56R | 79 | H87V | | |
| 86 | F9V | 83 | L56K | 20 | H92Y | | |
| 94 | F9A | 18 | L56Q | 38 | L96R | | |
| 96 | F9G | 46 | F57V | 31 | H99L | | |
| 91 | F9C | 65 | T59K | 95 | W100F | | |
| 80 | F9M | 32 | K61E | 54 | W100Y | | |
| 63 | Y14W | 45 | K61P | 41 | W100L | | |
| 89 | E15G | 47 | Q64W | 30 | R106L | | |
| 71 | S19R | 33 | Q64H | 27 | V107I | | |

LuxSit-i variants showing increased luciferase activity and/or increase stability relative to LuxSit-i are listed in Table 8. The substitutions are shown relative to the sequence of LuxSit-i set forth in SEQ ID NO:1. Table 8 lists double or triple mutants. Mutants were obtained from an Error Prone PCR.

TABLE 8

| SEQ ID NO | Mutations | SEQ ID NO | Mutations |
|---|---|---|---|
| 14 | L56Q H113Q | 77 | G32M Q82T |
| 72 | F9A H113W | 8 | M1T H92Y |
| 9 | F9L G74D | 11 | E47A R111C |
| 10 | F9S S58T | 15 | F43L A63V |
| 13 | F9S I35V | 21 | R50K G118D |
| 50 | F9V T59E | 23 | I35F F49S |
| 19 | F9L H101Y | 26 | Q5K R11C V33A |
| 75 | F9N H101R | 28 | L37Q N105S |
| 16 | H101Q V107A N115S | 34 | L28I R50K |
| 57 | T59Q H87M | 36 | V79T P116S |
| 12 | R50I Q64H H87Q | 56 | D23V K68M |
| 29 | Q5L G32D Q64H | 24 | S19R G32R |

Table 9 lists mutants sequences obtained from the manual combination of some of the most frequent mutations listed in Tables 7 and 8. Brightness and/or stability was improved relative to LuxSit-i and the single or double or triple mutants.

TABLE 9

| SEQ ID NO | Substitutions |
|---|---|
| 3 | A85F H87L W100F |
| 2 | Q64W A85F H87L W100F |
| 133 | F9S Q64K A85F H87V |
| 116 | F9K Q64V A85F H87R |
| 126 | F9L Q64Y A85F H87R |
| 4 | F9V Q64W A85F H87L |
| 6 | F9S Q64W A85F H87L |
| 5 | F9V A85F H87L |
| 7 | F9S A85F H87L |

Table 10 lists mutants obtained from a combinatorial library containing the most frequent mutations listed in Tables 7 and 8. MBIO-301 (having the sequence set forth in SEQ ID NO:104) had the highest luciferase activity.

TABLE 10

| SEQ ID NO | Subtitutions | SEQ ID NO | Subtitutions |
|---|---|---|---|
| 97 | F9S S19R G32D L56Q Q82K H99L W100F | 120 | F9K L56R K61P Q82T H99L T108D |
| | T108N H113Y | | H113F |
| 98 | F9S G32H L56R H99L W100F H113Y | 121 | F9D S19R L28F G32A L56R Q82K |
| | | | H99L T108D H113Y |
| 99 | F9G L28F G32Q L56R K61P Q82K H99L | 122 | F9L G32D L56R K61E Q82T H99L |
| | W100F T108D H113Y | | H113F |
| 100 | F9H G32D L56R Q82T W100F T108A H113F | 123 | F9C S19R L28F G32D L56R Q82T |
| | | | H99L T108N H113L |
| 101 | F9V L28F G32D L56R Q82K W100F T108N | 124 | F9S S19R G32D L56Q Q82K H99L |
| | H113L | | T108N H113Y |
| 102 | F9G S19R G32E L56M Q64S A85L H99L | 125 | F9E S19R G32D L56R K61Q Q82K |
| | W100F T108D | | H99L T108N H113F |
| 103 | F9L Q64F A85F H87R H99L W100F T108D | 127 | F9T L56R K61Q Q82K T108N H113F |
| | H113Y | | |
| 104 | F9N Q64L A85F H87R H99L W100F T108D | 128 | F9N Q64L A85F H87R H99L T108D |
| (MBIO-301) | H113Y | | H113Y |
| 105 | F9A S19R Q64N A85Y H87R W100F T108N | 120 | F9Y L56R K61P A85F H87V T108D |
| | H113Y | | H113L |
| 106 | F9L S19R L28F G32Q L56R K61T Q82T H99L | 130 | F9G Q64Y A85V H87V T108D H113Y |
| | W100F T108D H113L | | |
| 107 | F9D S19R G32H L56R K61Q Q82K H99L | 131 | F91 L56M Q64S A85L H87R H113Y |
| | W100F T108D H113Y | | |
| 108 | F9G L28F G32Q L56R K61P Q82K H99L | 132 | F91 S19R G32P L56R Q64Y A85L |
| | T108D H113Y | | H113F |
| 109 | F9V L28F G32D L56R Q82K T108N H113L | 134 | F9K D18E Q64H A85Y H87R T108D |
| | | | H113F |
| 110 | F9S G32H L56R H99L H113Y | 135 | F9E S19R G32Q L56R Q64N A85F |
| | | | H99L T108N H113Y |
| 111 | F9S G32D L56R K61T Q82T T108D H113L | 136 | F9A S19R Q64N A85Y H87R T108N |
| | | | H113Y |
| 112 | F9R S19R G32E L56R H99L T108A H113L | 137 | F9S S19R L56R K61Q Q82K A85F |
| | | | H87L H99L W100F T108N H113L |
| 113 | F9H G32D L56R Q82T T108A H113F | 138 | F9L S19R L285 G32A L56Q Q64W |
| | | | Q82K A85K H87D H99L W100F |
| | | | T108N H113Y |
| 114 | F9G S19R G32E L56M Q64S A85L H99L | 139 | F9W S19R G32E L56R Q82K H87L |
| | T108D | | H99L W100F T108D H113L |
| 115 | Q5E F9K L56R Q82T H99L T108N H113Y | 140 | F9A S19R L28F G32Q L56R Q64W |
| | | | Q82K A85Y H99L W100Y T108N |
| | | | H113L |
| 117 | F9L Q64F A85F H87R H99L T108D H113Y | 141 | F91 S19R G32A L56R K61Q Q64W |
| | | | Q82K A85Y H87D H99L W100F |
| | | | T108N H113L |
| 118 | F9S L56R K61P Q82T T108D H113Y | 142 | F9N S19R L28F G32E L56R Q64R |
| | | | Q82K A85F H87L W100F T108A |
| | | | H113L |
| 119 | F9K S19R G32E L56K Q82K T108N H113Y | 143 | F9M L28S G32E L56R K61P Q64W |
| | | | Q82K A85Y H99L W100L T108D |
| | | | H113F |
| 2663 | L28S G32H L56R Q64W Q82K A85L H87D | 2665 | F9E S19R G32Q L56R Q64N A85F |
| | W100Y T108D H113Y | | H99L W100F T108N H113Y |

Table 11 lists variants derived from MBIO-301. The listed histidine residues in MBIO-301 were substituted. MBIO-2466 containing the mutation H98Q in the MBIO-301 sequence showed more than 5× higher luciferase activity than MBIO-301. Table 11 lists the substitutions relative to SEQ ID NO:1.

TABLE 11

| MBIO-301 | |
|---|---|
| variant | Substitutions |
| MBIO-1688 | F9N H30A Q64L A85F H87R H99L W100F T108D H113Y |
| MBIO-1691 | F9N H30D Q64L A85F H87R H99L W100F T108D H113Y |
| MBIO-1693 | F9N H301 Q64L A85F H87R H99L W100F T108D H113Y |
| MBIO-1694 | F9N H30S Q64L A85F H87R H99L W100F T108D H113Y |
| MBIO-1695 | F9N H36T Q64L A85F H87R H99L W100F T108D H113Y |
| MBIO-1696 | F9N H36Y Q64L A85F H87R H99L W100F T108D H113Y |
| MBIO-1697 | F9N Q64L H80K A85F H87R H99L W100F T108D H113Y |
| MBIO-1698 | F9N Q64L H80T A85F H87R H99L W100F T108D H113Y |
| MBIO-1699 | F9N Q64L H80V A85F H87R H99L W100F T108D H113Y |
| MBIO-1700 | F9N Q64L H84S A85F H87R H99L W100F T108D H113Y |
| MBIO-1701 | F9N Q64L H84T A85F H87R H99L W100F T108D H113Y |
| MBIO-1702 | F9N Q64L A85F H87R H92K H99L W100F T108D H113Y |
| MBIO-1703 | F9N Q64L A85F H87R H92M H99L W100F T108D H113Y |
| MBIO-1704 | F9N Q64L A85F H87R H99L W100F H101T T108D H113Y |
| MBIO-1687 | F9N H30I H36T Q64L H80T H84S A85F H87R H92K H99L W100F H101T T108D H113Y |
| MBIO-1689 | F9N H30D H36T Q64L H80T H84S A85F H87R H92K H99L W100F H101T T108D H113Y |
| MBIO-1690 | F9N H30D H36T Q64L H80T H84S A85F H87R H92M H99L W100F H101T T108D H113Y |
| MBIO-1692 | F9N H30I H36T Q64L H80T H84S A85F H87R H92M H99L W100F H101T T108D H113Y |
| MBIO-1705 | F9N H30S H36T Q64L H80V H84T A85F H87R H92K H99L W100F H101T T108D H113Y |
| MBIO-1706 | F9N H30A H36Y Q64L H80K H84T A85F H87R H92K H99L W100F H101T T108D H113Y |
| MBIO-2465 | F9N H30D H36T Q64L H80T H84S A85F H87R H92S H99L W100F H101R T108D H113Y |
| MBIO-2466 | F9N H30D H36T Q64L H80T H84S A85F H87R H92S H98Q H99L W100F H101R T108D H113Y |

Table 12 lists variants derived from MBIO-2466. MBIO-3073 had the highest luciferase activity. Table 12 lists the substitutions relative to SEQ ID NO:1.

TABLE 12

| MBIO-2466 | |
|---|---|
| Variant | Substitutions |
| MBIO-3227 | F9N H30D H36T Q64L K68M H80T Q82H H84S A85F H87R H92S H98Q H99L W100F |
| | H101R T108D H113Y |
| MBIO-3230 | F9N H30D H36T R55P Q64L H80T H84S A85F H87R H92S H98Q H99L W100F H101R |
| | T108D H113Y |
| MBIO-3070 | F9N H30D P31Q H36T L56Q Q64L H80T H84S A85F H87R H92S H98Q H99L W100F |
| | H101R T108D H113Y |
| MBIO-3074 | F9D H30D H36T L56Q Q64L H80T H84S A85F H87R H92S H98Q H99L W100F H101R |
| | T108D H113Y |
| MBIO-3075 | F9N H30D H36T L56Q Q64L H80T H84S A85F H87R H92S H98Q H99L W100F H101R |
| | T108D H113Y |
| MBIO-4040 | F9N H30D H36T L56Q Q64L H80T V81I H84S A85F H87R H92S H98Q H99L W100F H101R |
| | T108D H113Y |
| MBIO-3076 | F9N D23G H30D H36T V41T R46V L56Q Q64L H80T H84S A85F H87R T97L H98Q H99L |
| | W100F H101K R103V T108V H113Y |
| MBIO-2859 | F9N H30D H36T V41T R46V Q64L H80T H84S A85F H87R T97L H98Q H99L W100F H101K |
| | R103V T108V H113Y |
| MBIO-3071 | F9N H30D H36T V41T R46V Q64L H80T H84S A85F H87R T97L H98Q H99L W100Y H101K |
| | R103V T108V H113Y |
| MBIO-3072 | F9N H30D H36T V41T R46V Q64L R73C H80T H84S A85F H87R T97L H98Q H99L W100F |
| | H101K R103V T108V H113Y |
| MBIO-3073 | F9N H30D H36T V41T R46V Q64L H80T H84S A85F H87R T97L H98Q H99L W100F H101K |
| | R103V T108V E109D H113Y I114T |
| MBIO-2467 | F9N H30D H36T V41D Q64L H80T H84S A85F H87R H92S T97G H98Q H99L W100F |
| | H101R T108D H113Y |

Table 13 lists variants derived from MBIO-2859. MBIO-4039 had the highest luciferase activity and was the most stable variant. Table 13 lists the substitutions relative to SEQ ID NO:1.

TABLE 13

| MBIO-2859 | |
|---|---|
| Variants | Substitutions |
| MBIO-3639 | F9N H30D H36T V41T R46V R55Y Q64L K68T H80T Q82A H84S A85F H87R T97L H98Q H99L |
| | W100F H101K R103V T108V E109F H113Y I114V |
| MBIO-3342 | F9N D23S H30D H36T V41T R46V R55Y L56N Q64L K68V H80T Q82R H84S A85F H87R T97L |
| | H98Q H99L W100F H101K R103V T108V E109F H113Y I114D |
| MBIO-3383 | F9N D23R H30D H36T V41T R46V R55K L56E Q64L K68V H80T Q82R H84S A85F H87R T97L |
| | H98Q H99L W100F H101K R103V T108V E109S H113Y I114E |
| MBIO-3384 | F9N D231 H30D H36T V41T R46V R55L L56Q Q64L K68S H80T Q82R H84S A85F H87R T97L |
| | H98Q H99L W100F H101K R103V T108V E109A H113Y I114V |
| MBIO-3385 | F9N R12S D23E H30D H36T V41T R46V R55H L56N Q64L K68T H80T Q82K H84S A85F H87R |
| | T97L H98Q H99L W100Y H101K R103V T108V E109Y H113Y I114H |
| MBIO-3386 | F9N D23N H30D H36T V41T R46V R55E L56R Q64L K68R H80T Q82Y H84S A85F H87R T97L |
| | H98Q H99L W100F H101K R103V T108V E109T H113Y I114V |
| MBIO-3387 | F9N D23L H30D H36T V41T R46V R55V L56Q Q64L K68V H80T Q82E H84S A85F H87R T97L |
| | H98Q H99L W100F H101K R103V T108V E109S H113Y I114W |
| MBIO-3388 | F9N D23E H30D H36T V41T R46V R55V L56S Q64L K68S H80T Q82K H84S A85F H87R T97L |
| | H98Q H99L W100Y H101K R103V T108V E109D H113Y I114A |
| MBIO-3638 | F9N D23Y H30D H36T V41T R46V R55V L56Q Q64L K68L H80T Q82T H84S A85F H87R T97L |
| | H98Q H99L W100F H101K R103V T108V E109S H113Y I114A |
| MBIO-3640 | F9N D23S H30D H36T V41T R46V R55Y L56N Q64L K68D H80T Q82S H84S A85F H87R T97L |
| | H98Q H99L W100F H101K R103V T108V E109S H113Y I114D |
| MBIO-3641 | F9N D23W H30D H36T V41T R46V R55N L56R Q64L K68L H80T Q82K H84S A85F H87R T97L |
| | H98Q H99L W100F H101K R103V T108V E109S H113Y I114D |
| MBIO-3643 | F9N D23T H30D H36T V41T R46V R55V L56S Q64L K68R H80T Q82G H84S A85F H87R T97L |
| | H98Q H99L W100F H101K R103V T108V E109H H113Y I114A |
| MBIO-3644 | F9N D23T H30D H36T V41T R46V R55L L56N Q64L K68T H80T Q82N H84S A85F H87R T97L |
| | H98Q H99L W100F H101K R103V T108V E109S H113Y I114V |
| MBIO-4039 | F9N D23I H30D H36T V41T R46V R55S L56Q Q64L K68S H80T Q82R H84S A85F H87R T97L |
| (SEQ ID | H98Q H99L W100F H101K R103V T108V E109A H113Y I114V |
| NO: 2730) | |
| MBIO-4042 | F9N D23I H30D H36T V41T R46V E54G R55L L56Q Q64L K68S H80T Q82R H84S A85F H87R |
| | T97L H98Q H99L W100F H101K R103V T108V E109A H113Y I114V |

Sequences of mutants of LuxSit-i are set forth in SEQ ID NOs: 2-91, 93-143, 2601-2604, 2663, 2665, and 2682-2732.

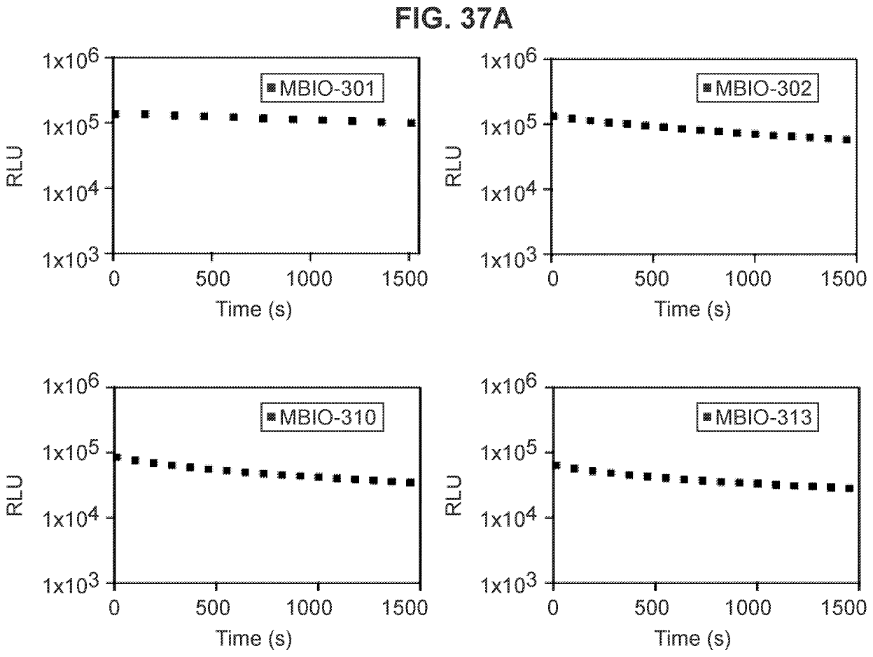

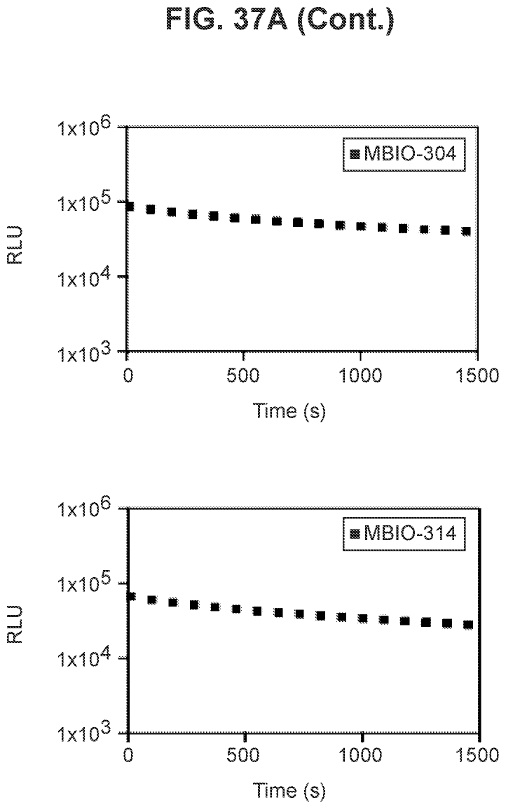

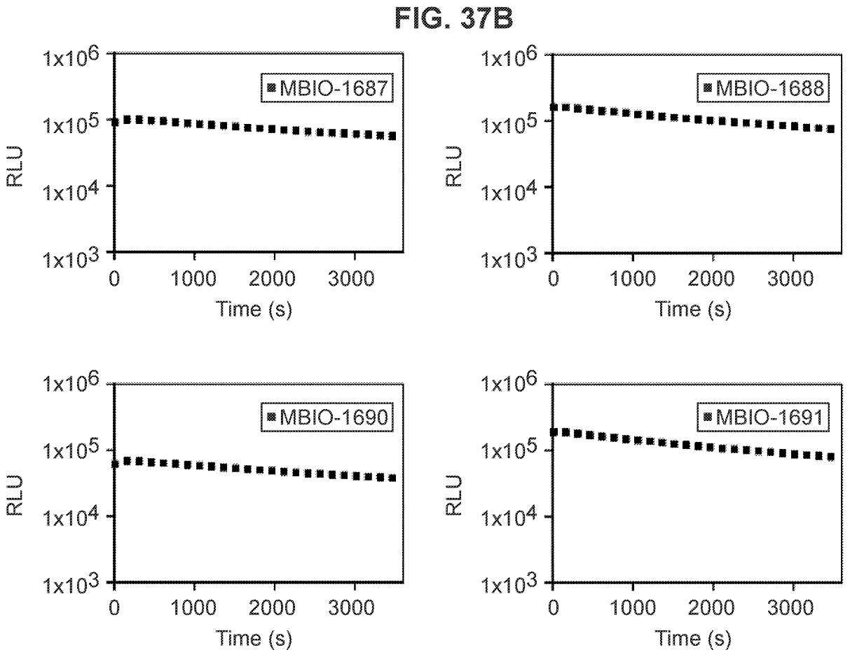

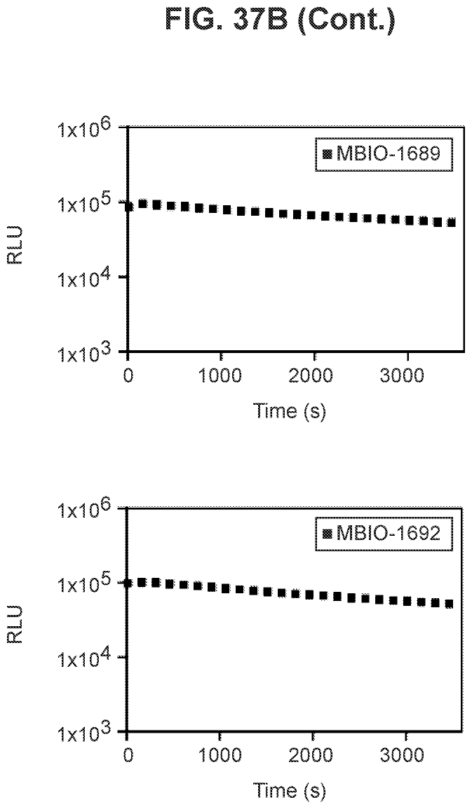

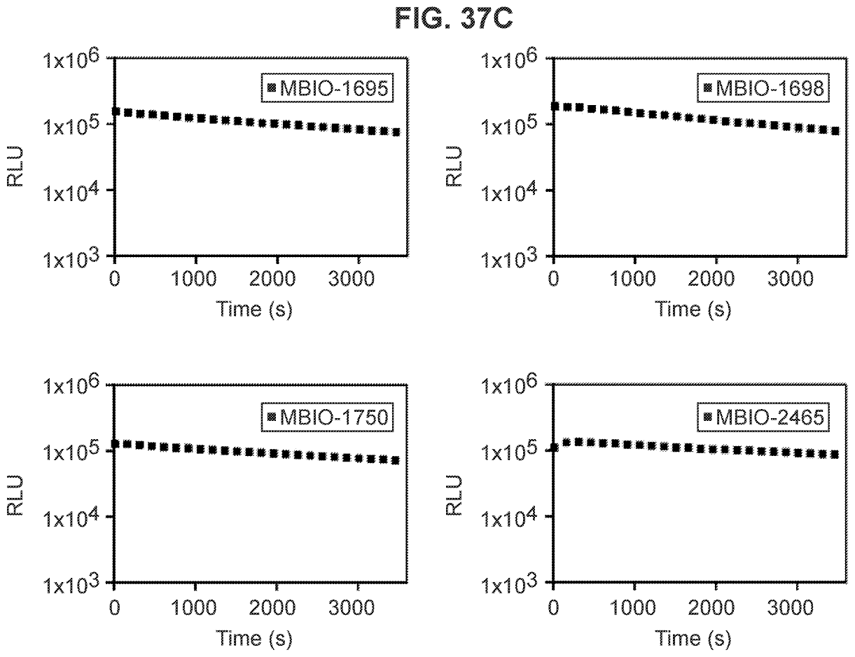

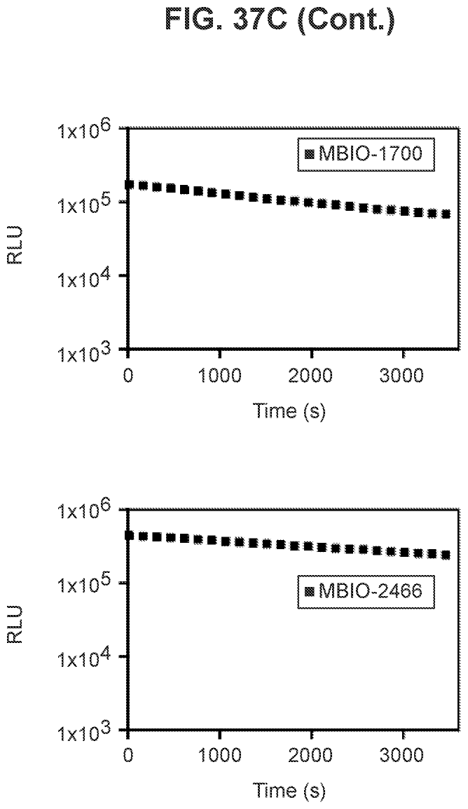

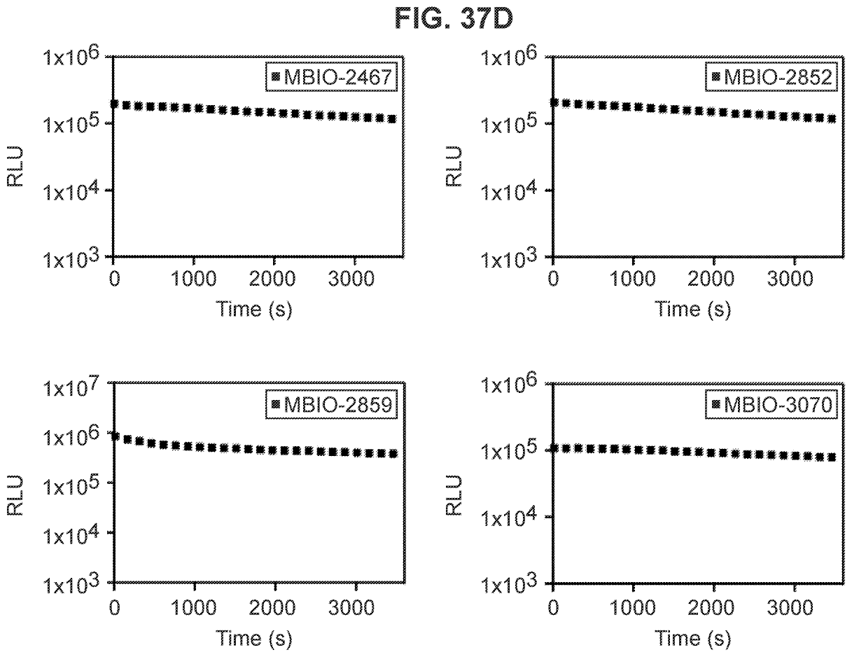

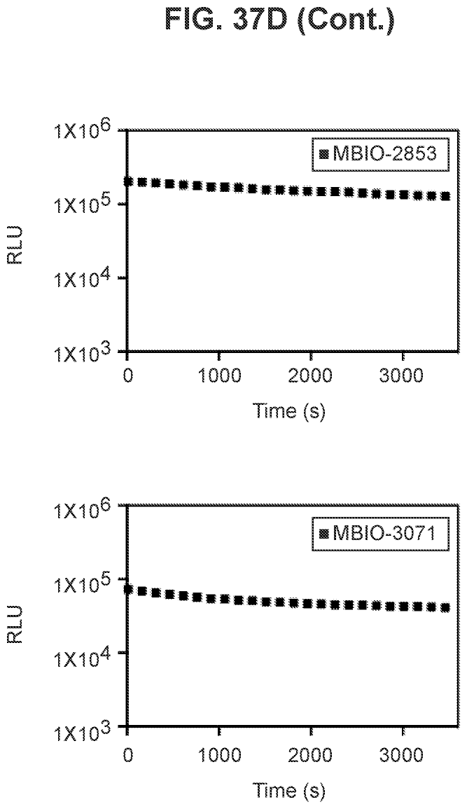

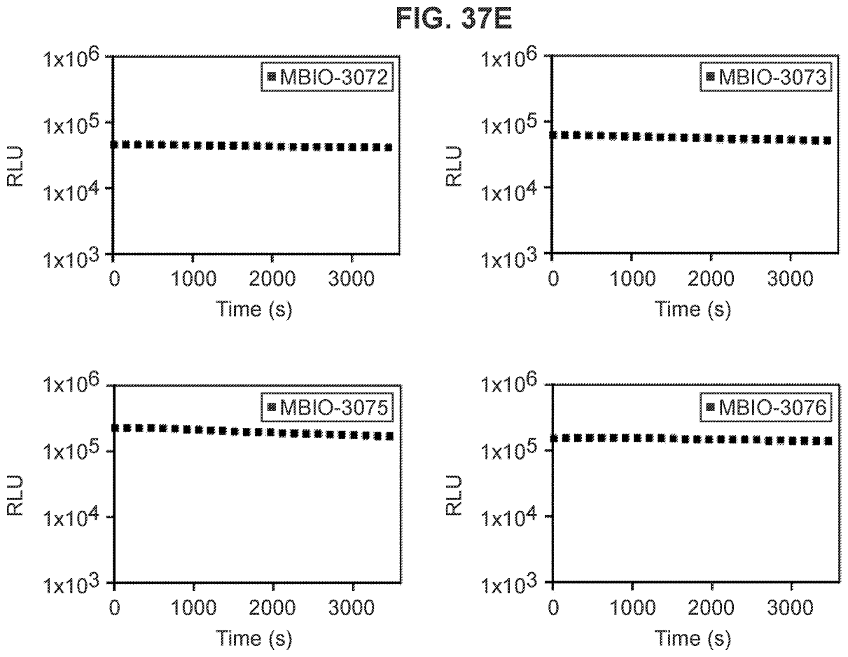

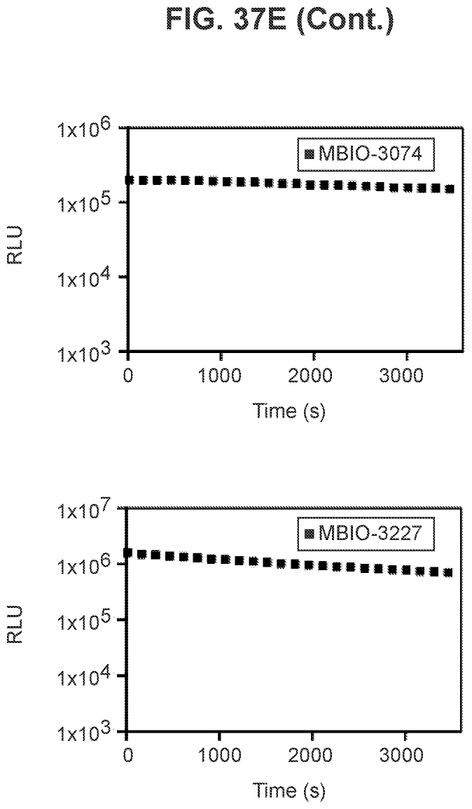

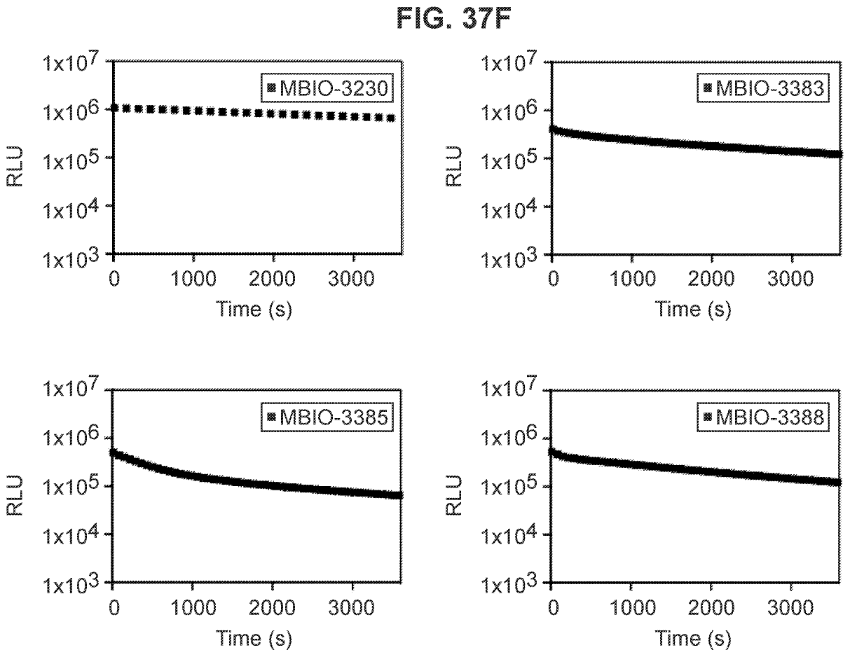

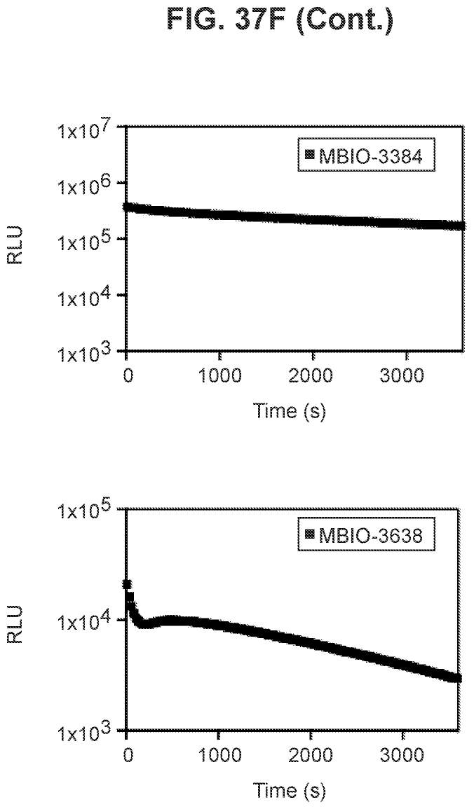

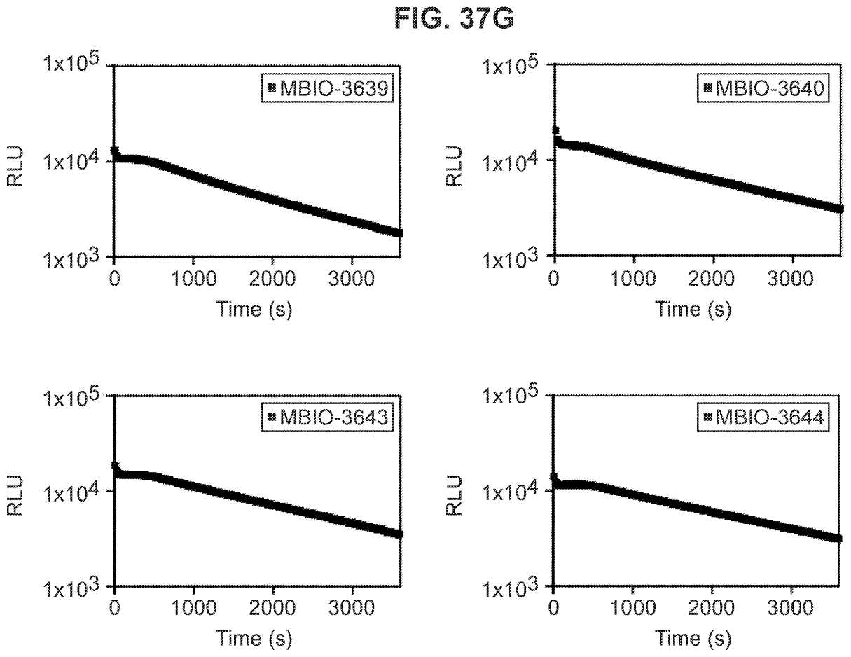

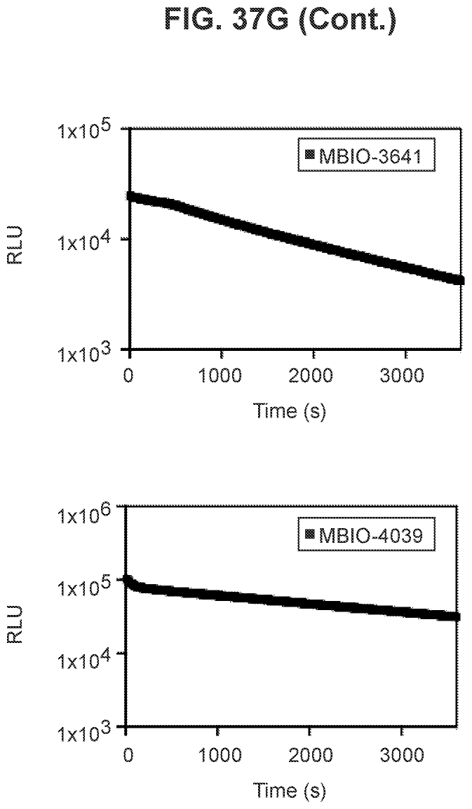

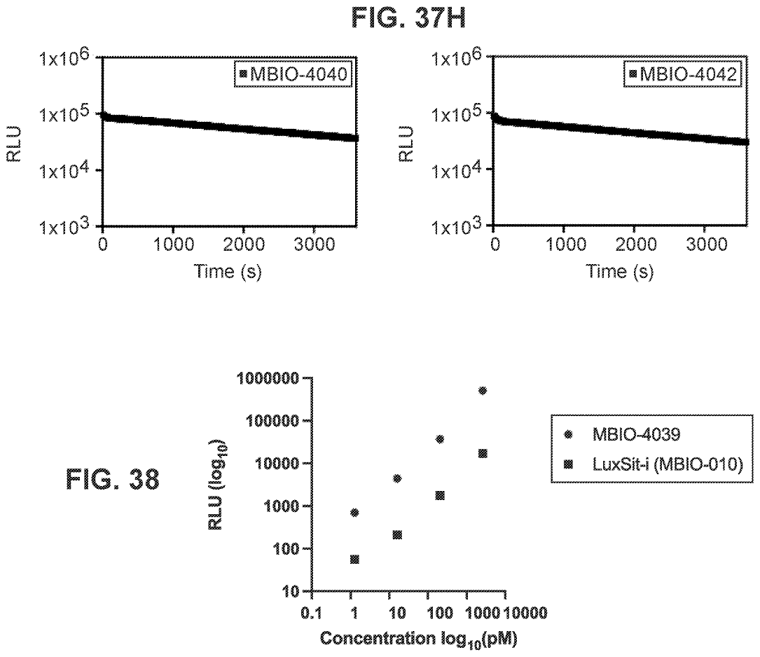

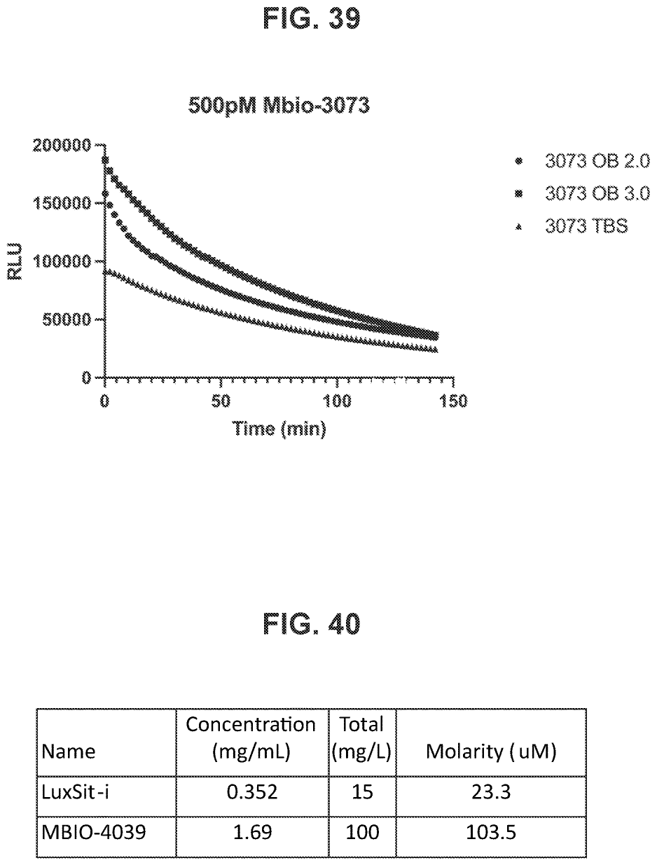

FIG. 37A-37H show enzymatic activity of listed LuxSit-i variants.

### Example 2: Circularly Permuted Proteins Having Luciferase Activity

Initial designs were generated using RosettaFold inpainting. Inpaints of lengths ranging 10-50 amino acids were generated, using MBIO-148/SEQ ID NO:2600 as input.

SEQ ID NO: 2600 (MBIO-148)—secondary structure arrangement is H1-L1-H2-L2-B1-L3-B2-L4-H3-L5-B3-L6-B4-L7-B5-L8-B6, where “H” is a helical domain, “L” is a loop domain, and “B” is a beta strand domain. The three types of domains are demarcated in the protein sequence:

MSEEQIRQFLRRFYEALDSGDADTAASLFHPGVTIHLWDGVTFTSREEFRE
WFERLFSTSKDAWREIKSLEVRGDTVEVHVQLHFTLNGQKHTVDLTHHFHF
RGNRVTEVRVHINPTG

The helical domains are indicated by the smaller font size. The loop domains are italicized and underlined. The beta strand domains are in bold.

The contigs specified for inpainting—i.e., the order in which SEQ ID NO:2600 domains were connected, were residues 89-117, LINKER, 1-88; where LINKER is a variable inpaint length of 10-50. The secondary structure of contigs was as follows: B5-L8-B6-LINKER-H1-L1-H2-L2-B1-L3-B2-L4-H3-L5-B3-L6-B4-(L7) (VII). The L7 domain in parenthesis is (i) present at the C-terminus, (ii) present at the N-terminus, (iii) split between the C-terminus and N-terminus, or (iii) absent.

In one design round, all beta sheet residues facing outward from the protein active site were mutated to valines prior to inpainting to facilitate inpaint structural packing against the rest of the protein.

After inpainting, each inpainted design was sequence-redesigned using Protein MPNN. MPNN was only permitted to change the inpainted residues and any mutated valine residues described above; the rest of the enzyme, including the active site residues, were retained. 20 sequences were generated for each inpainting output.

All MPNN-redesigned sequences were alphafolded using single-sequence prediction with 3 recycles. Top designs by pLDDT and low RMSD to the original SEQID:2600 structure were selected for further testing.

Select circularly permuted proteins each having the arrangement B5-L8-B6-LINKER-H1-L1-H2-L2-B1-L3-B2-L4-H3-L5-B3-L6-B4-(L7) (VII) are provided below. In these examples, the L7 domain in parenthesis is split between the C-terminus and N-terminus. Bold and underlined sequence indicates the linker sequence. The linker sequence is also referred to as inpaint. The helical domains are indicated by the smaller font size. The loop domains are italicized and underlined. The beta strand domains are in bold.

2209 - inpaint length 31:
GQKHVVVLIHVFRFRGNRVTEVEVRIFPAPVDDVEEVLARVLEEGERLVERLRAERPEASISEEQIRQFLRRFYEA
LDSGDADTAASLFHPGVTIHLWDGVTFTSREEFREWFERLFSTSKDAWRRIERLEVVGDTVVVVVVLEFTLN
263 - inpaint length 17
GQKHTVDLTHHFHFRGNRVTEVRVHITPTGEEPEKPEFKETFGPSSIPEEQIRQFLRRFYEALDSGDADTAASLFH
PGVTIHLWDGVTFTSREEFREWFERLFSTSKDAWREIKSLEVRGDTVEVHVQLHFTLN
2284 - inpaint length 35
GQKHLVVLVHLFRFRGNRVTEVEVRIFPVESEEELPAALARAEELGRELLERTLAEEGAGGPPEISEEQIRQFLRR
FYEALDSGDADTAASLFHPGVTIHLWDGVTFTSREEFREWFERLFSTSKDAWREIERLEVRGDTVVVVVRLRFTLN
2236 - inpaint length 32
GQKHVVVLVHTFRFRGNRVTEVRVEIIPAPSLDEESIEARVAEARRLAEERLAELGDPPPSISEEQIRQFLRRFYE
ALDSGDADTAASLFHPGVTIHLWDGVTFTSREEFREWFERLFSTSKDAWREIVELRVRGDTVVVVVVLHFTLN
259 - inpaint length 17
GQKHTVDLTHHFHFRGNRVTEVRVHIRPTGEEPEPPEFRERFGPSAIPEEQIRQFLRRFYEALDSGDADTAASLF
HPGVTIHLWDGVTFTSREEFREWFERLFSTSKDAWREIKSLEVRGDTVEVHVQLHFTLN
2221 - inpaint length 32
GQKHVVVLVHTFVFRGNRVTEVRVEIFPAPDLSPEAIEAAIAKALARADALLAELGAPPPSISEEQIRQFLRRFYE
ALDSGDADTAASLFHPGVTIHLWDGVTFTSREEFREWFERLFSTSKDAWREIVSLRVVGDTVVVVVVLHFTLN
256 - inpaint length 17
GQKHTVDLTHHFHFRGNRVTEVRVHIEPTGEEPERPEFVERFGPSSIPEEQIRQFLRRFYEALDSGDADTAASLF
HPGVTIHLWDGVTFTSREEFREWFERLFSTSKDAWREIKSLEVRGDTVEVHVQLHFTLN
2237 - inpaint length 32
GQKHVVVLVHTFVFRGNRVTEVRVEIFPVPSLDEAAIEAAIARARARADELLAELGAPPASISEEQIRQFLRRFYE
ALDSGDADTAASLFHPGVTIHLWDGVTFTSREEFREWFERLFSTSKDAWREIVSLRVVGDTVVVVVVLHFTLN
2227 - inpaint length 32
GQKHVVVLVHTFRFRGNRVTEVEVEIIPCPSLDEASIAAAIAEAEALAAERLAELGAPPPSISEEQIRQFLRRFYEA
LDSGDADTAASLFHPGVTIHLWDGVTFTSREEFREWFERLFSTSKDAWREIVSLRVVGDTVVVVVVLHFTLN
264 - inpaint length 17
GQKHTVDLTHHFHFRGNRVTEVRVHIEPTGEEPEPPEFRERFGPSSIPEEQIRQFLRRFYEALDSGDADTAASLF
HPGVTIHLWDGVTFTSREEFREWFERLFSTSKDAWREIKSLEVRGDTVEVHVQLHFTLN
2493 - inpaint length 45
GQKHTVILTHVFRFRGNRVTEVRVEIVPVPDPDEETRLAAAREALERAGVPEEMRRAALELLERGERELFRPSAI
PEEQIRQFLRRFYEALDSGDADTAASLFHPGVTIHLWDGVTFTSREEFREWFERLFSTSKDAWREILALVVDGDTVVVV
VRLDFTLN

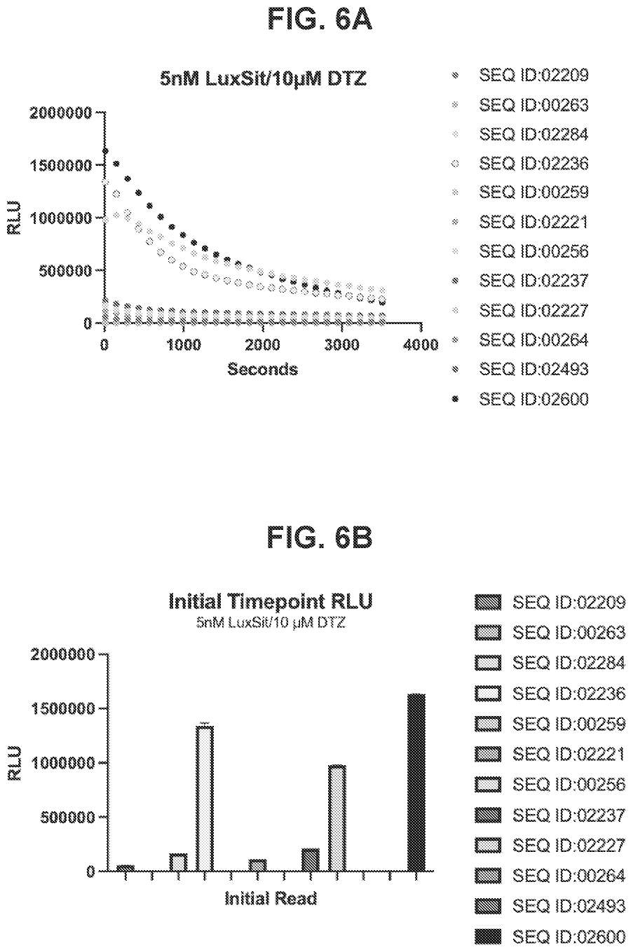

FIG. 6A provides kinetic profile over one hour of circularly permuted LuxSit-i variants. Variants were diluted in PBS and combined 1:1 with DTZ substrate, resulting in final in-well concentrations of 5 nM protein/variant and 10 μM DTZ.

FIG. 6B shows initial RLU values of circularly permuted LuxSit variants. Variants were diluted in PBS and combined 1:1 with DTZ substrate, resulting in final in-well concentrations of 5 nM protein and 10 μM DTZ.

Circularly permuted proteins having SEQ ID NOs: 2227 and 2236 have luciferase activity similar to SEQ ID NO:2600.

Additional examples of circularly permuted proteins are provide in Appendix B, which is herein incorporated by reference in its entirety.

### Example 3: Split LuxSit-i Variants

LuxSit-i variants, circularly permuted LuxSit-i, and circularly permuted LuxSit-i variants were split into two fragments. In some embodiments, the split point was placed such that two fragment of unequal length, a small fragment and a large fragment, were generated.

In certain embodiments, a circularly permuted LuxSit-i or a circularly permuted LuxSit-i variant having the secondary structure arrangement: B1-L3-B2-L4-H3-L5-B3-L6-B4-L7-B5-L8-B6-LINKER-H1-L1-H2-(L2) (II) or B2-L4-H3-L5-B3-L6-B4-L7-B5-L8-B6-LINKER-H1-L1-H2-L2-B1-(L3) (Ill) is split into two components. L domain in parenthesis is present at the C-terminus, or the N-terminus, or is split between the C-terminus and the N-terminus or is absent.

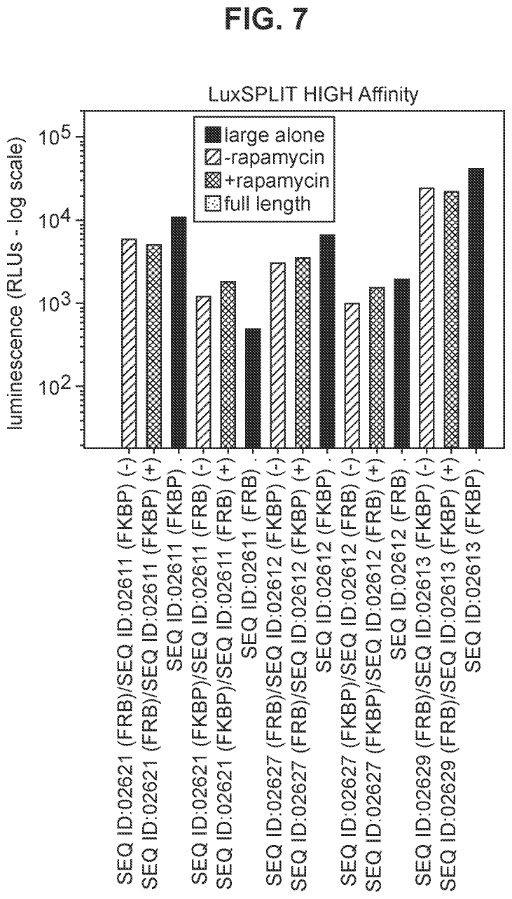

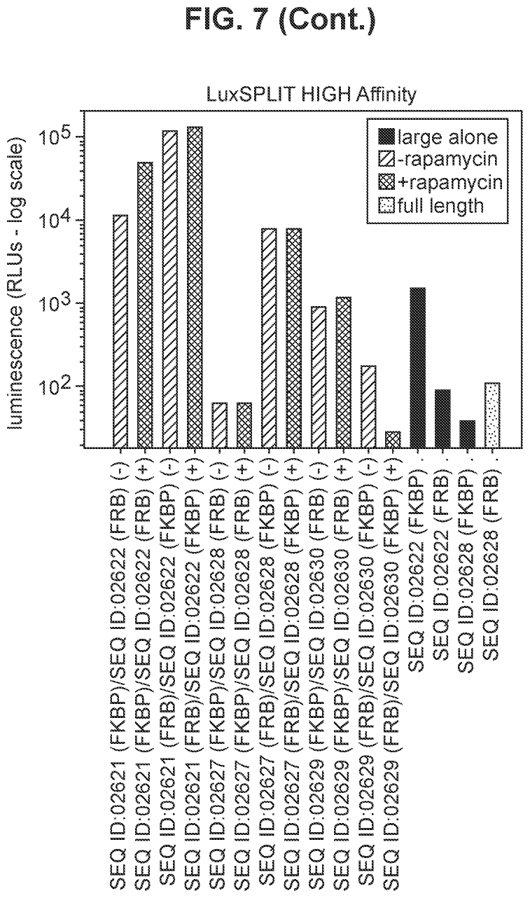

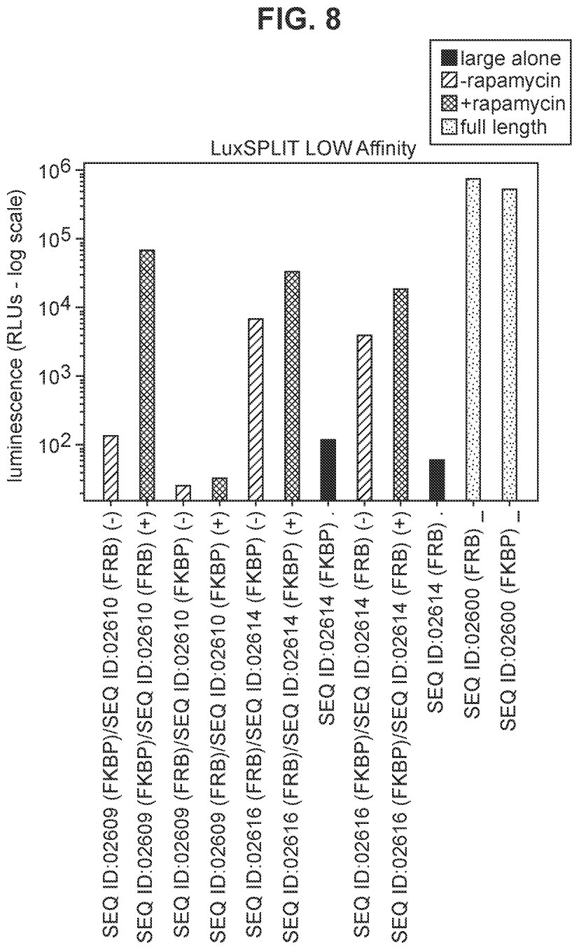

Luminescent activity of the high-affinity two-component luciferase variants fused to the rapamycin inducible FRB:FKBP system is shown in FIG. 7. Luminescent activity of the low-affinity two-component luciferase variants fused to the rapamycin inducible FRB:FKBP system is shown in FIG. 8.

Sequences of two-component luciferase variants are set forth in Table 13. These components are also referred to as fragments. In certain embodiments, sequences of two-component luciferase variants vary in size. In certain embodiments, the smallest small component is about 5 to 6 amino acids in size and the largest small component is about 30 to 40 amino acids in size. In certain embodiments, the smallest large component is about 70 to 80 amino acids in size and the largest large component is about around 110 amino acids in size.

Table 13A. Lists small and large components of two-component luciferase variants. “smLux” refers to the small component that is small relative to the component that complements it to increase luciferase activity. “IgLux” refers to the large component that complements the activity of the smLx.

TABLE 13A

| | | SEQ ID |
|---|---|---|
| Component | Sequence | NO |
| smLux_MBIO-148_cut44 1-44 | MSEEQIRQFLRRFYEALDSGDADTAASLFHPGVTIHLWDGVTFT | 2605 |
| lgLux_MBIO-148_cut44_45-117 | SREEFREWFERLFSTSKDAWREIKSLEVRGDTVEVHVQLHFTLNGQK | 2606 |
| | HTVDLTHHFHFRGNRVTEVRVHINPTGLE | |
| lgLux_MBIO-148_cut74_1-74 | MSEEQIRQFLRRFYEALDSGDADTAASLFHPGVTIHLWDGVTFTSRE | 2608 |
| | EFREWFERLFSTSKDAWREIKSLEVRG | |
| smLux_MBIO-148_cut74_75-117 | DTVEVHVQLHFTLNGQKHTVDLTHHFHFRGNRVTEVRVHINPTGLE | 2607 |
| lgLux_MBIO-148_cut104_1-104 | MSEEQIRQFLRRFYEALDSGDADTAASLFHPGVTIHLWDGVTFTSRE | 2610 |
| | EFREWFERLFSTSKDAWREIKSLEVRGDTVEVHVQLHFTLNGQKHTV | |
| | DLTHHFHFRG | |
| smLux_MBIO-148_cut104_105- | NRVTEVRVHINPTGLE | 2609 |
| 117 | | |
| MBIO-302_IgLux2 | PSISEEQIRQNLRRFYEALDSGDADTAASLFHPGVTIHLWDGVTFTSR | 2611 |
| | EEFREWFERLFSTSKDALREIVELRVRGDTVVVVVVLHFTRNGQKHV | |
| | VVLVHLWHFRGNRVDEVRVEIIPAP | |
| MBIO-302_IgLux5 | DGVTFTSREEFREWFERLFSTSKDALREIVELRVRGDTVVVVVVLHFT | 2612 |
| | RNGQKHVVVLVHTWRFRGNRVDEVRVEIIPAPSLDEESIEARVAEAR | |
| | RLAEERLAELGDPPPSISEEQIRQNLRRFYEALDSGDADTAASLFHP | |
| MBIO-302_IgLux6 | SREEFREWFERLFSTSKDALREIVELRVRGDTVVVVVVLHFTRNGQKH | 2613 |
| | VVVLVHTWRFRGNRVDEVRVEIIPAPSLDEESIEARVAEARRLAEERL | |
| | AELGDPPPSISEEQIRQNLRRFYEALDSGDADTAASLFHPGVTIHLW | |
| MBIO-301_IgLux104 | SEEQIRQNLRRFYEALDSGDADTAASLFHPGVTIHLWDGVTFTSREEF | 2614 |
| | REWFERLFSTSKDALREIKSLEVRGDTVEVHVQLHFTRNGQKHTVDL | |
| | THLFHFR | |
| MBIO-301_smLux104_no_PT | NRVDEVRVYIN | 2615 |
| MBIO-301_smLux104_PT | NRVDEVRVYINPT | 2616 |
| smLux_0_MBIO_367 | GQKHVVVLVHTFRFRG | 2617 |
| lgLux_0_MBIO_367 | NRVTEVRVEIIPAPSLDEESIEARVAEARRLAEERLAELGDPPPSISEEQI | 2618 |
| | RQFLRRFYEALDSGDADTAASLFHPGVTIHLWDGVTFTSREEFREWF | |
| | ERLFSTSKDAWREIVELRVRGDTVVVVVVLHFTLN | |
| smLux_1_MBIO_367 | NRVTEVRVEIIPAP | 2619 |
| lgLux_1_MBIO_367 | SLDEESIEARVAEARRLAEERLAELGDPPPSISEEQIRQFLRRFYEALDS | 2620 |
| | GDADTAASLFHPGVTIHLWDGVTFTSREEFREWFERLFSTSKDAWRE | |
| | IVELRVRGDTVVVVVVLHFTLNGQKHVVVLVHTFRFRG | |
| smLux_2_MBIO_367 | SLDEESIEARVAEARRLAEERLAELGDPP | 2621 |
| lgLux_2_MBIO_367 | PSISEEQIRQFLRRFYEALDSGDADTAASLFHPGVTIHLWDGVTFTSRE | 2622 |
| | EFREWFERLFSTSKDAWREIVELRVRGDTVVVVVVLHFTLNGQKHVV | |
| | VLVHTFRFRGNRVTEVRVEIIPAP | |
| smLux_3_MBIO_367 | PSISEEQIRQFLRRFYEALDSG | 2623 |
| lgLux_3_MBIO_367 | DADTAASLFHPGVTIHLWDGVTFTSREEFREWFERLFSTSKDAWREI | 2624 |
| | VELRVRGDTVVVVVVLHFTLNGQKHVVVLVHTFRFRGNRVTEVRVEI | |
| | IPAPSLDEESIEARVAEARRLAEERLAELGDPP | |
| smLux_4_MBIO_367 | DADTAASLFHP | 2625 |
| lgLux_4_MBIO_367 | GVTIHLWDGVTFTSREEFREWFERLFSTSKDAWREIVELRVRGDTVV | 2626 |
| | VVVVLHFTLNGQKHVVVLVHTFRFRGNRVTEVRVEIIPAPSLDEESIEA | |
| | RVAEARRLAEERLAELGDPPPSISEEQIRQFLRRFYEALDSG | |
| smLux_5_MBIO_367 | GVTIHLW | 2627 |
| lgLux_5_MBIO_367 | DGVTFTSREEFREWFERLFSTSKDAWREIVELRVRGDTVVVVVVLHF | 2628 |
| | TLNGQKHVVVLVHTFRFRGNRVTEVRVEIIPAPSLDEESIEARVAEAR | |
| | RLAEERLAELGDPPPSISEEQIRQFLRRFYEALDSGDADTAASLFHP | |
| smLux_6_MBIO_367 | DGVTFT | 2629 |
| lgLux_6_MBIO_367 | SREEFREWFERLFSTSKDAWREIVELRVRGDTVVVVVVLHFTLNGQK | 2630 |
| | HVVVLVHTFRFRGNRVTEVRVEIIPAPSLDEESIEARVAEARRLAEERL | |
| | AELGDPPPSISEEQIRQFLRRFYEALDSGDADTAASLFHPGVTIHLW | |
| smLux_7_MBIO_367 | SREEFREWFERLFSTSK | 2631 |
| lgLux_7_MBIO_367 | DAWREIVELRVRGDTVVVVVVLHFTLNGQKHVVVLVHTFRFRGNRV | 2632 |
| | TEVRVEIIPAPSLDEESIEARVAEARRLAEERLAELGDPPPSISEEQIRQF | |
| | LRRFYEALDSGDADTAASLFHPGVTIHLWDGVTFT | |
| smLux_8_MBIO_367 | DAWREIVELRVRG | 2633 |
| lgLux_8_MBIO_367 | DTVVVVVVLHFTLNGQKHVVVLVHTFRFRGNRVTEVRVEIIPAPSLDE | 2634 |
| | ESIEARVAEARRLAEERLAELGDPPPSISEEQIRQFLRRFYEALDSGDA | |
| | DTAASLFHPGVTIHLWDGVTFTSREEFREWFERLFSTSK | |
| smLux_9_MBIO_367 | DTVVVVVVLHFTLN | 2635 |
| lgLux_9_MBIO_367 | GQKHVVVLVHTFRFRGNRVTEVRVEIIPAPSLDEESIEARVAEARRLAE | 2636 |
| | ERLAELGDPPPSISEEQIRQFLRRFYEALDSGDADTAASLFHPGVTIHL | |
| | WDGVTFTSREEFREWFERLFSTSKDAWREIVELRVRG | |
| mpnn78_smlux2_104_trip | MSGNRVDEVRVYINPT | 2637 |
| mpnn78_smlux1_88-103_trip | MSGGERREVELTHLFTFRG | 2638 |
| mpnn78_lglux_87_trip | MSGSDAERAALLDRFYAALNAGDADAAAALFPPGVTIELWNGVVFRSR | 2639 |
| | EEFRAWFAELFARSPEARREVLSREIEGDRVRVRVRLTFVRD | |
| 3073_smlux2_104_trip | MSGNRVVDVRVYTNPT | 2640 |
| 3073_smlux1_88-103_trip | MSGGQKHTVDLLQLFKFVG | 2641 |
| 3073_smlux 111 11 | MSGVYTNPT | 2642 |
| 3073_smlux_109_10 | MSGVRVYTNPT | 2643 |
| 3073_smlux_107_9 | MSGVDVRVYTNPT | 2644 |
| 3073_smlux_105_7 | MSGRVVDVRVYTNPT | 2645 |
| 3073_smlux_104_8_classic | MSGNRVVDVRVYTNPT | 2646 |
| 3073_smlux_104_8_classic_V6A | MSGNRVVDARVYTNPT | 2647 |
| 3073_smlux_104_8_classic_N11E | MSGNRVVDVRVYTEPT | 2648 |
| 3073_smlux_104_8_classic_N11E_V | MSGNRVVDARVYTEPT | 2649 |
| 6A | | |
| 3073_smlux 103_6 | MSGGNRVVDVRVYTNPT | 2650 |
| 3073_smlux_101_5 | MSGFVGNRVVDVRVYTNPT | 2651 |
| 3073_smlux_99_4 | MSGFKFVGNRVVDVRVYTNPT | 2652 |
| 3073_smlux_97_3 | MSGQLFKFVGNRVVDVRVYTNPT | 2653 |
| 3073_smlux_95_2 | MSGLLQLFKFVGNRVVDVRVYTNPT | 2654 |
| 3073_smlux_89_1 | MSGQKHTVDLLQLFKFVGNRVVDVRVYTNPT | 2655 |
| 3073_Iglux_108_11 | MSGSEEQIRQNLRRFYEALDSGDADTAASLFDPGVTITLWDGTTFTSVE | 2656 |
| | EFREWFERLFSTSKDALREIKSLEVRGDTVEVTVQLSFTRNGQKHTVDLL | |
| | QLFKFVGNRVVD | |
| 3073_Iglux_106_10 | MSGSEEQIRQNLRRFYEALDSGDADTAASLFDPGVTITLWDGTTFTSVE | 2657 |
| | EFREWFERLFSTSKDALREIKSLEVRGDTVEVTVQLSFTRNGQKHTVDLL | |
| | QLFKFVGNRV | |
| 3073_lglux_104_9 | MSGSEEQIRQNLRRFYEALDSGDADTAASLFDPGVTITLWDGTTFTSVE | 2658 |
| | EFREWFERLFSTSKDALREIKSLEVRGDTVEVTVQLSFTRNGQKHTVDLL | |
| | QLFKFVGN | |
| 3073_lglux_103_8_classic | MSGSEEQIRQNLRRFYEALDSGDADTAASLFDPGVTITLWDGTTFTSVE | 2659 |
| | EFREWFERLFSTSKDALREIKSLEVRGDTVEVTVQLSFTRNGQKHTVDLL | |
| | QLFKFVG | |
| 3073_lglux_102_7 | MSGSEEQIRQNLRRFYEALDSGDADTAASLFDPGVTITLWDGTTFTSVE | 2660 |
| | EFREWFERLFSTSKDALREIKSLEVRGDTVEVTVQLSFTRNGQKHTVDLL | |
| | QLFKFV | |
| 3073_lglux_100_6 | MSGSEEQIRQNLRRFYEALDSGDADTAASLFDPGVTITLWDGTTFTSVE | 2661 |
| | EFREWFERLFSTSKDALREIKSLEVRGDTVEVTVQLSFTRNGQKHTVDLL | |
| | QLFK | |
| 3073_lglux_98_5 | MSGSEEQIRQNLRRFYEALDSGDADTAASLFDPGVTITLWDGTTFTSVE | 2662 |
| | EFREWFERLFSTSKDALREIKSLEVRGDTVEVTVQLSFTRNGQKHTVDLL | |
| | QL | |
| 3073_lglux_96_4 | MSGSEEQIRQNLRRFYEALDSGDADTAASLFDPGVTITLWDGTTFTSVE | 2679 |
| | EFREWFERLFSTSKDALREIKSLEVRGDTVEVTVQLSFTRNGQKHTVDLL | |
| 3073_lglux_94_3 | MSGSEEQIRQNLRRFYEALDSGDADTAASLFDPGVTITLWDGTTFTSVE | 2680 |
| | EFREWFERLFSTSKDALREIKSLEVRGDTVEVTVQLSFTRNGQKHTVD | |
| 3073_Iglux_92_2 | MSGSEEQIRQNLRRFYEALDSGDADTAASLFDPGVTITLWDGTTFTSVE | 2681 |
| | EFREWFERLFSTSKDALREIKSLEVRGDTVEVTVQLSFTRNGQKHT | |
| 3073_lglux_88_1 | MSGSEEQIRQNLRRFYEALDSGDADTAASLFDPGVTITLWDGTTFTSVE | 2666 |
| | EFREWFERLFSTSKDALREIKSLEVRGDTVEVTVQLSFTRNG | |
| 3073_lglux_87_trip | MSGSEEQIRQNLRRFYEALDSGDADTAASLFDPGVTITLWDGTTFTSVE | 2667 |
| | EFREWFERLFSTSKDALREIKSLEVRGDTVEVTVQLSFTRN | |
| 3073_FL_darkbit_V-Y | MSGSEEQIRQNLRRFYEALDSGDADTAASLFDPGVTITLWDGTTFTSVE | 2668 |
| | EFREWFERLFSTSKDALREIKSLEVRGDTVEVTVQLSFTRNGQKHTVDLL | |
| | QLFKFVGNRVVDYRVYTNPT | |
| 3073_FL_darkbit_V-R | MSGSEEQIRQNLRRFYEALDSGDADTAASLFDPGVTITLWDGTTFTSVE | 2669 |
| | EFREWFERLFSTSKDALREIKSLEVRGDTVEVTVQLSFTRNGQKHTVDLL | |
| | QLFKFVGNRVVDRRVYTNPT | |
| 3073_FL_darkbit_V-M | MSGSEEQIRQNLRRFYEALDSGDADTAASLFDPGVTITLWDGTTFTSVE | 2670 |
| | EFREWFERLFSTSKDALREIKSLEVRGDTVEVTVQLSFTRNGQKHTVDLL | |
| | QLFKFVGNRVVDMRVYTNPT | |
| 3073_FL_darkbit_V-D | MSGSEEQIRQNLRRFYEALDSGDADTAASLFDPGVTITLWDGTTFTSVE | 2671 |
| | EFREWFERLFSTSKDALREIKSLEVRGDTVEVTVQLSFTRNGQKHTVDLL | |
| | QLFKFVGNRVVDDRVYTNPT | |
| 301_smlux2_104_trip | MSGNRVDEVRVYINPT | 2672 |
| 301_smlux1_88-103_trip | MSGGQKHTVDLTHLFHFRG | 2673 |
| 301_lglux_87_trip | MSGSEEQIRQNLRRFYEALDSGDADTAASLFHPGVTIHLWDGVTFTSRE | 2674 |
| | EFREWFERLFSTSKDALREIKSLEVRGDTVEVHVQLHFTRN | |
| 4039_Iglux102 | SEEQIRQNLRRFYEALDSGDAITAASLFDPGVTITLWDGTTFTSVEEFRE | 2675 |
| | WFESQFSTSKDALREISSLEVRGDTVEVTVRLSFTRNGQKHTVDLLQLFK | |
| | FV | |
| 4039_smlux105 | RVVAVRVYVNPT | 2676 |
| 4040_Iglux102 | SEEQIRQNLRRFYEALDSGDADTAASLFDPGVTITLWDGVTFTSREEFRE | 2677 |
| | WFERQFSTSKDALREIKSLEVRGDTVEVTIQLSFTRNGQKSTVDLTQLFR | |
| | FR | |
| 4040_lglux105 | RVDEVRVYINPT | 2678 |

Table 13A also lists polypeptides that have the same length as the LuxSit-i variants disclosed herein and have one or more mutations that render them inactive. These polypeptides are referred to as darkbit in Table 13A. These polypeptides regain activity when associated with any of the small fragments disclosed herein, e.g., any smlux of Table 13A. In certain embodiments, the present disclosure provides a kit comprising a IgLux and a smLux as disclosed herein or a nucleic acid encoding a IgLux and a nucleic acid encoding a smLux, wherein the IgLux comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence of a IgLux disclosed herein (e.g., in Table 13A, Table 19, Table 20, Table 21, or Table 22), and the smLux comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence of a smLux disclosed herein (e.g., in Table 13A or Table 18).

Also provided herein are one or both of a first nucleic acid encoding a first protein and a second nucleic acid encoding a second protein, wherein:
(i) the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: MSEEQIRQFLRRFYEALDSGDADTAASLFHPGVTIHLWDGVTFT (SEQ ID NO:2605) and the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2606)
SREEFREWFERLFSTSKDAWREIKSLEVRGDTVEVHVQLHFTLNGQKHTV
DLTHHFHFRGNRVTEVRVHINPTGLE

(ii) the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: MSEEQIRQFLRRFYEALDSGDADTAASLFHPGVTIHLWDGVTFTSREEFREWFERLFSTSKDAWREIKSLEVRG (SEQ ID NO:2608) and the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: DTVEVHVQLHFTLNGQKHTVDLTHHFHFRGNRVTEVRVHINPTGLE (SEQ ID NO: 2607);
(iii) the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: MSEEQIRQFLRRFYEALDSGDADTAASLFHPGVTIHLWDGVTFTSREEFREWFERLFSTSKDAWREIKSLEVRGDTVEV HVQLHFTLNGQKHTVDLTHHFHFRG (SEQ ID NO:2610) and the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: NRVTEVRVHINPTGLE (SEQ ID NO: 2609);
(iv) the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: SEEQIRQNLRRFYEALDSGDADTAASLFHPGVTIHLWDGVTFTSREEFREWFERLFSTSKDALREIKSLEVRGDTVEVHV QLHFTRNGQKHTVDLTHLFHFR (SEQ ID NO:2614) and the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: NRVDEVRVYIN (SEQ ID NO: 2615) or NRVDEVRVYINPT (SEQ ID NO:2616);
(v) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: GQKHVVVLVHTFRFRG (SEQ ID NO:2617) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2618)
NRVTEVRVEIIPAPSLDEESIEARVAEARRLAEERLAELGDPPPSISEEQ
IRQFLRRFYEALDSGDADTAASLFHPGVTIHLWDGVTFTSREEFREWFER
LFSTSKDAWREIVELRVRGDTVVVVVVLHFTLN;

(vi) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: NRVTEVRVEIIPAP (SEQ ID NO:2619) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2620)
SLDEESIEARVAEARRLAEERLAELGDPPPSISEEQIRQFLRRFYEALDS
GDADTAASLFHPGVTIHLWDGVTFTSREEFREWFERLFSTSKDAWREIVE
LRVRGDTVVVVVVLHFTLNGQKHVVVLVHTFRFRG;

(vii) the first protein comprises at least 50%, 55%, 60%, 65%, 70%, 75%, 80%, 85%, 90%, 91%, 92%, 93% 94%, 95%, 96%, 97%, 98%, or 99% identity to the amino acid sequence: SLDEESIEARVAEARRLAEERLAELGDPP (SEQ ID NO:2621) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2622)
PSISEEQIRQFLRRFYEALDSGDADTAASLFHPGVTIHLWDGVTFTSREE
FREWFERLFSTSKDAWREIVELRVRGDTVVVVVVLHFTLNGQKHVVVLVH
TFRFRGNRVTEVRVEIIPAP;

(viii) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: PSISEEQIRQFLRRFYEALDSG (SEQ ID NO:2623) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2624)
DADTAASLFHPGVTIHLWDGVTFTSREEFREWFERLFSTSKDAWREIVEL
RVRGDTVVVVVVLHFTLNGQKHVVVLVHTFRFRGNRVTEVRVEIIPAPSL
DEESIEARVAEARRLAEERLAELGDPP;

(ix) the first protein comprises at least 50%, 55%, 60%, 65%, 70%, 75%, 80%, 85%, 90%, 91%, 92%, 93% 94%, 95%, 96%, 97%, 98%, or 99% identity to the amino acid sequence: DADTAASLFHP (SEQ ID NO:2625) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2626)
GVTIHLWDGVTFTSREEFREWFERLFSTSKDAWREIVELRVRGDTVVVVV
VLHFTLNGQKHVVVLVHTFRFRGNRVTEVRVEIIPAPSLDEESIEARVAE
ARRLAEERLAELGDPPPSISEEQIRQFLRRFYEALDSG;

(x) the first protein comprises at least 50%, 55%, 60%, 65%, 70%, 75%, 80%, 85%, 90%, 91%, 92%, 93% 94%, 95%, 96%, 97%, 98%, or 99% identity to the amino acid sequence: GVTIHLW (SEQ ID NO:2627) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2628)
DGVTFTSREEFREWFERLFSTSKDAWREIVELRVRGDTVVVVVVLHFTLN
GQKHVVVLVHTFRFRGNRVTEVRVEIIPAPSLDEESIEARVAEARRLAEE
RLAELGDPPPSISEEQIRQFLRRFYEALDSGDADTAASLFHP;

(xi) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: DGVTFT (SEQ ID NO:2629) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2630)
SREEFREWFERLFSTSKDAWREIVELRVRGDTVVVVVVLHFTLNGQKHVV
VLVHTFRFRGNRVTEVRVEIIPAPSLDEESIEARVAEARRLAEERLAELG
DPPPSISEEQIRQFLRRFYEALDSGDADTAASLFHPGVTIHLW;

(xii) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: SREEFREWFERLFSTSK (SEQ ID NO:2631) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2632)
DAWREIVELRVRGDTVVVVVVLHFTLNGQKHVVVLVHTFRFRGNRVTEVR
VEIIPAPSLDEESIEARVAEARRLAEERLAELGDPPPSISEEQIRQFLRR
FYEALDSGDADTAASLFHPGVTIHLWDGVTFT;

(xiii) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: DAWREIVELRVRG (SEQ ID NO:2633) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2634)
DTVVVVVVLHFTLNGQKHVVVLVHTFRFRGNRVTEVRVEIIPAPSLDEES
IEARVAEARRLAEERLAELGDPPPSISEEQIRQFLRRFYEALDSGDADTA
ASLFHPGVTIHLWDGVTFTSREEFREWFERLFSTSK;

(xiv) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: DTVVVVVVLHFTLN (SEQ ID NO:2635) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:

(SEQ ID NO: 2636)
GQKHVVVLVHTFRFRGNRVTEVRVEIIPAPSLDEESIEARVAEARRLAEE
RLAELGDPPPSISEEQIRQFLRRFYEALDSGDADTAASLFHPGVTIHLWD
GVTFTSREEFREWFERLFSTSKDAWREIVELRVRG,
or

(xv) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: MSGMSEEQIRQNLRRFYEALDSGDAITAASLFDPGVTITLWDGTTFTSVEEFREWFESQFSTSKDALREISSLEVRGDTV EVTVRLSFTRNGQKHTVDLLQLFKFV (SEQ ID NO:xx) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: MSGGNRVVAVRVYVNPT (SEQ ID NO: 2734).

Each fragment of the bipartite luciferase variants was fused to FRB FKBP proteins and incubated with its complementary part at a 1:1 ratio. Full length luciferase variants and the large split fragment alone were included as controls. All constructs were tested in E. coli cell lysates. Rapamycin was added to induce reconstitution of the two fragments and Diphenylterazine (DTZ) was added as luminescent substrate.

The small fragments listed in Table 13 can complement the listed large fragments to form a complex that has higher enzymatic activity than either the small fragment or the larger fragment by itself.

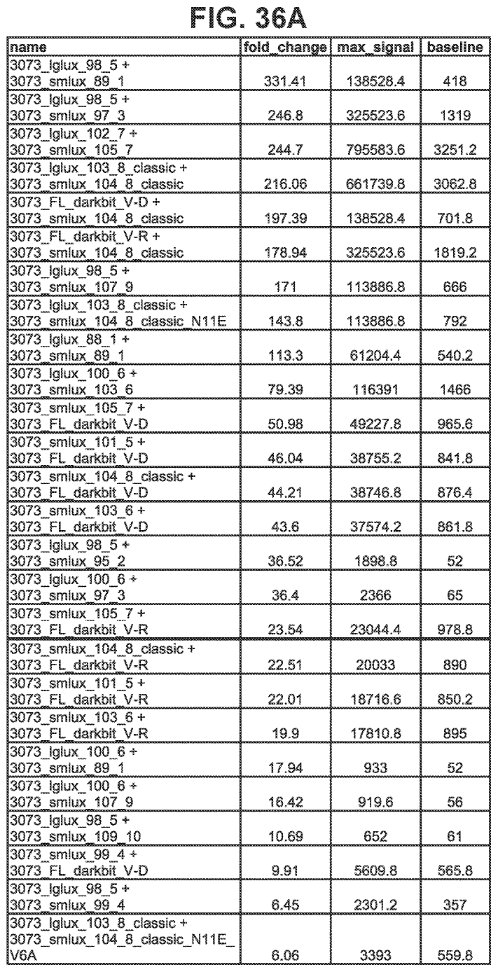

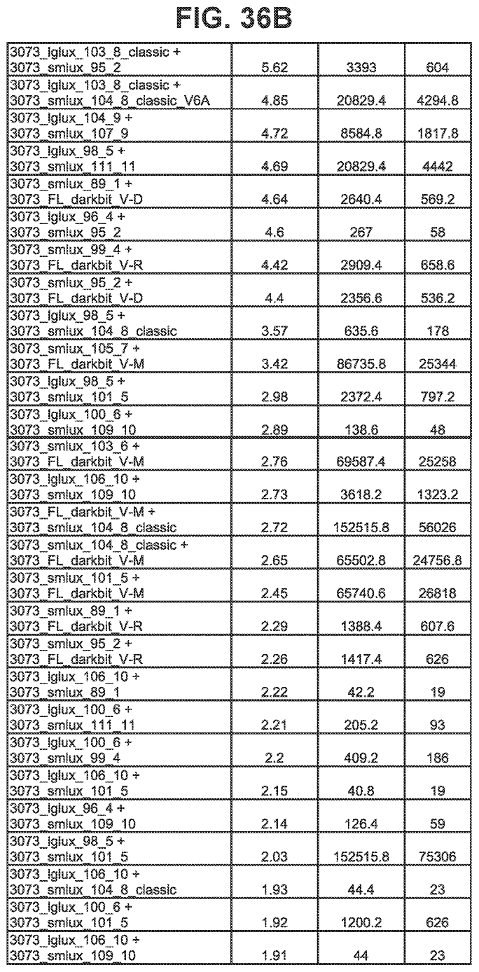

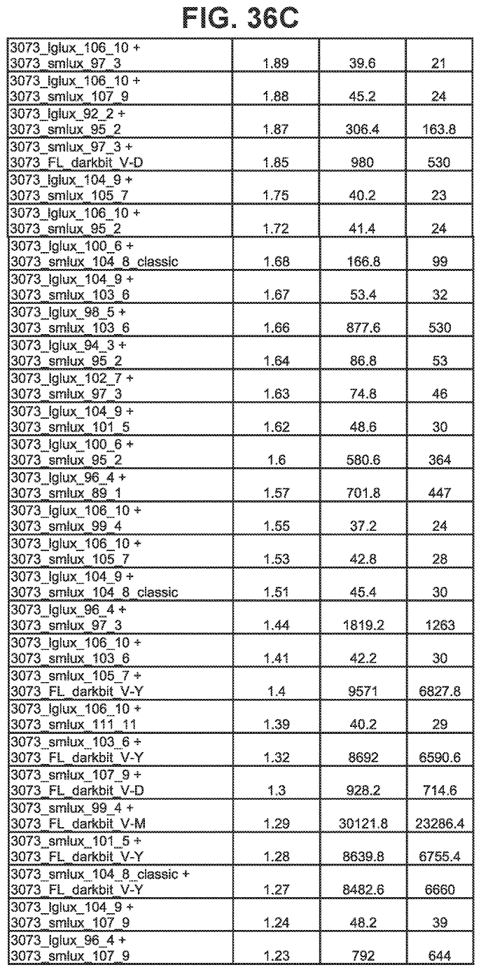

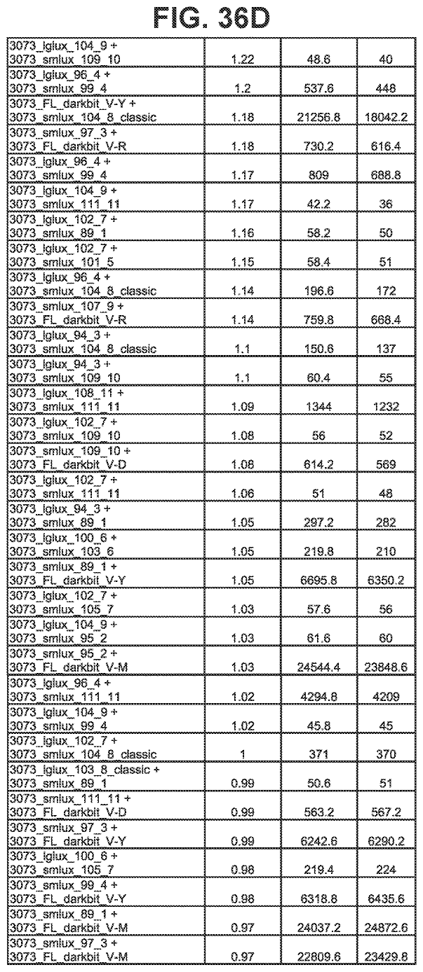

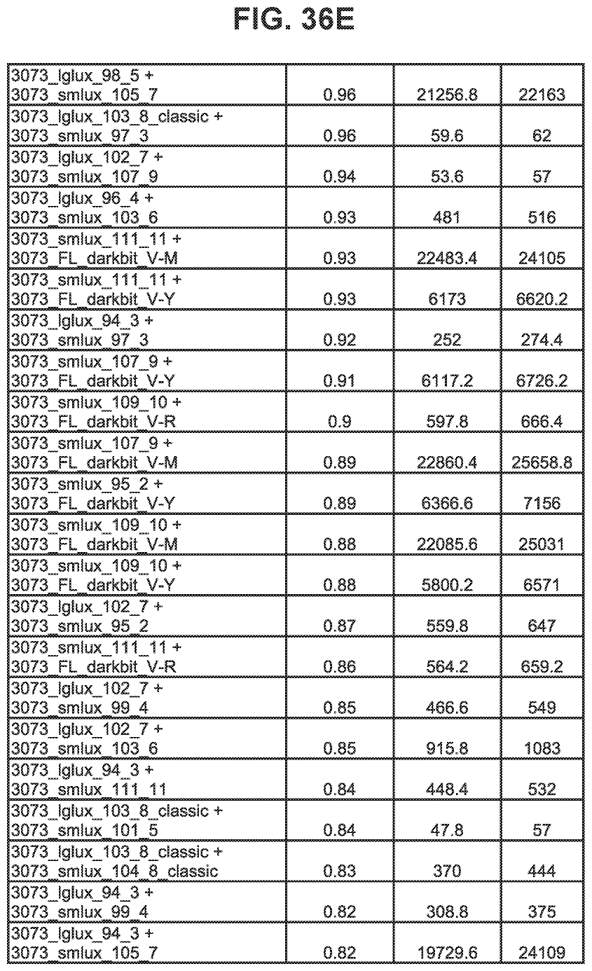

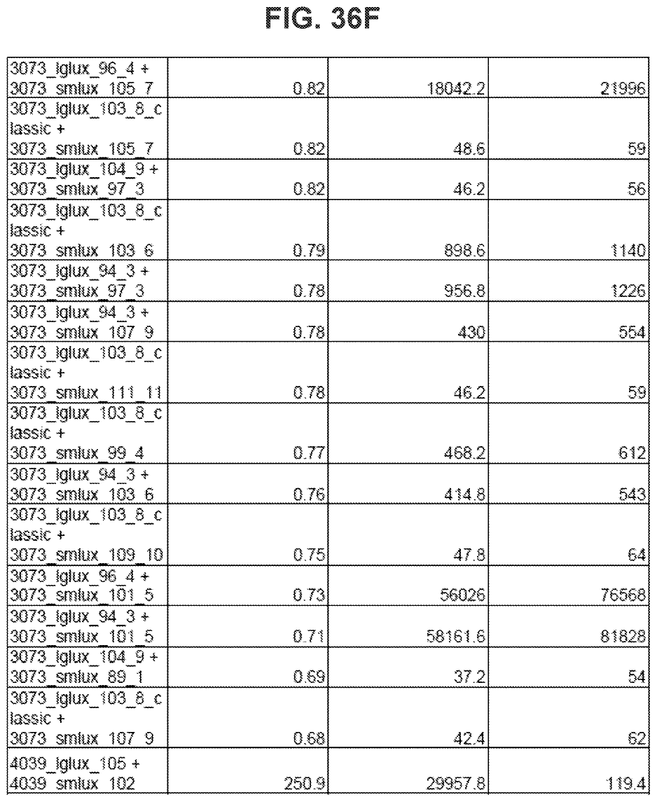

FIGS. 36A-36F provide results from pairs of split luxsit variants screened for rapamycin-induced luminescence. All Iglux constructs are in the format mcherry-28x-linker-FRB-33x-linker-Iglux-His_tag, and all smlux constructs are in the format mcherry-FKBP-smlux-His_tag. 28x and 33x linkers are flexible GS sequences. The concentration of each sample was calculated using mcherry fluorescence. Each sample was measured at 1 nM Iglux+1 nM smlux. Fold change was calculated by taking the difference in signal 15 mins after the addition of 20 uM rapamycin or PBS to each sample. Substrate was added at a concentration of 50 uM per sample. Max signal is reported for the (+) rapamycin condition 15 mins after rapamycin addition, and baseline is reported for the (−) rapamycin condition 15 mins after PBS addition. Data were collected in Corning 3600 opaque 96-well plates on a BioTek plate reader.

### Example 4: Optimized (“OB” or “OPT”) Buffer for Measuring Luciferase Activity

Assay buffers that increase and stabilize the signal output of luciferase assay were tested. Inclusion of Imidazole increased the brightness of LuxSit at pH8. An optimized buffer (OPT1.0) having the effect of increasing and stabilizing the signal output of luciferase has the composition: 1×PBS, 0.5% Propylene Glycol, 0.1% Anti-Foam, 10 mM Ascorbic Acid, 35 mM Thiourea, 100 mM Imidazole, pH 8.0.

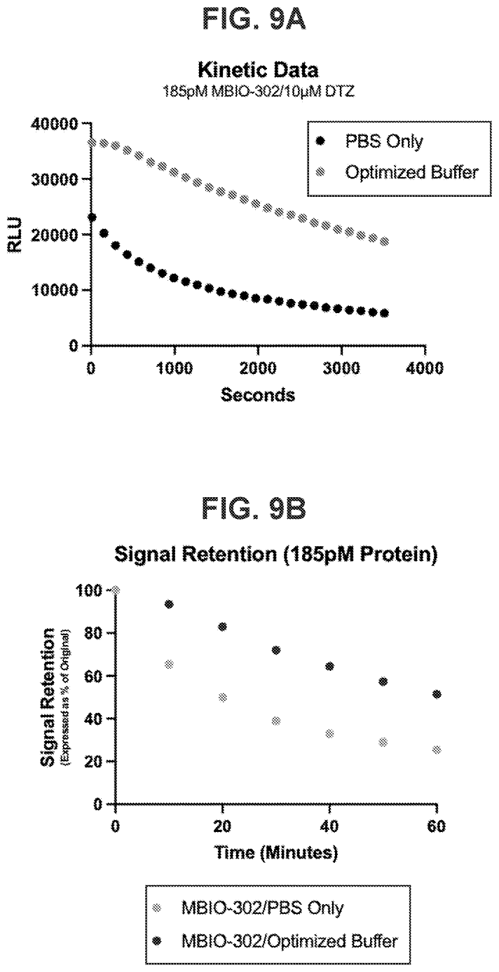

FIG. 9A shows kinetic RLU values over the course of an hour comparing MBIO-302 diluted in PBS only or Optimized Buffer. In-well concentrations were 185 pM of enzyme and 10 μM DTZ. FIG. 9B shows signal retention expressed as a percentage of initial RLU value over the course of one hour for 185 pM MBIO-302 diluted in PBS only or in Optimized Buffer. In-well concentrations of DTZ was 10 μM.

Additional optimized buffers, OB3.0 and OB2.0 have been formulated in 1×PBS:

TABLE 14
OB3.0

| | Molarity | | |
|---|---|---|---|
| Reagent | (mM) | % w/v | % v/v |
| Imidazole | 170 | 1.1574 | — |
| Glycine | 375 | 2.8151 | — |
| Propylene Glycol | — | — | 0.5 |
| Antifoam 204 | — | — | 0.1 |

TABLE 15
OB2.0

| | Molarity | | |
|---|---|---|---|
| Reagent | (mM) | % w/v | % v/v |
| Imidazole | 10 | 0.0681 | — |
| Glycine | 250 | 1.8768 | — |
| Propylene Glycol | — | — | 0.5 |
| Antifoam 204 | — | — | 0.1 |

### Example 5: Bioluminescence Emission Spectra of Synthetic Luciferin Substrates with MBIO-301 Enzyme

Bioluminescence emission spectra of synthetic luciferin substrates was measured. The luciferin substrates were incubated with MBIO-301 enzyme at 500 μM. Each of the molecules were incubated with 1× Phosphate-buffered saline (PBS), 0.5% Propylene Glycol, 0.05% Anti-foam, 100 mM Imidazole, 10 mM Ascorbic Acid buffer and emission spectra was obtained using a Synergy H1 plate reader.

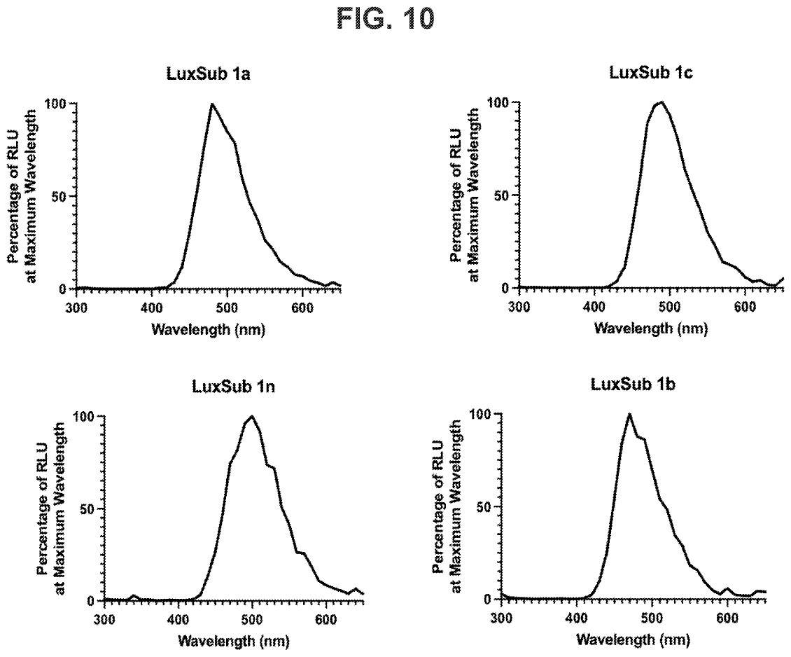

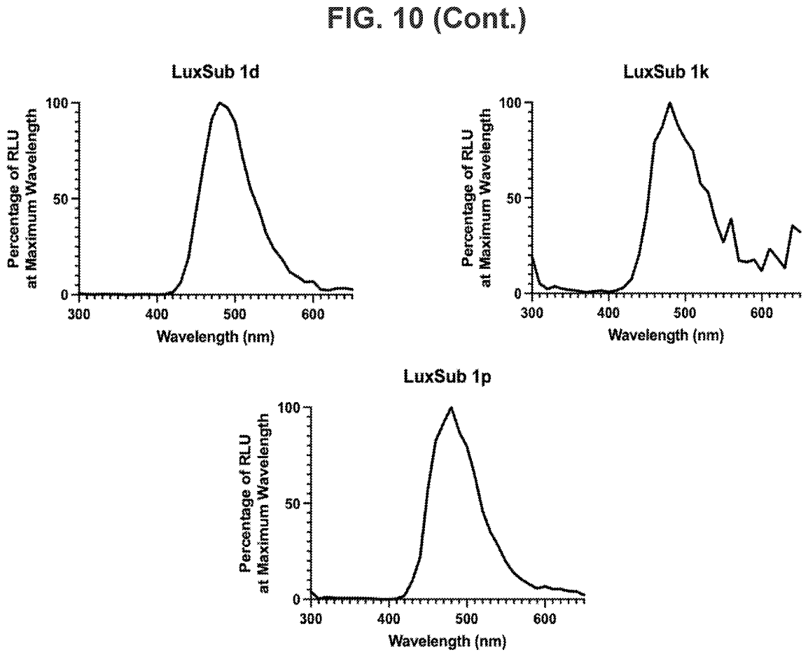

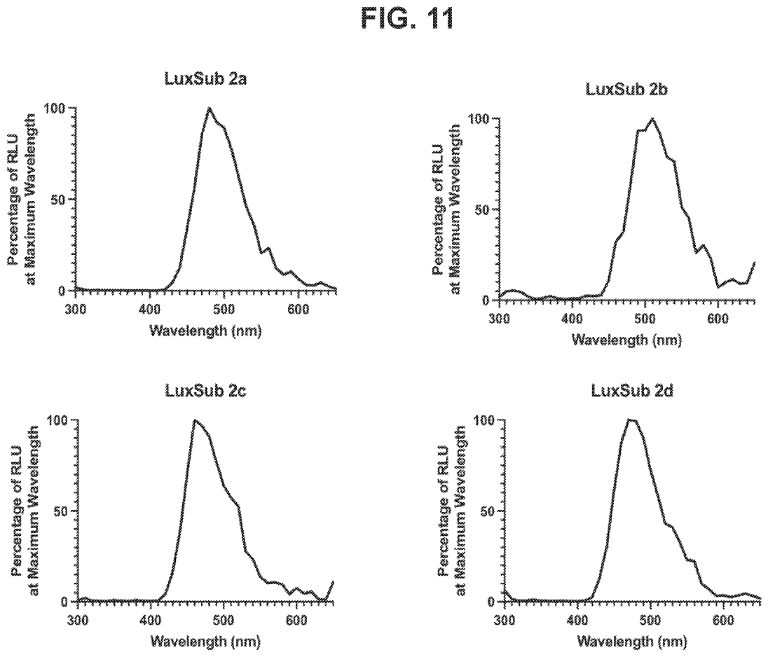

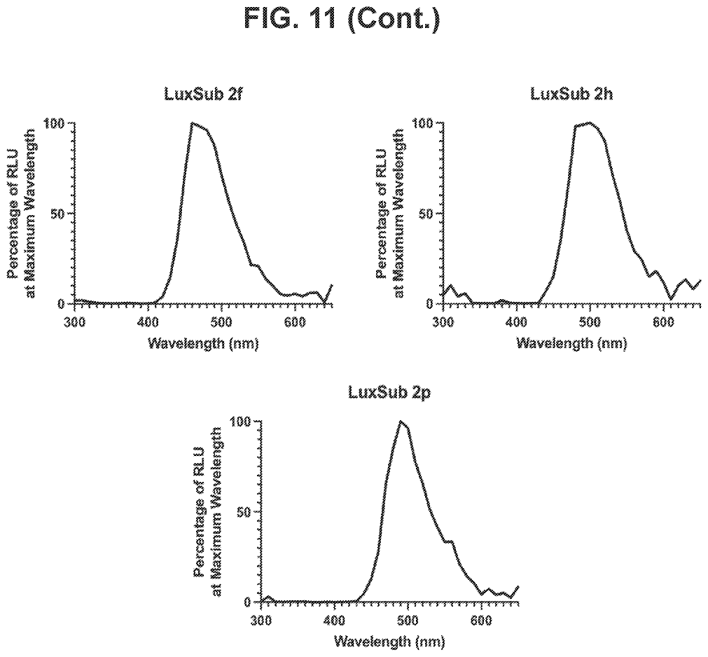

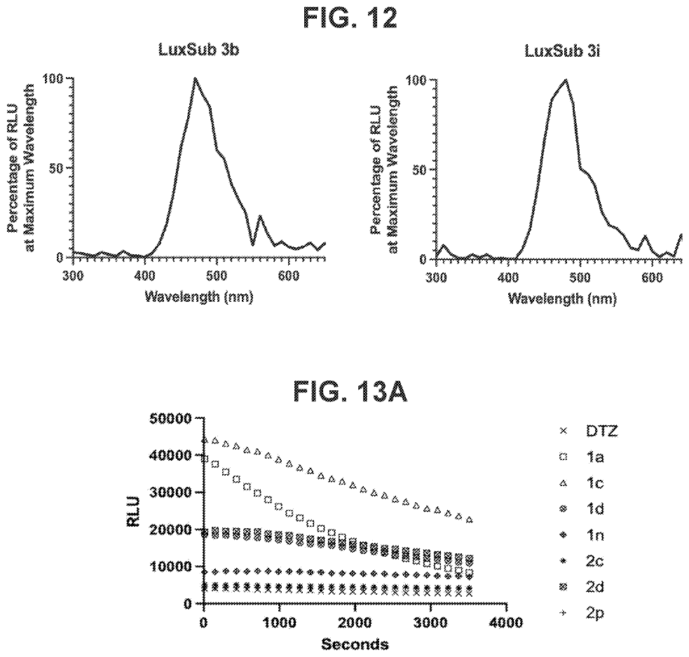

FIG. 10 shows bioluminescence emission spectra of synthetic luciferin substrates 1a, 1b, 1c, 1d, 1k, 1n, and 1p incubated with MBIO-301 enzyme at 500 μM. FIG. 11 shows bioluminescence emission spectra of synthetic luciferin substrates 2a, 2b, 2c, 2d, 2f, 2h, and 2p, incubated with MBIO-301 enzyme. FIG. 12 shows bioluminescence emission spectra of synthetic luciferin substrates 3b and 3i incubated with MBIO-301 enzyme.

### Example 6: Bioluminescence Emission Spectra of Synthetic Luciferin Substrates in Presence of 20% Human Serum

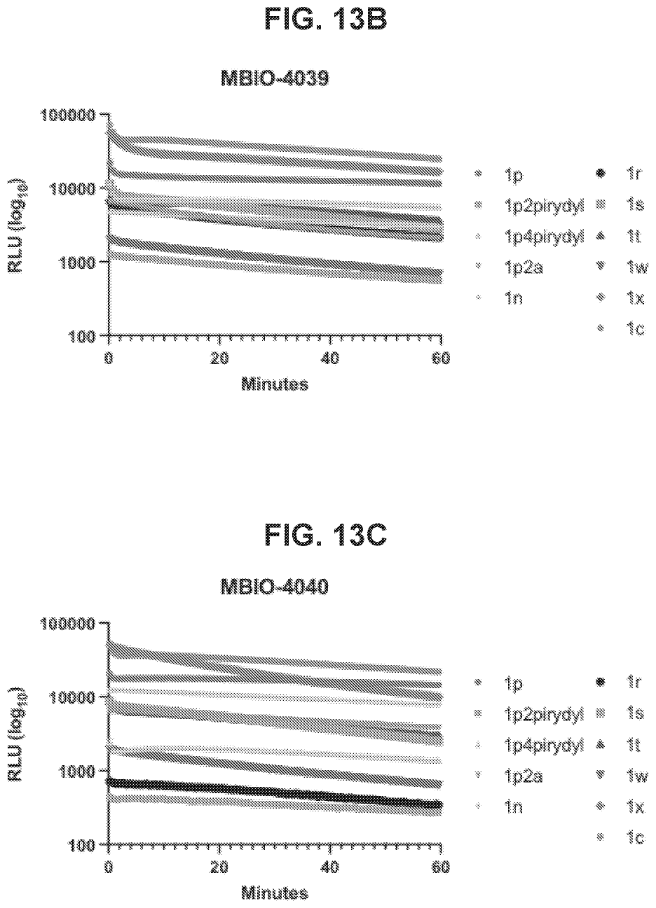

Bioluminescence emission of synthetic luciferin substrates was measured. The luciferin substrates were incubated with 500 μM MBIO-301 enzyme in presence of 20% human serum. Each of the molecules was incubated with human serum diluted to 20% in 1×PBS, 0.5% Propylene Glycol, 0.05% Anti-foam, 100 mM Imidazole, 10 mM Ascorbic Acid buffer. Bioluminescence was measured with a Synergy H1 plate reader. FIG. 13 shows bioluminescence emission of synthetic luciferin substrates incubated with 500 μM MBIO-301 enzyme in presence of 20% human serum.

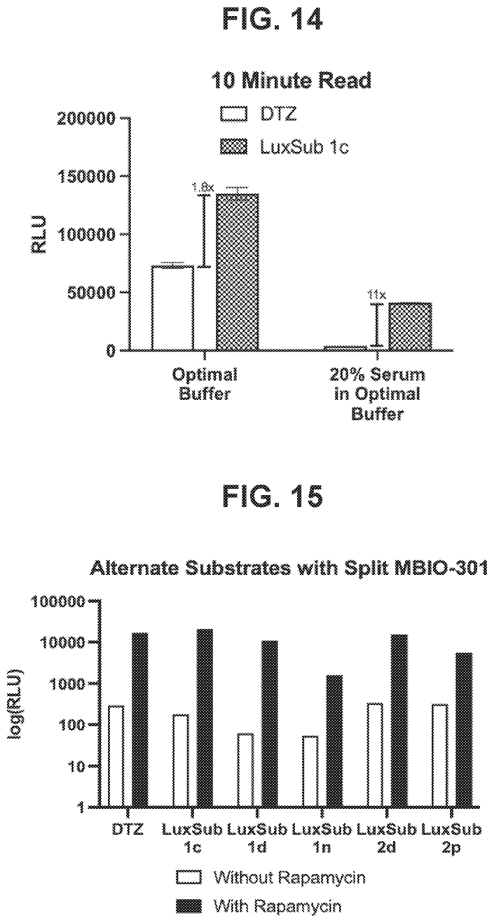

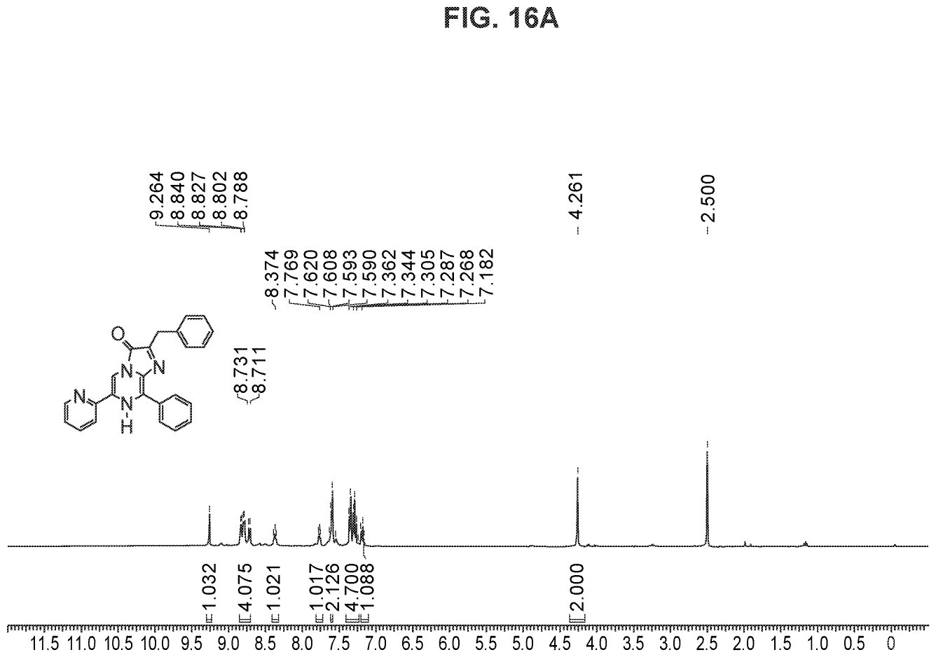

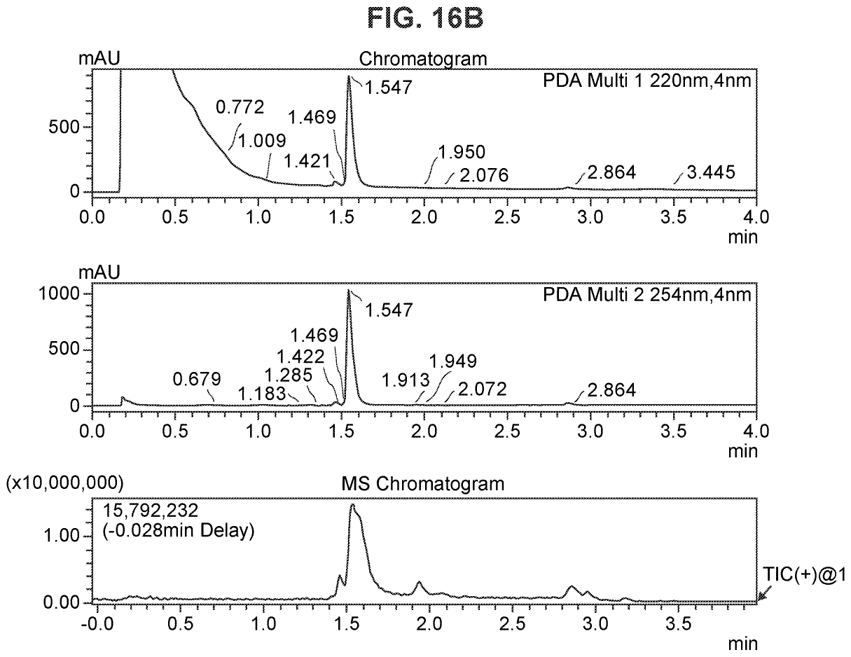

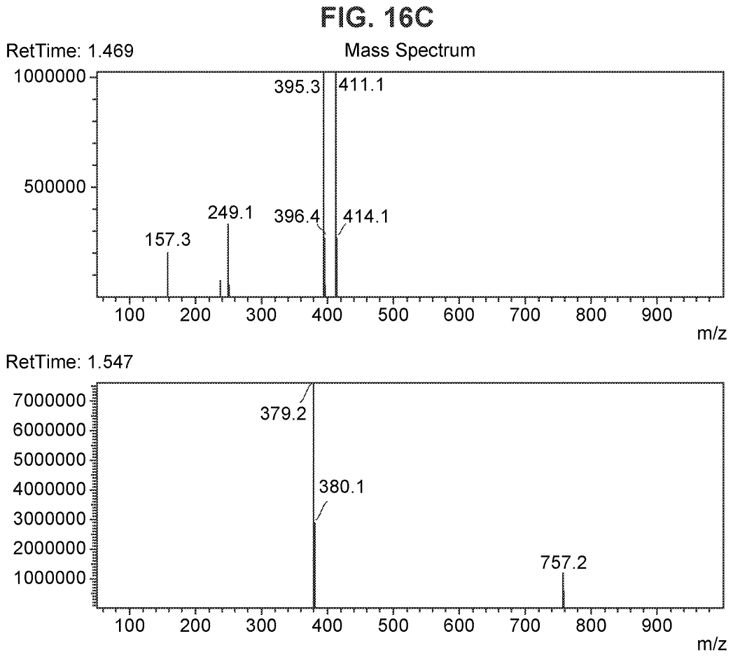

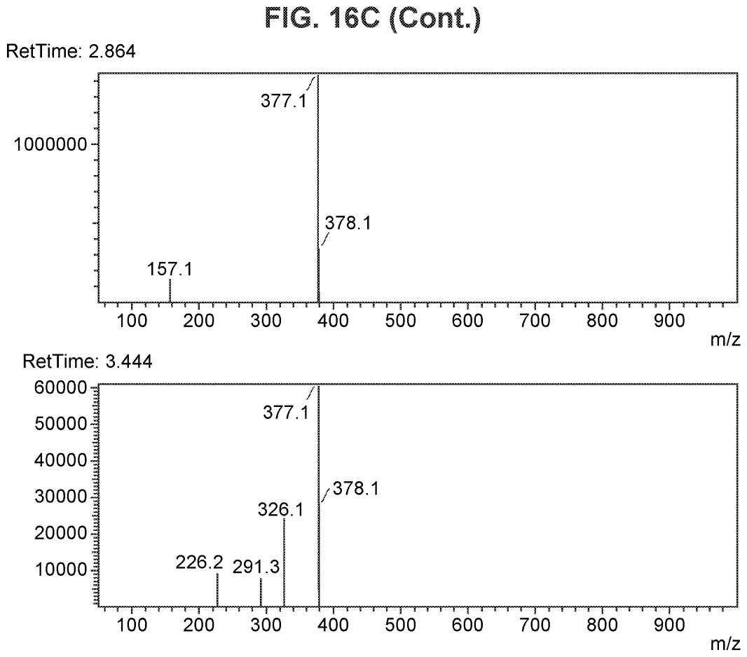

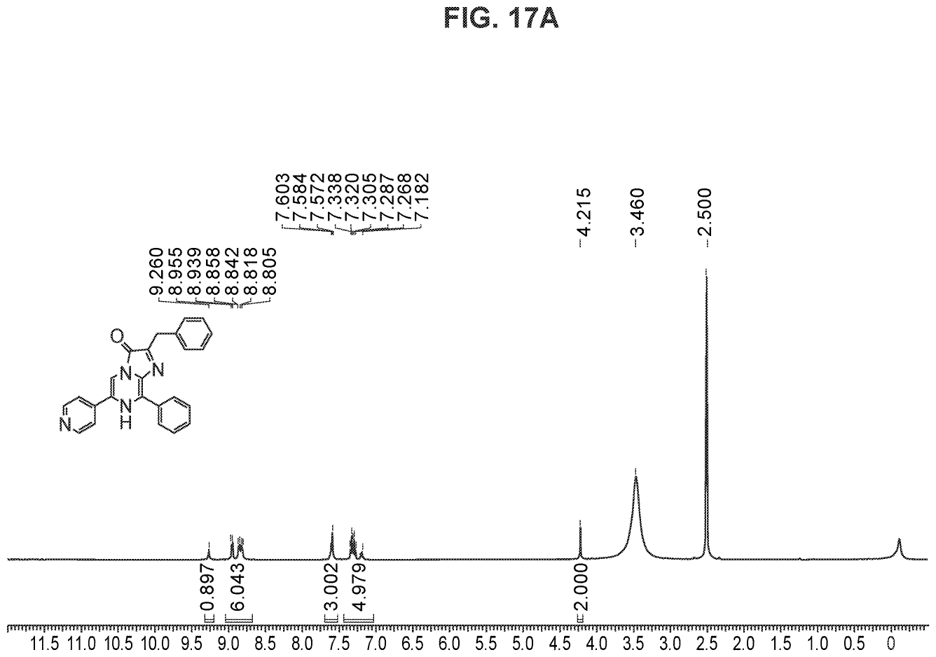

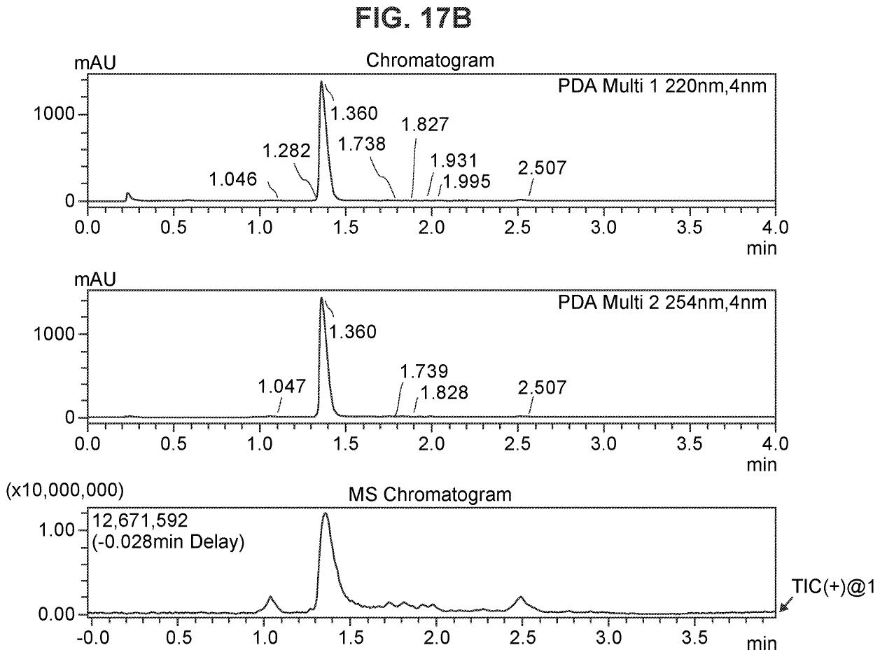

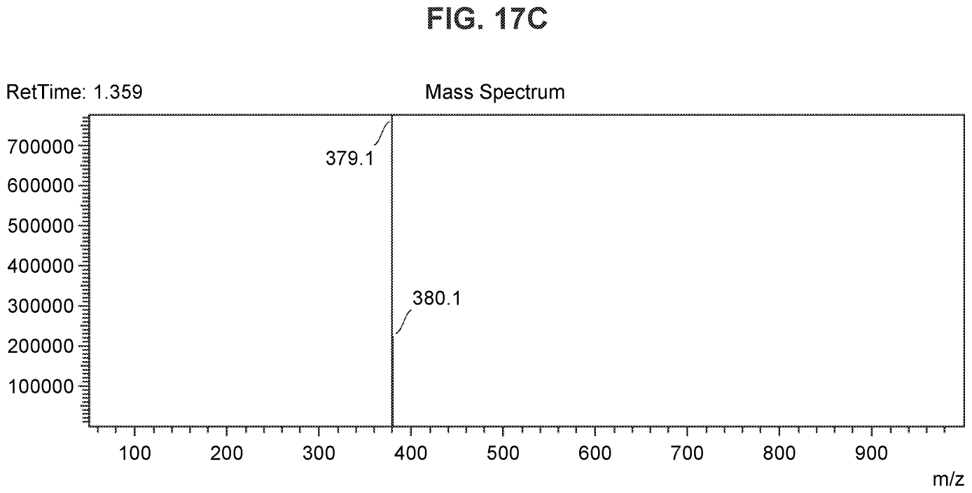

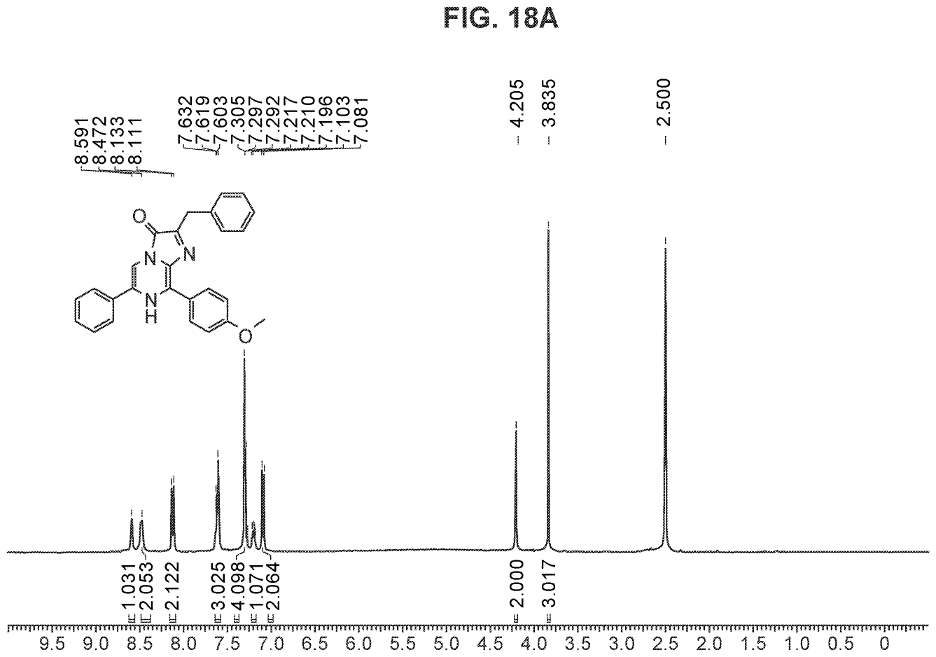

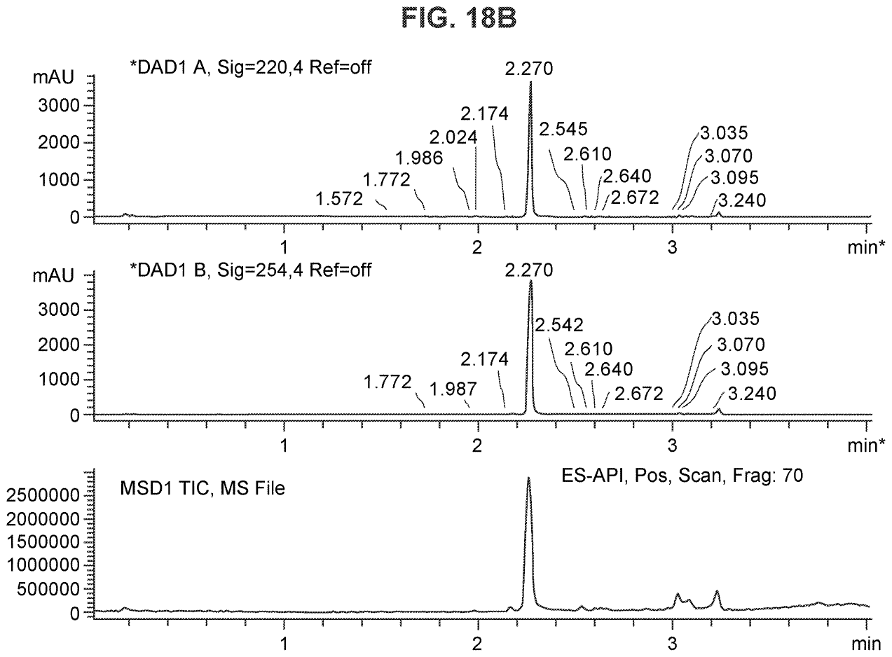

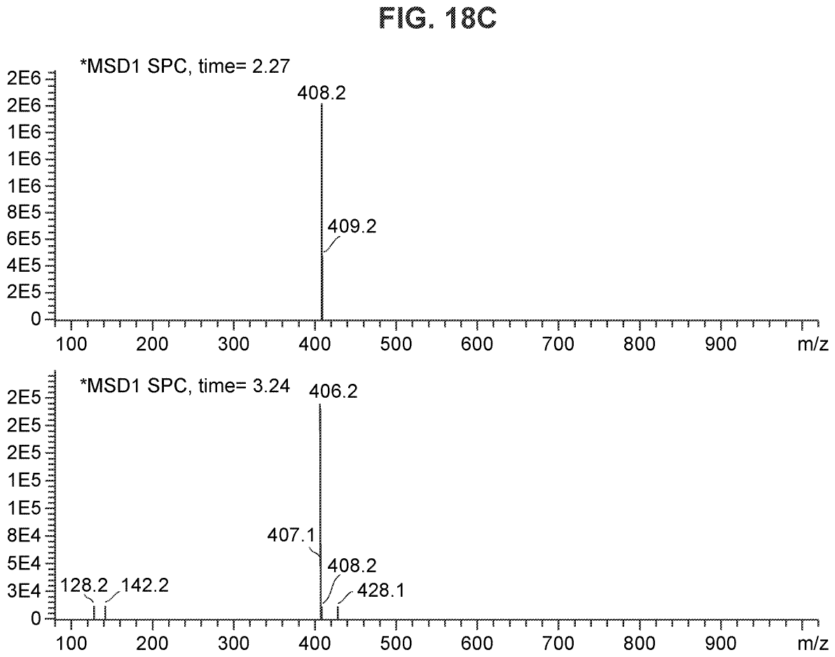

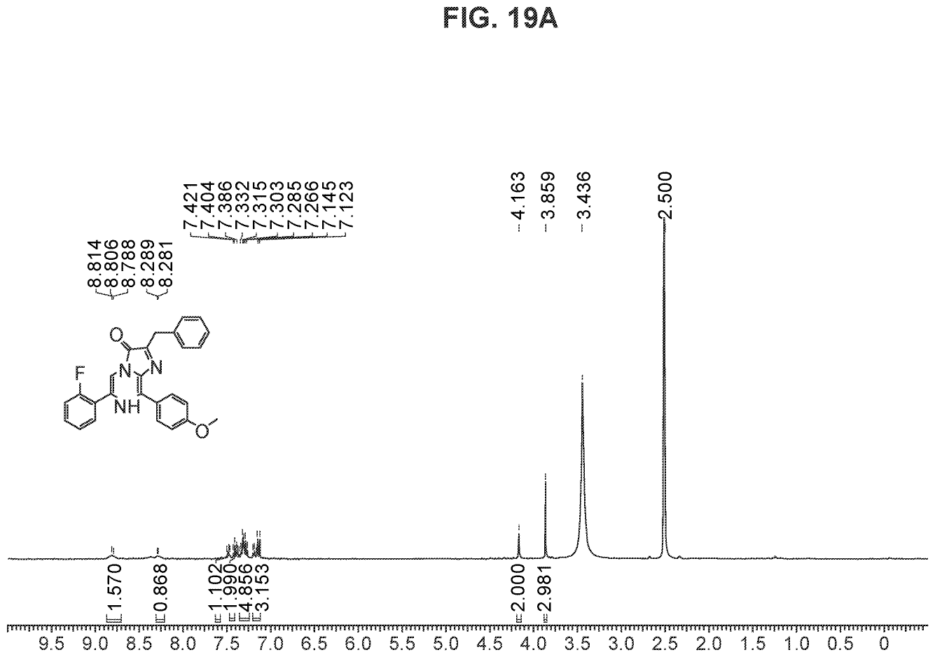

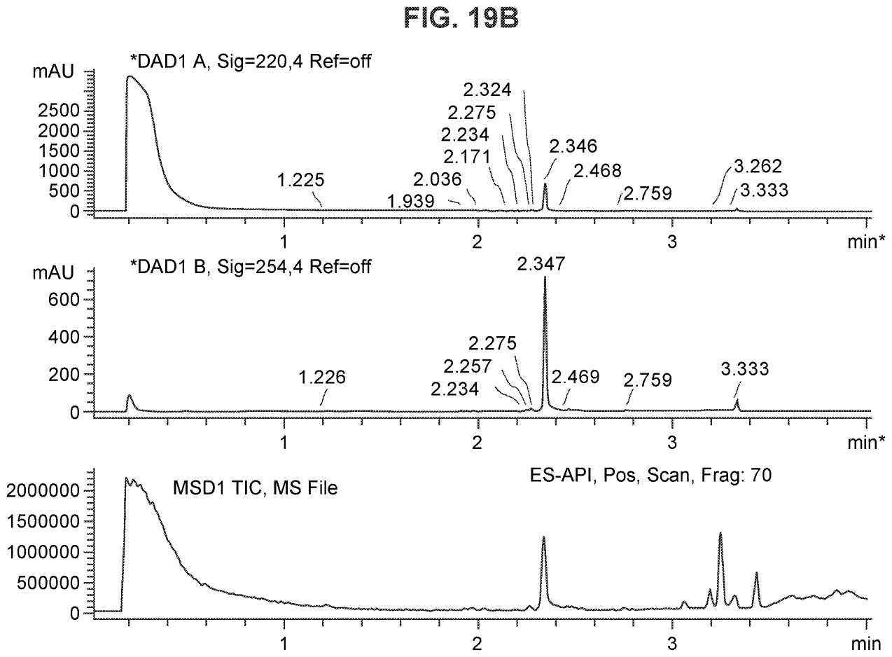

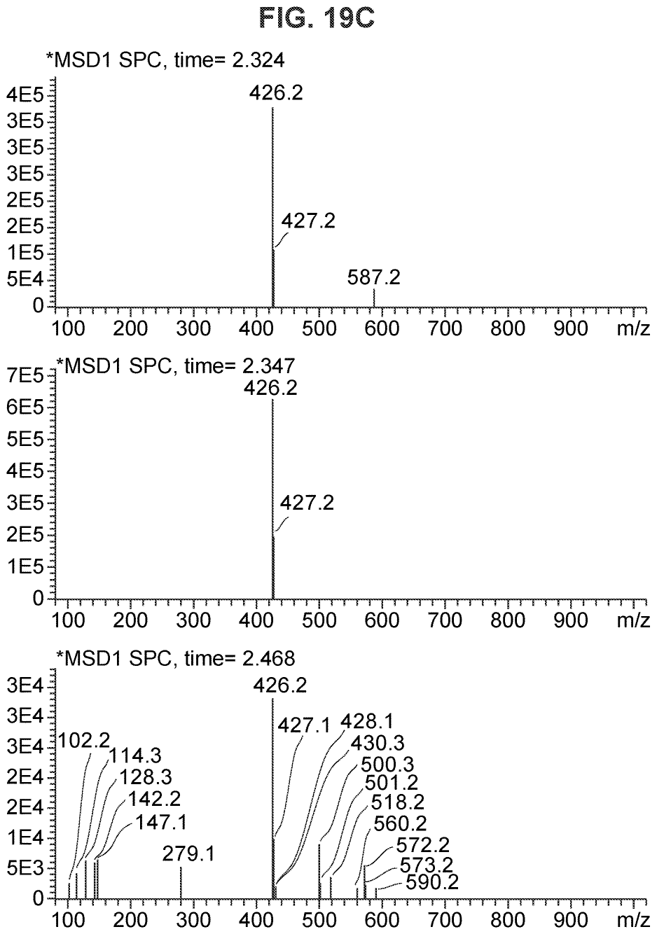

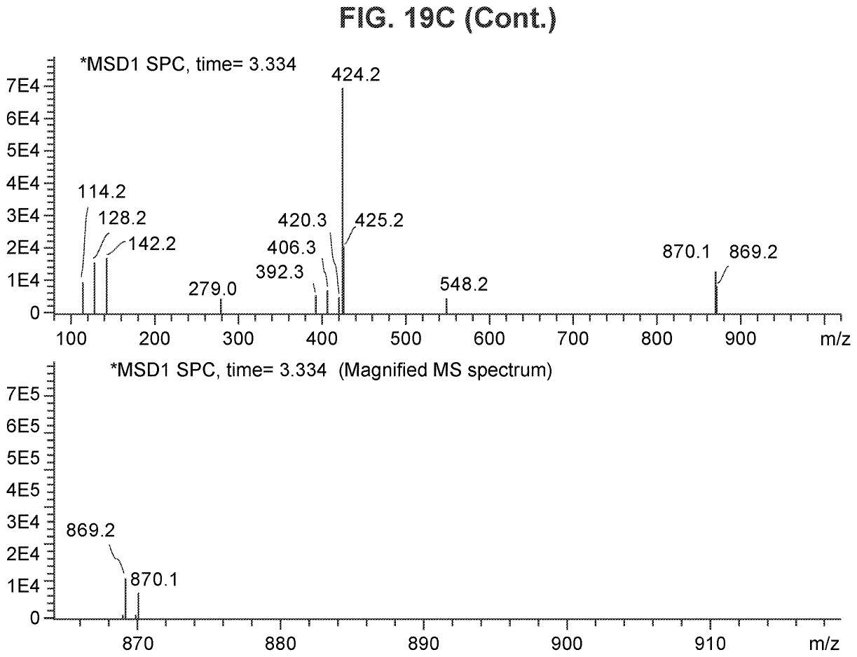

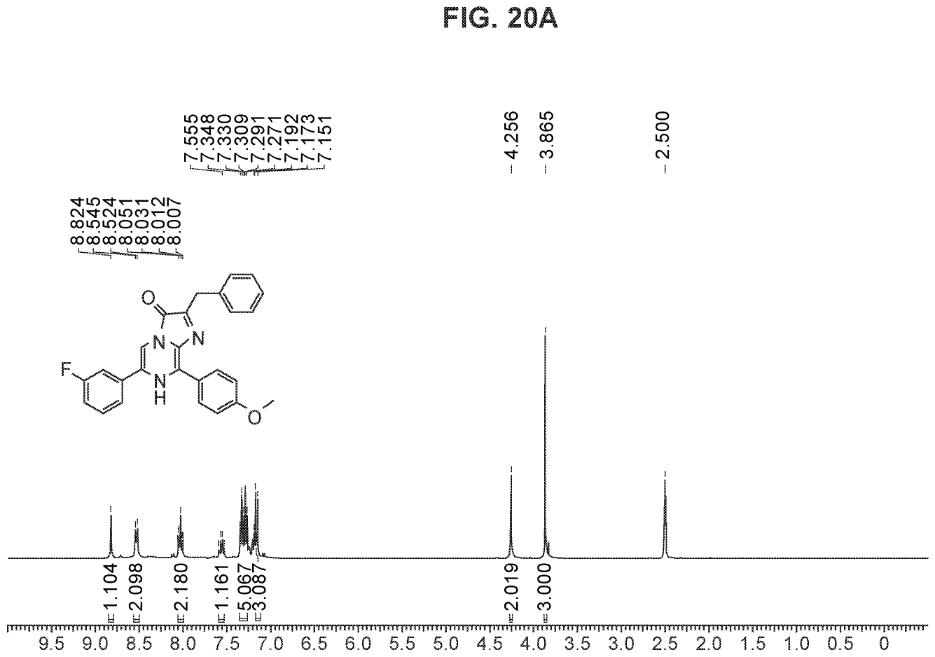

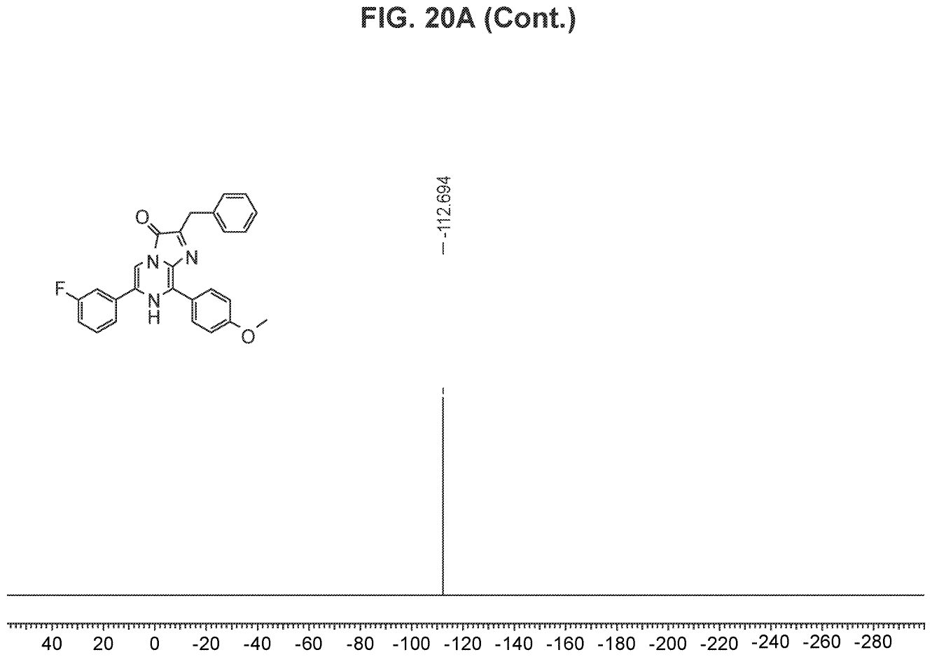

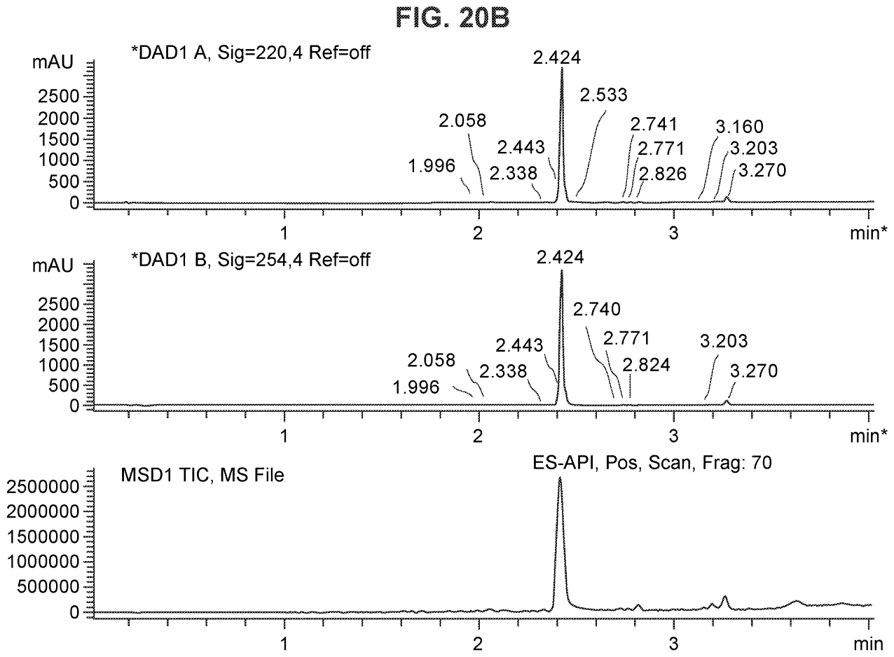

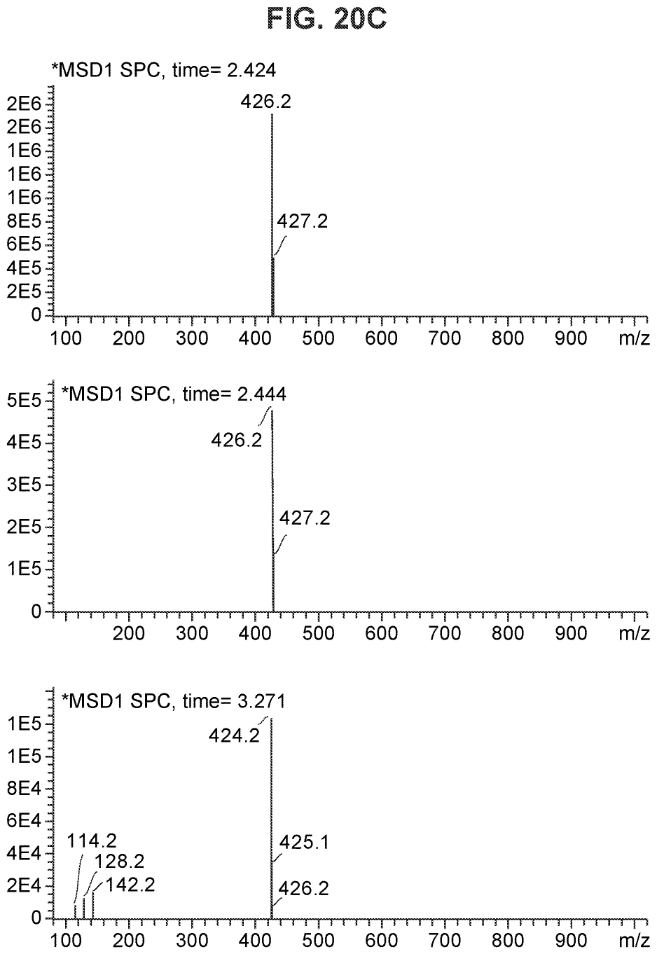

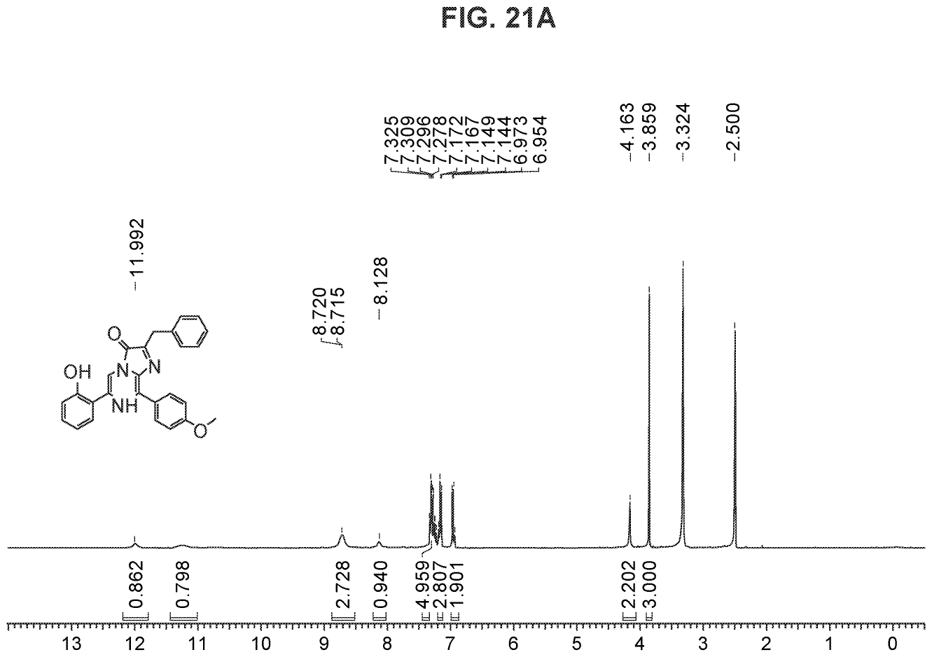

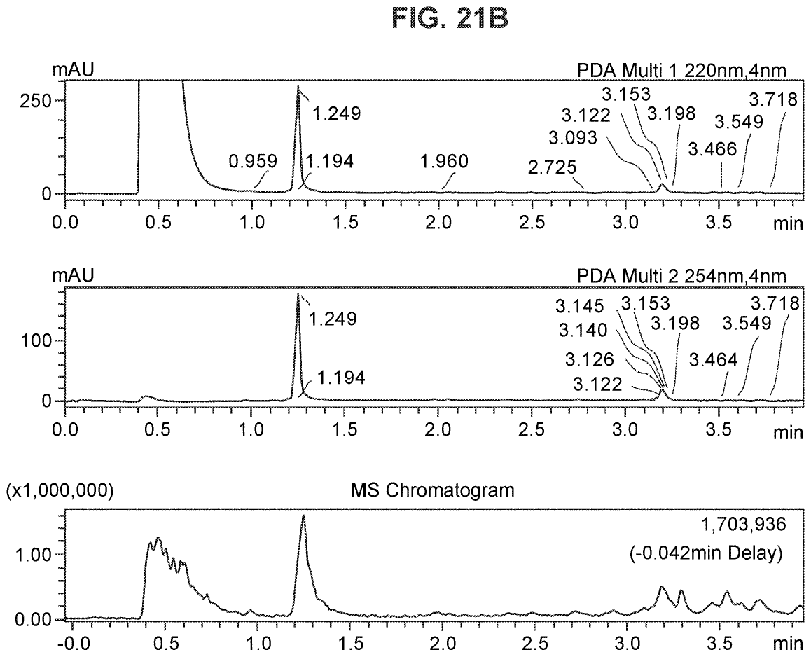

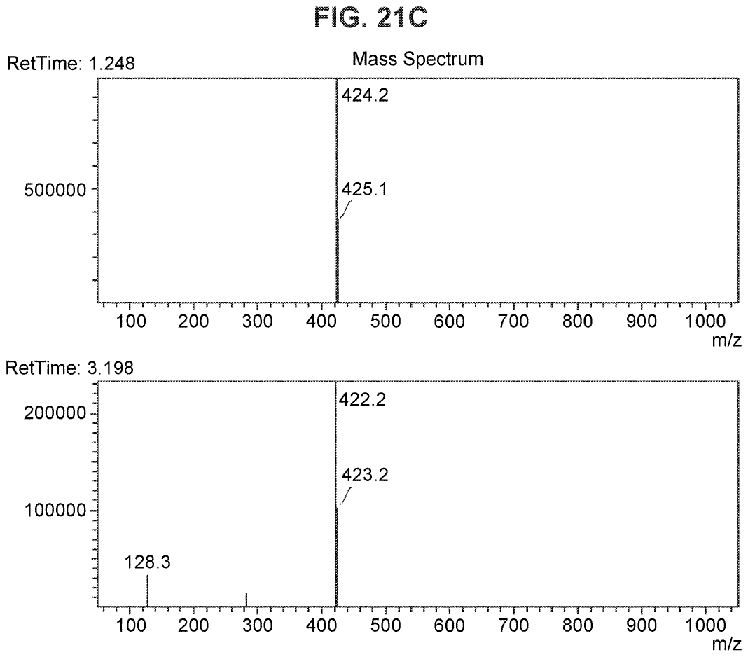

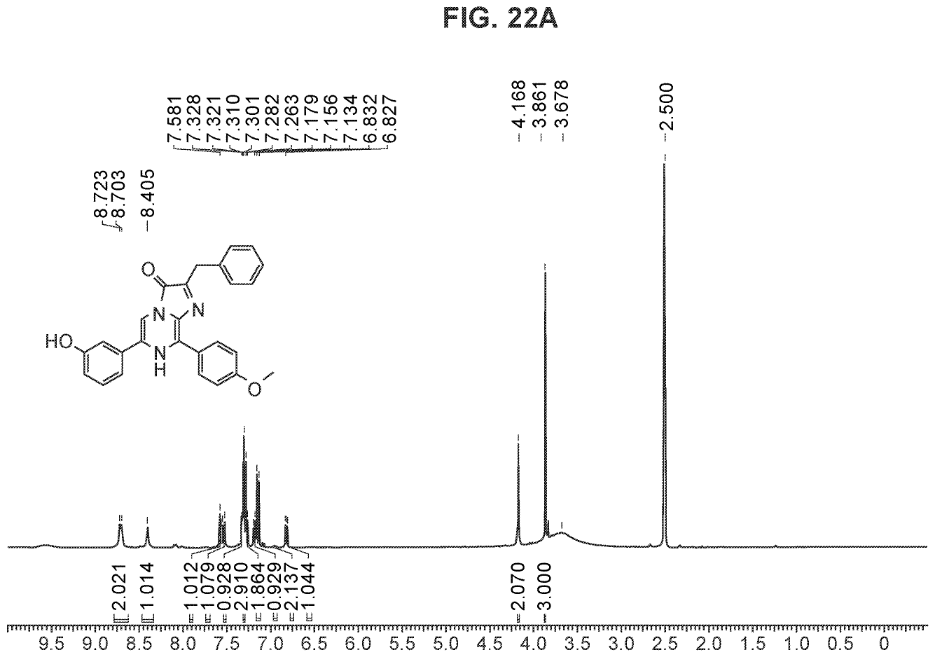

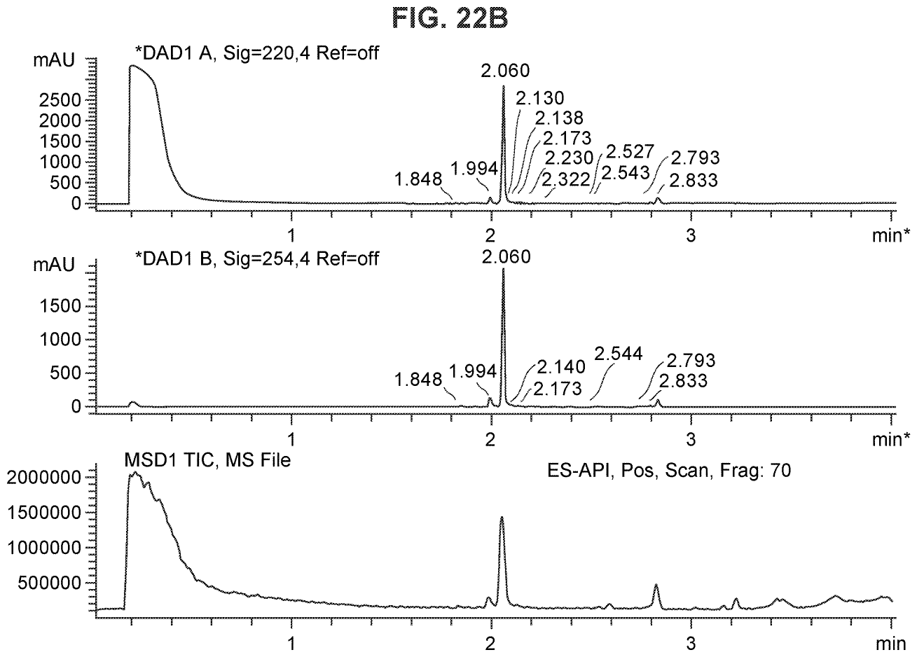

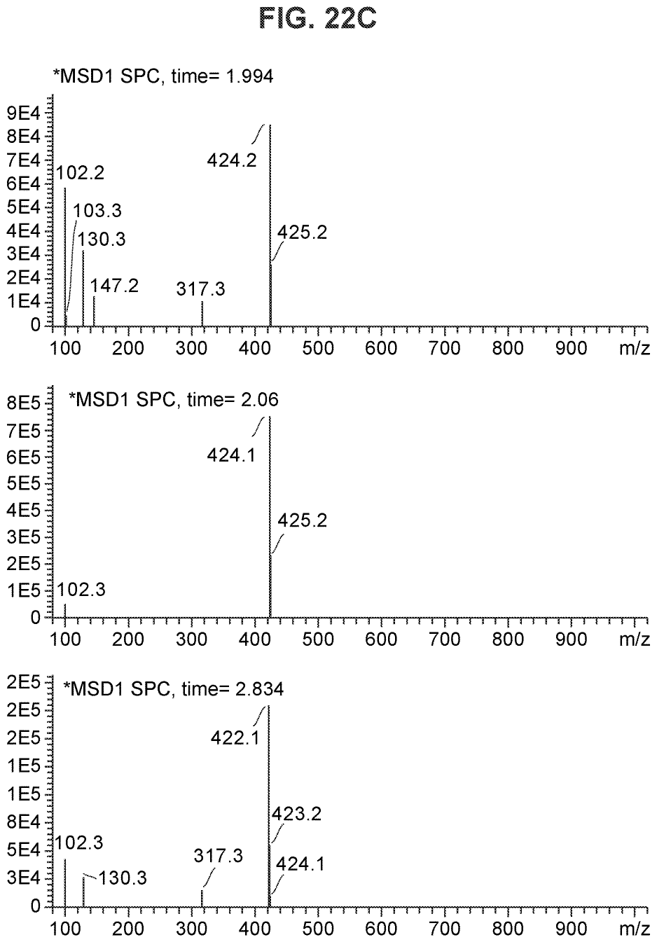

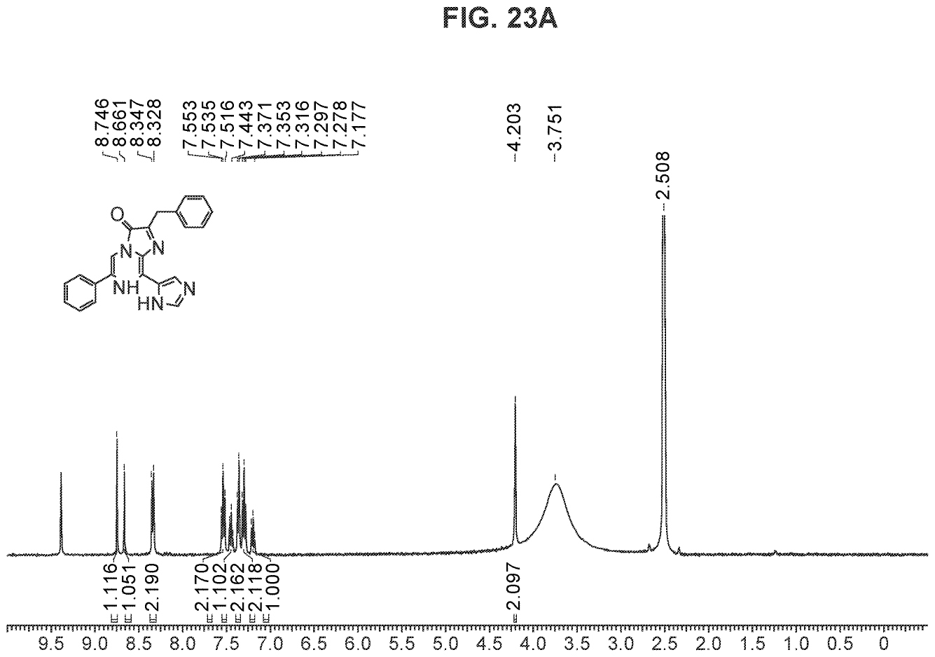

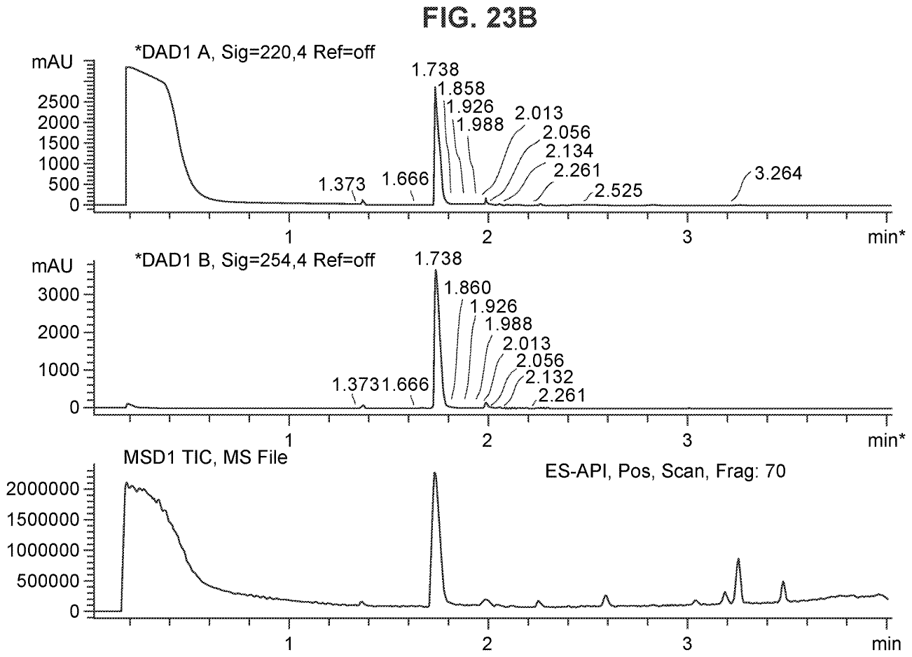

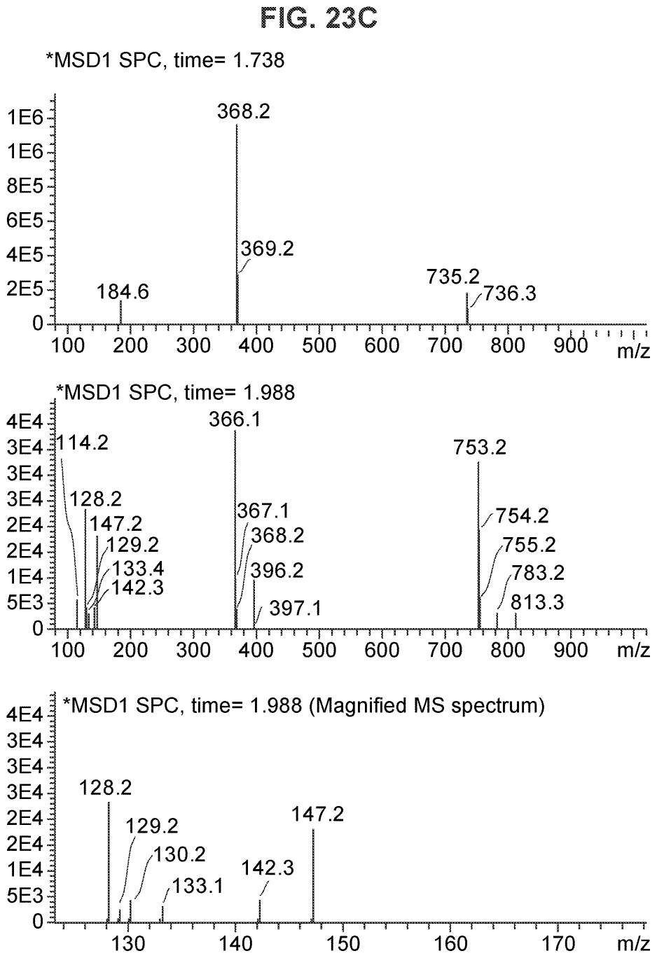

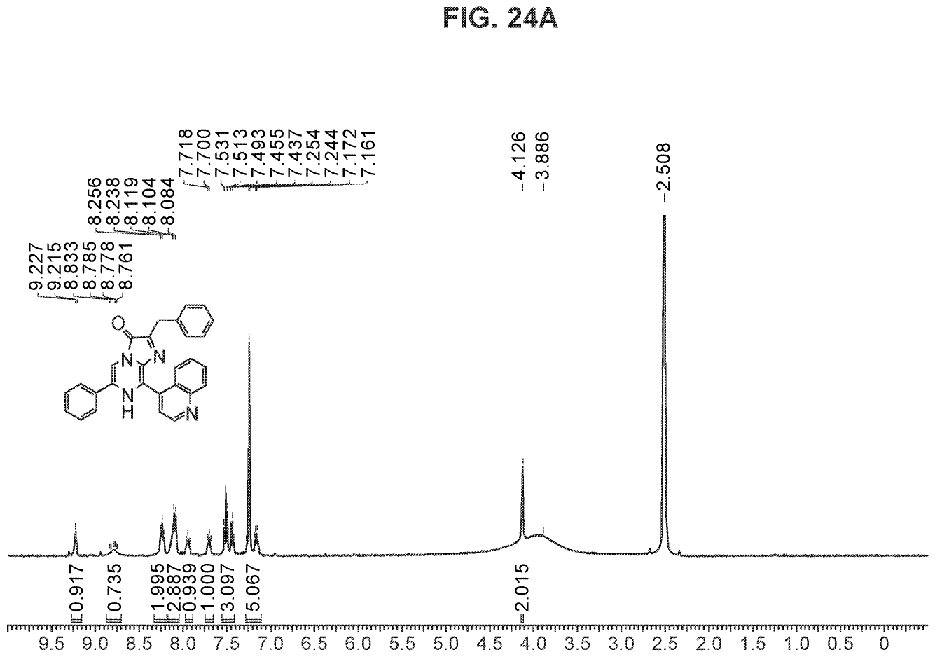

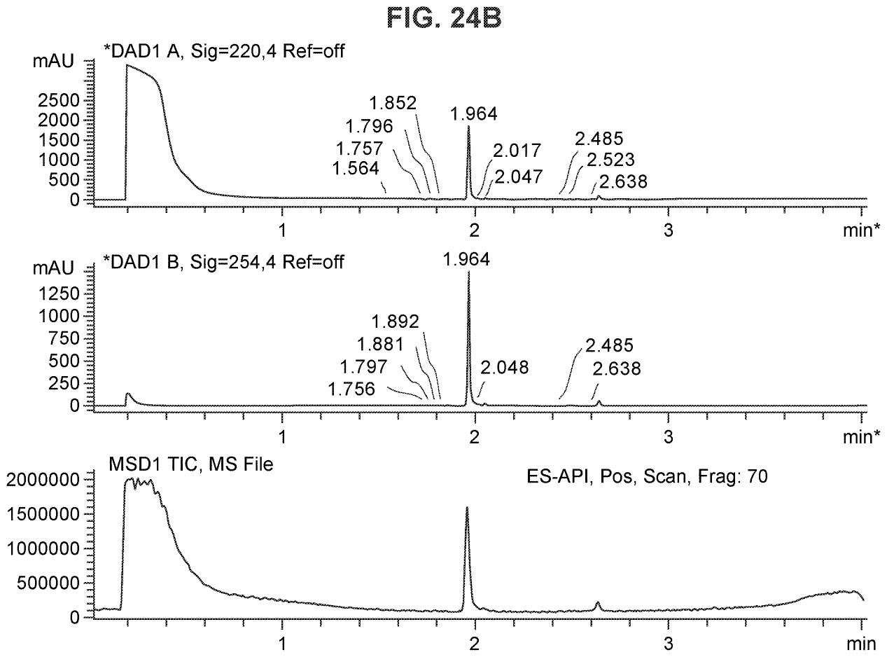

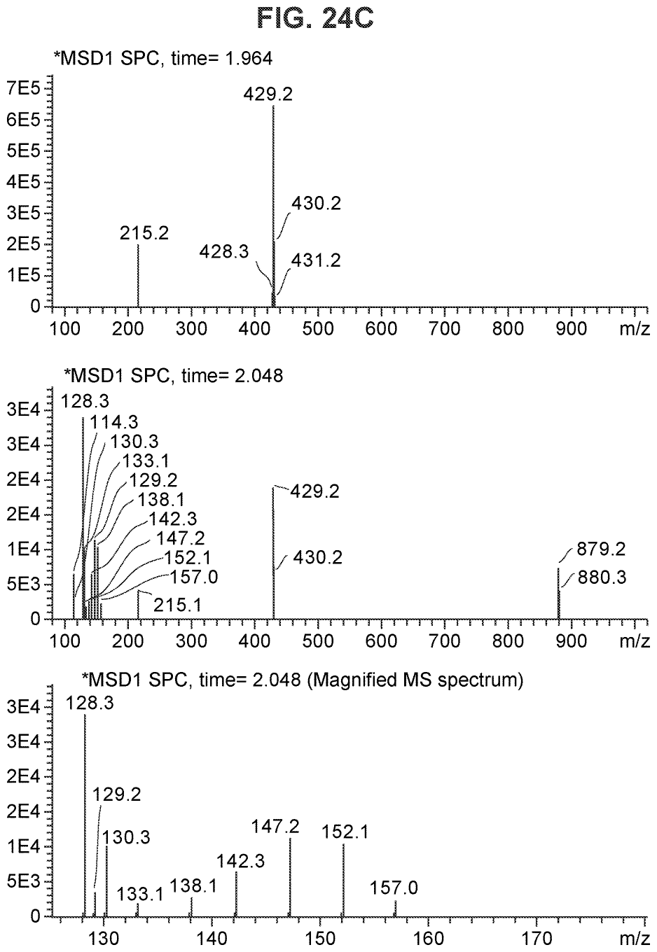

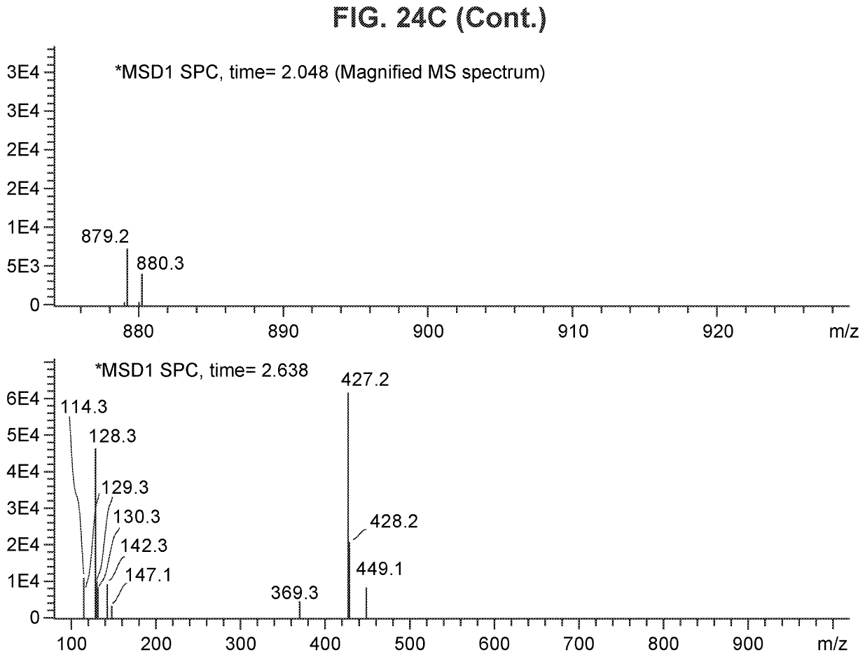

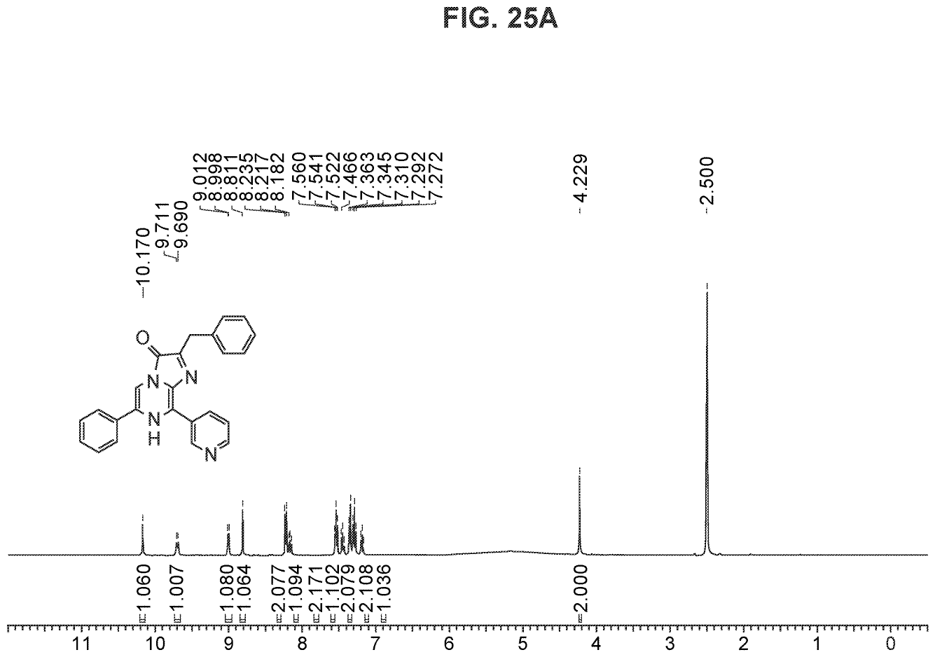

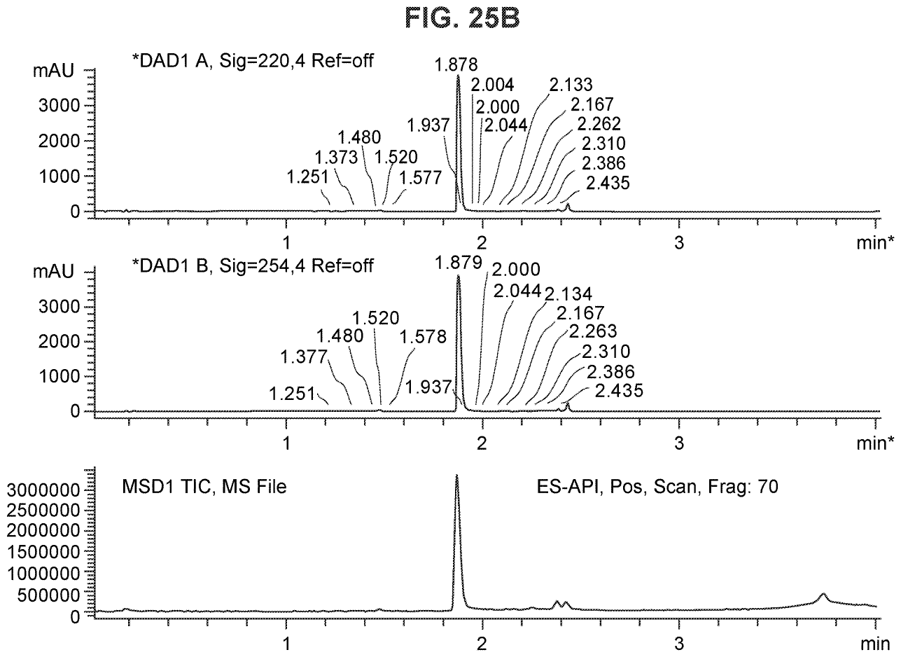

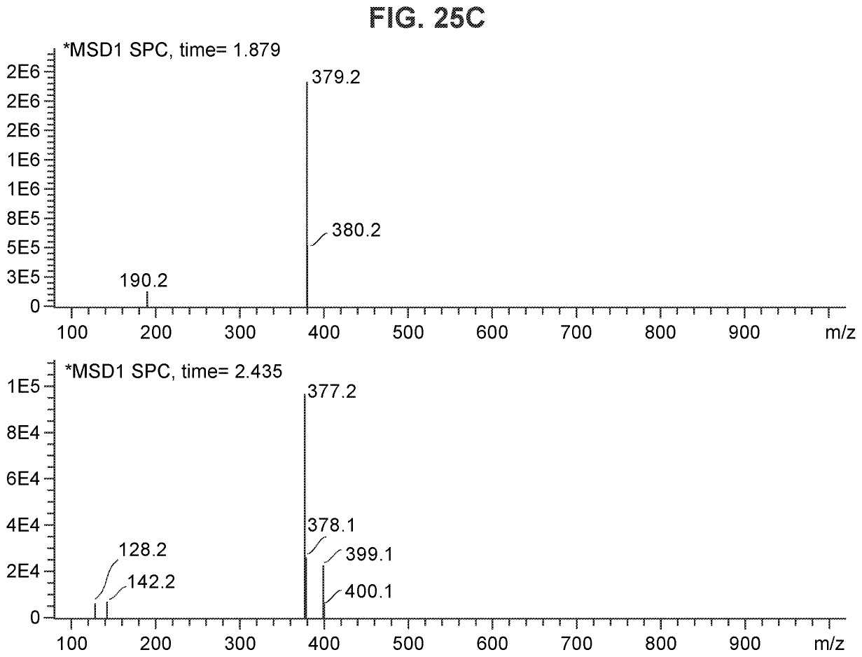

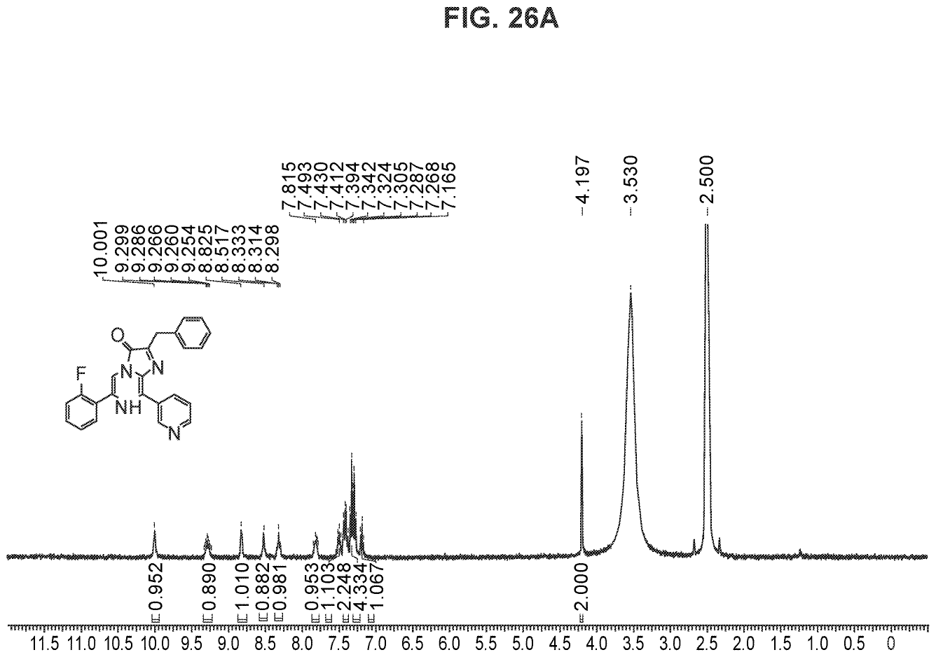

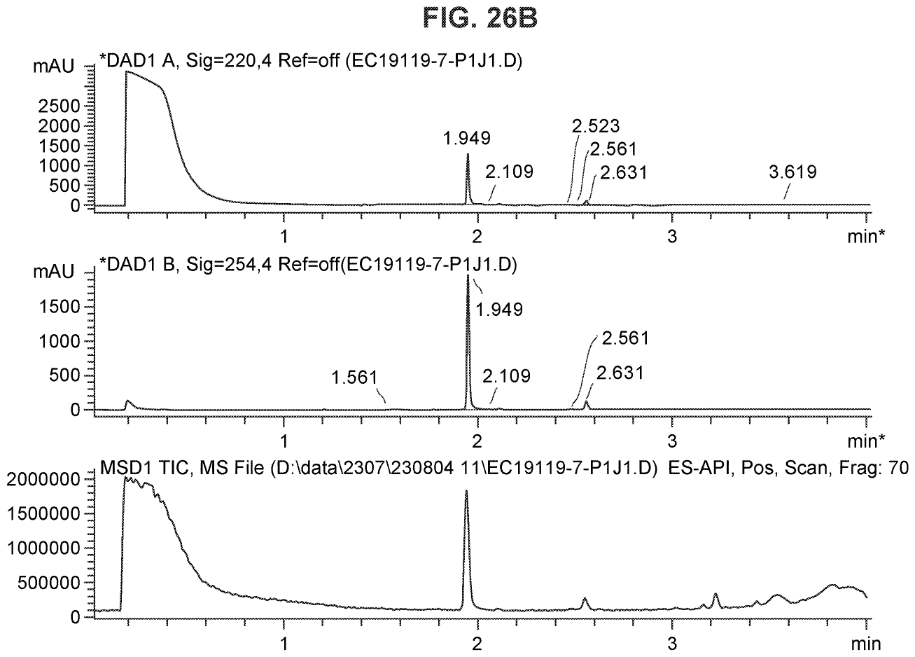

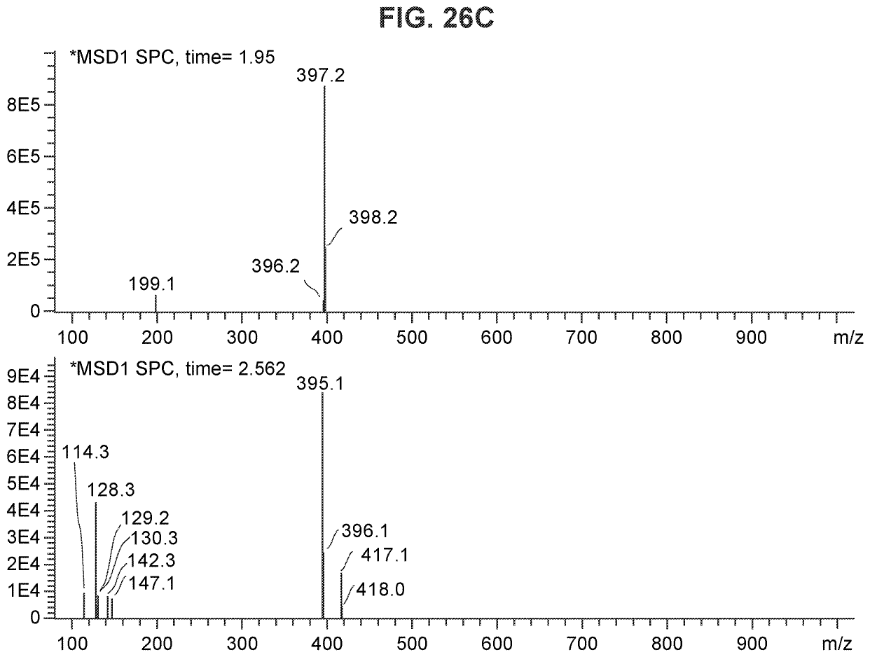

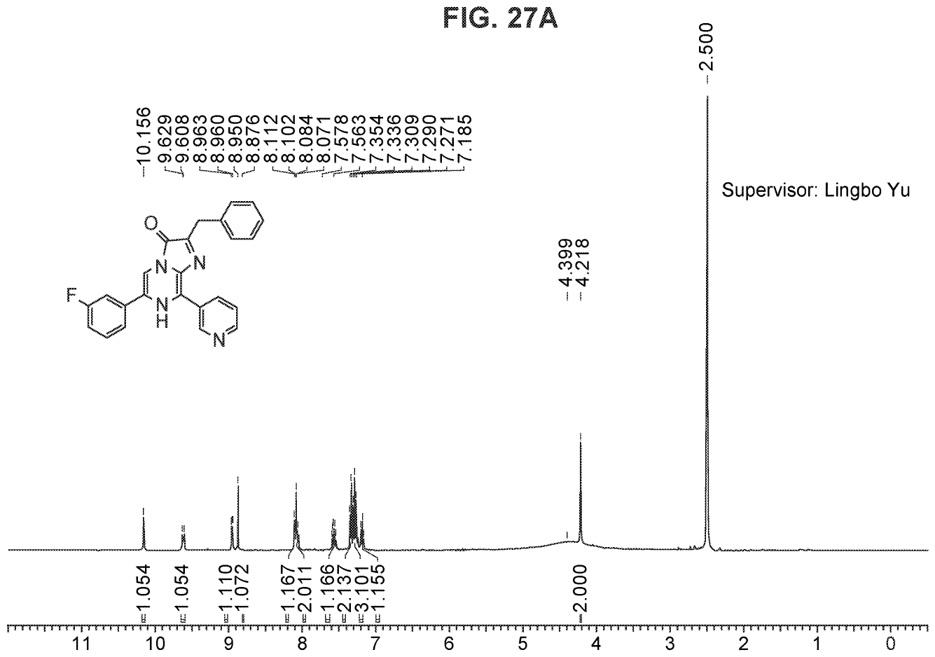

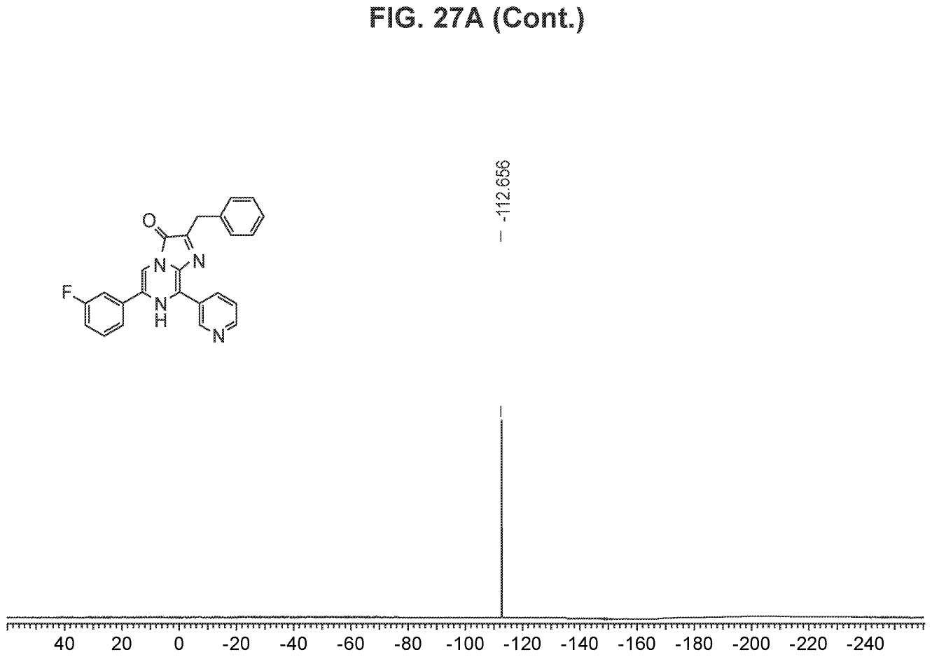

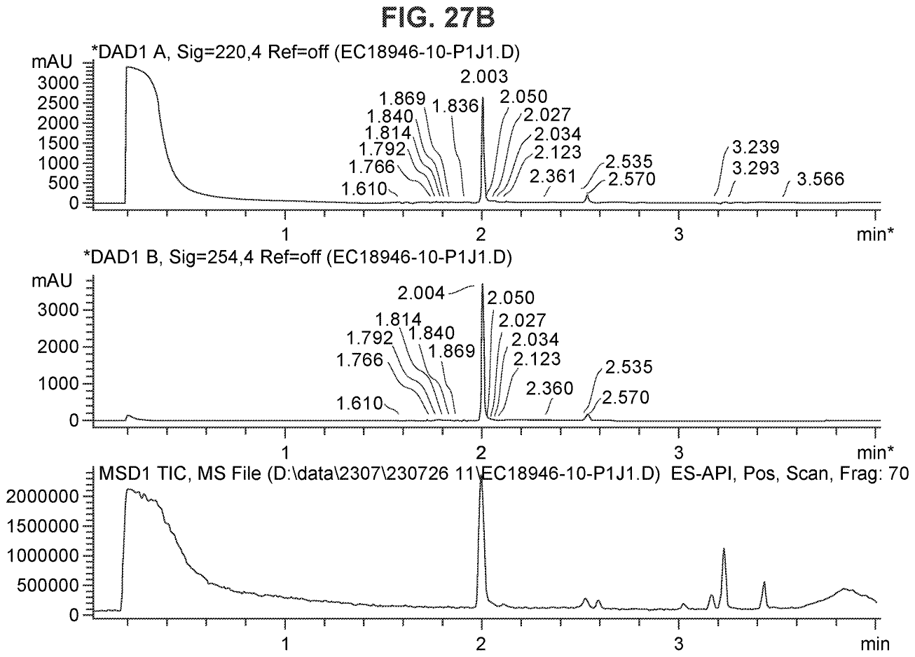

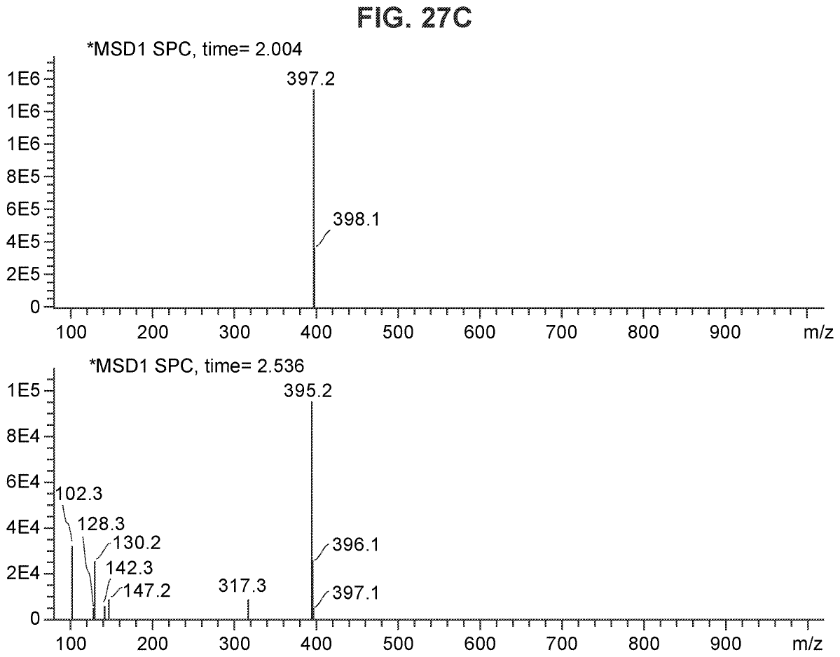

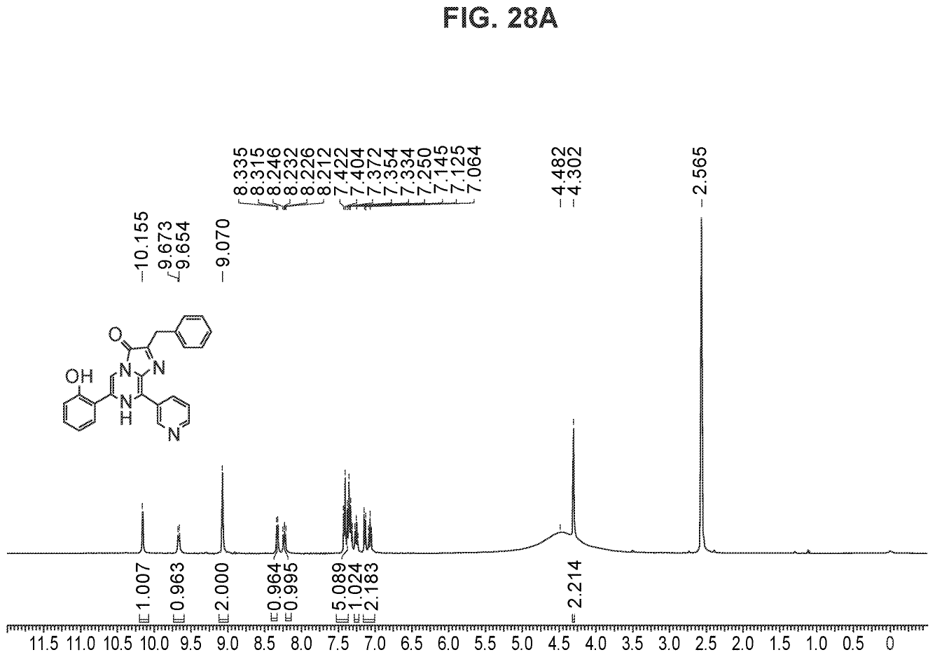

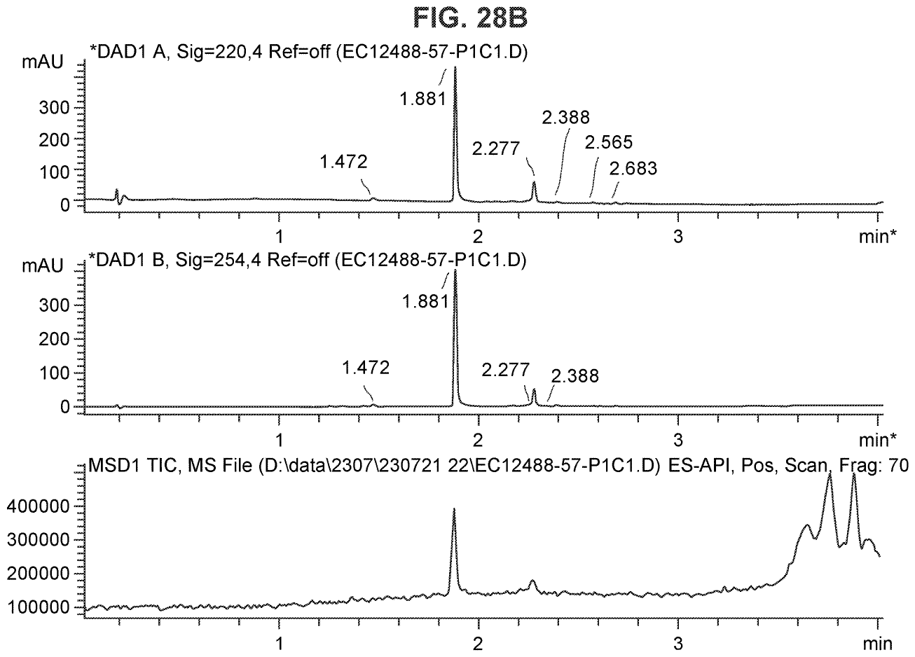

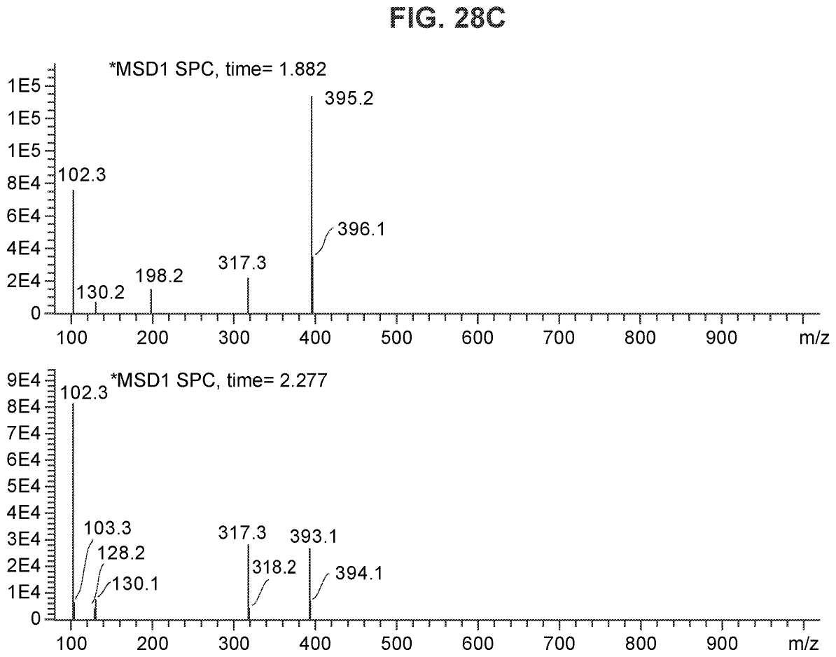

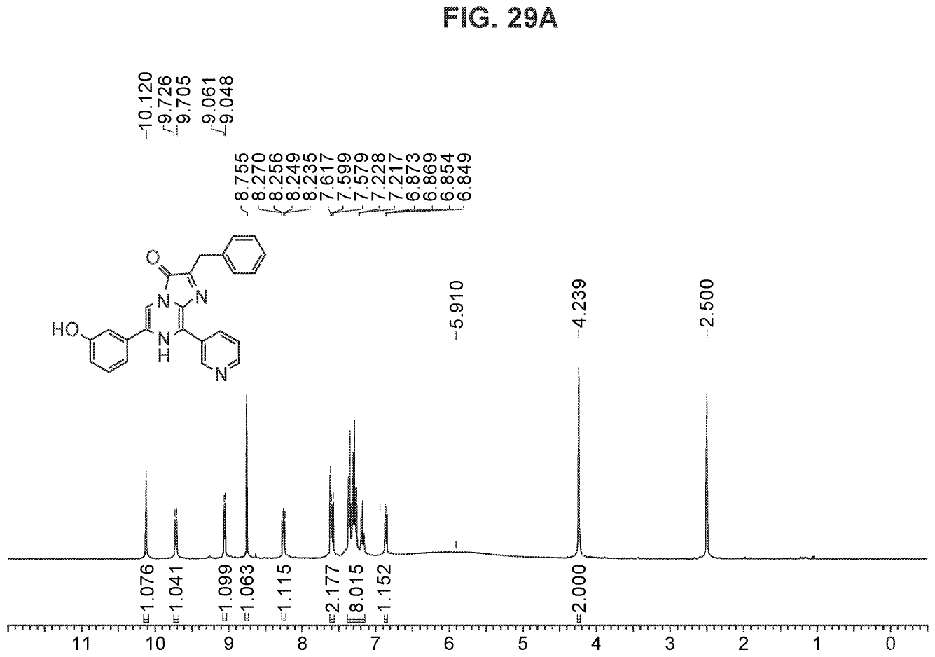

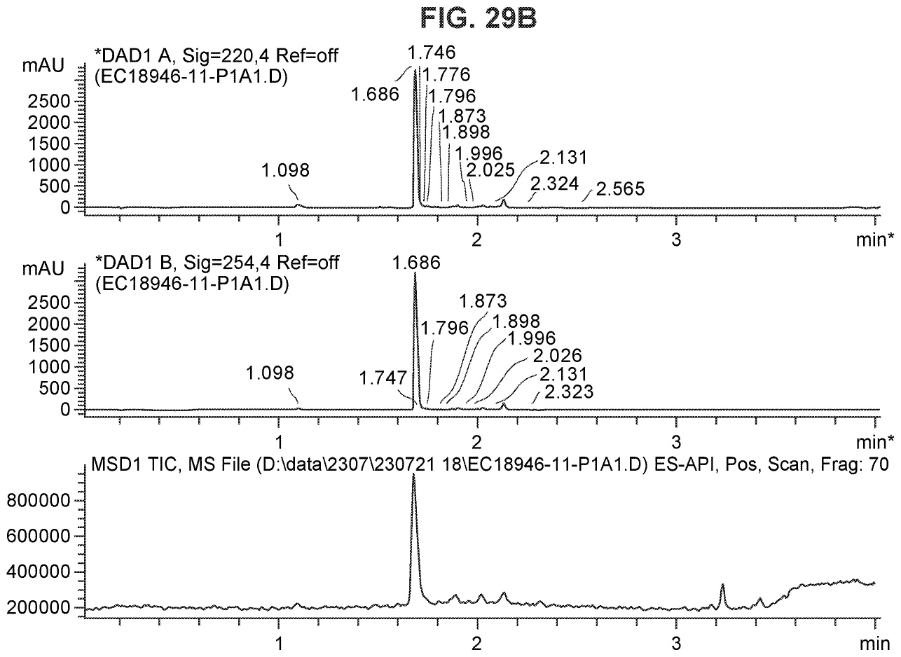

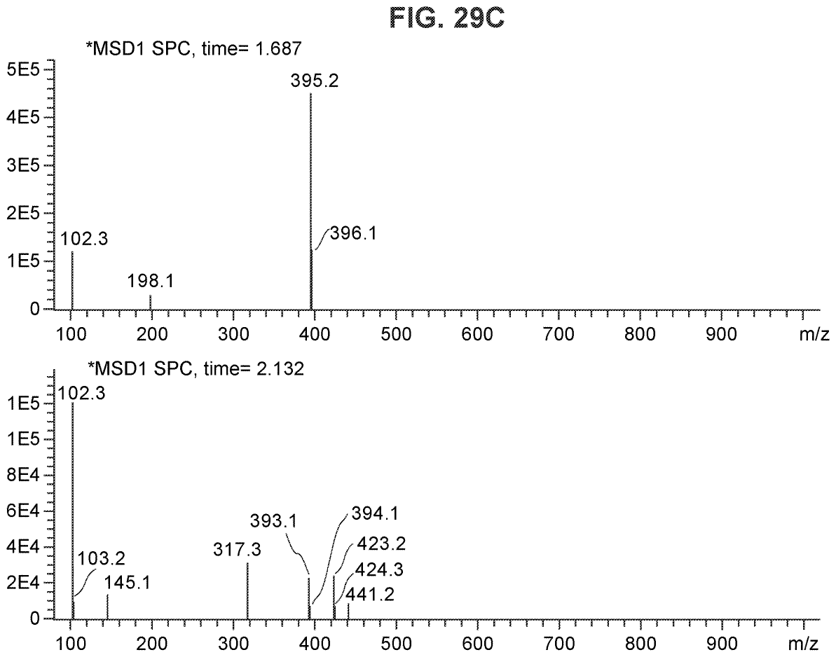

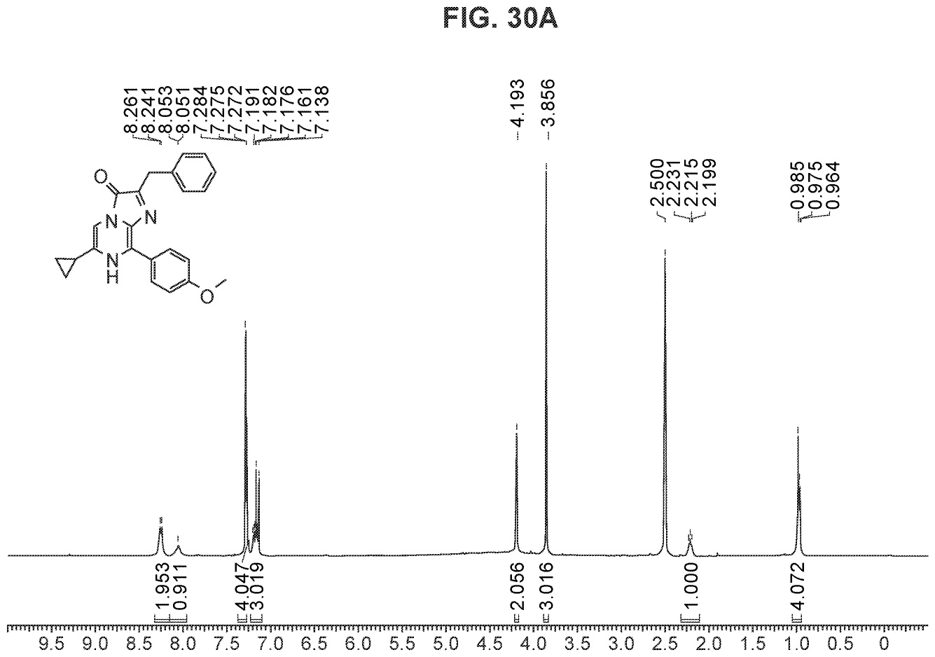

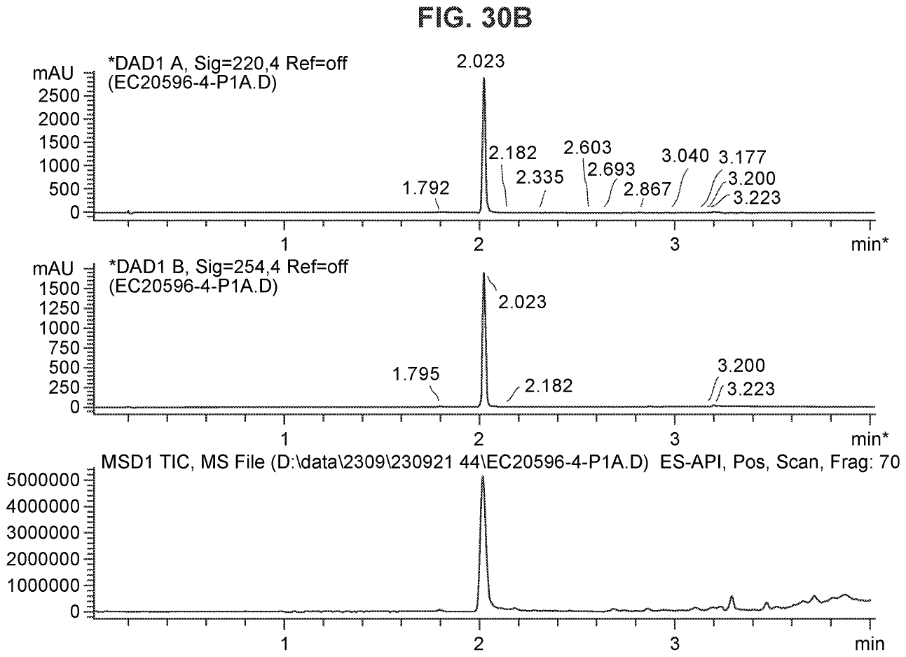

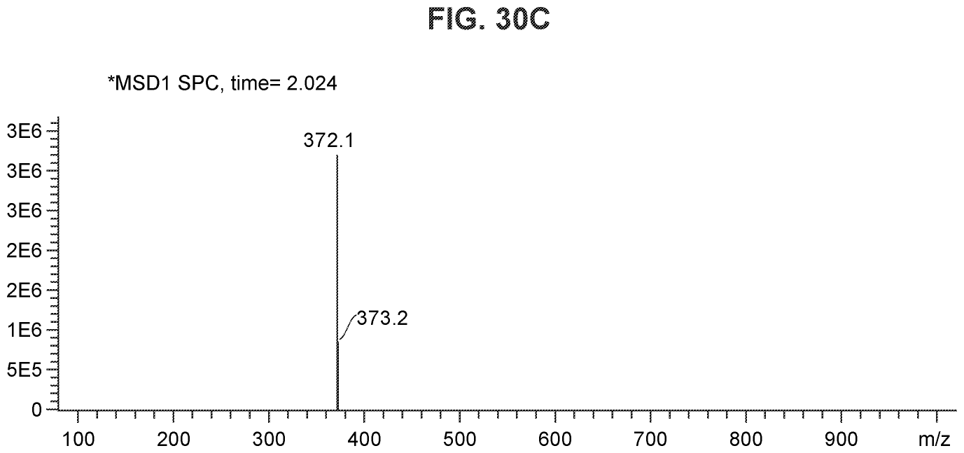

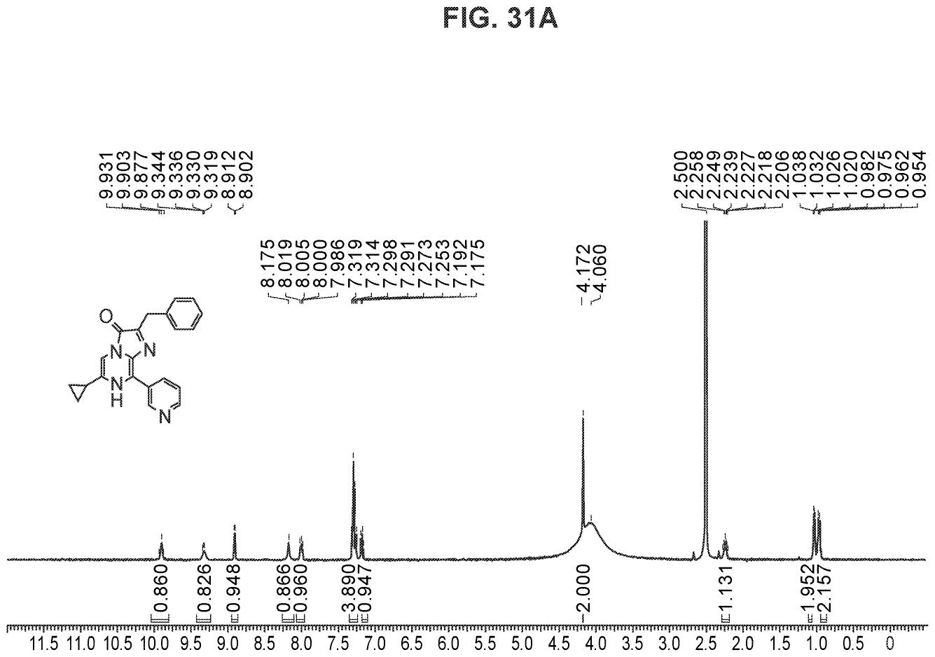

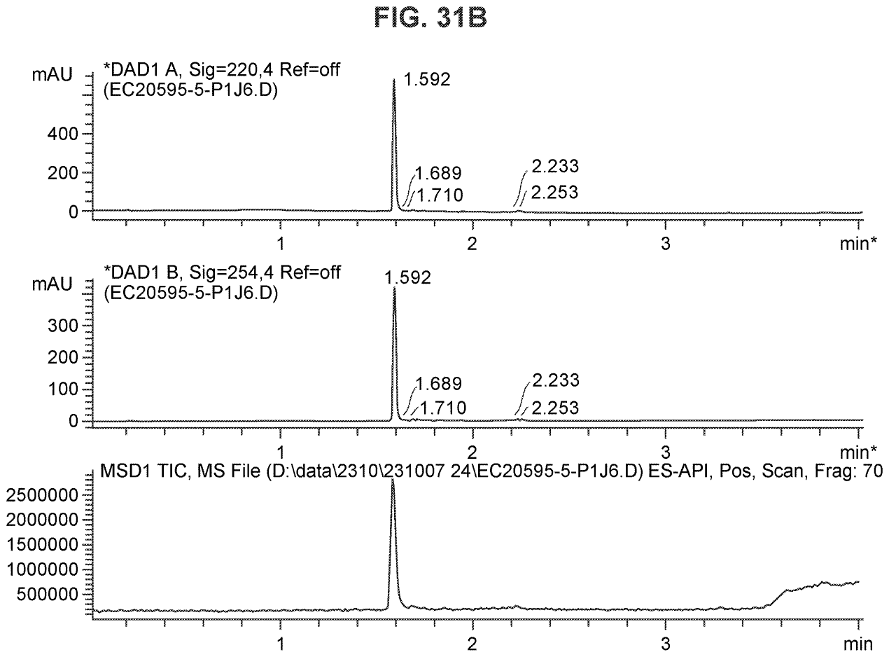

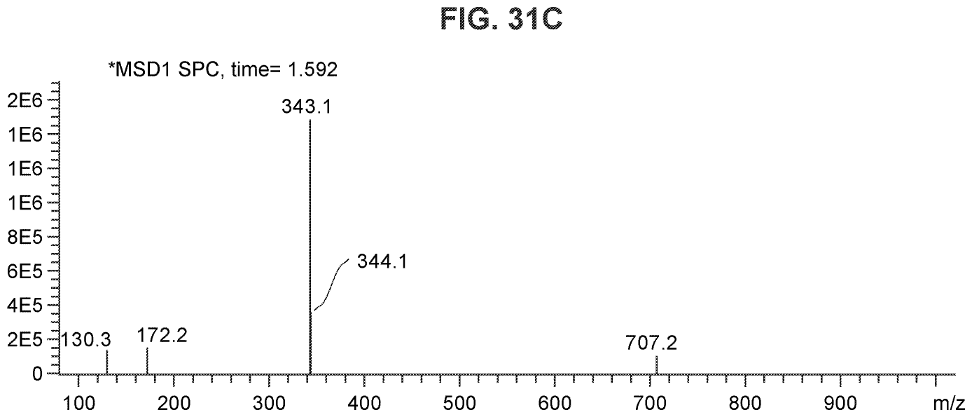

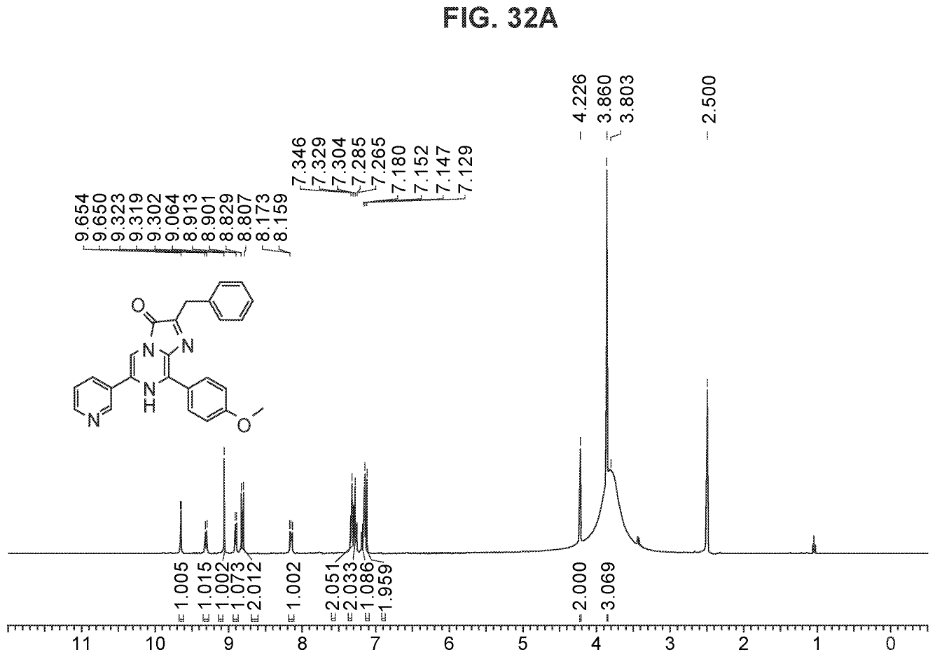

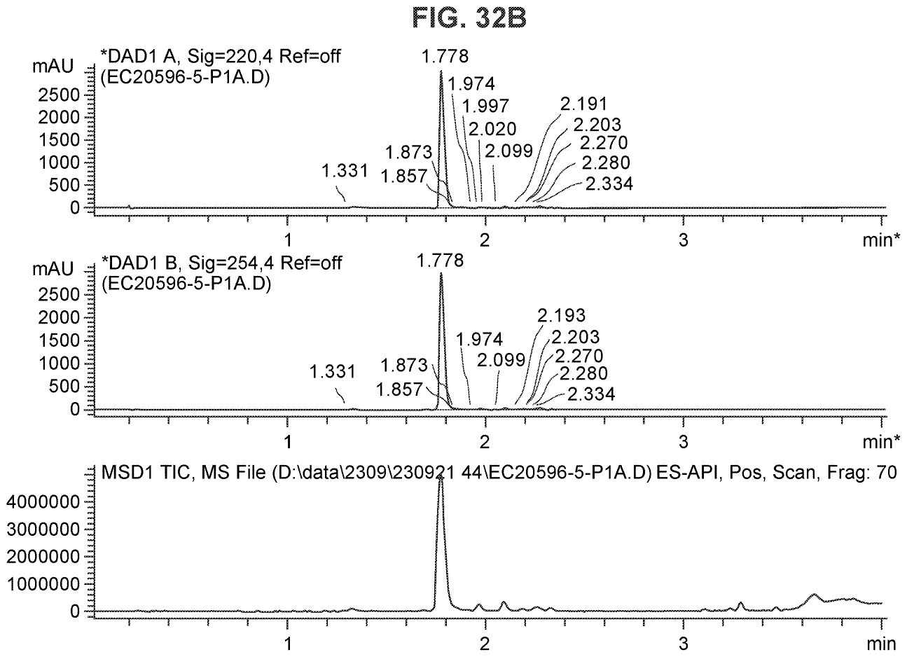

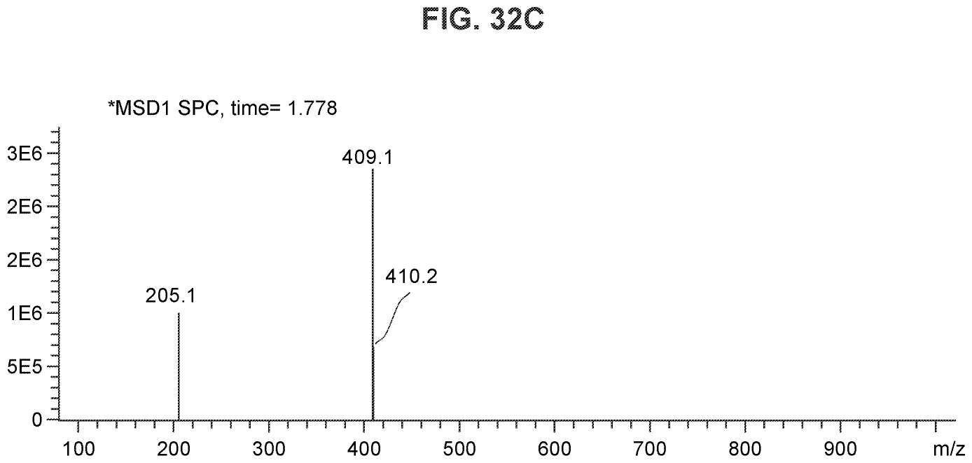

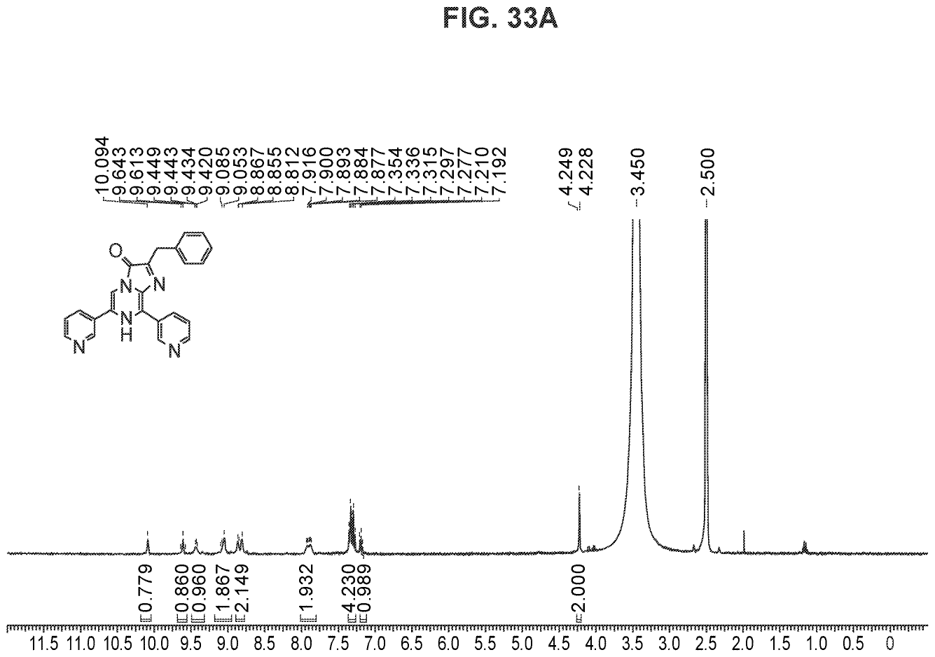

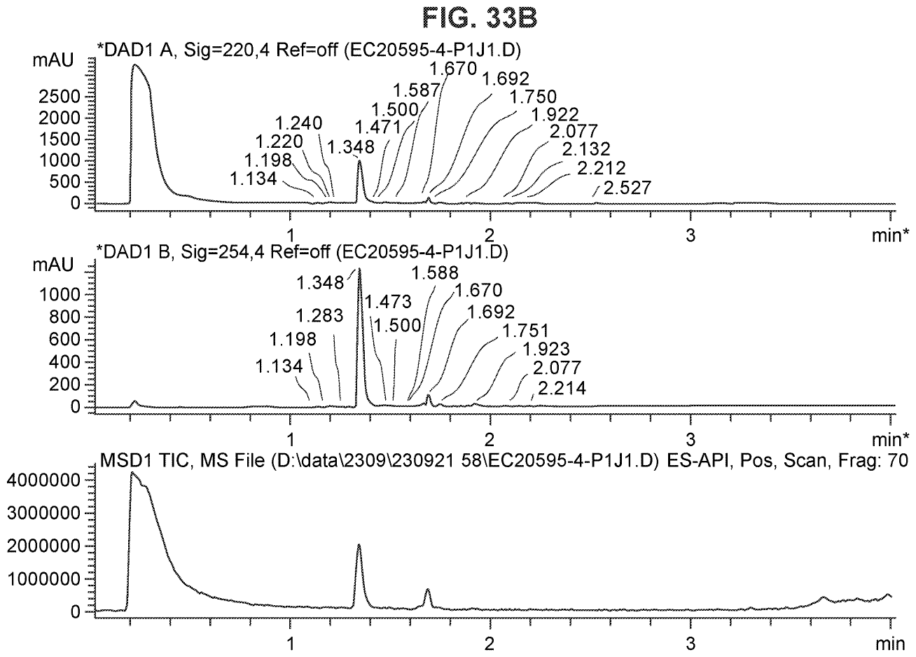

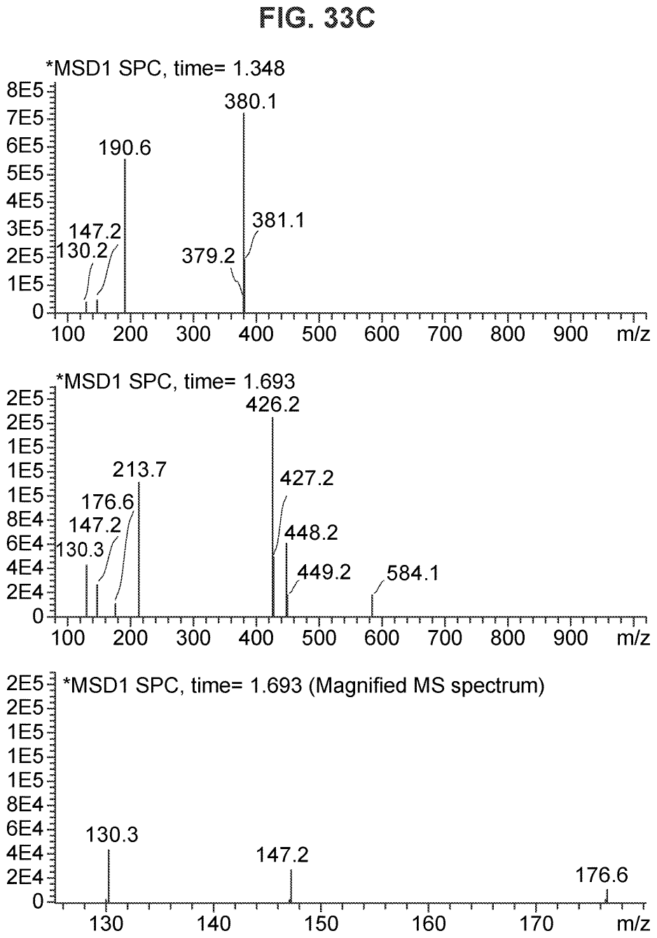

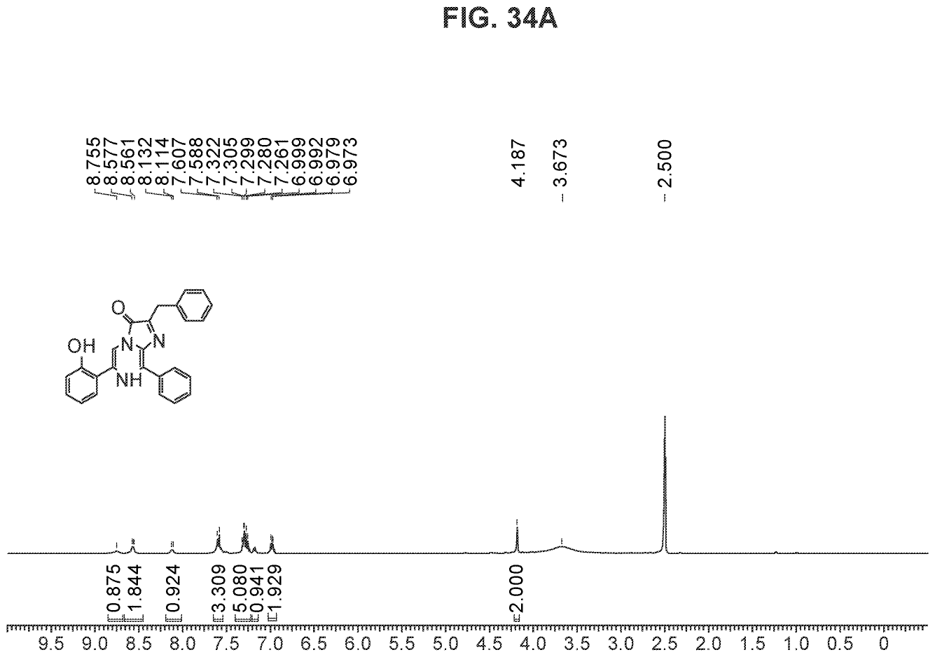

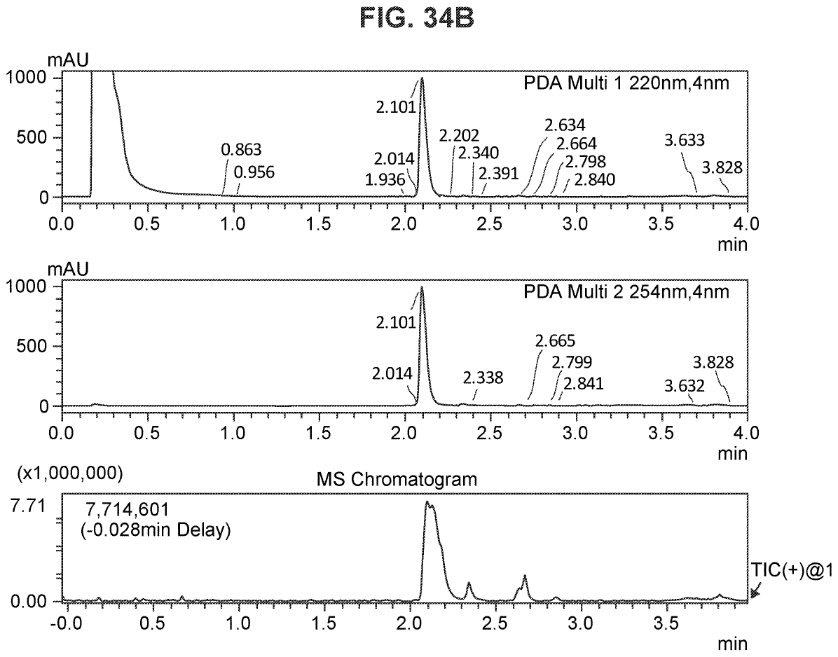

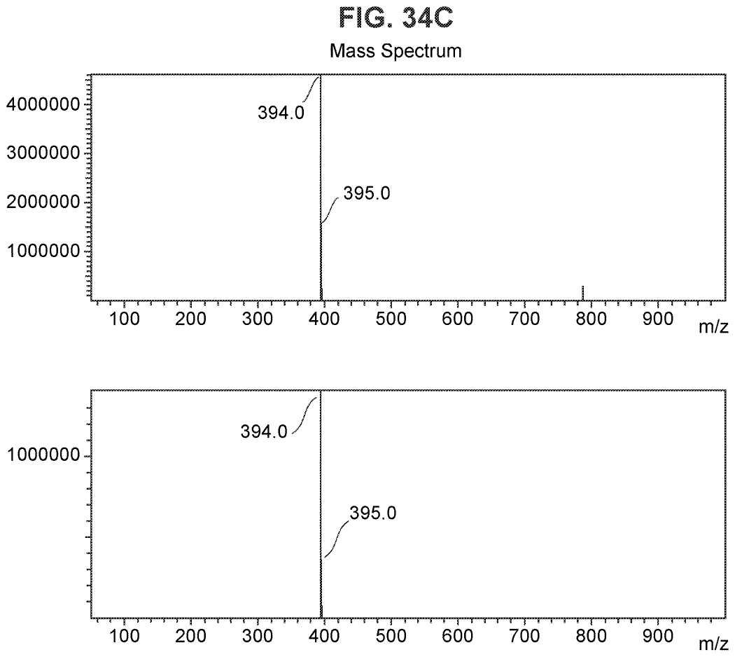

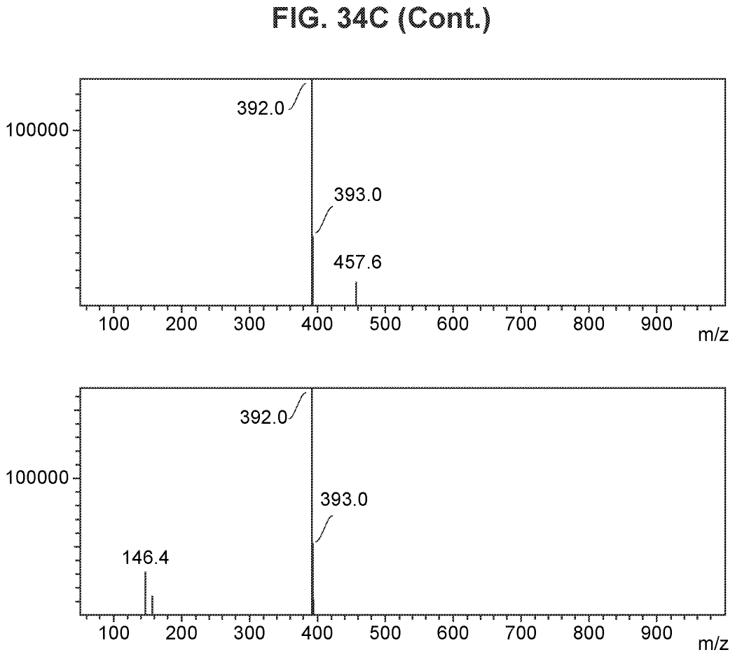

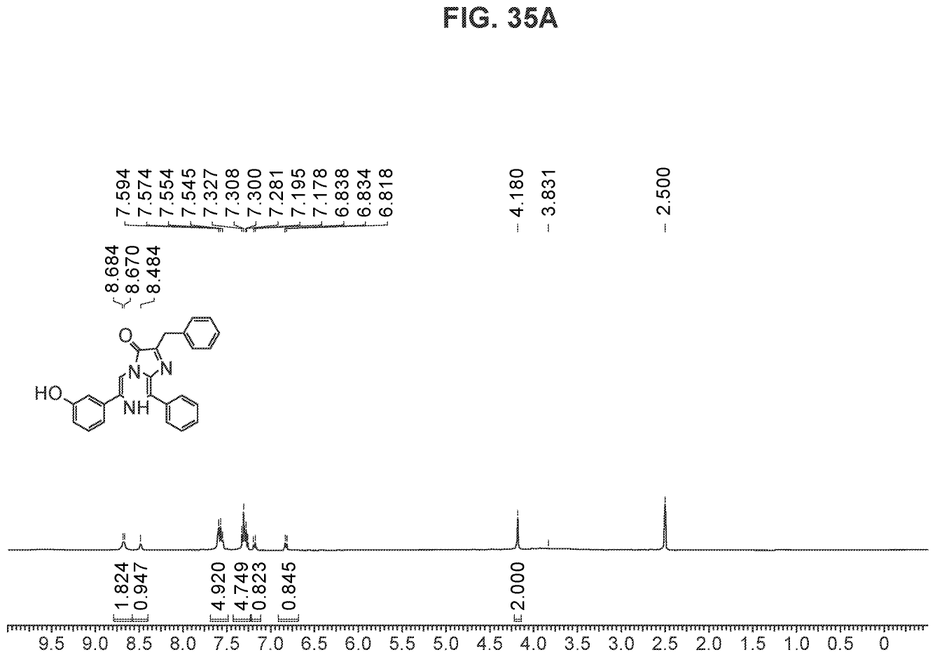

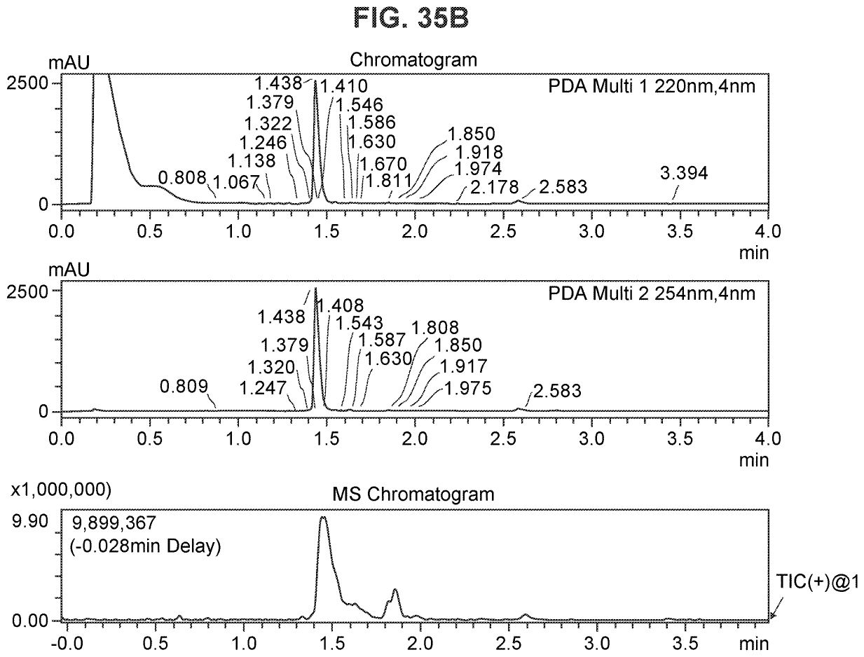

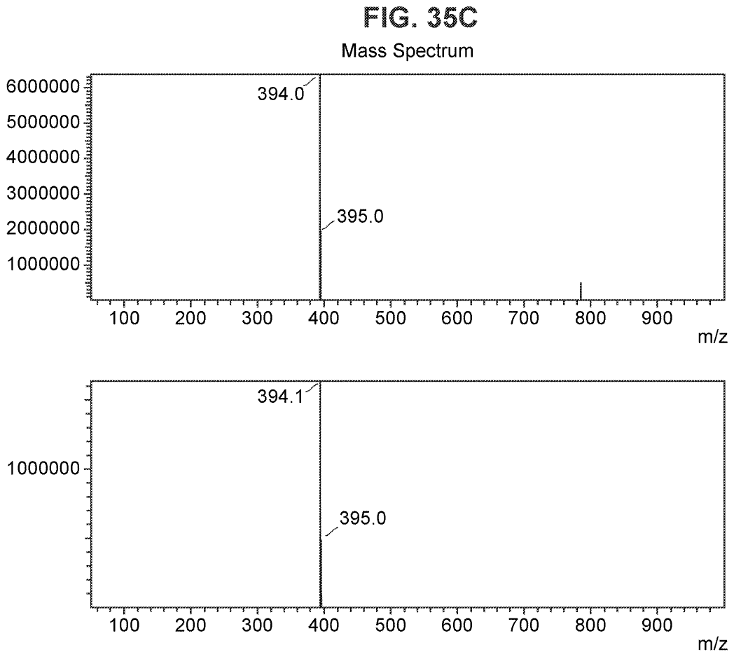

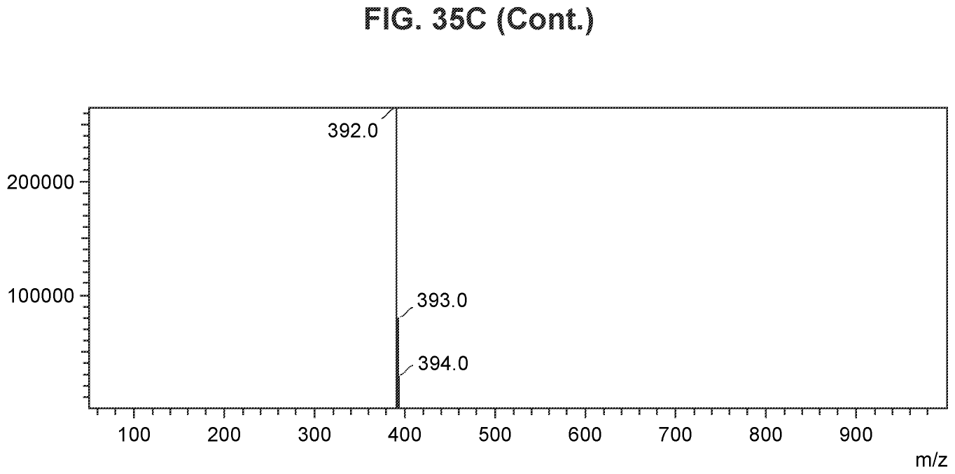

FIG. 14 compares bioluminescence emission of the synthetic luciferin substrate 1c to DTZ incubated with MBIO-301 in two assay conditions: 100% assay buffer (1X PBS, 0.5% Propylene Glycol, 0.05% Anti-foam, 100 mM Imidazole, 10 mM Ascorbic Acid buffer), and 20% human serum+80% assay buffer.

### Example 7: Bioluminescence Emission Spectra of Synthetic Luciferin Substrates with MBIO-301-Derived Split Enzyme

The MBIO-301 enzyme was split into two components: MBIO-557 (Small portion) and MBIO-563 (Large portion).
MBIO-557 has the following sequence: NRVDEVRVYINGSGS (SEQ ID NO:2735)
MBIO-563 has the following sequence:

(SEQ ID NO: 2736)
SEEQIRQNLRRFYEALDSGDADTAASLFHPGVTIHLWDGVTFTSREEFRE
WFERLFSTSKDALREIKSLEVRGDTVEVHVQLHFTRNGQKHTVDLTHLFH
FRG

Bioluminescence emission of synthetic luciferin substrates was measured. The luciferin substrates were incubated with MBIO-301-derived split enzyme. The two-component MBIO-301 split luciferase enzyme was fused to the rapamycin inducible FRB:FKBP system and incubated with its complementary part at a 1:1 ratio. Each of the molecules was incubated with 1×PBS, 0.5% Propylene Glycol, 0.05% Anti-foam, 100 mM Imidazole, 10 mM Ascorbic Acid buffer, 100 nM Rapamycin and emission signal was measured using a Synergy H1 plate reader. FIG. 15 shows bioluminescence emission of synthetic luciferin substrates incubated with MBIO-301-derived split enzyme.

In-well concentrations: 1 nM MBIO-557 (EGFP-FRB-Small portion), 1 nM MBIO-563 (EGFP-FKBP-Large portion), 10 μM Substrate, 100 nM Rapamycin.

TABLE 16

| | DTZ | 1c | 1d | 1n-2 | 2d | 2p |
|---|---|---|---|---|---|---|
| Fold Change with | 58.4 | 116.8 | 171.6 | 29.5 | 46.2 | 17.1 |
| Rapamycin Addition | | | | | | |
| at Initial Read | | | | | | |

### Example 8: LuxSit-i Variants Lacking Lysine Residues

LuxSit-i variants in which all of the lysine residues were substituted with another amino acid were generated. An example LuxSit-i variant that lacks lysine residues and retains activity was generated starting with the amino acid sequence of MBIO-4039 and has the following sequence:

### MBIO-4039_No Lysine Residues

(SEQ ID NO: 2737)
MSGMSEEQIRQNLRRFYEALDSGDAITAASLFDPGVTITLWDGTTFTSVE
EFREWFESQFSTSRDALREISSLEVRGDTVEVTVRLSFTRNGQRHTVDLL
QLFRFVGNRVVAVRVYVNPT

This lysine-less variant has only 76.9% sequence identity to LuxSit-i (SEQ ID NO:1).

This MBIO-4039 was used to generate two fragments that associate to form a functional enzyme:

### MBIO-4039_LgLux_No Lysine Residues

(SEQ ID NO: 2734)
MSGGNRVVAVRVYVNPT

### MBIO-4039_SmLux_No Lysine Residues

(SEQ ID NO: 2733)
MSGMSEEQIRQNLRRFYEALDSGDAITAASLFDPGVTITLWDGTTFTSVE
EFREWFESQFSTSKDALREISSLEVRGDTVEVTVRLSFTRNGQKHTVDLL
QLFKFV

These lysine-less variants are useful for applications such as ubiquitination analysis and assaying protein degradation.

### Example 9: LuxSit-I Variants Generated from MBIO-4039

LuxSit-i variant, MBIO-4039 (SEQ ID NO: 2730) includes the following mutations relative to LuxSit-i: F9N, D23′, H30D, H36T, V41T, R46V, R55S, L56Q, Q4L, K68S, H85T, Q82R, H84S, A85F, H87R, T97L, H98Q, H99L, W100F, H101K, R103V, T18V, E109A, H113Y, I114V.

Table 17 lists the variants generated starting from the sequence of MBIO-4039 (SEQ ID NO: 2730).

TABLE 17

| | SEQ ID | |
|---|---|---|
| Variant Name | NO | Mutations (relative to LuxSit-i) |
| MBIO_4517 | 2753 | [‘F9N’, ‘D23I’, ‘H30D’, ‘H36T’, ‘V41T’, ‘R46V’, ‘R55S’, ‘L56Q’, ‘Q64L’, ‘K68S’, |
| | | ‘H80T’, ‘Q82R’, ‘H84S’, ‘A85F’, ‘H87R’, ‘D95E’, ‘T97L’, ‘H98Q’, ‘H99L’, ‘W100F’, |
| | | ‘H101K’, ‘R103V’, ‘T108V’, ‘E109A’, ‘H113Y’, ‘I114V’] |
| MBIO_4518 | 2756 | [‘F9N’, ‘D23V’, ‘H30D’, ‘H36T’, ‘V41T’, ‘R46V’, ‘R55S’, ‘L56Q’, ‘Q64L’, ‘K68S’, |
| | | ‘H80T’, ‘Q82R’, ‘H84S’, ‘A85F’, ‘H87R’, ‘T97L’, ‘H98Q’, ‘H99L’, ‘W100F’, ‘H101K’, |
| | | ‘R103V’, ‘T108V’, ‘E109A’, ‘H113Y’, ‘I114V’] |
| MBIO_4519 | 2759 | [‘F9N’, ‘D23I’, ‘H30D’, ‘H36T’, ‘V41T’, ‘R46V’, ‘R55S’, ‘L56Q’, ‘Q64L’, ‘K68S’, |
| | | ‘H80T’, ‘Q82R’, ‘H84S’, ‘A85F’, ‘H87R’, ‘T97L’, ‘H98Q’, ‘H99L’, ‘W100F’, ‘H101K’, |
| | | ‘R103V’, ‘T108V’, ‘E109A’, ‘H113Y’, ‘I114V’, ‘P116A’] |
| MBIO_4520 | 2762 | [‘F9N’, ‘D23I’, ‘H30D’, ‘H36T’, ‘V41T’, ‘R46V’, ‘R55S’, ‘L56Q’, ‘Q64L’, ‘K68S’, |
| | | ‘V72M’, ‘H80T’, ‘Q82R’, ‘H84S’, ‘A85F’, ‘H87R’, ‘T97L’, ‘H98Q’, ‘H99L’, ‘W100F’, |
| | | ‘H101K’, ‘R103V’, ‘T108V’, ‘E109A’, ‘H113Y’, ‘I114V’] |
| MBIO_4521 | 2765 | [‘F9N’, ‘R12P’, ‘D23I’, ‘H30D’, ‘H36T’, ‘V41T’, ‘R46V’, ‘R55S’, ‘L56Q’, ‘Q64L’, ‘K68L’, |
| | | ‘H80T’, ‘Q82R’, ‘H84S’, ‘A85F’, ‘H87R’, ‘T97L’, ‘H98Q’, ‘H99L’, ‘W100F’, ‘H101K’, |
| | | ‘R103V’, ‘T108V’, ‘E109A’, ‘H113Y’, ‘I114D’] |
| MBIO_4522 | 2768 | [‘F9N’, ‘D23I’, ‘H30D’, ‘H36T’, ‘G40D’, ‘V41T’, ‘R46V’, ‘R55S’, ‘L56Q’, ‘Q64L’, |
| | | ‘K68S’, ‘H80T’, ‘Q82R’, ‘H84S’, ‘A85F’, ‘H87R’, ‘T97L’, ‘H98Q’, ‘H99L’, ‘W100F’, |
| | | ‘H101K’, ‘R103V’, ‘T108V’, ‘E109A’, ‘H113Y’, ‘I114V’] |

MBIO-4517 exhibited the best properties amongst the variants generated from MBIO-4039.

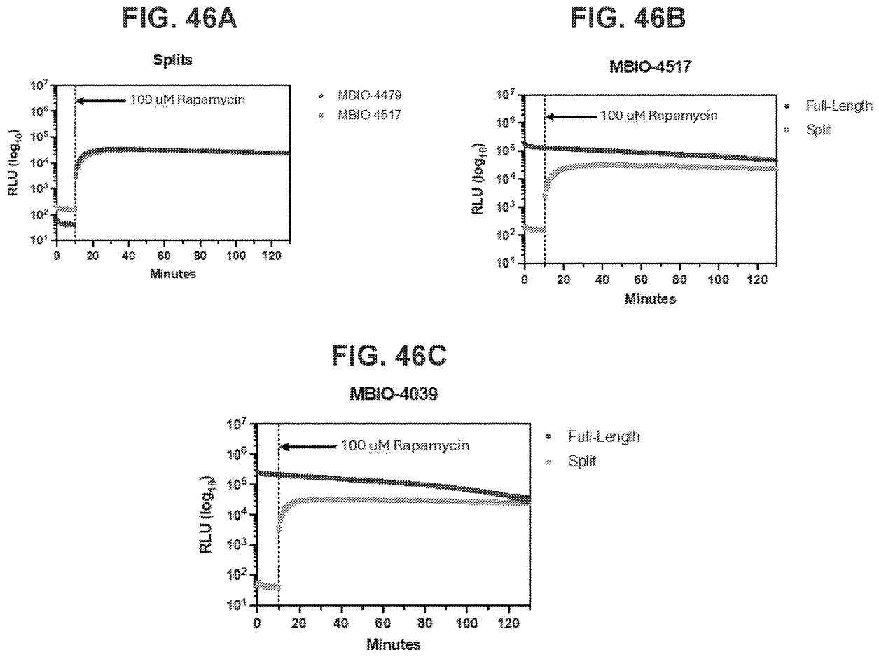

MBIO-4517 and MBIO-4039 were split into large and small fragments and conjugated to the proteins that bind the molecule Rapamycin (FKBP and FRB). Luciferase activity before and after rapamycin addition is shown in FIG. 46A. A plot comparing luciferase activity of the split luciferase version to full length version for MBIO-4517 and for MBIO-4039 is shown in FIG. 46B and FIG. 46C, respectively.

### Example 10: LuxSit Split Optimization

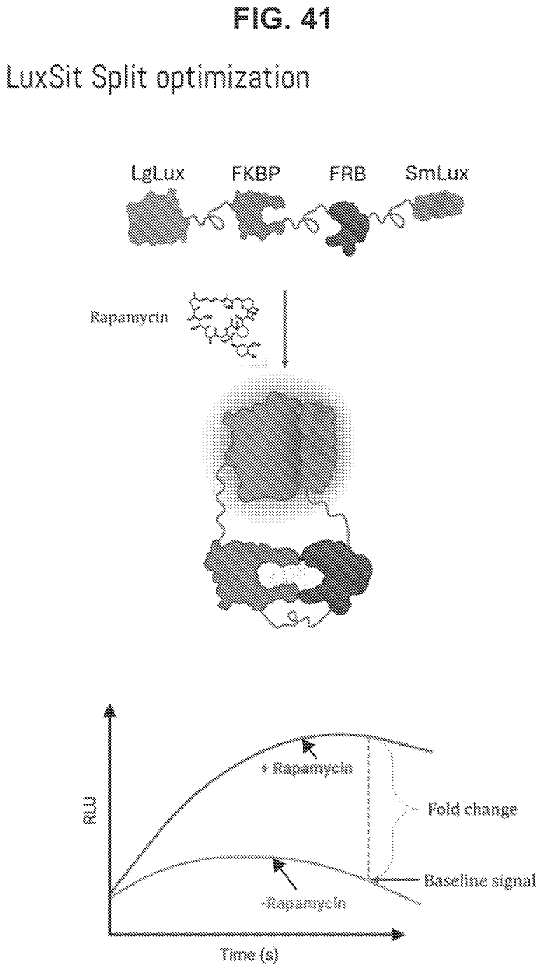

To optimize the function of the LuxSit Splits, a construct containing Small Lux (SmLux), Large Lux (LgLux) and the proteins that bind the molecule Rapamycin (FKBP and FRB), connected by GS linkers, was expressed on the surface of yeast. When 50 uM of substrate 1c is added to yeast expressing this construct, a baseline signal is observed due to SmLux and LgLux reconstitution. When 20 uM of Rapamycin is added, SmLux and LgLux are brought closer to each other by FKBP and FRB, enhancing their reconstitution. As a result, an increase in signal is observed. See FIG. 41.

Libraries of SmLux and LgLux were assembled and tested independently to identify variants with lower baseline and higher fold change.

SmLux optimization was performed on SmLux_4039 (RVVAVRVYVNPTG; SEQ ID NO:2770). SmLux_4039 was mutated to generate MBIO-5343 (SEQ ID NO:2771) and MBIO-5344 (SEQ ID NO:2772). Table 18 shows that both mutants exhibited lower baseline activity and a higher activity in the presence of rapamycin as compared to SmLux_4039:

| | | | Baseline Signal | Fold change |
|---|---|---|---|---|
| | | | (Normalized by | upon Rap |
| MBIO | Sequence | Mutations | expression) | addition |
| SmLux_4039 | RVVAVRVYVNPTG | | 122526.2 | 1.5 |
| MBIO-5343 | RVVIVRVYVNPTG | A4I | 20247.3 | 1.8 |
| MBIO-5344 | HVVAVRVYVNPTG | R1H | 23882 | 2 |

LgLux optimization was performed on LgLux_4039 (SEQ ID NO:2773). Sequences and properties of the generated variants (SEQ ID NO:2797 through SEQ ID NO:2805) are provided below in Table 19.

TABLE 19

| | | | | Baseline | |
|---|---|---|---|---|---|
| | | | | Signal | Fold |
| | | | | (Normalized | change |
| | | | | by | upon Rap |
| MBIO | Sequence | SEQ ID NO | Mutations | expression) | addition |
| LgLux_4039 | MSEEQIRQNLRRFYEALDSGDAITAASLFDPGVTI | 2773 | | 22064.6 | 1.9 |
| | TLWDGTTFTSVEEFREWFESQFSTSKDALREISSL | | | | |
| | EVRGDTVEVTVRLSFTRNGQKHTVDLLQLFKFV | | | | |
| MBIO-5335 | MCEEQIRQNLLRFYEALDSGDAITAASLFDPGVTI | 2797 | S2C, R11L, | 23769.5 | 1.9 |
| | TLWDGTTFTSIEEFREWFDSQFSTSKDALREISSL | | V46I, E54D | | |
| | EVRGDTVEVTVRLSFTRNGQKHTVDLLQLFKFV | | | | |
| MBIO-5336 | MSEEQIRQNLLRFYEALDSGDAITAASLFDPGVTI | 2798 | R11L | 16379.8 | 2.1 |
| | TLWDGTTFTSVEEFREWFESQFSTSKDALREISSL | | | | |
| | EVRGDTVEVTVRLSFTRNGQKHTVDLLQLFKFV | | | | |
| MBIO-5337 | MSEEQIRQNLLRFYEALDSGDAITAASLFNPGVTI | 2799 | R11L, D30N | 14853.7 | 2.1 |
| | TLWDGTTFTSVEEFREWFESQFSTSKDALREISSL | | | | |
| | EVRGDTVEVTVRLSFTRNGQKHTVDLLQLFKFV | | | | |
| MBIO-5338 | MSEEQIRQNLRRFYEALDSGDAITAASLFDPGVTI | 2800 | T41A, H92Q | 18945.1 | 2.2 |
| | TLWDGATFTSVEEFREWFESQFSTSKDALREISSL | | | | |
| | EVRGDTVEVTVRLSFTRNGQKQTVDLLQLFKFV | | | | |
| MBIO-5339 | MSEEQIRQNLLRFYEALDSGDAVTAASLFDPGVT | 2801 | R11L, I23V | 28286 | 1.9 |
| | ITLWDGTTFTTVEEFREWFESQFSTSKDALREISS | | | | |
| | LEVRGDTVEVTVRLSFTRNGQKHTVDLLQLFKFV | | | | |
| MBIO-5342 | MSEEQIRQNLRRFYEALDSGDAITAASLFDPGVTI | 2802 | E54G, S55T, | 3361.4 | 2 |
| | TLWDGTTFTSVEEFREWFGTQFSTSKDALREISSL | | R87S | | |
| | EVRGDTVEVTVRLSFTSNGQKHTVDLLQLFKFV | | | | |
| MBIO-5341 | MSEEQIRQNLRRFYEALDSGDAITAASLFDSGVTI | 2803 | P31S | 10433.7 | 2.2 |
| | TLWDGTTFTSVEEFREWFESQFSTSKDALREISSL | | | | |
| | EVRGDTVEVTVRLSFTRNGQKHTVDLLQLFKFV | | | | |
| MBIO-5340 | MTEEQLRQNLLRFYEALDSGDAITAASLFDPGVT | 2804 | S2T, I6L, | 6666.1 | 2.2 |
| | ITLWDGTTFTSVEKFREWFESQFSTSKDALREISS | | R11L, E48K | | |
| | LEVRGDTVEVTVRLSFTRNGQKHTVDLLQLFKFV | | | | |
| | MSEEQIRQNLRRFYEALDCGDAITAASLFDPGVT | 2805 | S19C | 3667.6 | 2 |
| | ITLWDGTTFTSVEEFREWFESQFSTSKDALREISS | | | | |
| | LEVRGDTVEVTVRLSFTRNGQKHTVDLLQLFKFV | | | | |

### Example 11: Luciferase Activity of LuxSit-i Variants

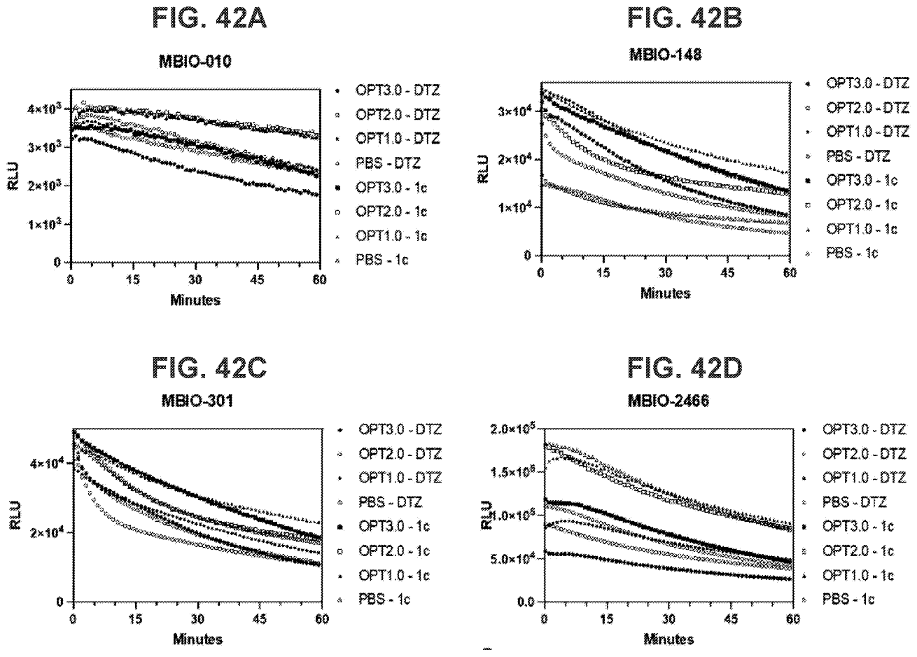

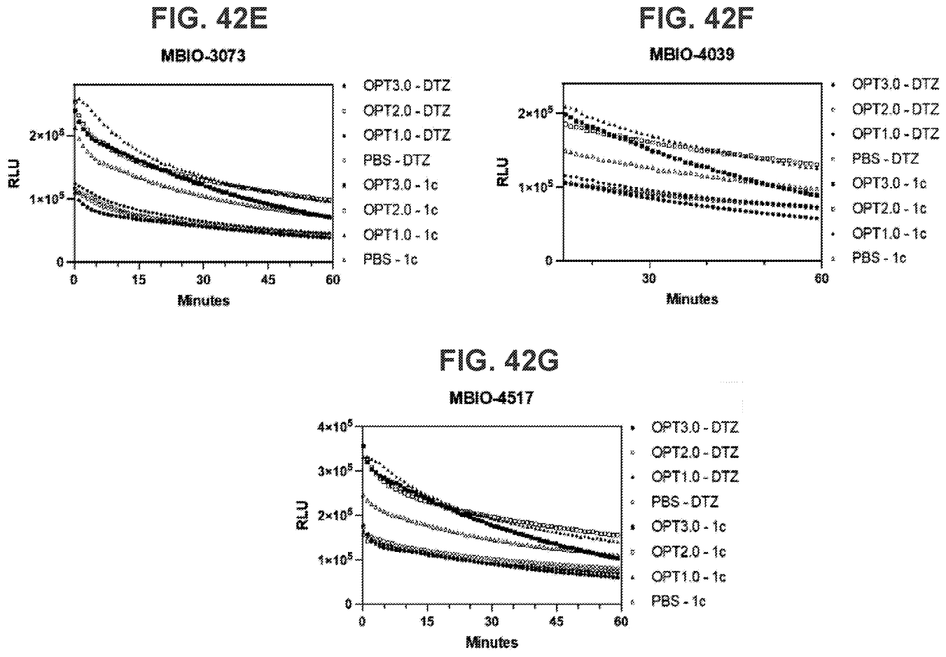

The luminescence of the LuxSit variants MBIO-148 (SEQ ID NO:2600), MBIO-301 (SEQ ID NO:2603), MBIO-2466 (SEQ ID NO:2703), MBIO-3073 (SEQ ID NO:2711), MBIO-4039 (SEQ ID NO:2730), MBIO-4517 (SEQ ID NO:2753), obtained along the optimization process were tested with substrate DTZ or substrate 1c, using buffers PBS, OPT1.0, OPT2.0, or OPT3.0 (see Example 4—“OB” and “OPT” are used interchangeably). FIGS. 42A-42G show enzymatic activity of LuxSit-i variants measured in different buffers: phosphate-buffered saline (PBS), OPT1.0, OPT2.0, and OPT3.0. MBIO-010 is LuxSit-i, SEQ ID NO:1).

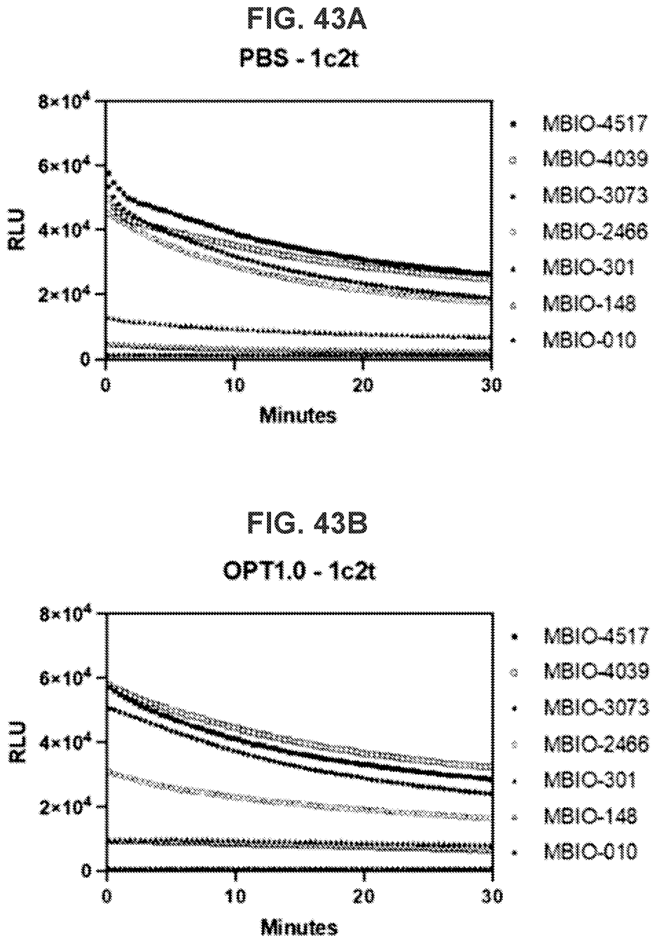

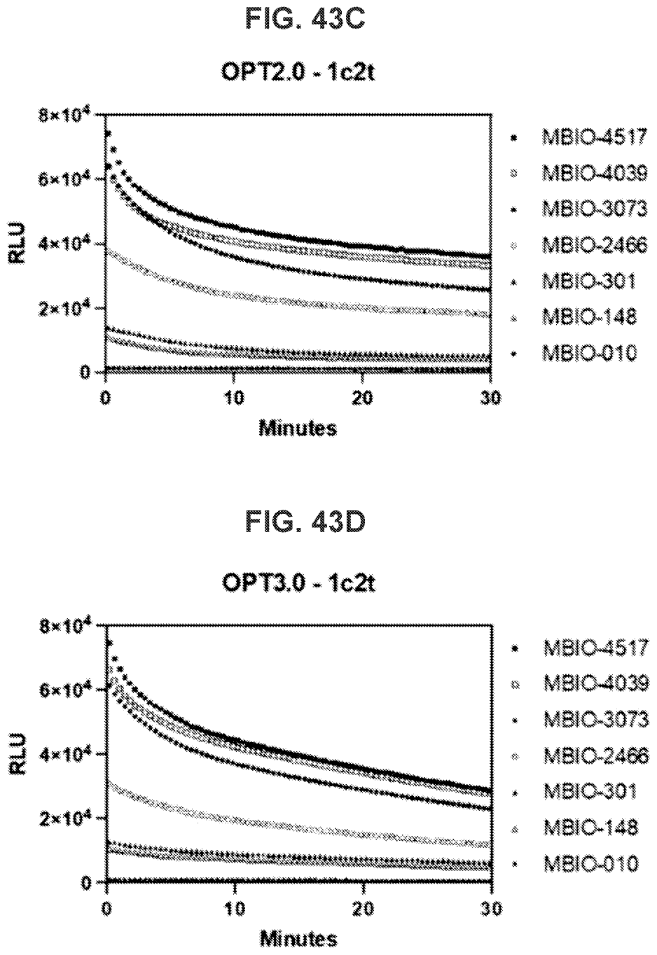

FIGS. 43A-43D show enzymatic activity of the listed LuxSit-i variants measured in different buffers: phosphate-buffered saline (PBS), OPT1.0, OPT2.0, and OPT3.0, using substrate 1c2t.

While the subject proteins have been particularly shown and described with references to certain embodiments thereof, it will be understood by those skilled in the art that various changes in form and details may be made therein without departing from the scope of the invention encompassed by the appended claims.

### Example 12: LgLux Stability Optimization

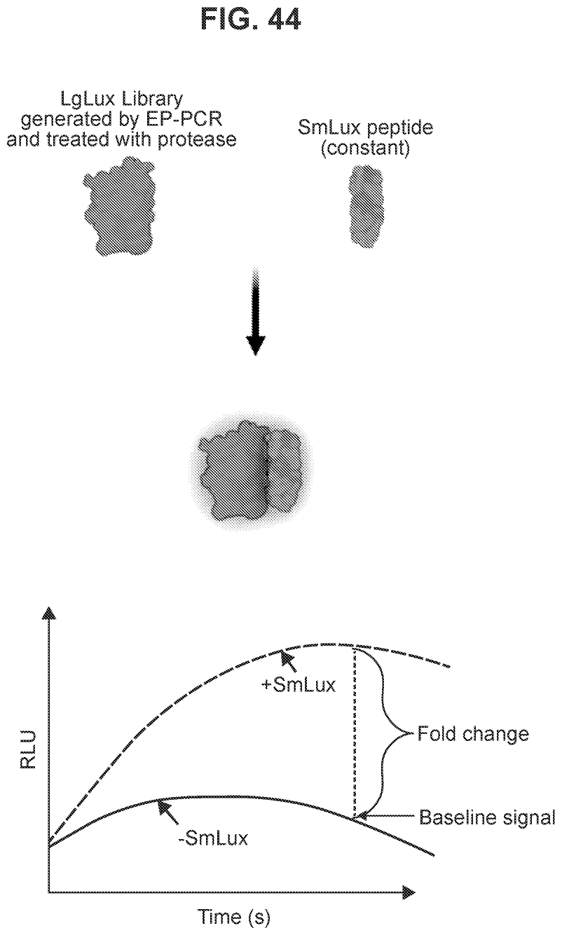

LgLux mutants were generated by error-prone PCR (EP-PCR) and treated with protease and their activity measured as depicted schematically in FIG. 44.

Yeast expressing a library of the LgLux mutants were incubated with proteases and screened to select variants that were stable enough to resist proteolysis. Functional variants were selected by adding 50 uM of substrate 1c and 200 nM of SmLux.

The nucleotide sequence encoding LgLux_4039 was subjected to EP-PCR to identify protease-stable mutants. The sequences (SEQ ID NOs: 2806-2814) and activity of the mutants are summarized below in Table 20.

TABLE 20

| | | | | | Fold |
|---|---|---|---|---|---|
| | | | | Baseline | change |
| | | | | Signal | upon |
| | | | | (Normalized | SmLux |
| | | | SEQ ID | by | peptide |
| MBIO | Sequence | Mutations | NO | expression) | addition |
| LgLux_4039 | MSEEQIRQNLRRFYEALDSGDAITAASLFDPGVTITLWD | | 2773 | 417.8 | 78.5 |
| | GTTFTSVEEFREWFESQFSTSKDALREISSLEVRGDTVEV | | | | |
| | TVRLSFTRNGQKHTVDLLQLFKFV | | | | |
| | MSEEQIRQSLRRFYEALDSGDAITAASLFDPGVTITLWD | N9S | 2806 | 973.7 | 126.7 |
| | GTTFTSVEEFREWFESQFSTSKDALREISSLEVRGDTVEV | | | | |
| | TVRLSFTRNGQKHTVDLLQLFKFV | | | | |
| | MSEEQIRQNLRRFYEALDSGDAITAASLFDPGVTITLWD | L99Q | 2807 | 738.4 | 83.3 |
| | GTTFTSVEEFREWFESQFSTSKDALREISSLEVRGDTVEV | | | | |
| | TVRLSFTRNGQKHTVDLLQQFKFV | | | | |
| | MSEEQIRQNLRRFYEALDSGDAITAASLFDPGVTITLWD | K101E | 2808 | 228.3 | 86.8 |
| | GTTFTSVEEFREWFESQFSTSKDALREISSLEVRGDTVEV | | | | |
| | TVRLSFTRNGQKHTVDLLQLFEFV | | | | |
| | MSEEQIRQDLRRFYEALDSGDAITAASLFDPGVTITLWD | N9D, V103I | 2809 | 638.6 | 170 |
| | GTTFTSVEEFREWFESQFSTSKDALREISSLEVRGDTVEV | | | | |
| | TVRLSFTRNGQKHTVDLLQLFKFI | | | | |
| | MSEEQIRQNLRRFYEALDSGDAITAASLFDPGVTITLWD | F43L | 2810 | 327.2 | 81.7 |
| | GTTLTSVEEFREWFESQFSTSKDALREISSLEVRGDTVEV | | | | |
| | TVRLSFTRNGQKHTVDLLQLFKFV | | | | |
| | MSEEQIRQILRRFYEALDSGDAITAASLFDPGVTITLWDG | N9I, N88D | 2811 | 232.7 | 55.5 |
| | TTFTSVEEFREWFESQFSTSKDALREISSLEVRGDTVEVT | | | | |
| | VRLSFTRDGQKHTVDLLQLFKFV | | | | |
| | MSEEQIRQNLRRFYEALDSGDAITAASLFDPGVTITLWD | L64F | 2812 | 233.2 | 61.7 |
| | GTTFTSVEEFREWFESQFSTSKDAFREISSLEVRGDTVEV | | | | |
| | TVRLSFTRNGQKHTVDLLQLFKFV | | | | |
| | MSEEQIRQNLRRFYEALDSGDAITAASLFDPGVIITLWD | T34I | 2813 | 241.6 | 52 |
| | GTTFTSVEEFREWFESQFSTSKDALREISSLEVRGDTVEV | | | | |
| | TVRLSFTRNGQKHTVDLLQLFKFV | | | | |
| | MSEEQIRQNLRRFYEALDSGDAITAASLFDPGVTITLWD | E51V, K61T | 2814 | 934.2 | 53.1 |
| | GTTFTSVEEFRVWFESQFSTSTDALREISSLEVRGDTVEV | | | | |
| | TVRLSFTRNGQKHTVDLLQLFKFV | | | | |

### Example 13: LgLux Stability Optimization

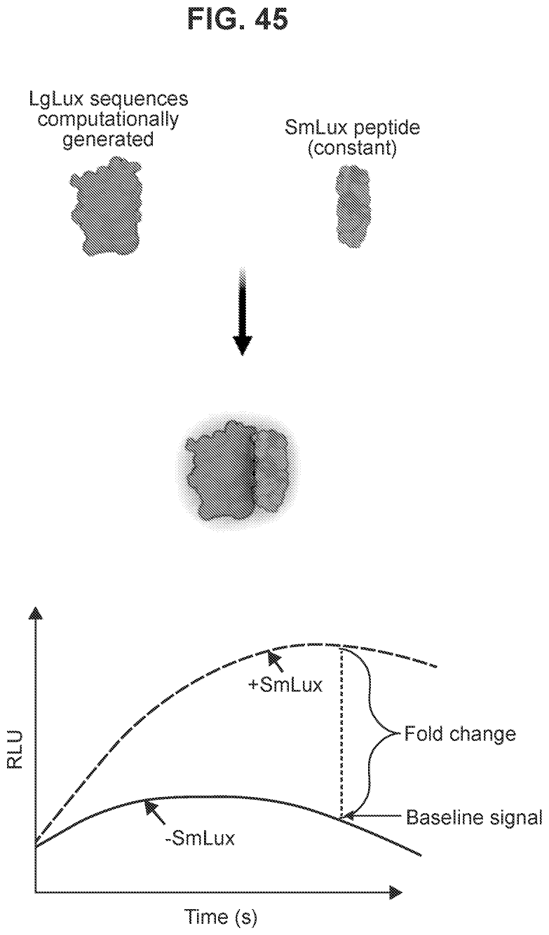

LgLux sequences were designed computationally, and their activity measured as depicted schematically in FIG. 45.

A library of the computationally generated LgLux variants were expressed in E. coli and tested in lysates. 20 uM of SmLux and 50 uM of substrate 1c was used for the screening.

The activity of the mutants is summarized in the tables below:

LgLux_4039 sequence is as set forth in SEQ ID NO:2773. MBIO-5103 through MBIO-5124, MBIO-5261 through MBIO-5295, MBIO-5101, and MBIO-5102 (SEQ ID NO:2815-SEQ ID NO:2873, respectively) are computationally generated LgLux. The sequences and activity of these LgLux are shown below in Table 21.

TABLE 21

| | | | Baseline | |
|---|---|---|---|---|
| | | | Signal | Fold change |
| | | SEQ | (Normalized | upon SmLux |
| | | ID | by | peptide |
| MBIO | Sequence | NO | expression) | addition |
| LgLux_4039 | MSEEQIRQNLRRFYEALDSGDAITAASLFDPGVTITLWDGT | 2773 | 299.2 | 214.43 |
| | TFTSVEEFREWFESQFSTSKDALREISSLEVRGDTVEVTVRL | | | |
| | SFTRNGQKHTVDLLQLFKFV | | | |
| 5103 | MTPEQRIANVRAFYAALASGDLEAAKALFDPGVTITLWDG | 2815 | 193.2 | 0.94 |
| | TTFTSLEEFLAWFEKQFDASKDAKREIVDIEVEGDVVRVLV | | | |
| | RLTYTKDGKEKVVDLLQLLKFV | | | |
| 5104 | MTPEEIRANVRAFYAALASGDLAAAKALFDPGTTITLWDG | 2816 | 180.8 | 0.97 |
| | TTFTSLEEFLAWFEKQFKASKDAKREIVSLEVEGDTVRVLVR | | | |
| | LTYTVDGKEKVVDLLQLLKFV | | | |
| 5105 | MTPEEIIANVKKFYEALASGDLATAKSLFDPGTTITLWDGTT | 2817 | 167.4 | 1 |
| | FTSLEEFLAWFEKQFKASKDAKREIVSIEVDGNVVKVLVRLT | | | |
| | YTVDGKEKVVDLLQLLKFV | | | |
| 5106 | MTEEEIIANVEKFYAALASGDLETAKALFDPGTTITLWDGT | 2818 | 169.8 | 1 |
| | TFTSLEEFLAWFEKQFKASKDAKREIVSIEVEGDTVKVLVRL | | | |
| | TYTVDGKEKVVDLLQLLKFV | | | |
| 5107 | MTPEEIKANVEAFYAALASGDLEKAKALFDPGTTITLWDGT | 2819 | 167.8 | 0.99 |
| | TFTSLEEFLAWFEKQFKASKDAKREIVSMKVEGDTVRVLVR | | | |
| | LTYTKDGKEQVVDLLQLIKVV | | | |
| 5108 | MTPEEIIENVKAFYAALASGDLEAAKALFDPGTTITLWDGT | 2820 | 167.4 | 0.92 |
| | TFTSLEEFLEWFEKQFEASKDAKREIKSIEVEGNVVKVLVRL | | | |
| | TYVKDGKEKVVDLLQLLKFV | | | |
| 5109 | MTPEEIRANVKAFYAALASGDLEKAKALFDPGTTITLWDGT | 2821 | 169 | 0.98 |
| | TFTSLEEFLAWFEKQFKASKDAKREIVSMEINGDTVRVLVR | | | |
| | LTYTVDGEEKVVDLLQLLKFV | | | |
| 5110 | MTPEEIIENVKKFYEALASGDLETAKSLFDPGTTITLWDGTT | 2822 | 167.6 | 0.97 |
| | FTSLEEFLEWFEKQFEASKDAKREILSIEVEGNVVKVLVRLTY | | | |
| | TKDGKEKVVDLLQLLKFV | | | |
| 5111 | MTPEEIRANVEAFYAALASGDLEAAKALFDPGTTITLWDGT | 2823 | 166.2 | 1.01 |
| | TFTSLEEFLAWFEKQFETSKDAKREIVSLEIEGDTVRVLVRLT | | | |
| | YTKNGEVKEVDLLQLLKFV | | | |
| 5112 | MTPEEIRANVRAFYAALASGDLEAAKALFDPGTTITLWDG | 2824 | 174.4 | 0.93 |
| | TTFTSLEEFLAWFEKQFKASKDAKREIVSLEVEGDVVRVLVR | | | |
| | LTYVKDGKEEVVDLLQLLKFV | | | |
| 5113 | MTPEEIRANVEAFYAALASGDLEAAKALFDPGTTITLWDGT | 2825 | 180.6 | 1.15 |
| | TFTSLEEFLAWFEKQFEASKDAKREILSLEIEGNTVRVLVRLT | | | |
| | YTKDGKEQVVDLLQLLKFV | | | |
| 5114 | MTPEEIIANVKNFYAALASGDLEAAKALFDPGTTITLWDGT | 2826 | 271.8 | 1.38 |
| | TFTSLEEFLAWFEKQFKASKDAKREIVSMEVEGNVVKVLVR | | | |
| | LTYTVDGEEKVVDLLQLLKFV | | | |
| 5115 | MTPEEIIANVKKFYAALASGDLETAKALFDPGTTITLWDGT | 2827 | 165 | 3.07 |
| | TFTSLEEFLAWFEKQFEASKDAKREIVSIEVKGNTVKVLVRL | | | |
| | TYVKDGKEQVVDLLQLLKFV | | | |
| 5116 | MTPEEIRANVERFYAALASGDLDTAKALFDPGTTITLWDGT | 2828 | 162.6 | 1.11 |
| | TFTSLEEFLAWFEKQFKASKDAKREIVELEIEGNVVRVLVRL | | | |
| | TYVKDGKEQVVDLLQLLKFV | | | |
| 5117 | MTDEEIRANVRAFYAALASGDLETAKALFDPGTTITLWDG | 2829 | 154.8 | 1.05 |
| | TTFTSLEEFLAWFEKQFKTSKDAKREIVSLEVEGDTVRVLVR | | | |
| | LTYTKDGKEEVVDLLQLLKFV | | | |
| 5118 | MTPEEIIANVKAFYAALASGDLAAAKALFDPGTTITLWDGT | 2830 | 152 | 1.05 |
| | TFTSLDEFLAWFEKQFKASKDAKREILDIEVEGDVVRVLVRL | | | |
| | TYTVDGKEKVVDLLQLLKFV | | | |
| 5119 | MTPEQIRANVERFYAALASGDLETAKALFDPGTTITLWDG | 2831 | 155.6 | 0.98 |
| | TTFTSLEEFLAWFEKQFKASKDAKREIVSLEIEGDTVRVLVRL | | | |
| | TYTVDGEVKEVDLLQLLKFV | | | |
| 5120 | MTPEEIRENVLRFYEALASGDLETAKALFDPGTTITLWDGT | 2832 | 148.4 | 1.09 |
| | TFTSLEEFLAWFEEQFDTSKDAKREILSLEIEGDVVRVLVRLT | | | |
| | YVKDGEEKVVDLLQLLKWV | | | |
| 5121 | MTPEEIKENVKKFYEALASGDLETAKSLFDPGTTITLWDGT | 2833 | 126.6 | 1.29 |
| | TFTSLEEFLAWFEKQFDASKDAKREIVSMEIKGNEVKVLVR | | | |
| | LTYTKDGKEQTVDLLQLLKFV | | | |
| 5122 | MTPEEIFANVKAFYAALASGDLAAAKALFDPGTTITLWDG | 2834 | 146.2 | 1.04 |
| | TTFTSLDEFLAWFEKQFKASKDAKREILSMEVEGDTVRVLV | | | |
| | RLTYVKDGKEEVVDLLQLIKWV | | | |
| 5123 | MTPEKIRENVENFYAALASGDLEKAKALFDPGTTITLWDGT | 2835 | 145.6 | 1.09 |
| | TFTSLEEFLAWFEKQFKASKDAKREIKSLEIEGDTVKVLVRLT | | | |
| | YTVNGKEKTVDLLQLLKFV | | | |
| 5124 | MTPEEIIANVRAFYAALASGDLEAAKALFDPGTTITLWDGT | 2836 | 187.6 | 1.07 |
| | TFTSLEEFLAWFEKQFDASKDAKREIVSIEVEGDTVRVLVRL | | | |
| | TYTKDGKEQVVDLLQLLKFV | | | |
| 5261 | MTPEEIRANVRAFYAALASGDLAAARALFDPGVTITLWDG | 2837 | 176.8 | 2.16 |
| | TTFTSVEEFRAWFEEQFQTSKDALREIESIEVEGDTVRVLVR | | | |
| | LTFTRDGVTQEVDLLQLFKFV | | | |
| 5262 | MTPEERIENVRAFYAALASGDWAAARALFDPGVTITLWD | 2838 | 139.6 | 2.29 |
| | GTTFTSVEEFRAWFEKQFKTSKDALREIESIEVEGDTVRVLV | | | |
| | RLTFTRDGKEEEVDLLQLFKFV | | | |
| 5263 | MTPEEIRANVLAFYAALASGDLAAAEALFDPGVTITLWDG | 2839 | 137.4 | 1.38 |
| | TTFTSVAEFRAWFEAQFQTSKDALREIVDMRVEGDVVRVL | | | |
| | VRLTFVRDGEEQVVDLLQLFKFV | | | |
| 5264 | MTPEEIRANVEAFYAALASGDWEAARALFDPGVTITLWD | 2840 | 127.6 | 1.26 |
| | GTTFTSVEEFRAWFEEQFKTSKDALREIVDLEVEGDVVRVL | | | |
| | VRLTFVRDGKEEVVDLLQLFKFV | | | |
| 5265 | MTPEQIRANVRAFYAALASGDWAAAEALFDPGVTITLWD | 2841 | 126.4 | 2.8 |
| | GTTFTSVAEFRAWFEEQFKTSKDALREIVSMEVEGDTVRVL | | | |
| | VRLTFVRDGEEKVVDLLQLFKFV | | | |
| 5266 | MTPEEIRANVRAFYAALASGDLAAAKALFDPGVTITLWDG | 2842 | 129.8 | 1.09 |
| | TTFTSVEEFRAWFEEQFQTSKDALREIVSLRVEGDTVRVLV | | | |
| | RLTFTRDGKVQEVDLLQLFKFV | | | |
| 5267 | MTPEEILANVRAFYAALASGDLAAAKALFDPGVTITLWDG | 2843 | 130.6 | 1.42 |
| | TTFTSVEEFRAWFEEQFKTSKDALREIVSAEVEGDTVRVLV | | | |
| | RLTFTRDGKVEEVDLLQLFKFV | | | |
| 5268 | MTPEEIRENVRRFYAALASGDLEAAKSLFDPGVTITLWDGT | 2844 | 133 | 1.05 |
| | TFTSVEEFRAWFEEQFQTSKDALREIVSLEVEGDVVRVLVR | | | |
| | LTFVRDGEVQEVDLLQLFKFV | | | |
| 5269 | MTPEEIRENVRAFYAALASGDLAAAKALFDPGVTITLWDG | 2845 | 137.2 | 1.1 |
| | TTFTSVEEFRAWFEKQFKTSKDALREIVSLEVEGDTVRVLVR | | | |
| | LTFVRDGKEEVVDLLQLFKFV | | | |
| 5270 | MTPEAIIANVKAFYAALASGDWAAARALFDPGVTITLWDG | 2846 | 135.2 | 1.25 |
| | TTFTSVEEFRAWFEAQFETSKDALREIVSMEVEGDTVRVLV | | | |
| | RLTFVRNGKEEVVDLLQLFKFV | | | |
| 5271 | MTPEEIRENVRNFYAALASGDLEAAKALFDPGVTITLWDG | 2847 | 145 | 10.11 |
| | TTFTSVEEFRAWFEEQFKTSKDALREIVSMEIEGDTVRVLV | | | |
| | RLTFTRDGVEQVVDLLQLFKFV | | | |
| 5272 | MTPEERIANVRAFYAALASGDWAAAEALFDPGVTITLWD | 2848 | 141.4 | 11.86 |
| | GTTFTSVAEFRAWFEQQFQTSKDALREILDIEVEGDVVRVL | | | |
| | VRLTFVRDGKEQVVDLLQLFKFV | | | |
| 5273 | MTPEAIRENVRAFYAALASGDWEAAKALFDPGVTITLWD | 2849 | 140.4 | 1.01 |
| | GTTFTSVEEFRAWFEKQFKTSKDALREIVSLEVEGDVVRVL | | | |
| | VRLTFVRDGKEEVVDLLQLFKFV | | | |
| 5274 | MTPEEIRANVRAFYAALASGDWAAAEALFDPGVTITLWD | 2850 | 125.2 | 1.07 |
| | GTTFTSVAEFRAWFEAQFQTSKDALREIVSLEVEGDVVRVL | | | |
| | VRLTFVRDGRTEEVDLLQLFKFV | | | |
| 5275 | MTPEEIKENVLNFYAALASGDLEAAKALFDPGVTITLWDGT | 2851 | 122.8 | 1.35 |
| | TFTSVEEFRAWFEKQFKTSKDALREIVSMEVEGDVVRVLVR | | | |
| | LTFTRDGKVEEVDLLQLFKFV | | | |
| 5276 | MTDEEIRANVRAFYAALASGDLEAAKALFDPGVTITLWDG | 2852 | 125.6 | 6.45 |
| | TTFTSVEEFRAWFEKQFKTSKDALREIESLEVEGDTVRVLVR | | | |
| | LTFVRDGKEQVVDLLQLFKFV | | | |
| 5277 | MTPEEIRANVRAFYAALASGDLEAAKALFDPGVTITLWDG | 2853 | 120.6 | 1.19 |
| | TTFTSVEEFRKWFEEQFKTSKDALREIVEMRIEGDVVRVLV | | | |
| | RLTFVRDGKEQVVDLLQLFKFV | | | |
| 5278 | MTPEAIRENVEAFYAALASGDWEAAKALFDPGVTITLWD | 2854 | 121.2 | 1.61 |
| | GTTFTSVEEFRAWFEEQFQTSKDALREIVSMRIEGDTVRVL | | | |
| | VRLTFTRDGETQVVDLLQLFKFV | | | |
| 5279 | MTPEEIRENVRAFYAALASGDLAAAKALFDPGVTITLWDG | 2855 | 111.8 | 1.12 |
| | TTFTSVEEFRAWFEEQFQTSKDALREIVSLEIEGDVVRVLVR | | | |
| | LTFTRNGETQVVDLLQLFKFV | | | |
| 5280 | MTPEEIRANVRAFYAALASGDWEAARALFDPGVTITLWD | 2856 | 129.2 | 1.07 |
| | GTTFTSVEEFRAWFEKQFQTSKDALREIVALEVEGDVVRVL | | | |
| | VRLTFVRDGVEQEVDLLQLFKFV | | | |
| 5281 | MTPEEIRANVEAFYAALASGDLEAAKSLFDPGVTITLWDGT | 2857 | 125.6 | 3.02 |
| | TFTSVEEFRKWFEEQFKTSKDALREIVSMEIEGDTVRVLVRL | | | |
| | TFVRDGKVEEVDLLQLFKFV | | | |
| 5282 | MTPEEIIANVRAFYAALASGDLEAAKALFDPGVTITLWDGT | 2858 | 131.8 | 1.18 |
| | TFTSVEEFRAWFEQQFQTSKDALREIVSIEVEGDVVRVLVR | | | |
| | LTFVRDGEEQVVDLLQLFKFV | | | |
| 5283 | MTPEEIRANVERFYAALASGDLETAKALFDPGVTITLWDGT | 2859 | 141.4 | 1.14 |
| | TFTSVEEFRKWFEEQFETSKDALREIVSMEVEGDTVRVLVR | | | |
| | LTFVRNGEEKVVDLLQLFKFV | | | |
| 5284 | MTPEERFANVRAFYEALASGDLEAAKALFDPGVTITLWDG | 2860 | 186.8 | 5.16 |
| | TTFTSVEEFRAWFEEQFQTSKDALREIVDMEVEGDTVRVL | | | |
| | VRLTFTRNGETQEVDLLQLFKFV | | | |
| 5285 | MTPEEIRANVERFYAALASGDWATAEALFDPGVTITLWDG | 2861 | 188 | 1.26 |
| | TTFTSVAEFRAWFEEQFKTSKDALREIVSLEINGDVVRVLVR | | | |
| | LTFTRDGKEEVVDLLQLFKFV | | | |
| 5286 | MTPEERIANVRAFYAALASGDLEAAKALFDPGVTITLWDG | 2862 | 196 | 33.33 |
| | TTFTSVEEFRAWFEEQFKTSKDALREIESIEVDGDTVRVLVR | | | |
| | LTFVRDGEEKVVDLLQLFKFV | | | |
| 5287 | MTPEAIRENVRAFYAALASGDWAAAKALFDPGVTITLWD | 2863 | 174.8 | 1.1 |
| | GTTFTSVEEFRAWFEKQFKTSKDALREIVSMEVEGDVVRVL | | | |
| | VRLTFVRDGKEQVVDLLQLFKFV | | | |
| 5288 | MTPEAIIANVKAFYAALASGDLAAAKALFDPGVTITLWDGT | 2864 | 170.8 | 1.6 |
| | TFTSVEEFRAWFEEQFKTSKDALREIESMEVEGDTVRVLVR | | | |
| | LTFVRDGKEEVVDLLQLFKFV | | | |
| 5289 | MTPEEIRANVERFYAALASGDLAAAKALFDPGVTITLWDG | 2865 | 173.8 | 1.92 |
| | TTFTSVEEFRAWFEEQFQTSKDALREIVSMEIDGDTVRVLV | | | |
| | RLTFVRDGEEQEVDLLQLFKFV | | | |
| 5290 | MTPEEIIENVRRFYAALASGDLETAKSLFDPGVTITLWDGTT | 2866 | 162.2 | 1.11 |
| | FTSVEEFRAWFEAQFQTSKDALREIVSIEVEGDVVRVLVRLT | | | |
| | FTRDGQTQEVDLLQLFKFV | | | |
| 5291 | MTPEEIRENVRRFYAALASGDLETAKALFDPGVTITLWDGT | 2867 | 170.2 | 1.02 |
| | TFTSVEEFRAWFEEQFQTSKDALREIVSMEIEGDVVRVLVR | | | |
| | LTFVRDGKEEVVDLLQLFKFV | | | |
| 5292 | MTPEEIIENVKNFYAALASGDLEKAKALFDPGVTITLWDGT | 2868 | 168.6 | 6.75 |
| | TFTSVEEFRKWFEEQFKTSKDALREIKSIEVEGDVVRVLVRL | | | |
| | TFVRDGEVQEVDLLQLFKFV | | | |
| 5293 | MTPEEIRANVRRFYAALASGDLATAEALFDPGVTITLWDG | 2869 | 166.2 | 3.78 |
| | TTFTSVAEFRAWFEKQFKTSKDALREIESMEIEGDTVRVLV | | | |
| | RLTFVRDGKEQVVDLLQLFKFV | | | |
| 5294 | MTPEEIRANVRAFYAALASGDLEAAKALFDPGVTITLWDG | 2870 | 175.2 | 1.46 |
| | TTFTSVEEFRAWFEEQFQTSKDALREIESLEVEGDVVRVLV | | | |
| | RLTFTRDGKVEEVDLLQLFKFV | | | |
| 5295 | MTPEEIRANVRAFYAALASGDLAAAKALFDPGVTITLWDG | 2871 | 186.4 | 1.28 |
| | TTFTSVEEFRAWFEEQFETSKDALREIVSMEVEGDTVRVLV | | | |
| | RLTFTRNGEEKVVDLLQLFKFV | | | |
| 5101 | MSEEQIRQNLRRFYEALDSGDAITAASLFDPGVTITLWDGT | 2872 | 229.6 | 0.87 |
| | TFTSVEEFREWFESQFSTSKDALREISSLEVRGDTVEVTVRL | | | |
| | SFTRNGQKHTVDLLQLFKFVGGGGGGNRVVADRVYVNPTG | | | |
| 5102 | MSEEQIRQNLRRFYEALDSGDAITAASLFDPGVTITLWDGT | 2873 | 2169.2 | 2.2 |
| | TFTSVEEFREWFESQFSTSKDALREISSLEVRGDTVEVTVRL | | | |
| | SFTRNGQKHTVDLLQLFKFVGNRVVADRVYVNPTG | | | |

MBIO-5358 and MBIO-5360 through MBIO-5381 (SEQ ID NO:2774-SEQ ID NO:2796, respectively) are computationally generated LgLux. Most mutants exhibited lower baseline activity and similar or a higher activity in the presence of rapamycin as compared to LgLux_4039 as shown in Table 22.

TABLE 22

| | | | | Fold change |
|---|---|---|---|---|
| | | | Baseline Signal | upon SmLux |
| | | | (Normalized by | peptide |
| MBIO | Sequence | SEQ ID NO | expression) | addition |
| LgLux_4039 | MSEEQIRQNLRRFYEALDSGDAITAASLFDPGV | 2773 | 1766.6 | 360.49 |
| | TITLWDGTTFTSVEEFREWFESQFSTSKDALREI | | | |
| | SSLEVRGDTVEVTVRLSFTRNGQKHTVDLLQLF | | | |
| | KFV | | | |
| 5358 | MSEEQIRQDLLRFYEALDSGDAITAASLFNPGV | 2774 | 440.6 | 243.66 |
| | TITLWDGTTFTSVEEFREWFESQFSTSKDALREI | | | |
| | SSLEVRGDTVEVTVRLSFTRNGQKHTVDLLQQ | | | |
| | FKFI | | | |
| 5360 | MTPEELIANVLAFYAALASGDLVAAKALFNPGVT | 2775 | 150.2 | 3.64 |
| | ITLWDGTTFTSVEEFRAWFETQFKTSKDALREIE | | | |
| | SIEVDGDTVRVLVRLTFVRDGEEQVVDLLQLFK | | | |
| | FV | | | |
| 5361 | MTPEELIANVLAFYAALASGDLVAAKALFNSGVT | 2776 | 156.4 | 5.17 |
| | ITLWDGATFTSIEKFRAWFDTQFKTSKDALREIE | | | |
| | SIEVDGDTVRVLVRLTFVSDGEEQVVDLLQLFK | | | |
| | FV | | | |
| 5362 | MTPEERIANVLAFYAALASGDLEAAAALFNPGVT | 2777 | 906.4 | 27.24 |
| | ITLWDGTTFTSVEEFRAWFEEQFKTSKDALREIE | | | |
| | SIEVDGDTVRVLVRLSFTRNGQKHTVDLLQLFK | | | |
| | FV | | | |
| 5363 | MTPEERIANVLAFYAALASGDLEAAKALFNPGVT | 2778 | 339.2 | 32.23 |
| | ITLWDGTTFTSVEEFRAWFEEQFKTSKDALREIE | | | |
| | SIEVDGDTVRVLVRLSFTRNGQKHTVDLLQLFK | | | |
| | FV | | | |
| 5364 | MTPEERIANVRAFYAALASGDLEAAKALFDPGV | 2779 | 376.8 | 172.59 |
| | TITLWDGTTFTSVEEFRAWFEEQFKTSKDALREI | | | |
| | ESIEVDGDTVRVLVRLSFTRNGQKHTVDLLQLF | | | |
| | KFV | | | |
| 5365 | MTPEELRQNLLRFYEALDSGDAVAAASLFNPGV | 2780 | 1452.6 | 359.8 |
| | TITLWDGTTFTSVEEFREWFETQFKTSKDALREI | | | |
| | ESLEVDGDTVRVTVRLTFTRDGEEQVVDLLQLF | | | |
| | KFV | | | |
| 5366 | MTPEELRQNLLRFYEALDSGDAVAAKSLFNPGV | 2781 | 931.2 | 32.61 |
| | TITLWDGTTFTSVEEFREWFETQFKTSKDALREI | | | |
| | ESLEVDGDTVRVTVRLTFTRDGEEQVVDLLQLF | | | |
| | KFV | | | |
| 5367 | MTPEELRQNLLRFYEALDSGDAVAAASLFNSGV | 2782 | 231.6 | 6.08 |
| | TITLWDGATFTSIEKFREWFDTQFKTSKDALREI | | | |
| | ESLEVDGDTVRVTVRLTFTSDGEEQVVDLLQLF | | | |
| | KFV | | | |
| 5368 | MTPEELRQNLLRFYEALDSGDAVAAKSLFNSGV | 2783 | 366.8 | 4.95 |
| | TITLWDGATFTSIEKFREWFDTQFKTSKDALREI | | | |
| | ESLEVDGDTVRVTVRLTFTSDGEEQWVDLLQLF | | | |
| | KFV | | | |
| 5369 | MTPEEIRQNLLRFYEALDSGDAEAAKSLFNPGV | 2784 | 760 | 289.49 |
| | TITLWDGTTFTSVEEFREWFEEQFKTSKDALREI | | | |
| | ESLEVDGDTVRVTVRLTFTRDGEEKVVDLLQLF | | | |
| | KFV | | | |
| 5370 | MTPEEIRQNLRRFYEALDSGDAEAAKSLFDPGV | 2785 | 4567 | 390.25 |
| | TITLWDGTTFTSVEEFREWFEEQFKTSKDALREI | | | |
| | ESLEVDGDTVRVTVRLTFTRDGEEKVVDLLQLF | | | |
| | KFV | | | |
| 5371 | MTPEERIADVLAFYAALASGDLEAAKALFNPGVT | 2786 | 234.6 | 48.58 |
| | ITLWDGTTFTSVEEFRAWFEEQ FKTSKDALREIE | | | |
| | SIEVDGDTVRVLVRLSFTRNGQKHTVDLLQQFK | | | |
| | FI | | | |
| 5372 | MTPEERIADVLAFYAALASGDLEAAKALFNPGVT | 2787 | 210.6 | 20.86 |
| | ITLWDGTTFTSVEEFRAWFEEQFKTSKDALREIE | | | |
| | SIEVDGDTVRVLVRLTFVRDGEEKVVDLLQQFK | | | |
| | FI | | | |
| 5373 | MTPEERIANVLAFYAALASGDLEAAAALFNPGVT | 2788 | 388.2 | 151.01 |
| | ITLWDGTTFTSVEEFRAWFEEQ FKTSKDALREIE | | | |
| | SIEVDGDTVRVLVRLTFVRDGEEKVVDLLQLFK | | | |
| | FV | | | |
| 5374 | MTPEERIANVRAFYAALASGDLEAAAALFDPGV | 2789 | 796 | 240.47 |
| | TITLWDGTTFTSVEEFRAWFEEQFKTSKDALREI | | | |
| | ESIEVDGDTVRVLVRLTFVRDGEEKVVDLLQLF | | | |
| | KFV | | | |
| 5375 | MTPEERIADVRAFYAALASGDLEAAKALFDPGV | 2790 | 207.6 | 98.41 |
| | TITLWDGTTFTSVEEFRAWFEEQFKTSKDALREI | | | |
| | ESIEVDGDTVRVLVRLSFTRNGQKHTVDLLQLF | | | |
| | KFV | | | |
| 5376 | MTPEERIADVRAFYAALASGDLEAAKALFDPGV | 2791 | 409.4 | 245.29 |
| | TITLWDGTTFTSVEEFRAWFEEQFKTSKDALREI | | | |
| | ESIEVDGDTVRVLVRLSFTRNGQKHTVDLLQLF | | | |
| | KEI | | | |
| 5377 | MTPEERIADVRAFYAALASGDLEAAKALFDPGV | 2792 | 324.4 | 205.44 |
| | TITLWDGTTFTSVEEFRAWFEEQFKTSKDALREI | | | |
| | ESIEVDGDTVRVLVRLSFTRNGQKHTVDLLQQF | | | |
| | KFI | | | |
| 5378 | MTPEERIADVRAFYAALASGDLEAAKALFDPGV | 2793 | 197 | 85.83 |
| | TITLWDGTTFTSVEEFRAWFEEQFKTSKDALREI | | | |
| | ESIEVDGDTVRVLVRLTFVRDGEEKVVDLLQQF | | | |
| | KFI | | | |
| 5379 | MTPEERIADVRAFYAALASGDLEAAKALFDPGV | 2794 | 338.4 | 176.25 |
| | TITLWDGTTFTSVEEFRAWFEEQFKTSKDALREI | | | |
| | ESIEVDGDTVRVLVRLTFVRDGEEKVVDLLQLF | | | |
| | KFI | | | |
| 5380 | MTPEERIADVRAFYAALASGDLEAAKALFDPGV | 2795 | 322.6 | 157.38 |
| | TITLWDGTTFTSVEEFRAWFEEQFKTSKDALREI | | | |
| | ESIEVDGDTVRVLVRLTFVRDGEEKVVDLLQLF | | | |
| | KFV | | | |
| 5381 | MTPEERIANVLAFYAALASGDLEAAKALFNPGVT | 2796 | 158.6 | 22.28 |
| | ITLWDGTTFTSVEEFRAWFEEQ FKTSKDALREIE | | | |
| | SIEVDGDTVRVLVRLTFVRDGEEKVVDLLQLFK | | | |
| | FV | | | |

What is claimed is:

1. A protein having luciferase activity, comprising the secondary structure arrangement H1-L1-H2-L2-B1-L3-B2-L4-H3-L5-B3-L6-B4-L7-B5-L8-B6, wherein “H” is a helical domain, “L” is a loop domain, and “B” is a beta strand domain, wherein:
(a) the H1 domain is at least 18 or 19 amino acids in length; residue 9 of the H1 domain is T, S, H, R, C, L, V, A, Q, G, E, K, I, N or M,
or
(a) the B5 domain is at least 11, 12, 13, or 14 amino acids in length and residue 11 of the B5 domain is F, Y, or L; and
(b) the B4 domain is at least 12 amino acids in length and
residue 10 of the B4 domain is F, Y, L, I, K or M; and/or
residue 12 of the B4 domain is F, L, R, D, M, Q or V; and/or
wherein the protein lacks any lysine residues.

2. The protein of claim 1, wherein residue 10 of the B4 domain is F, Y, L, I, K or M and residue 12 of the B4 domain is L, R, D, M, Q or V.

3. The protein of claim 1 or 2, wherein residue 11 of the B5 domain is F or Y.

4. The protein of any one of claims 1-3, wherein residue 10 of the B4 domain is F or L, and residue 12 of the B4 domain is D, F or L.

5. The protein of any one of claims 1-4, wherein the B3 domain is at least 6, 7, 8, 9, or 10 amino acids in length and residue 1 of the B3 domain is W/L/H; optionally wherein:
(i) residue 9 of the H1 domain is D, K, L, N, R, S, T, Q, V, or Y;
(ii) residue 2 of the L2 domain is not H and further optionally wherein the residue 2 of the L2 domain is D, F, K, L, N, R, S, T, Q, V, or Y;
(iii) residue 3 of the B1 domain is not H and further optionally wherein the residue 3 of the B1 domain is D, F, K, L, N, R, S, T, Q, V, or Y;
(iv) residue 2 of the B2 domain is D, F, K, L, N, R, S, T, Q, or Y;
(v) residue 1 of the H3 domain is D, F, K, L, N, S, T, Q, V, or Y
(vi) residue 5 of the B4 domain is not H and further optionally wherein the residue 5 of the B4 domain is D, F, K, L, N, R, S, T, Q, V, or Y;
(vii) residue 9 of the B4 domain is not H and further optionally wherein the residue 9 of the B4 domain is D, F, K, L, N, R, S, T, Q, V, or Y;
(viii) residue 8 of the B5 domain is D, F, K, L, N, R, S, Q, V, or Y;
(ix) residue 9 of the B5 domain is not H and further optionally wherein the residue 9 of the B5 domain is D, F, K, L, N, R, S, T, Q, V, or Y;
(x) residue 10 of the B5 domain is not H and further optionally wherein the residue 10 of the B5 domain is D, F, K, L, N, R, S, T, Q, V, or Y;
(xi) residue 12 of the B5 domain is not H and further optionally wherein the residue 12 of the B5 domain is D, F, K, L, N, R, S, T, Q, V, or Y;
(xii) residue 14 of the B5 domain is D, F, K, L, N, S, T, Q, V, or Y;
(xiii) residue 3 of the B6 domain is D, F, K, L, N, R, S, Q, V, or Y;
(xiv) residue 4 of the B6 domain is D, F, K, L, N, R, S, T, Q, V, or Y;
(xv) residue 8 of the B6 domain is not H and further optionally wherein the residue 8 of the B6 domain is D, F, K, L, N, R, S, T, Q, V, or Y; and/or
(xvi) residue 9 of the B6 domain is D, F, K, L, N, R, S, T, Q, V, or Y.

6. The protein of claim 5, wherein residue 1 of the B3 domain is W/L.

7. The protein of claim 1, wherein:
(i) the H1 domain is at least 18 or 19 amino acids in length; residue 9 of the H1 domain is N, T, S, H, R, C, L, D, V, A, Q, G, E, K, I, N or M;
(ii) residue 1 of the B3 domain is L, W, or H;
(iii) residue 10 of the B4 domain is F, Y, L, I, K or M;
(iv) residue 12 of the B4 domain is F, L, R, D, M, Q or V;
(v) residue 10 of the B5 domain is L;
(vi) residue 3 of B6 domain is D or N;
(vii) residue 8 of B6 domain is Y, F, or L; and/or
(viii) residue 11 of the B5 domain is F or Y; and
optionally wherein:
(ix) residue 2 of the L2 domain is not H and further optionally wherein the residue 2 of the L2 domain is D, F, L, Q, R, S, T, W or Y;
(x) residue 3 of the B1 domain is not H and further optionally wherein the residue 3 of the B1 domain is D, F, L, Q, R, S, T, W or Y;
(xi) residue 5 of the B4 domain is not H and further optionally wherein the residue 5 of the B4 domain is D, F, L, Q, R, S, T, W or Y;
(xii) residue 9 of the B4 domain is not H and further optionally wherein the residue 9 of the B4 domain is D, F, L, Q, R, S, T, W or Y;
(xiii) residue 3 of the B5 domain is not H and further optionally wherein the residue 3 of the B5 domain is D, F, L, Q, R, S, T, W or Y;
(xiv) residue 9 of the B5 domain is not H and further optionally wherein the residue 9 of the B5 domain is D, F, L, Q, R, S, T, W or Y;
(xv) residue 10 of the B5 domain is not H and further optionally wherein the residue 10 of the B5 domain is D, F, L, Q, R, S, T, W or Y;
(xvi) residue 12 of the B5 domain is not H and further optionally wherein the residue 12 of the B5 domain is D, F, L, Q, R, S, T, W or Y; and/or
(xvii) residue 8 of the B6 domain is not H and further optionally wherein the 8 residue of the B6 domain is D, F, L, Q, R, S, T, W or Y; and/or
optionally wherein:
(ix) residue 2 of the H2 domain is A, F, I, K, L, N, R, S, T, Q, V, or Y;
(x) residue 2 of the L2 domain is not H and further optionally wherein the 2 residue of the L2 domain is A, D, F, I, K, L, N, R, S, T, Q, V, or Y;
(xi) residue 3 of the B1 domain is not H and further optionally wherein the 3 residue of the B1 domain is A, D, F, I, K, L, N, R, S, T, Q, V, or Y;
(xii) residue 2 of the B2 domain is A, D, F, I, K, L, N, R, S, T, Q, or Y;
(xiii) residue 1 of the H3 domain is A, D, F, I, K, L, N, S, T, Q, V, or Y;
(xiv) residue 10 of the H3 domain is A, D, F, I, K, L, N, S, T, Q, V, or Y;
(xv) residue 11 of the H3 domain is A, D, F, I, K, N, R, S, T, Q, V, or Y;
(xvi) residue 5 of the B3 domain is A, D, F, I, L, N, R, S, T, Q, V, or Y;
(xvii) residue 5 of the B4 domain is is not H and further optionally wherein the residue 5 of B4 is A, D, F, I, K, L, N, R, S, T, Q, V, or Y;
(xviii) residue 7 of the B4 domain is A, D, F, I, L, N, R, S, T, V, or Y;
(xix) residue 9 of the B4 domain is not H and further optionally wherein the residue 9 of B4 is A, D, F, I, L, K, N, R, S, T, Q, V, or Y;
(xx) residue 8 of the B5 domain is A, D, F, I, L, K, N, R, S, Q, or Y;
(xxi) residue 9 of the B5 domain is not H and further optionally wherein the residue 9 of B5 is A, D, F, I, L, K, N, R, S, Q, V, or Y;
(xxii) residue 12 of the B5 domain is not H and further optionally wherein the residue 12 of B5 is A, D, F, I, L, K, N, R, S, Q, V, or Y;
(xxiii) residue 14 of the B5 domain is A, D, F, I, L, K, N, S, Q, V, or Y;
(xxiv) residue 3 of the B6 domain is A, D, F, I, L, K, N, R, S, Q, V, or Y;
(xxv) residue 4 of the B6 domain is A, D, F, I, L, K, N, R, S, Q, V, or Y; and/or
(xxvi) residue 9 of the B6 domain is A, D, F, L, K, N, R, S, Q, V, or Y.

8. The protein of claim 7, wherein:
(i) residue 9 of the H1 domain is N, T, S, H, R, C, L, D, V, A, Q, G, E, K, I, N or M;
(ii) residue 1 of the B3 domain is L, W, or H;
(iii) residue 10 of the B4 domain is F, Y, L, I, K or M;
(iv) residue 12 of the B4 domain is F, D, Y, L, I, K or M;
(v) residue 10 of the B5 domain is L;
(vi) residue 3 of B6 domain is D or N;
(vii) residue 8 of B6 domain is Y, F, or L; and
(viii) residue 11 of the B5 domain is F or Y.

9. The protein of claim 1, wherein:
(i) residue 7 of the H2 domain is S;
(ii) residue 4 of L2 domain is H;
(iii) residue 10 of B3 domain is R;
(iv) residue 1 of the B3 domain is L, W, or H;
(v) residue 7 of B4 domain is K;
(vi) residue 10 of the B4 domain is F, Y, L, I, K or M;
(vii) residue 12 of the B4 domain is F, D, Y, L, I, K or M;
(viii) residue 3 of B6 domain is D or N;
(ix) residue 8 of B6 domain is Y, F, or L; and
(x) residue 11 of the B5 domain is W, Y or F.

10. A protein having luciferase activity, comprising the secondary structure arrangement H1-L1-H2-L2-B1-L3-B2-L4-H3-L5-B3-L6-B4-L7-B5-L8-B6, wherein “H” is a helical domain, “L” is a loop domain, and “E” is a beta strand domain, wherein:
the H1 domain is at least 18 or 19 amino acids in length; residue 9 of the H1 domain is T, S, H, R, C, L, V, A, Q, G, E, K, I, N or M.

11. The protein of claim 10, wherein the B4 domain is at least 12 amino acids in length and residue 10 of the B4 domain is F, Y, L, I, K or M; and/or
residue 12 of the B4 domain is L, R, D, M, Q, or V.

12. The protein of claim 11, wherein residue 9 of the H1 domain is V, residue 10 of the B4 domain is F, Y, L, I, K or M and residue 12 of the B4 domain is L, R, D, M, Q or V.

13. The protein of claim 11, wherein residue 9 of the H1 domain is V, residue 10 of the B4 domain is F, and residue 12 of the B4 domain is L.

14. The protein of any one of claims 10-13, wherein the B3 domain is at least 6, 7, 8, 9, or 10 amino acids in length and residue 1 of the B3 domain is W or H.

15. The protein of claim 14, wherein residue 1 of the B3 domain is W.

16. The protein of claim 10, wherein:
(i) residue 19 of the H1 domain is R;
(ii) residue 4 of L2 is Q, P, or H;
(iii) residue 11 of H3 is R or M;
(iv) residue 1 of the B3 domain is N, H, K, S, L, Q or Y;
(v) residue 10 of the B4 domain is F, Y, L, V, I, K or M;
(vi) residue 10 of the B5 domain is L;
(vii) residue 3 of B6 domain is D or N; and/or
(viii) residue 8 of B6 domain is Y, F, or L.

17. The protein of claim 16, wherein:
(i) residue 19 of the H1 domain is R;
(ii) residue 4 of L2 is Q;
(iii) residue 11 of H3 is R;
(iv) residue 1 of the B3 domain is N;
(v) residue 10 of the B4 domain is F;
(vi) residue 10 of the B5 domain is L;
(vii) residue 3 of B6 domain is N; and/or
(viii) residue 8 of B6 domain is Y.

18. The protein of claim 16, wherein:
(i) residue 19 of the H1 domain is R;
(ii) residue 4 of L2 is Q;
(iii) residue 11 of H3 is R;
(iv) residue 1 of the B3 domain is N;
(v) residue 10 of the B4 domain is F;
(vi) residue 10 of the B5 domain is L;
(vii) residue 3 of B6 domain is N; and
(viii) residue 8 of B6 domain is Y.

19. The protein of any one of claims 16-18, wherein residue 11 of the B5 domain is F or Y.

20. The protein of claim 19, wherein residue 11 of the B5 domain is F.

21. The protein of any one of claims 16-20, wherein residue 9 of the H1 domain is E.

22. The protein of claim 10, wherein:
(i) residue 9 of the H1 domain is A;
(ii) residue 19 of the H1 domain is R;
(iii) residue 1 of the B3 domain is N, H, K, S, L, Q or Y;
(iv) residue 10 of the B4 domain is F, Y, L, V, I, K or M;
(v) residue 12 of the B4 domain is L, R, D, M, Q, or V;
(vi) residue 3 of B6 domain is D or N; and/or
(vii) residue 8 of B6 domain is Y, F, or L.

23. The protein of claim 10, wherein:
(i) residue 9 of the H1 domain is A;
(ii) residue 19 of the H1 domain is R;
(iii) residue 1 of the B3 domain is N;
(iv) residue 10 of the B4 domain is Y;
(v) residue 12 of the B4 domain is R;
(vi) residue 3 of B6 domain is D or N; and/or
(vii) residue 8 of B6 domain is Y.

24. The protein of claim 22, wherein:
(i) residue 9 of the H1 domain is A;
(ii) residue 19 of the H1 domain is R;
(iii) residue 1 of the B3 domain is N;
(iv) residue 10 of the B4 domain is Y;
(v) residue 12 of the B4 domain is R;
(vi) residue 3 of B6 domain is D or N; and
(vii) residue 8 of B6 domain is Y.

25. The protein of claim 10, wherein:
(i) residue 18 of H1 domain is E;
(ii) residue 1 of the B3 domain is N, H, K, S, L, Q or Y;
(iii) residue 10 of the B4 domain is F, Y, L, V, I, K or M;
(iv) residue 12 of the B4 domain is L, R, D, M, Q, or V;
(v) residue 3 of B6 domain is D or N; and/or
(vi) residue 8 of B6 domain is Y, F, or L.

26. The protein of claim 25, wherein:
(i) residue 18 of H1 domain is E;
(ii) residue 1 of the B3 domain is H;
(iii) residue 10 of the B4 domain is Y;
(iv) residue 12 of the B4 domain is R;
(v) residue 3 of B6 domain is D; and/or
(vi) residue 8 of B6 domain is F.

27. The protein of claim 25, wherein:
(i) residue 18 of H1 domain is E;
(ii) residue 1 of the B3 domain is H;
(iii) residue 10 of the B4 domain is Y;
(iv) residue 12 of the B4 domain is R;
(v) residue 3 of B6 domain is D; and
(vi) residue 8 of B6 domain is F.

28. The protein of any one of claims 25-27, wherein residue 9 of the H1 domain is K.

29. The protein of claim 10, wherein:
(i) residue 1 of the B3 domain is N, H, K, S, L, Q or Y;
(ii) residue 10 of the B4 domain is F, Y, L, V, I, K or M; and/or
(iii) residue 12 of the B4 domain is L, R, D, M, Q, or V.

30. The protein of claim 29, wherein:
(i) residue 1 of the B3 domain is K or Y;
(ii) residue 10 of the B4 domain is F; and/or
(iii) residue 12 of the B4 domain is V or R.

31. The protein of claim 29, wherein:
(i) residue 1 of the B3 domain is K;
(ii) residue 10 of the B4 domain is F; and
(iii) residue 12 of the B4 domain is V
or
(i) residue 1 of the B3 domain is Y;
(ii) residue 10 of the B4 domain is F; and
(iii) residue 12 of the B4 domain is R.

32. The protein of any one of claims 29-31, wherein residue 9 of the H1 domain is S or L.

33. The protein of claim 10, wherein:
(i) residue 1 of the B3 domain is N, H, K, S, L, Q or Y;
(ii) residue 10 of the B4 domain is F, Y, L, V, I, K or M;
(iii) residue 12 of the B4 domain is L, R, D, M, Q, or V; and/or
(iv) residue 8 of B6 domain is Y, F, or L.

34. The protein of claim 33, wherein residue 3 of B6 domain is D or N.

35. The protein of claim 33 or 34, wherein
(i) residue 1 of the B3 domain is Y;
(ii) residue 10 of the B4 domain is V;
(iii) residue 12 of the B4 domain is V;
(iv) residue 8 of B6 domain is Y, F, or L; and
(v) residue 3 of B6 domain is D.

36. The protein of claim 33 or 34, wherein
(i) residue 1 of the B3 domain is L;
(ii) residue 10 of the B4 domain is F;
(iii) residue 12 of the B4 domain is F;
(iv) residue 8 of B6 domain is Y, F, or L;
(v) residue 3 of B6 domain is D; and
(vi) residue 10 of the B5 domain is L.

37. The protein of claim 33, wherein:
(i) residue 11 of H3 is R or M;
(ii) residue 1 of the B3 domain is N, H, K, S, L, Q or Y;
(iii) residue 10 of the B4 domain is F, Y, L, V, I, K or M;
(iv) residue 12 of the B4 domain is L, R, D, M, Q, or V; and/or
(v) residue 8 of B6 domain is Y, F, or L.

38. The protein of claim 37, wherein:
(i) residue 11 of H3 is M;
(ii) residue 1 of the B3 domain is S;
(iii) residue 10 of the B4 domain is L;
(iv) residue 12 of the B4 domain is R; and
(v) residue 8 of B6 domain is Y.

39. The protein of any one of claims 33-38, wherein residue 9 of the H1 domain is G, N, or I.

40. The protein of claim 10, wherein:
(i) residue 19 of the H1 domain is R;
(ii) residue 4 of L2 is Q, P, or H;
(iii) residue 11 of H3 is R or M;
(iv) residue 1 of the B3 domain is N, H, K, S, L, Q or Y;
(v) residue 10 of the B4 domain is F, Y, L, V, I, K or M; and/or
(vi) residue 8 of B6 domain is Y, F, or L.

41. The protein of claim 40, wherein:
(vii) residue 10 of the B5 domain is L; and
(viii) residue 3 of B6 domain is D or N.

42. The protein of claim 40 or 41, wherein residue 9 of the H1 domain is E or I.

43. The protein of claim 10, wherein:
(i) residue 10 of B3 domain is R;
(ii) residue 2 of L5 is Q;
(iii) residue 3 of B6 domain is D or N; and/or
(iv) residue 8 of B6 domain is Y, F, or L.

44. The protein of claim 43, wherein residue 7 of B4 domain is K.

45. The protein of claim 43 or 44, wherein:
i) residue 10 of B3 domain is R;
(ii) residue 2 of L5 is Q;
(iii) residue 3 of B6 domain is D or N;
(iv) residue 8 of B6 domain is Y, F, or L; and
(v) residue 7 of B4 domain is K.

46. The protein of claim 43, wherein the B4 domain is at least 12 amino acids in length and residue 10 of the B4 domain is F, Y, L, I, K or M; and/or
residue 12 of the B4 domain is F, L, R, D, M, Q or V.

47. The protein of claim 46, wherein:
i) residue 10 of B3 domain is R;
(ii) residue 2 of L5 is Q;
(iii) residue 3 of B6 domain is D or N;
(iv) residue 8 of B6 domain is Y, F, or L;
(v) residue 10 of the B4 domain is F, Y, L, I, K or M; and
(vi) residue 12 of the B4 domain is F, L, R, D, M, Q or V.

48. The protein of any one of claims 43-47, wherein residue 9 of the H1 domain is T or Y.

49. A protein having luciferase activity, comprising the secondary structure arrangement H1-L1-H2-L2-B1-L3-B2-L4-H3-L5-B3-L6-B4-L7-B5-L8-B6, wherein “H” is a helical domain, “L” is a loop domain, and “B” is a beta strand domain, wherein:
the B3 domain is at least 6, 7, 8, 9, or 10 amino acids in length and residue 1 of the B3 domain is W or H.

50. The protein of claim 49, wherein residue 1 of the B3 domain is W.

51. A protein having luciferase activity, comprising the secondary structure arrangement H1-L1-H2-L2-B1-L3-B2-L4-H3-L5-B3-L6-B4-L7-B5-L8-B6, wherein “H” is a helical domain, “L” is a loop domain, and “B” is a beta strand domain, wherein:
the B4 domain is at least 12 amino acids in length and residue 10 of the B4 domain is F, Y, L, I, K or M.

52. The protein of claim 51, wherein residue 10 of the B4 domain is F.

53. A protein having luciferase activity, comprising the secondary structure arrangement H1-L1-H2-L2-B1-L3-B2-L4-H3-L5-B3-L6-B4-L7-B5-L8-B6, wherein “H” is a helical domain, “L” is a loop domain, and “B” is a beta strand domain, wherein:
the B4 domain is at least 12 amino acids in length and residue 12 of the B4 domain is L, R, D, M, Q or V.

54. The protein of claim 53, wherein residue 12 of the B4 domain is L.

55. The protein of any one of claim 1-54, wherein
the H1 domain is at least 18 or 19 amino acids in length; residue 14 of the H1 domain is Y, D, or E, and residue 9 of the H1 domain is D or E;
the B3 domain is at least 6, 7, 8, 9, or 10 amino acids in length and residue 2 of the B3 domain is R; and
the B5 domain is at least 11, 12, 13, or 14 amino acids in length and residue 9 of the B5 domain is H or N.

56. The protein of claim 55, wherein residue 7 of the B5 domain is L.

57. The protein of claim 55 or 56 wherein the B6 domain is at least 9, 10, 11, 12, or 13 amino acids in length and wherein residue 5 of the B6 domain is V.

58. The protein of any one of claims 55-57, wherein residue 1 of the L5 domain is S.

59. The protein of any one of claims 55-58, wherein residue 7 of the B5 domain is L and residue 5 of the B6 domain is V.

60. The protein of any one of claims 55-59, wherein residue 7 of the B5 domain is L, residue 5 of the B6 domain is V, and residue 1 of the L5 domain is S.

61. The protein of any one of claims 53-60, wherein the H2 domain is at least 5, 6, or 7 amino acids in length, the H3 domain is at least 9, 10, 11, 12, 13, or 14 amino acids in length, the B1 domain is at least 3 or 4 amino acids in length, the B2 domain is at least 3 or 4 amino acids in length, and/or the B4 domain is at least 12 amino acids in length.

62. The protein of any one of claims 55-61, wherein:
the H1 domain is at least or up to 19 amino acids in length;
the H2 domain is at least or up to 7 amino acids in length;
the B1 domain is at least or up to 4 amino acids in length;
the B2 domain is at least or up to 4 amino acids in length;
the H3 domain is at least or up to 14 amino acids in length;
the B3 domain is at least or up to 10 amino acids in length;
the B4 domain is at least or up to 12 amino acids in length;
the B5 domain is at least or up to 14 amino acids in length; and
the B6 domain is at least or up to 12 or 13 amino acids in length.

63. The protein of any one of claims 55-62, wherein:
residue 13 of domain H1 is F;
residue 1 of domain L3 is W;
residue 5 of domain B5 is V or another hydrophobic residue; and/or
residue 8 of domain B5 is A or L or another hydrophobic residue.

64. The protein of any one of claims 62 or 63, wherein:
residue 2 of domain B1 is I or another hydrophobic residue;
residue 4 of domain H3 is F;
residue 6 of domain B4 is V or another hydrophobic residue;
residue 8 of domain B4 is L or another hydrophobic residue;
residue 5 of domain B6 is M or V or another hydrophobic residue; and/or
residue 7 of domain B6 is V or another hydrophobic residue.

65. The protein of any one of claims 1-64, wherein:
the H1 domain comprises an amino acid sequence having at least 50%, 55%, 60%, 65%, 70%, 75%, 80%, 85%, 90%, 91%, 92%, 93%, 94%, 95%, 96%, 97%, 98%, 99%, or 100% identity to the amino acid sequence: MSEEQIRQFLRRFYEALDS (SEQ ID NO:2738) or SISEEQIRQFLRRFYEALDS (SEQ ID NO:2739) or IPEEQIRQFLRRFYEALDS (SEQ ID NO:2740) or EISEEQIRQFLRRFYEALDS (SEQ ID NO:2741).

66. The protein of any one of claims 1-65, wherein:
the H2 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: ADTAASL.

67. The protein of any one of claims 1-66, wherein:
the B1 domain comprises an amino acid sequence having at least 50%, 75%, or 100% identity to the amino acid sequence: TIHL.

68. The protein of any one of claims 1-67, wherein:
the B2 domain comprises an amino acid sequence having at least 50%, 75%, or 100% identity to the amino acid sequence: GVTF.

69. The protein of any one of claims 1-68, wherein:
the H3 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence:
REEFREWFERLFST.

70. The protein of any one of claims 1-69, wherein:
the B3 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence:
WREIKSLEVR.

71. The protein of any one of claims 1-70, wherein:
the B4 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence:
TVEVHVQLHFTL
or
TVVVVVRLDFTL.

72. The protein of any one of claims 1-71, wherein:
the B5 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: QKHTVDLTHHFHFR or QKHTVILTHVFRFR.

73. The protein of any one of claims 1-72, wherein:
the B6 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: RVTEVRVHINPTG or RVTEVRVEIVPV.

74. The protein of any one of claims 1-73, wherein the L1, L2, L3, L4, L5, L6, L7, and L8 domains are at least 1, 2, 3, 4, or 5 amino acids in length and comprise any amino acid and optionally are up to 5 amino acids in length.

75. The protein of any one of claims 1-74, wherein the protein comprises an amino acid sequence having at least 50%, 60%, 70%, 75%, 80%, 85%, 90%, 91%, 92%, 93%, 94%, 95%, 96%, 97%, 98%, or 99% identity to the amino acid sequence:
(SEQ ID NO: 1)
MSEEQIRQFLRRFYEALDSGDADTAASLFHPGVTIHLWDGVTFTSREEFR
EWFERLFSTSKDAQREIKSLEVRGDTVEVHVQLHATHNGQKHTVDLTHHW
HFRGNRVTEVRVHINPTG.

76. A protein having luciferase activity, comprising an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:1,
wherein:
(i) a) residue 100 is F, Y, or L; and
b) residue 85 is F, Y, L, I, K or M; and
c) residue 87 is L, R, D, M, Q, F or V; or
(ii) a) residue 100 is F, Y, or L; and
b) residue 87 is L, R, D, M, Q, F or V; or
(iii) a) residue 100 is F, Y, or L; and
b) residue 85 is F, Y, L, I, K or M; or
(iv) wherein the protein does not include lysine residues and includes another amino acid instead of the lysine residue present in SEQ ID NO:1 and optionally the protein comprises the residues as set forth in any one of (i)-(iii).

77. The protein of claim 76, comprising the substitution W100F relative to SEQ ID NO:1.

78. The protein of claim 76 or 77, comprising the substitution A85F relative to SEQ ID NO:1.

79. The protein of any one of claims 76-78, comprising the substitution H87L relative to SEQ ID NO:1.

80. The protein of any one of claims 76-79, further comprising the substitution Q64W, Q64L or Q64H, relative to SEQ ID NO:1.

81. The protein of any one of claims 1-80, further comprising an additional polypeptide domain fused to the protein.

82. The protein of claim 81, wherein the additional polypeptide domain is present at the N-terminus or the C-terminus of the protein.

83. A self-complementing multipartite protein having luciferase activity, comprising at least a first polypeptide component and a second polypeptide component, wherein the at least first polypeptide component and the second polypeptide component are not covalently linked or are covalently linked via a cleavable linker, wherein in total the first polypeptide component and the second polypeptide component comprise the secondary structure arrangement H1-L1-H2-L2-B1-L3-B2-L4-H3-L5-B3-L6-B4-L7-B5-L8-B6, wherein each domain is as defined in any one of claims 1-74;
wherein (a) each H and B domain is fully present within one polypeptide component of either the first polypeptide component or the second polypeptide component, (b) the first polypeptide component and the second polypeptide component do not include all of the H and B domains, (c) the relative order of the H, L, and B domains when present in either of the first polypeptide component or the second polypeptide component is unchanged with reference to the protein as defined in any one of claims 1-74, and (d) the first component and the second component when not present in the self-complementing multipartite protein do not possess detectable luciferase activity or have luciferase activity lower than the luciferase activity of the self-complementing multipartite protein.

84. The self-complementing multipartite protein of claim 83, wherein the first polypeptide component and the second polypeptide component comprise the secondary structure arrangement as set forth in Table 1:
TABLE 1

| first polypeptide component | second polypeptide component |
|---|---|
| H1-(L1) | (L1)-H2-L2-B1-L3-B2-L4-H3-L5-B3-L6-B4-L7-B5- |
| | L8-B6 |
| H1-L1-H2-(L2) | (L2)-B1-L3-B2-L4-H3-L5-B3-L6-B4-L7-B5-L8-B6 |
| H1-L1-H2-L2-B1-(L3) | (L3)-B2-L4-H3-L5-B3-L6-B4-L7-B5-L8-B6 |
| H1-L1-H2-L2-B1-L3-B2-(L4) | (L4)-H3-L5-B3-L6-B4-L7-B5-L8-B6 |
| H1-L1-H2-L2-B1-L3-B2-L4-H3-(L5) | (L5)-B3-L6-B4-L7-B5-L8-B6 |
| H1-L1-H2-L2-B1-L3-B2-L4-H3-L5-B3-(L6) | (L6)-B4-L7-B5-L8-B6 |
| H1-L1-H2-L2-B1-L3-B2-L4-H3-L5-B3-L6-B4-(L7) | (L7)-B5-L8-B6 |
| H1-L1-H2-L2-B1-L3-B2-L4-H3-L5-B3-L6-B4-L7- | (L8)-B6 |
| B5-(L8) | |
| (L1)-H2-L2-B1-L3-B2-L4-H3-L5-B3-L6-B4-L7-B5- | H1-(L1) |
| L8-B6 | |
| (L2)-B1-L3-B2-L4-H3-L5-B3-L6-B4-L7-B5-L8-B6 | H1-L1-H2-(L2) |
| (L3)-B2-L4-H3-L5-B3-L6-B4-L7-B5-L8-B6 | H1-L1-H2-L2-B1-(L3) |
| (L4)-H3-L5-B3-L6-B4-L7-B5-L8-B6 | H1-L1-H2-L2-B1-L3-B2-(L4) |
| (L5)-B3-L6-B4-L7-B5-L8-B6 | H1-L1-H2-L2-B1-L3-B2-L4-H3-(L5) |
| (L6)-B4-L7-B5-L8-B6 | H1-L1-H2-L2-B1-L3-B2-L4-H3-L5-B3-(L6) |
| (L7)-B5-L8-B6 | H1-L1-H2-L2-B1-L3-B2-L4-H3-L5-B3-L6-B4-(L7) |
| (L8)-B6 | H1-L1-H2-L2-B1-L3-B2-L4-H3-L5-B3-L6-B4-L7- |
| | B5-(L8) |

wherein the L domain in parenthesis is (i) present in one but not both of the first and second components, (ii) is split between the first and second components, or (iii) absent.

85. The self-complementing multipartite protein of claim 83 or 84, wherein (i) one or both of the first component and the second component comprises an additional domain, (ii) one or both of the first component and the second component comprises an additional domain covalently linked to one or both of the first component and the second component, (iii) the first component is a fusion protein comprising a first domain and the second component is a fusion protein comprising a second domain, or (iv) the self-complementing multipartite protein comprises from N-terminus to C-terminus, (a) the first component, a linker, and the second component or (b) the second component, a linker, and the first component, wherein the self-complementing multipartite protein has luciferase activity and wherein upon cleavage of the linker, the self-complementing multipartite protein has substantially reduced cleavage activity or substantially undeteactable cleavage activity.

86. A circularly permuted polypeptide having luciferase activity, wherein:
the N-terminus and the C-terminus of the circularly permuted polypeptide are different from the N-terminus and C-terminus, respectively, of a protein having luciferase activity and comprising the secondary structure arrangement H1-L1-H2-L2-B1-L3-B2-L4-H3-L5-B3-L6-B4-L7-B5-L8-B6, “H” is a helical domain, “L” is a loop domain, and “B” is a beta strand domain, and
in the circularly permuted polypeptide, the N-terminus and C-terminus of the protein having luciferase activity are joined by a linker sequence and the circularly permuted polypeptide comprises the secondary structure arrangement:
H2-L2-B1-L3-B2-L4-H3-L5-B3-L6-B4-L7-B5-L8-B6-LINKER-H1-(L1) (I),
B1-L3-B2-L4-H3-L5-B3-L6-B4-L7-B5-L8-B6-LINKER-H1-L1-H2-(L2) (II),
B2-L4-H3-L5-B3-L6-B4-L7-B5-L8-B6-LINKER-H1-L1-H2-L2-B1-(L3) (III),
H3-L5-B3-L6-B4-L7-B5-L8-B6-LINKER-H1-L1-H2-L2-B1-L3-B2-(L4) (IV),
B3-L6-B4-L7-B5-L8-B6-LINKER-H1-L1-H2-L2-B1-L3-B2-L4-H3-(L5) (V),
B4-L7-B5-L8-B6-LINKER-H1-L1-H2-L2-B1-L3-B2-L4-H3-L5-B3-(L6) (VI),
B5-L8-B6-LINKER-H1-L1-H2-L2-B1-L3-B2-L4-H3-L5-B3-L6-B4-(L7) (VII), or
B6-LINKER-H1-L1-H2-L2-B1-L3-B2-L4-H3-L5-B3-L6-B4-L7-B5-(L8) (VIII),
wherein the L domain in parenthesis is present at the C-terminus, or the N-terminus, or is split between the C-terminus and the N-terminus or is absent.

87. The circularly permuted polypeptide of claim 86, wherein the linker comprises the secondary structure H4-L9.

88. The circularly permuted polypeptide of claim 86, wherein the linker comprises the secondary structure H4-L9-H5-L10.

89. The circularly permuted polypeptide of claim 86, wherein the linker comprises the secondary structure H4-L9-H5-L10-H6-L11.

90. The circularly permuted polypeptide of any one of claims 86-89, wherein the H1, L1, H2, L2, B1, L3, B2, L4, H3, L5, B3, L6, B4, L7, B5, L8, and B6 are as set forth in any one of claims 1-74.

91. The circularly permuted polypeptide of any one of claims 86-87, wherein the B5 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: QKHTVDLTHHWHFR or QKHTVILTHVFRFR.

92. The circularly permuted polypeptide of any one of claims 86-91, wherein the B4 domain comprises an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence: TVEVHVQLHATH or TVVVVVRLDFTL.

93. The circularly permuted polypeptide of any one of claims 86-92, comprising an amino acid sequence having at least 50%, 60%, 70%, 75%, 80%, 85%, 90%, 91%, 92%, 93%, 94%, 95%, 96%, 97%, 98%, 99%, or 100% identity to the amino acid sequence set forth in any one of SEQ ID NOs: 144-2599.

94. A fusion protein comprising the circularly permuted polypeptide of any one of claims 86-93 fused to an additional functional domain.

95. A self-complementing multipartite protein having luciferase activity, comprising at least a first polypeptide component and a second polypeptide component, wherein the at least first polypeptide component and the second polypeptide component are not covalently linked or are covalently linked via a linker, optionally a cleavable linker, wherein in total the first polypeptide component and the second polypeptide component comprise the secondary structure arrangement of a circularly permuted polypeptide as set forth in any one of claims 86-94,
wherein (a) each H and B domain is fully present within one polypeptide component of either the first polypeptide component or the second polypeptide component, (b) the first polypeptide component and the second polypeptide component individually do not include all of the H and B domains, (c) the relative order of the H, L, and B domains when present in either of the first polypeptide component or the second polypeptide component is unchanged relative to the circularly permuted polypeptide as set forth in any one of claims 86-94, and (d) the first component and the second component individually do not possess detectable luciferase activity or individually have luciferase activity lower than the luciferase activity of the self-complementing multipartite protein.

96. The self-complementing multipartite protein of claim 95, wherein the H and B domains of the circularly permuted polypeptide are separated into the first polypeptide component and the second polypeptide component at an L domain, wherein the point of separation is the N-terminus of the L domain, the C-terminus of the L domain or in the L domain.

97. The self-complementing multipartite protein of claim 96, wherein the H and B domains of the circularly permuted polypeptide (1) are separated into the first polypeptide component and the second polypeptide component at the L2 domain, the L3 domain, the L4 domain, the L5 domain, the L6 domain, the L7 domain, the L8 domain, or the Linker.

98. The self-complementing multipartite protein of claim 96, wherein the H and B domains of the circularly permuted polypeptide (II) are separated into the first polypeptide component and the second polypeptide component at the L3 domain, the L4 domain, the L5 domain, the L6 domain, the L7 domain, the L8 domain, the Linker or the L1 domain.

99. The self-complementing multipartite protein of claim 96, wherein the H and B domains of the circularly permuted polypeptide (Ill) are separated into the first polypeptide component and the second polypeptide component at the L4 domain, the L5 domain, the L6 domain, the L7 domain, the L8 domain, the Linker, the L1 domain, or the L2 domain.

100. The self-complementing multipartite protein of claim 96, wherein the H and B domains of the circularly permuted polypeptide (IV) are separated into the first polypeptide component and the second polypeptide component at the L5 domain, the L6 domain, the L7 domain, the L8 domain, the Linker, the L1 domain, the L2 domain, or the L3 domain.

101. The self-complementing multipartite protein of claim 96, wherein the H and B domains of the circularly permuted polypeptide (V) are separated into the first polypeptide component and the second polypeptide component at the L6 domain, the L7 domain, the L8 domain, the Linker, the L1 domain, the L2 domain, the L3 domain, or the L4 domain.

102. The self-complementing multipartite protein of claim 96, wherein the H and B domains of the circularly permuted polypeptide (VI) are separated into the first polypeptide component and the second polypeptide component at the L7 domain, the L8 domain, the Linker, the L1 domain, the L2 domain, the L3 domain, the L4 domain, or the L5 domain.

103. The self-complementing multipartite protein of claim 96, wherein the H and B domains of the circularly permuted polypeptide (VII) are separated into the first polypeptide component and the second polypeptide component at the L8 domain, the Linker, the L1 domain, the L2 domain, the L3 domain, the L4 domain, the L5 domain, or the L6 domain.

104. The self-complementing multipartite protein of claim 96, wherein the H and B domains of the circularly permuted polypeptide (Vill) are separated into the first polypeptide component and the second polypeptide component at the Linker, the L1 domain, the L2 domain, the L3 domain, the L4 domain, the L5 domain, the L6 domain, or the L7 domain.

105. The self-complementing multipartite protein of any one of claims 95-104, wherein one or both of the first polypeptide component and the second polypeptide component is fused to an additional functional domain, optionally wherein the additional domain is covalently linked to one or both of the first component and the second component, optionally wherein the first component is a fusion protein comprising a first domain and the second component is a fusion protein comprising a second domain.

106. A self-complementing multipartite protein having luciferase activity, comprising at least a first polypeptide component, a second polypeptide component, and a third polypeptide component, wherein the at least first polypeptide component, the second polypeptide component, and the third polypeptide component are not covalently linked or are covalently linked via one or two linkers, optionally, one or two cleavable linkers, wherein in total the first polypeptide component, the second polypeptide component, and the third polypeptide component comprise the secondary structure arrangement H1-L1-H2-L2-B1-L3-B2-L4-H3-L5-B3-L6-B4-L7-B5-L8-B6, wherein each domain is as defined in any one of claims 1-74;
wherein (a) each H and B domain is fully present within one polypeptide component of the first polypeptide component, the second polypeptide component, or the third polypeptide component, (b) the first polypeptide component, the second polypeptide component, and the third polypeptide component do not include all of the H and B domains, (c) the relative order of the H, L, and B domains when present in the first polypeptide component, the second polypeptide component, or the third polypeptide component is unchanged with reference to the protein as defined in any one of claims 1-74, and (d) the first component, the second polypeptide component, and the third polypeptide component when not present in the self-complementing multipartite protein do not possess detectable luciferase activity or have luciferase activity lower than the luciferase activity of the self-complementing multipartite protein.

107. The self-complementing multipartite protein of claim 106, wherein the H and B domains of the protein as set forth in any one of claims 1-74 are separated into the first polypeptide component, the second polypeptide component, and the third polypeptide component at an L domain, wherein the point of separation is the N-terminus of the L domain, the C-terminus of the L domain or in the L domain, and optionally the L domain is absent from the first polypeptide component, the second polypeptide component, and the third polypeptide component.

108. The self-complementing multipartite protein of claim 106 or 107, wherein at least one of the first component, the second component, and the third component comprises an additional domain, optionally wherein the additional domain is covalently linked to at least one of the first component, the second component, and the third component, optionally wherein the first component is a fusion protein comprising a first domain, the second component is a fusion protein comprising a second domain, and the third component is a fusion protein comprising a third domain.

109. A self-complementing multipartite protein having luciferase activity, comprising at least a first polypeptide component, a second polypeptide component, and a third component wherein the at least first polypeptide component, the second polypeptide component, and the third component are not covalently linked or are covalently linked via one or two linkers, optionally, one or two cleavable linkers, wherein in total the first polypeptide component, the second polypeptide component, and the third component comprise the secondary structure arrangement of a circularly permuted polypeptide as set forth in any one of claims 86-94,
wherein (a) each H and B domain is fully present within one polypeptide component of the first polypeptide component, the second polypeptide component, or the third component, (b) the first polypeptide component, the second polypeptide component, and the third component individually do not include all of the H and B domains, (c) the relative order of the H, L, and B domains when present in the first polypeptide component, the second polypeptide component, or the third component is unchanged relative to the circularly permuted polypeptide as set forth in any one of claims 86-94, and (d) the first component, the second component, and the third component individually do not possess detectable luciferase activity or individually have luciferase activity lower than the luciferase activity of the self-complementing multipartite protein.

110. The self-complementing multipartite protein of claim 109, wherein the H and B domains of the circularly permuted polypeptide are separated into the first polypeptide component, the second polypeptide component, and the third component at an L domain, wherein the point of separation is the N-terminus of the L domain, the C-terminus of the L domain or in the L domain.

111. The self-complementing multipartite protein of claim 109 or 110, wherein one or more of the first polypeptide component, the second polypeptide component, and the third component is fused to an additional functional domain, optionally wherein the additional domain is covalently linked to one or more of the first component, the second polypeptide component, and the third component, optionally wherein the first component is a fusion protein comprising a first domain, the second component is a fusion protein comprising a second domain, and the third component is a fusion protein comprising a third domain.

112. A protein having luciferase activity and comprising an amino acid sequence having at least 30%, 40%, 50%, 60%, 70%, 75%, 80%, 85%, 90%, 91%, 92%, 93%, 94%, 95%, 96%, 97%, 98%, or 99% identity to the amino acid sequence of SEQ ID NO:1 and
(i) comprising an amino acid substitution at one or more of the following positions relative to SEQ ID NO:1:
E3, I6, Y14, E15, S19, L28, G32, T42, F43, S45, L56, F57, T59, K61, Q64, V77, E78 Q82, A85, T86, H92, L96, H99, W100, R106, T108, and H113; and/or
(ii) comprising another amino acid instead of a lysine residue present in SEQ ID NO:1.

113. The protein of claim 112, comprising:
(i) one or more of the substitutions E3D, I6T/K, Y14W, E15G, S19R, L28S/F, G32R/D/A/E/H, T421, F43L/G, S45A, L56R/K/Q, L56R/K/Q, F57V, T59K, Q64W/H, K61P/E, V77Y, E78W, Q82T/K, A85F/Y/L/l/M, T86A, H87L/V, H99L, W100F/Y/L, R106L, V1071, T108N/D, and H113F; or
(ii) lacking lysine residues and comprising an amino acid substitution at one or more of the following positions relative to SEQ ID NO:1:
F9, D23, H30, H36, V41, R46, R55, L56, Q64, K68, H80, Q82, H84, A85, H87, H92, T97, H98, H99, W100, H101, R103, T108, E109, H113, and I114, optionally wherein the lysine residues are replaced with arginine, further optionally, the amino acid sequence comprises all of the following substitutions:
F9V/S/N, D231, H30D, H36T, V41T, R46V, R55S, L56Q, Q64L, K68S, H80T, Q82R, H84S, A85F, H87R, H92S, T97L, H98Q, H99L, W100F, H101K/R, R103V, T108D/V, E109A, H113Y, and I114V.

114. A nucleic acid comprising a nucleotide sequence encoding the protein, polypeptide component, or fusion protein of any preceding claim.

115. An expression vector comprising the nucleic acid of claim 114 operatively linked to an expression control element.

116. A recombinant host cell comprising the protein, polypeptide component, fusion protein, nucleic acid, and/or expression vector of any preceding claim.

117. A protein having luciferase activity and comprising an amino acid sequence having at least 50%, 60%, 70%, 75%, 80%, 85%, 90%, 91%, 92%, 93%, 94%, 95%, 96%, 97%, 98%, 99% identity, or 100% identity to the amino acid sequence of any one of SEQ ID NOs: 2-91, 93-143, 2601-2604, 2663, 2665, 2682-2732.

118. A protein having luciferase activity and comprising an amino acid sequence at least 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:1, wherein the amino acid at position 9 is any amino acid other than F, wherein the position 9 is numbered based on SEQ ID NO:1.

119. The protein of claim 118, wherein the amino acid at position 9 is D, E, Q, R, S, T, H, I, L, V, A, G, C, N, K, or M.

120. The protein of claim 118 or 119, further comprising a substitution at position Q64, wherein the position Q64 is numbered based on SEQ ID NO:1, optionally, wherein the substitution is Q64L/F/W/Q.

121. The protein of any one of claims 118-120, further comprising a substitution at position A85, wherein the position A85 is numbered based on SEQ ID NO:1, optionally, wherein the substitution is A85F/Y/F/M/L/A.

122. The protein of any one of claims 118-121, further comprising a substitution at position H87, wherein the position H87 is numbered based on SEQ ID NO:1, optionally, wherein the substitution is H87R/L/V/G/K.

123. The protein of any one of claims 118-122, further comprising a substitution at position H99, wherein the position H99 is numbered based on SEQ ID NO:1, optionally, wherein the substitution is H99L.

124. The protein of any one of claims 118-123, further comprising a substitution at position W100, wherein the position W100 is numbered based on SEQ ID NO:1, optionally, wherein the substitution is W100F/Y/L.

125. The protein of any one of claims 118-124, further comprising a substitution at position T108, wherein the position T108 is numbered based on SEQ ID NO:1, optionally, wherein the substitution is T108N/D.

126. The protein of any one of claims 118-125, further comprising a substitution at position H113, wherein the position H113 is numbered based on SEQ ID NO:1, optionally, wherein the substitution is H113F/Y.

127. The protein of any one of claims 118-126, further comprising a substitution at position H30, H36, H80, H84, H87, H92, H98, H99, or H101.

128. The protein of claim 127, comprising one or more of the substitutions H30D, H36T, H80T, H84S, H87R, H92S, H98Q, H99L, and H101R.

129. The protein of any one of claims 118-128, further comprising a substitution at one or more of position V41, R46, T97, R103, E109, and I114.

130. The protein of any one of claims 118-129, further comprising one or more of the substitutions V41T, R46V, T97L, R103V, E109D, and I114T.

131. The protein of any one of claims 118-130, wherein the protein comprises the substitution F9V/S/N, and optionally comprises one or more of the substitutions Q64W, A85F, and H87L.

132. The protein of any one of claims 118-130, wherein the protein comprises the substitution F9N, and optionally comprises one or more of the substitutions Q64L, A85F, H87R, H99L, W100F, T108D, and H113Y.

133. The protein of claim 118, wherein the protein comprises the substitution F9N, and optionally comprises one or more of the substitutions H30D, H36T, Q64L, H80T, H84S, A85F, H87R, H92S, H98Q, H99L, W100F, H101R, T108D, and H113Y.

134. The protein of claim 118, wherein the protein comprises the substitution F9N, and optionally comprises one or more of the substitutions H30D, H36T, L56Q, Q64L, H80T, H84S, A85F, H87R, H92S, H98Q, H99L, W100F, H101K/R, T108D, and H113Y.

135. The protein of claim 118, wherein the protein comprises the substitution F9N, and optionally comprises one or more of the substitutions D231, H30D, H36T, V41T, R46V, R55S, L56Q, Q64L, K68S, H80T, Q82R, H84S, A85F, H87R, H92S, T97L, H98Q, H99L, W100F, H101K/R, R103V, T108D/V, E109A, H113Y, and I114V, further optionally, wherein the amino acid sequence does not include lysine.

136. A fusion protein comprising the protein of any one of claims 1-85 and 94-135 fused to another protein.

137. The protein of any one of claims 1-85 and 94-136 or the polypeptide of any one of claims 86-93, comprising one or more non-naturally occurring amino acids.

138. The protein or polypeptide of claim 137, wherein the one or more non-naturally occurring amino acids are a chemically modified version of one or more naturally occurring amino acids.

139. The protein or polypeptide of claim 138, wherein the one or more chemically modified amino acids comprise a chemical modification that introduces a chemical handle.

140. The protein of any one of claims 1-85 and 94-136 or the polypeptide of any one of claims 86-93, comprising one or more cysteine residues inserted at an N-terminus or a C-terminus or substitutions of one or more amino acids with a cysteine residue.

141. A protein comprising an amino acid sequence comprising at least 50%, 55%, 60%, 65%, 70%, 75%, 80%, 85%, 90%, 91%, 92%, 93%, 94%, 95%, 96%, 97%, 98%, or 99% identity to any one of the amino acid sequences set forth below:

| Sequence | SEQ ID NO |
|---|---|
| MSEEQIRQFLRRFYEALDSGDADTAASLFHPGVTIHLWDGVTFT | 2605 |
| SREEFREWFERLFSTSKDAWREIKSLEVRGDTVEVHVQLHFTLNGQKHTVQLTHHFHFRGNRVTEVRVH | 2606 |
| INPTGLE | |
| MSEEQIRQFLRRFYEALDSGDADTAASLFHPGVTIHLWDGVTFTSREEFREWFERLFSTSKDAWREIKSLE | 2608 |
| VRG | |
| DTVEVHVQLHFTLNGQKHTVDLTHHFHFRGNRVTEVRVHINPTGLE | 2607 |
| MSEEQIRQFLRRFYEALDSGDADTAASLFHPGVTIHLWDGVTFTSREEFREWFERLFSTSKDAWREIKSLE | 2610 |
| VRGDTVEVHVQLHFTLNGQKHTVDLTHHFHFRG | |
| NRVTEVRVHINPTGLE | 2609 |
| PSISEEQIRQNLRRFYEALDSGDADTAASLFHPGVTIHLWDGVTFTSREEFREWFERLFSTSKDALREIVEL | 2611 |
| RVRGDTVVVVVVLHFTRNGQKHVVVLVHLWHFRGNRVDEVRVEIIPAP | |
| DGVTFTSREEFREWFERLFSTSKDALREIVELRVRGDTVVVVVVLHFTRNGQKHVVVLVHTWRFRGNRV | 2612 |
| DEVRVEIIPAPSLDEESIEARVAEARRLAEERLAELGDPPPSISEEQIRQNLRRFYEALDSGDADTAASLFHP | |
| SREEFREWFERLFSTSKDALREIVELRVRGDTVVVVVVLHFTRNGQKHVVVLVHTWRFRGNRVDEVRVEII | 2613 |
| PAPSLDEESIEARVAEARRLAEERLAELGDPPPSISEEQIRQNLRRFYEALDSGDADTAASLFHPGVTIHLW | |
| SEEQIRQNLRRFYEALDSGDADTAASLFHPGVTIHLWDGVTFTSREEFREWFERLFSTSKDALREIKSLEVR | 2614 |
| GDTVEVHVQLHFTRNGQKHTVDLTHLFHFR | |
| NRVDEVRVYIN | 2615 |
| NRVDEVRVYINPT | 2616 |
| GQKHVVVLVHTFRFRG | 2617 |
| NRVTEVRVEIIPAPSLDEESIEARVAEARRLAEERLAELGDPPPSISEEQIRQFLRRFYEALDSGDADTAASLF | 2618 |
| HPGVTIHLWDGVTFTSREEFREWFERLFSTSKDAWREIVELRVRGDTVVVVVVLHFTLN | |
| NRVTEVRVEIIPAP | 2619 |
| SLDEESIEARVAEARRLAEERLAELGDPPPSISEEQIRQFLRRFYEALDSGDADTAASLFHPGVTIHLWDGV | 2620 |
| TFTSREEFREWFERLFSTSKDAWREIVELRVRGDTVVVVVVLHFTLNGQKHVVVLVHTFRFRG | |
| SLDEESIEARVAEARRLAEERLAELGDPP | 2621 |
| PSISEEQIRQFLRRFYEALDSGDADTAASLFHPGVTIHLWDGVTFTSREEFREWFERLFSTSKDAWREIVEL | 2622 |
| RVRGDTVVVVVVLHFTLNGQKHVVVLVHTFRFRGNRVTEVRVEIIPAP | |
| PSISEEQIRQFLRRFYEALDSG | 2623 |
| DADTAASLFHPGVTIHLWDGVTFTSREEFREWFERLFSTSKDAWREIVELRVRGDTVVVVVVLHFTLNG | 2624 |
| QKHVVVLVHTFRFRGNRVTEVRVEIIPAPSLDEESIEARVAEARRLAEERLAELGDPP | |
| DADTAASLFHP | 2625 |
| GVTIHLWDGVTFTSREEFREWFERLFSTSKDAWREIVELRVRGDTVVVVVVLHFTLNGQKHVVVLVHTF | 2626 |
| RFRGNRVTEVRVEIIPAPSLDEESIEARVAEARRLAEERLAELGDPPPSISEEQIRQFLRRFYEALDSG | |
| GVTIHLW | 2627 |
| DGVTFTSREEFREWFERLFSTSKDAWREIVELRVRGDTVVVVVVLHFTLNGQKHVVVLVHTFRFRGNRV | 2628 |
| TEVRVEIIPAPSLDEESIEARVAEARRLAEERLAELGDPPPSISEEQIRQFLRRFYEALDSGDADTAASLFHP | |
| DGVTFT | 2629 |
| SREEFREWFERLFSTSKDAWREIVELRVRGDTVVVVVVLHFTLNGQKHVVVLVHTFRFRGNRVTEVRVEI | 2630 |
| IPAPSLDEESIEARVAEARRLAEERLAELGDPPPSISEEQIRQFLRRFYEALDSGDADTAASLFHPGVTIHLW | |
| SREEFREWFERLFSTSK | 2631 |
| DAWREIVELRVRGDTVVVVVVLHFTLNGQKHVVVLVHTFRFRGNRVTEVRVEIIPAPSLDEESIEARVAE | 2632 |
| ARRLAEERLAELGDPPPSISEEQIRQFLRRFYEALDSGDADTAASLFHPGVTIHLWDGVTFT | |
| DAWREIVELRVRG | 2633 |
| DTVVVVVVLHFTLNGQKHVVVLVHTFRFRGNRVTEVRVEIIPAPSLDEESIEARVAEARRLAEERLAELGD | 2634 |
| PPPSISEEQIRQFLRRFYEALDSGDADTAASLFHPGVTIHLWDGVTFTSREEFREWFERLFSTSK | |
| DTVVVVVVLHFTLN | 2635 |
| GQKHVVVLVHTFRFRGNRVTEVRVEIIPAPSLDEESIEARVAEARRLAEERLAELGDPPPSISEEQIRQFLRR | 2636 |
| FYEALDSGDADTAASLFHPGVTIHLWDGVTFTSREEFREWFERLFSTSKDAWREIVELRVRG | |
| MSGNRVDEVRVYINPT | 2637 |
| MSGGERREVELTHLFTFRG | 2638 |
| MSGSDAERAALLDRFYAALNAGDADAAAALFPPGVTIELWNGVVFRSREEFRAWFAELFARSPEARREVLS | 2639 |
| REIEGDRVRVRVRLTFVRD | |
| MSGNRVVDVRVYTNPT | 2640 |
| MSGGQKHTVDLLQLFKFVG | 2641 |
| MSGVYTNPT | 2642 |
| MSGVRVYTNPT | 2643 |
| MSGVDVRVYTNPT | 2644 |
| MSGRVVDVRVYTNPT | 2645 |
| MSGNRVVDVRVYTNPT | 2646 |
| MSGNRVVDARVYTNPT | 2647 |
| MSGNRVVDVRVYTEPT | 2648 |
| MSGNRVVDARVYTEPT | 2649 |
| MSGGNRVVDVRVYTNPT | 2650 |
| MSGFVGNRVVDVRVYTNPT | 2651 |
| MSGFKFVGNRVVDVRVYTNPT | 2652 |
| MSGQLFKFVGNRVVDVRVYTNPT | 2653 |
| MSGLLQLFKFVGNRVVDVRVYTNPT | 2654 |
| MSGQKHTVDLLQLFKFVGNRVVDVRVYTNPT | 2655 |
| MSGSEEQIRQNLRRFYEALDSGDADTAASLFDPGVTITLWDGTTFTSVEEFREWFERLFSTSKDALREIKSLE | 2656 |
| VRGDTVEVTVQLSFTRNGQKHTVDLLQLFKFVGNRVVD | |
| MSGSEEQIRQNLRRFYEALDSGDADTAASLFDPGVTITLWDGTTFTSVEEFREWFERLFSTSKDALREIKSLE | 2657 |
| VRGDTVEVTVQLSFTRNGQKHTVDLLQLFKFVGNRV | |
| MSGSEEQIRQNLRRFYEALDSGDADTAASLFDPGVTITLWDGTTFTSVEEFREWFERLFSTSKDALREIKSLE | 2658 |
| VRGDTVEVTVQLSFTRNGQKHTVDLLQLFKFVGN | |
| MSGSEEQIRQNLRRFYEALDSGDADTAASLFDPGVTITLWDGTTFTSVEEFREWFERLFSTSKDALREIKSLE | 2659 |
| VRGDTVEVTVQLSFTRNGQKHTVDLLQLFKFVG | |
| MSGSEEQIRQNLRRFYEALDSGDADTAASLFDPGVTITLWDGTTFTSVEEFREWFERLFSTSKDALREIKSLE | 2660 |
| VRGDTVEVTVQLSFTRNGQKHTVDLLQLFKFV | |
| MSGSEEQIRQNLRRFYEALDSGDADTAASLFDPGVTITLWDGTTFTSVEEFREWFERLFSTSKDALREIKSLE | 2661 |
| VRGDTVEVTVQLSFTRNGQKHTVDLLQLFK | |
| MSGSEEQIRQNLRRFYEALDSGDADTAASLFDPGVTITLWDGTTFTSVEEFREWFERLFSTSKDALREIKSLE | 2662 |
| VRGDTVEVTVQLSFTRNGQKHTVDLLQL | |
| MSGSEEQIRQNLRRFYEALDSGDADTAASLFDPGVTITLWDGTTFTSVEEFREWFERLFSTSKDALREIKSLE | 2679 |
| VRGDTVEVTVQLSFTRNGQKHTVDLL | |
| MSGSEEQIRQNLRRFYEALDSGDADTAASLFDPGVTITLWDGTTFTSVEEFREWFERLFSTSKDALREIKSLE | 2680 |
| VRGDTVEVTVQLSFTRNGQKHTVD | |
| MSGSEEQIRQNLRRFYEALDSGDADTAASLFDPGVTITLWDGTTFTSVEEFREWFERLFSTSKDALREIKSLE | 2681 |
| VRGDTVEVTVQLSFTRNGQKHT | |
| MSGSEEQIRQNLRRFYEALDSGDADTAASLFDPGVTITLWDGTTFTSVEEFREWFERLFSTSKDALREIKSLE | 2666 |
| VRGDTVEVTVQLSFTRNG | |
| MSGSEEQIRQNLRRFYEALDSGDADTAASLFDPGVTITLWDGTTFTSVEEFREWFERLFSTSKDALREIKSLE | 2667 |
| VRGDTVEVTVQLSFTRN | |
| MSGSEEQIRQNLRRFYEALDSGDADTAASLFDPGVTITLWDGTTFTSVEEFREWFERLFSTSKDALREIKSLE | 2668 |
| VRGDTVEVTVQLSFTRNGQKHTVDLLQLFKFVGNRVVDYRVYTNPT | |
| MSGSEEQIRQNLRRFYEALDSGDADTAASLFDPGVTITLWDGTTFTSVEEFREWFERLFSTSKDALREIKSLE | 2669 |
| VRGDTVEVTVQLSFTRNGQKHTVDLLQLFKFVGNRVVDRRVYTNPT | |
| MSGSEEQIRQNLRRFYEALDSGDADTAASLFDPGVTITLWDGTTFTSVEEFREWFERLFSTSKDALREIKSLE | 2670 |
| VRGDTVEVTVQLSFTRNGQKHTVDLLQLFKFVGNRVVDMRVYTNPT | |
| MSGSEEQIRQNLRRFYEALDSGDADTAASLFDPGVTITLWDGTTFTSVEEFREWFERLFSTSKDALREIKSLE | 2671 |
| VRGDTVEVTVQLSFTRNGQKHTVDLLQLFKFVGNRVVDDRVYTNPT | |
| MSGNRVDEVRVYINPT | 2672 |
| MSGGQKHTVDLTHLFHFRG | 2673 |
| MSGSEEQIRQNLRRFYEALDSGDADTAASLFHPGVTIHLWDGVTFTSREEFREWFERLFSTSKDALREIKSLE | 2674 |
| VRGDTVEVHVQLHFTRN | |
| SEEQIRQNLRRFYEALDSGDAITAASLFDPGVTITLWDGTTFTSVEEFREWFESQFSTSKDALREISSLEVRGD | 2675 |
| TVEVTVRLSFTRNGQKHTVDLLQLFKFV | |
| RVVAVRVYVNPT | 2676 |
| SEEQIRQNLRRFYEALDSGDADTAASLFDPGVTITLWDGVTFTSREEFREWFERQFSTSKDALREIKSLEVRG | 2677 |
| DTVEVTIQLSFTRNGQKSTVDLTQLFRFR | |
| RVDEVRVYINPT | 2678 |
| MSGMSEEQIRQNLRRFYEALDSGDAITAASLFDPGVTITLWDGTTFTSVEEFREWFESQFSTSKDALREISSL | 2733 |
| EVRGDTVEVTVRLSFTRNGQKHTVDLLQLFKFV | |
| MSGGNRVVAVRVYVNPT | 2734 |
| RVVAVRVYVNPTG | 2770 |
| RVVIVRVYVNPTG | 2771 |
| HVVAVRVYVNPTG | 2772 |
| MSEEQIRQNLRRFYEALDSGDAITAASLFDPGVTITLWDGTTFTSVEEFREWFESQFSTSKDALREISSLEVR | 2773 |
| GDTVEVTVRLSFTRNGQKHTVDLLQLFKFV | |
| MCEEQIRQNLLRFYEALDSGDAITAASLFDPGVTITLWDGTTFTSIEEFREWFDSQFSTSKDALREISSLEVRG | 2797 |
| DTVEVTVRLSFTRNGQKHTVDLLQLFKFV | |
| MSEEQIRQNLLRFYEALDSGDAITAASLFDPGVTITLWDGTTFTSVEEFREWFESQFSTSKDALREISSLEVRG | 2798 |
| DTVEVTVRLSFTRNGQKHTVDLLQLFKFV | |
| MSEEQIRQNLLRFYEALDSGDAITAASLFNPGVTITLWDGTTFTSVEEFREWFESQFSTSKDALREISSLEVR | |
| GDTVEVTVRLSFTRNGQKHTVDLLQLFKFV | 2799 |
| MSEEQIRQNLRRFYEALDSGDAITAASLFDPGVTITLWDGATFTSVEEFREWFESQFSTSKDALREISSLEVR | 2800 |
| GDTVEVTVRLSFTRNGQKQTVDLLQLFKFV | |
| MSEEQIRQNLLRFYEALDSGDAVTAASLFDPGVTITLWDGTTFTTVEEFREWFESQFSTSKDALREISSLEVR | 2801 |
| GDTVEVTVRLSFTRNGQKHTVDLLQLFKFV | |
| MSEEQIRQNLRRFYEALDSGDAITAASLFDPGVTITLWDGTTFTSVEEFREWFGTQFSTSKDALREISSLEVR | 2802 |
| GDTVEVTVRLSFTSNGQKHTVDLLQLFKFV | |
| MSEEQIRQNLRRFYEALDSGDAITAASLFDSGVTITLWDGTTFTSVEEFREWFESQFSTSKDALREISSLEVR | 2803 |
| GDTVEVTVRLSFTRNGQKHTVDLLQLFKFV | |
| MTEEQLRQNLLRFYEALDSGDAITAASLFDPGVTITLWDGTTFTSVEKFREWFESQFSTSKDALREISSLEVR | 2804 |
| GDTVEVTVRLSFTRNGQKHTVDLLQLFKFV | |
| MSEEQIRQNLRRFYEALDCGDAITAASLFDPGVTITLWDGTTFTSVEEFREWFESQFSTSKDALREISSLEVR | 2805 |
| GDTVEVTVRLSFTRNGQKHTVDLLQLFKFV | |
| MSEEQIRQSLRRFYEALDSGDAITAASLFDPGVTITLWDGTTFTSVEEFREWFESQFSTSKDALREISSLEVRG | 2806 |
| DTVEVTVRLSFTRNGQKHTVDLLQLFKFV | |
| MSEEQIRQNLRRFYEALDSGDAITAASLFDPGVTITLWDGTTFTSVEEFREWFESQFSTSKDALREISSLEVR | 2807 |
| GDTVEVTVRLSFTRNGQKHTVDLLQQFKFV | |
| MSEEQIRQNLRRFYEALDSGDAITAASLFDPGVTITLWDGTTFTSVEEFREWFESQFSTSKDALREISSLEVR | 2808 |
| GDTVEVTVRLSFTRNGQKHTVDLLQLFEFV | |
| MSEEQIRQDLRRFYEALDSGDAITAASLFDPGVTITLWDGTTFTSVEEFREWFESQFSTSKDALREISSLEVR | 2809 |
| GDTVEVTVRLSFTRNGQKHTVDLLQLFKFI | |
| MSEEQIRQNLRRFYEALDSGDAITAASLFDPGVTITLWDGTTLTSVEEFREWFESQFSTSKDALREISSLEVR | 2810 |
| GDTVEVTVRLSFTRNGQKHTVDLLQLFKFV | |
| MSEEQIRQILRRFYEALDSGDAITAASLFDPGVTITLWDGTTFTSVEEFREWFESQFSTSKDALREISSLEVRG | 2811 |
| DTVEVTVRLSFTRDGQKHTVDLLQLFKFV | |
| MSEEQIRQNLRRFYEALDSGDAITAASLFDPGVTITLWDGTTFTSVEEFREWFESQFSTSKDAFREISSLEVR | 2812 |
| GDTVEVTVRLSFTRNGQKHTVDLLQLFKFV | |
| MSEEQIRQNLRRFYEALDSGDAITAASLFDPGVIITLWDGTTFTSVEEFREWFESQFSTSKDALREISSLEVRG | 2813 |
| DTVEVTVRLSFTRNGQKHTVDLLQLFKFV | |
| MSEEQIRQNLRRFYEALDSGDAITAASLFDPGVTITLWDGTTFTSVEEFRVWFESQFSTSTDALREISSLEVR | 2814 |
| GDTVEVTVRLSFTRNGQKHTVDLLQLFKFV | |

or a protein comprising an amino acid sequence comprising at least 50%, 55%, 60%, 65%, 70%, 75%, 80%, 85%, 90%, 91%, 92%, 93%, 94%, 95%, 96%, 97%, 98%, or 99% identity to any one of the amino acid sequences set forth in Table 13A, SEQ ID NO:2733, SEQ ID NO:2736, SEQ ID NO:2734, SEQ ID NO:2735, SEQ ID NO:2770, SEQ ID NO:2771, and SEQ ID NO:2772, Table 19, Table 20, Table 21 and Table 22.

142. A fusion protein comprising the protein of claim 141 fused to another protein.

143. A nucleic acid comprising a nucleotide sequence encoding the protein of claim 141 or 142.

144. An expression vector comprising the nucleic acid of claim 143 operatively linked to an expression control element.

145. A recombinant host cell comprising one or more proteins of claim 141 or 142, the nucleic acid of claim 143, and/or the expression vector of claim 144.

146. One or both of a first nucleic acid encoding a first protein and a second nucleic acid encoding a second protein,
wherein the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to any one the amino acid sequences set forth below:

| Sequence | SEQ ID NO |
|---|---|
| MSEEQIRQFLRRFYEALDSGDADTAASLFHPGVTIHLWDGVTFTSREEFREWFERLFSTSKDAWREIKSLE | 2608 |
| VRG | |
| MSEEQIRQFLRRFYEALDSGDADTAASLFHPGVTIHLWDGVTFTSREEFREWFERLFSTSKDAWREIKSLE | 2610 |
| VRGDTVEVHVQLHFTLNGQKHTVDLTHHFHFRG | |
| PSISEEQIRQNLRRFYEALDSGDADTAASLFHPGVTIHLWDGVTFTSREEFREWFERLFSTSKDALREIVE | 2611 |
| LRVRGDTVVVVVVLHFTRNGQKHVVVLVHLWHFRGNRVDEVRVEIIPAP | |
| DGVTFTSREEFREWFERLFSTSKDALREIVELRVRGDTVVVVVVLHFTRNGQKHVVVLVHTWRFRGNRVDE | 2612 |
| VRVEIIPAPSLDEESIEARVAEARRLAEERLAELGDPPPSISEEQIRQNLRRFYEALDSGDADTAASLFHP | |
| SREEFREWFERLFSTSKDALREIVELRVRGDTVVVVVVLHFTRNGQKHVVVLVHTWRFRGNRVDEVRVEII | 2613 |
| PAPSLDEESIEARVAEARRLAEERLAELGDPPPSISEEQIRQNLRRFYEALDSGDADTAASLFHPGVTIHL | |
| W | |
| SEEQIRQNLRRFYEALDSGDADTAASLFHPGVTIHLWDGVTFTSREEFREWFERLFSTSKDALREIKSLEV | 2614 |
| RGDTVEVHVQLHFTRNGQKHTVDLTHLFHFR | |
| NRVTEVRVEIIPAPSLDEESIEARVAEARRLAEERLAELGDPPPSISEEQIRQFLRRFYEALDSGDADTAA | 2618 |
| SLFHPGVTIHLWDGVTFTSREEFREWFERLFSTSKDAWREIVELRVRGDTVVVVVVLHFTLN | |
| SLDEESIEARVAEARRLAEERLAELGDPPPSISEEQIRQFLRRFYEALDSGDADTAASLFHPGVTIHLWDG | 2620 |
| VTFTSREEFREWFERLFSTSKDAWREIVELRVRGDTVVVVVVLHFTLNGQKHVVVLVHTFRFRG | |
| PSISEEQIRQFLRRFYEALDSGDADTAASLFHPGVTIHLWDGVTFTSREEFREWFERLFSTSKDAWREIVE | 2622 |
| LRVRGDTVVVVVVLHFTLNGQKHVVVLVHTFRFRGNRVTEVRVEIIPAP | |
| DADTAASLFHPGVTIHLWDGVTFTSREEFREWFERLFSTSKDAWREIVELRVRGDTVVVVVVLHFTLNGQK | 2624 |
| HVVVLVHTFRFRGNRVTEVRVEIIPAPSLDEESIEARVAEARRLAEERLAELGDPP | |
| GVTIHLWDGVTFTSREEFREWFERLFSTSKDAWREIVELRVRGDTVVVVVVLHFTLNGQKHVVVLVHTFRF | 2626 |
| RGNRVTEVRVEIIPAPSLDEESIEARVAEARRLAEERLAELGDPPPSISEEQIRQFLRRFYEALDSG | |
| DGVTFTSREEFREWFERLFSTSKDAWREIVELRVRGDTVVVVVVLHFTLNGQKHVVVLVHTFRFRGNRVTE | 2628 |
| VRVEIIPAPSLDEESIEARVAEARRLAEERLAELGDPPPSISEEQIRQFLRRFYEALDSGDADTAASLFHP | |
| SREEFREWFERLFSTSKDAWREIVELRVRGDTVVVVVVLHFTLNGQKHVVVLVHTFRFRGNRVTEVRVEII | 2630 |
| PAPSLDEESIEARVAEARRLAEERLAELGDPPPSISEEQIRQFLRRFYEALDSGDADTAASLFHPGVTIHL | |
| W | |
| DAWREIVELRVRGDTVVVVVVLHFTLNGQKHVVVLVHTFRFRGNRVTEVRVEIIPAPSLDEESIEARVAEA | 2632 |
| RRLAEERLAELGDPPPSISEEQIRQFLRRFYEALDSGDADTAASLFHPGVTIHLWDGVTFT | |
| DTVVVVVVLHFTLNGQKHVVVLVHTFRFRGNRVTEVRVEIIPAPSLDEESIEARVAEARRLAEERLAELGD | 2634 |
| PPPSISEEQIRQFLRRFYEALDSGDADTAASLFHPGVTIHLWDGVTFTSREEFREWFERLFSTSK | |
| GQKHVVVLVHTFRFRGNRVTEVRVEIIPAPSLDEESIEARVAEARRLAEERLAELGDPPPSISEEQIRQFL | 2636 |
| RRFYEALDSGDADTAASLFHPGVTIHLWDGVTFTSREEFREWFERLFSTSKDAWREIVELRVRG | |
| MSGSDAERAALLDRFYAALNAGDADAAAALFPPGVTIELWNGVVFRSREEFRAWFAELFARSPEARREVLS | 2639 |
| REIEGDRVRVRVRLTFVRD | |
| MSGSEEQIRQNLRRFYEALDSGDADTAASLFDPGVTITLWDGTTFTSVEEFREWFERLFSTSKDALREIKS | 2656 |
| LEVRGDTVEVTVQLSFTRNGQKHTVDLLQLFKFVGNRVVD | |
| MSGSEEQIRQNLRRFYEALDSGDADTAASLFDPGVTITLWDGTTFTSVEEFREWFERLFSTSKDALREIKS | 2657 |
| LEVRGDTVEVTVQLSFTRNGQKHTVDLLQLFKFVGNRV | |
| MSGSEEQIRQNLRRFYEALDSGDADTAASLFDPGVTITLWDGTTFTSVEEFREWFERLFSTSKDALREIKS | 2658 |
| LEVRGDTVEVTVQLSFTRNGQKHTVDLLQLFKFVGN | |
| MSGSEEQIRQNLRRFYEALDSGDADTAASLFDPGVTITLWDGTTFTSVEEFREWFERLFSTSKDALREIKS | 2659 |
| LEVRGDTVEVTVQLSFTRNGQKHTVDLLQLFKFVG | |
| MSGSEEQIRQNLRRFYEALDSGDADTAASLFDPGVTITLWDGTTFTSVEEFREWFERLFSTSKDALREIKS | 2660 |
| LEVRGDTVEVTVQLSFTRNGQKHTVDLLQLFKFV | |
| MSGSEEQIRQNLRRFYEALDSGDADTAASLFDPGVTITLWDGTTFTSVEEFREWFERLFSTSKDALREIKS | 2661 |
| LEVRGDTVEVTVQLSFTRNGQKHTVDLLQLFK | |
| MSGSEEQIRQNLRRFYEALDSGDADTAASLFDPGVTITLWDGTTFTSVEEFREWFERLFSTSKDALREIKS | 2662 |
| LEVRGDTVEVTVQLSFTRNGQKHTVDLLQL | |
| MSGSEEQIRQNLRRFYEALDSGDADTAASLFDPGVTITLWDGTTFTSVEEFREWFERLFSTSKDALREIKS | 2679 |
| LEVRGDTVEVTVQLSFTRNGQKHTVDLL | |
| MSGSEEQIRQNLRRFYEALDSGDADTAASLFDPGVTITLWDGTTFTSVEEFREWFERLFSTSKDALREIKS | 2680 |
| LEVRGDTVEVTVQLSFTRNGQKHTVD | |
| MSGSEEQIRQNLRRFYEALDSGDADTAASLFDPGVTITLWDGTTFTSVEEFREWFERLFSTSKDALREIKS | 2681 |
| LEVRGDTVEVTVQLSFTRNGQKHT | |
| MSGSEEQIRQNLRRFYEALDSGDADTAASLFDPGVTITLWDGTTFTSVEEFREWFERLFSTSKDALREIKS | 2666 |
| LEVRGDTVEVTVQLSFTRNG | |
| MSGSEEQIRQNLRRFYEALDSGDADTAASLFDPGVTITLWDGTTFTSVEEFREWFERLFSTSKDALREIKS | 2667 |
| LEVRGDTVEVTVQLSFTRN | |
| MSGSEEQIRQNLRRFYEALDSGDADTAASLFDPGVTITLWDGTTFTSVEEFREWFERLFSTSKDALREIKS | 2668 |
| LEVRGDTVEVTVQLSFTRNGQKHTVDLLQLFKFVGNRVVDYRVYTNPT | |
| MSGSEEQIRQNLRRFYEALDSGDADTAASLFDPGVTITLWDGTTFTSVEEFREWFERLFSTSKDALREIKS | 2669 |
| LEVRGDTVEVTVQLSFTRNGQKHTVDLLQLFKFVGNRVVDRRVYTNPT | |
| MSGSEEQIRQNLRRFYEALDSGDADTAASLFDPGVTITLWDGTTFTSVEEFREWFERLFSTSKDALREIKS | 2670 |
| LEVRGDTVEVTVQLSFTRNGQKHTVDLLQLFKFVGNRVVDMRVYTNPT | |
| MSGSEEQIRQNLRRFYEALDSGDADTAASLFDPGVTITLWDGTTFTSVEEFREWFERLFSTSKDALREIKS | 2671 |
| LEVRGDTVEVTVQLSFTRNGQKHTVDLLQLFKFVGNRVVDDRVYTNPT | |
| MSGSEEQIRQNLRRFYEALDSGDADTAASLFHPGVTIHLWDGVTFTSREEFREWFERLFSTSKDALREIKS | 2674 |
| LEVRGDTVEVHVQLHFTRN | |
| SEEQIRQNLRRFYEALDSGDAITAASLFDPGVTITLWDGTTFTSVEEFREWFESQFSTSKDALREISSLEV | 2675 |
| RGDTVEVTVRLSFTRNGQKHTVDLLQLFKFV | |
| SEEQIRQNLRRFYEALDSGDADTAASLFDPGVTITLWDGVTFTSREEFREWFERQFSTSKDALREIKSLEV | 2677 |
| RGDTVEVTIQLSFTRNGQKSTVDLTQLFRFR | |
| MSGMSEEQIRQNLRRFYEALDSGDAITAASLFDPGVTITLWDGTTFTSVEEFREWFESQFSTSKDALREIS | 2733 |
| SLEVRGDTVEVTVRLSFTRNGQKHTVDLLQLFKFV | |

or wherein the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to any one the IgLux amino acid sequences set forth in Table 13A, SEQ ID NO:2733, SEQ ID NO:2736, Table 19, Table 20, Table 21, and Table 22;
and
wherein the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to any one the amino acid sequences set forth below:

| | SEQ |
|---|---|
| Sequence | ID NO |
| MSEEQIRQFLRRFYEALDSGDADTAASLFHPGVTIH | 2605 |
| LWDGVTFT | |
| DTVEVHVQLHFTLNGQKHTVDLTHHFHFRGNRVTEV | 2607 |
| RVHINPTGLE | |
| NRVDEVRVYIN | 2615 |
| NRVDEVRVYINPT | 2616 |
| GQKHVVVLVHTFRFRG | 2617 |
| NRVTEVRVEIIPAP | 2619 |
| SLDEESIEARVAEARRLAEERLAELGDPP | 2621 |
| PSISEEQIRQFLRRFYEALDSG | 2623 |
| DADTAASLFHP | 2625 |
| GVTIHLW | 2627 |
| DGVTFT | 2629 |
| SREEFREWFERLFSTSK | 2631 |
| DAWREIVELRVRG | 2633 |
| DTVVVVVVLHFTLN | 2635 |
| MSGNRVDEVRVYINPT | 2637 |
| MSGGERREVELTHLFTFRG | 2638 |
| MSGNRVVDVRVYTNPT | 2640 |
| MSGGQKHTVDLLQLFKFVG | 2641 |
| MSGVYTNPT | 2642 |
| MSGVRVYTNPT | 2643 |
| MSGVDVRVYTNPT | 2644 |
| MSGRVVDVRVYTNPT | 2645 |
| MSGNRVVDVRVYTNPT | 2646 |
| MSGNRVVDARVYTNPT | 2647 |
| MSGNRVVDVRVYTEPT | 2648 |
| MSGNRVVDARVYTEPT | 2649 |
| MSGGNRVVDVRVYTNPT | 2650 |
| MSGFVGNRVVDVRVYTNPT | 2651 |
| MSGFKFVGNRVVDVRVYTNPT | 2652 |
| MSGQLFKFVGNRVVDVRVYTNPT | 2653 |
| MSGLLQLFKFVGNRVVDVRVYTNPT | 2654 |
| MSGQKHTVDLLQLFKFVGNRVVDVRVYTNPT | 2655 |
| MSGNRVDEVRVYINPT | 2672 |
| MSGGQKHTVDLTHLFHFRG | 2673 |
| RVVAVRVYVNPT | 2676 |
| RVDEVRVYINPT | 2678 |
| MSGGNRVVAVRVYVNPT | 2734 |

or wherein the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to any one the smLux amino acid sequences set forth in Table 13A, SEQ ID NO:2734, SEQ ID NO:2735, SEQ ID NO:2770, SEQ ID NO:2771, and SEQ ID NO:2772.

147. One or both of a first nucleic acid encoding a first protein and a second nucleic acid encoding a second protein,
wherein
(i) the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: MSEEQIRQFLRRFYEALDSGDADTAASLFHPGVTIHLWDGVTFT (SEQ ID NO:2605) and the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2606)
SREEFREWFERLFSTSKDAWREIKSLEVRGDTVEVHVQLHFTLNGQKH
TVDLTHHFHFRGNRVTEVRVHINPTGLE;
(ii) the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: MSEEQIRQFLRRFYEALDSGDADTAASLFHPGVTIHLWDGVTFTSREEFREWFERLFSTSKDAWREIKSLE VRG (SEQ ID NO:2608) and the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2607)
DTVEVHVQLHFTLNGQKHTVDLTHHFHFRGNRVTEVRVHINPTGLE;
(iii) the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: MSEEQIRQFLRRFYEALDSGDADTAASLFHPGVTIHLWDGVTFTSREEFREWFERLFSTSKDAWREIKSLE VRGDTVEVHVQLHFTLNGQKHTVDLTHHFHFRG (SEQ ID NO:2610) and the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: NRVTEVRVHINPTGLE (SEQ ID NO: 2609);
(iv) the first protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: SEEQIRQNLRRFYEALDSGDADTAASLFHPGVTIHLWDGVTFTSREEFREWFERLFSTSKDALREIKSLEVR GDTVEVHVQLHFTRNGQKHTVDLTHLFHFR (SEQ ID NO:2614) and the second protein comprises an amino acid sequence having at least 50%, 55%, 60%, 65%, 70%, 75%, 80%, 85%, 90%, 91%, 92%, 93%, 94%, 95%, 96%, 97%, 98%, or 99% identity to the amino acid sequence: NRVDEVRVYIN (SEQ ID NO: 2615) or NRVDEVRVYINPT (SEQ ID NO:2616);
(v) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: GQKHVVVLVHTFRFRG (SEQ ID NO:2617) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2618)
NRVTEVRVEIIPAPSLDEESIEARVAEARRLAEERLAELGDPPPSISE
EQIRQFLRRFYEALDSGDADTAASLFHPGVTIHLWDGVTFTSREEFRE
WFERLFSTSKDAWREIVELRVRGDTVVVVVVLHFTLN;
(vi) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: NRVTEVRVEIIPAP (SEQ ID NO:2619) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2620)
SLDEESIEARVAEARRLAEERLAELGDPPPSISEEQIRQFLRRFYEAL
DSGDADTAASLFHPGVTIHLWDGVTFTSREEFREWFERLFSTSKDAWR
EIVELRVRGDTVVVVVVLHFTLNGQKHVVVLVHTFRFRG;
(vii) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: SLDEESIEARVAEARRLAEERLAELGDPP (SEQ ID NO:2621) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2622)
PSISEEQIRQFLRRFYEALDSGDADTAASLFHPGVTIHLWDGVTFTSR
EEFREWFERLFSTSKDAWREIVELRVRGDTVVVVVVLHFTLNGQKHVV
VLVHTFRFRGNRVTEVRVEIIPAP;
(viii) the first protein comprises at least 50%, 55%, 60%, 65%, 70%, 75%, 80%, 85%, 90%, 91%, GP-100,DNA 92%, 93%, 94%, 95%, 96%, 97%, 98%, or 99% identity to the amino acid sequence: PSISEEQIRQFLRRFYEALDSG (SEQ ID NO:2623) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2624)
DADTAASLFHPGVTIHLWDGVTFTSREEFREWFERLFSTSKDAWREIV
ELRVRGDTVVVVVVLHFTLNGQKHVVVLVHTFRFRGNRVTEVRVEIIP
APSLDEESIEARVAEARRLAEERLAELGDPP;
(ix) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: DADTAASLFHP (SEQ ID NO:2625) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2626)
GVTIHLWDGVTFTSREEFREWFERLFSTSKDAWREIVELRVRGDTVVV
VVVLHFTLNGQKHVVVLVHTFRFRGNRVTEVRVEIIPAPSLDEESIEA
RVAEARRLAEERLAELGDPPPSISEEQIRQFLRRFYEALDSG;
(x) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: GVTIHLW (SEQ ID NO:2627) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2628)
DGVTFTSREEFREWFERLFSTSKDAWREIVELRVRGDTVVVVVVLHFT
LNGQKHVVVLVHTFRFRGNRVTEVRVEIIPAPSLDEESIEARVAEARR
LAEERLAELGDPPPSISEEQIRQFLRRFYEALDSGDADTAASLFHP;
(xi) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: DGVTFT (SEQ ID NO:2629) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2630)
SREEFREWFERLFSTSKDAWREIVELRVRGDTVVVVVVLHFTLNGQKH
VVVLVHTFRFRGNRVTEVRVEIIPAPSLDEESIEARVAEARRLAEERL
AELGDPPPSISEEQIRQFLRRFYEALDSGDADTAASLFHPGVTIHLW;
(xii) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: SREEFREWFERLFSTSK (SEQ ID NO:2631) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2632)
DAWREIVELRVRGDTVVVVVVLHFTLNGQKHVVVLVHTFRFRGNRVTE
VRVEIIPAPSLDEESIEARVAEARRLAEERLAELGDPPPSISEEQIRQ
FLRRFYEALDSGDADTAASLFHPGVTIHLWDGVTFT;
(xiii) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: DAWREIVELRVRG (SEQ ID NO:2633) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2634)
DTVVVVVVLHFTLNGQKHVVVLVHTFRFRGNRVTEVRVEIIPAPSLDE
ESIEARVAEARRLAEERLAELGDPPPSISEEQIRQFLRRFYEALDSGD
ADTAASLFHPGVTIHLWDGVTFTSREEFREWFERLFSTSK;
(xiv) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: DTVVVVVVLHFTLN (SEQ ID NO:2635) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence:
(SEQ ID NO: 2636)
GQKHVVVLVHTFRFRGNRVTEVRVEIIPAPSLDEESIEARVAEARRLA
EERLAELGDPPPSISEEQIRQFLRRFYEALDSGDADTAASLFHPGVTI
HLWDGVTFTSREEFREWFERLFSTSKDAWREIVELRVRG,
or
(xv) the first protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: MSGMSEEQIRQNLRRFYEALDSGDAITAASLFDPGVTITLWDGTTFTSVEEFREWFESQFSTSKDALREIS SLEVRGDTVEVTVRLSFTRNGQKHTVDLLQLFKFV (SEQ ID NO:2733) and the second protein comprises at least 50%, 55%, 60%, 65%, 70%, 75%, 80%, 85%, 90%, 91%, 92%, 93%, 94%, 95%, 96%, 97%, 98%, or 99% identity to the amino acid sequence: MSGGNRVVAVRVYVNPT (SEQ ID NO: 2734).

148. The one or both of the first nucleic acid and the second nucleic acid of claim 147, wherein the first protein is a fusion protein comprising a first domain and the second protein is a fusion protein comprising a second domain, wherein the first domain and the second domain are capable of associating with each other.

149. The one or both of the first nucleic acid and the second nucleic acid of claim 148, wherein the first domain and the second domain associate with each other in the presence of a molecule that binds to either the first domain, the second domain, or both.

150. An expression vector comprising one of both of the first nucleic acid and the second nucleic acid of any one of claims 146-149 or a first expression vector comprising the first nucleic acid of any one of claims 146-149 and a second expression vector comprising the second nucleic acid of any one of claims 146-149.

151. A kit comprising:
(a) the protein of any one of claims 1-85, 112-113, 117-135, 137-141,
(b) the polypeptide of any one of claims 86-93,
(c) the fusion protein of claim 94, 136, or 142,
(d) the polypeptide component of any one of claims 95-111,
(e) the nucleic acid of claim 114, 143,
(f) the expression vector of claim 115, 144,
(g) the host cell of claim 116, 145, and/or
(h) the first and the second nucleic acid of any one of claims 146-149, and/or
(i) the first and the second expression vector of claim 150.

152. The kit of claim 151, further comprising a substrate, optionally wherein the substrate is a luciferin analog, optionally wherein the luciferin analog is a compound of formula (I), or a stereoisomer, a tautomer or a salt thereof, a compound of formula (Ia), a compound of formula (Ib), a compound of formula (Ic), a compound of formula (Id), a compound of formula (Ie), or a compound set forth in claim 194.

153. The kit of claim 152, wherein the compound of Formula (I) is: 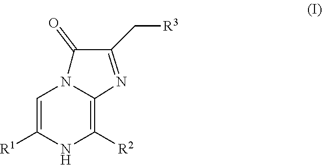
or a stereoisomer, a tautomer or a salt thereof, wherein:
R¹, R², and R³ are independently selected from: a C₃₋₆ cycloalkyl; an aryl; an aryl substituted with at least one of C₁₃ alkyl, halogen, C₁3 haloalkyl, hydroxyl, alkoxy, nitro or amino alcohol; 5-10 membered heteroaryl having 1-3 ring heteroatoms independently selected from O, S, Se and N, and a heterocycle; wherein:
if R¹ is an aryl, then R² and R³ are independently selected from: a C₃₋₆ cycloalkyl; an aryl; an aryl substituted with at least one of C₁₋₃ alkyl, halogen, C₁₋₃ haloalkyl, hydroxy, alkoxy or nitro; and 5-10 membered heteroaryl having 1-3 ring heteroatoms independently selected from O, S, Se and N;
if R³ is an aryl, then R¹ and R² are independently selected from: a C₃₋₆ cycloalkyl; an aryl; an aryl substituted with at least one of C₁₋₃ alkyl, halogen, C₁₋₃ haloalkyl, hydroxy, alkoxy, nitro or amino alcohol; 5 membered heteroaryl having 1-3 ring heteroatoms independently selected from O, S, Se and N; and 10 membered heteroaryl having 1-3 ring heteroatoms independently selected from O, S, and Se; 6 membered heteroaryl having 1-3 ring heteroatoms independently selected from O, S, Se, and N; a heterocycle and
if R² is an aryl, then Rand R³ are independently selected from: a C₃₋₆ cycloalkyl; an aryl; an aryl substituted with at least one of C₁₋₃ alkyl, halogen, C₁₋₃ haloalkyl, hydroxy, alkoxy, nitro or amino alcohol; 5 membered heteroaryl having 1-3 ring heteroatoms independently selected from O, S, Se and N; 10 membered heteroaryl having 1-3 ring heteroatoms independently selected from O, S, Se and N, and a heterocycle.

153. A compound of formula (Ia): 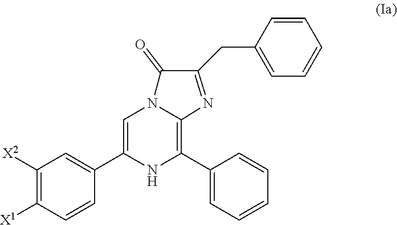
or a stereoisomer, a tautomer or a salt thereof, wherein:
X¹-X² are independently selected from a group consisting of: halogen, hydroxyl, haloalkyl, alkyl or nitro;
with proviso that:
when X² is hydrogen, then X¹ is selected from a group consisting of: haloalkyl, alkyl or nitro;
when X¹ is hydroxyl, then X² is selected from halogen.

154. The compound of claim 153, wherein X² is hydrogen and X¹ is haloalkyl.

155. The compound of claim 154, wherein X² is hydrogen and X¹ is trifluoromethyl.

156. The compound of claim 153, wherein X² is hydrogen and X¹ is alkyl.

157. The compound of claim 156, wherein X² is hydrogen and X¹ is methyl.

158. The compound of claim 153, wherein X² is hydrogen and X¹ is nitro.

159. The compound of claim 153, wherein X¹ is hydroxyl and X² is selected from fluorine, chlorine, bromine or iodine.

160. The compound of claim 159, wherein X¹ is hydroxyl and X² is fluorine.

161. A compound of Formula (Ib) is: 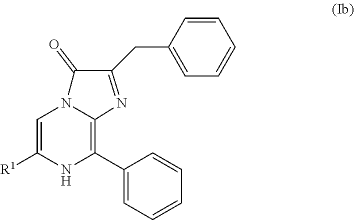
or a stereoisomer, a tautomer or a salt thereof, wherein:
R¹ is selected from: a cycloalkyl, 5 membered heteroaryl having 1-3 ring heteroatoms independently selected from O, S, Se and N; and 10 membered heteroaryl having 1-3 ring heteroatoms independently selected from O, S, Se and N;
or R¹ is selected from:

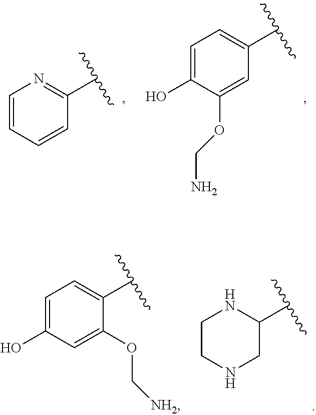 [SMILES: *c1ccccn1 ; *c1ccc(O)c(OCN)c1 ; *c1ccc(O)cc1OCN ; *C1CNCCN1]

162. The compound of claim 161, wherein R¹ is a cycloalkyl.

163. The compound of claim 162, wherein R¹ is a cyclopropyl.

164. The compound of claim 161, wherein R¹ is a 5 membered heteroaryl having 1-3 ring heteroatoms independently selected from O, S, Se and N.

165. The compound of claim 164, wherein R¹ is selected from pyrrole, furan, thiophene, selenophene, imidazole, thiazole and oxazole.

166. The compound of claim 161, wherein R¹ is a 10 membered heteroaryl having 1-3 ring heteroatoms independently selected from O, S, Se and N.

167. The compound of claim 166, wherein R¹ is quinoline.

168. The compound of claim 161, wherein R¹ is

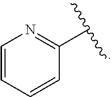 [SMILES: *c1ccccn1]

169. The compound of claim 161, wherein R¹ is

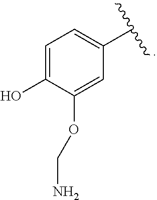 [SMILES: *c1ccc(O)c(OCN)c1]

170. The compound of claim 161, wherein R¹ is

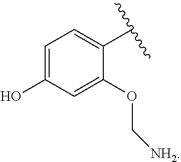 [SMILES: *c1ccc(O)cc1OCN]

171. The compound of claim 161, wherein R¹ is

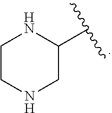 [SMILES: *C1CNCCN1]

172. A compound of formula (Ic): 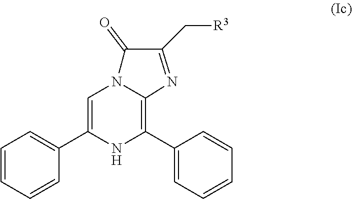
or a stereoisomer, a tautomer or a salt thereof, wherein:
R³ is selected from: a cycloalkyl, 5 membered heteroaryl having 1-3 ring heteroatoms independently selected from O, S, Se and N; and 10 membered heteroaryl having 1-3 ring heteroatoms independently selected from O, S, Se and N.

173. The compound of claim 172, wherein R³ is a cycloalkyl.

174. The compound of claim 173, wherein R³ is a cyclopropyl.

175. The compound of claim 172, wherein R³ is a 5 membered heteroaryl having 1-3 ring heteroatoms independently selected from O, S, Se and N.

176. The compound of claim 175, wherein R³ is selected from pyrrole, furan, thiophene, selenophene, imidazole, thiazole and oxazole.

177. The compound of claim 172, wherein R³ is a 10 membered heteroaryl having 1-3 ring heteroatoms independently selected from O, S, Se and N.

178. The compound of claim 177, wherein R³ is quinoline.

179. A compound of formula (Id): 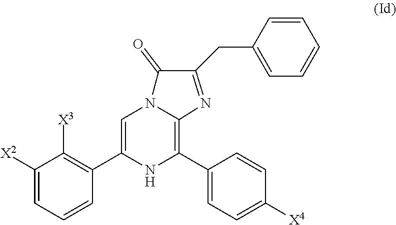
or a stereoisomer, a tautomer or a salt thereof, wherein:
X²-X³ are independently selected from: hydrogen, halogen, or hydroxy,
X⁴ is alkoxy;
with proviso that either one of X²-X³ is hydrogen.

180. The compound of claim 179, wherein X² is hydrogen and X³ is a halogen.

181. The compound of claim 180, wherein X² is hydrogen and X³ is selected from fluorine, chlorine, bromine or iodine.

182. The compound of claim 181, wherein X² is hydrogen and X³ is fluorine.

183. The compound of claim 179, wherein X² is hydrogen and X³ is hydroxy.

184. The compound of claim 179, wherein X³ is hydrogen and X² is a halogen.

185. The compound of claim 184, wherein X³ is hydrogen and X² is selected from fluorine, chlorine, bromine or iodine.

186. The compound of claim 185, wherein X³ is hydrogen and X² is fluorine.

187. The compound of claim 179, wherein X³ is hydrogen and X² is hydroxy.

188. A compound of Formula (Ie) is: 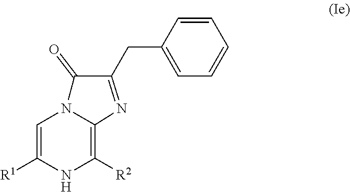
or a stereoisomer, a tautomer or a salt thereof, wherein: 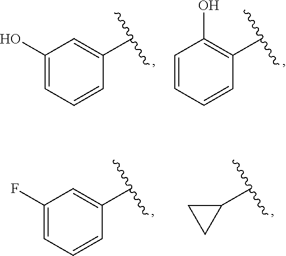
R¹ is selected from: 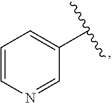 [SMILES: *c1cccnc1]
or R² is selected from:

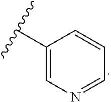 [SMILES: *c1cccnc1]

189. The compound of claim 188, wherein R¹ is

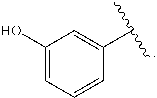 [SMILES: *c1cccc(O)c1]

190. The compound of claim 188, wherein R¹ is

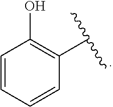 [SMILES: *c1ccccc1O]

191. The compound of claim 188, wherein R¹ is

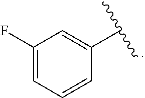 [SMILES: *c1cccc(F)c1]

192. The compound of claim 188, wherein R¹ is

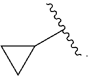 [SMILES: *C1CC1]

193. The compound of claim 188, wherein R¹ is

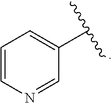 [SMILES: *c1cccnc1]

194. A compound selected from:

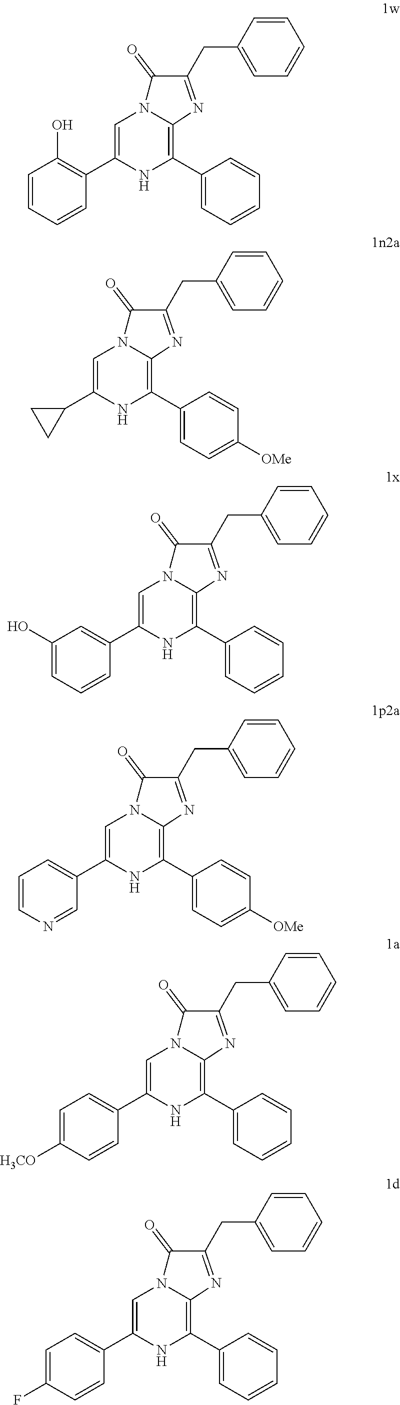

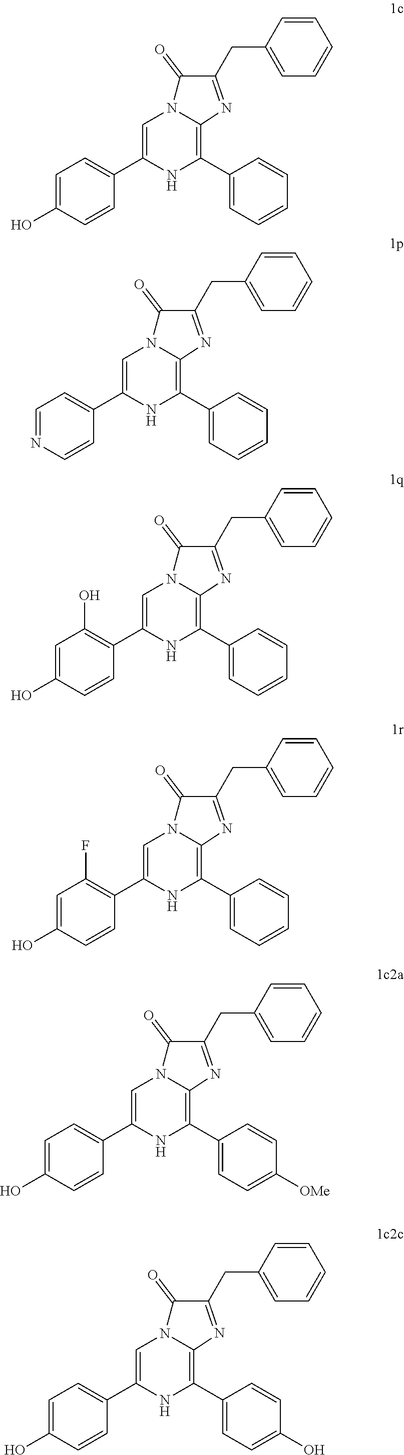

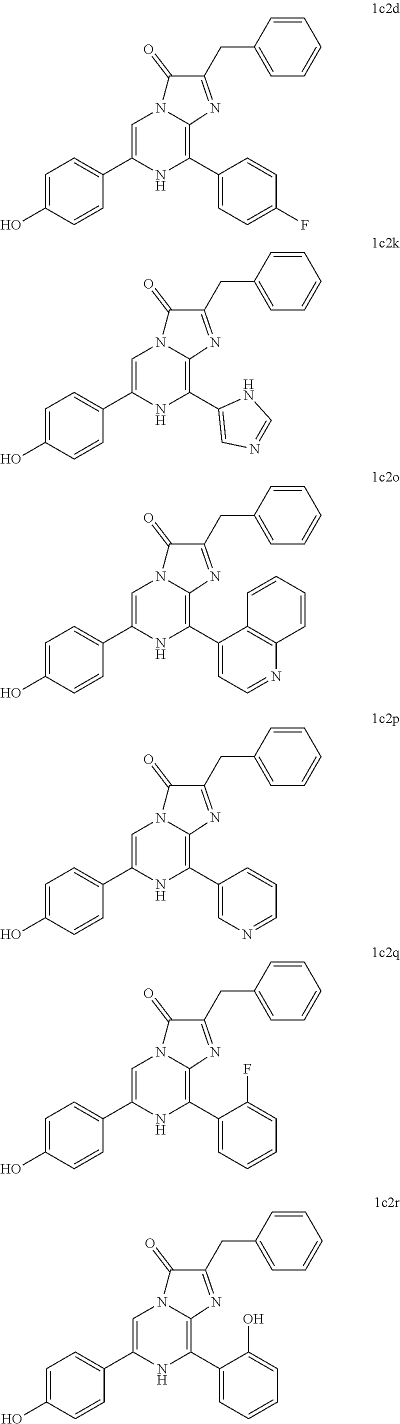

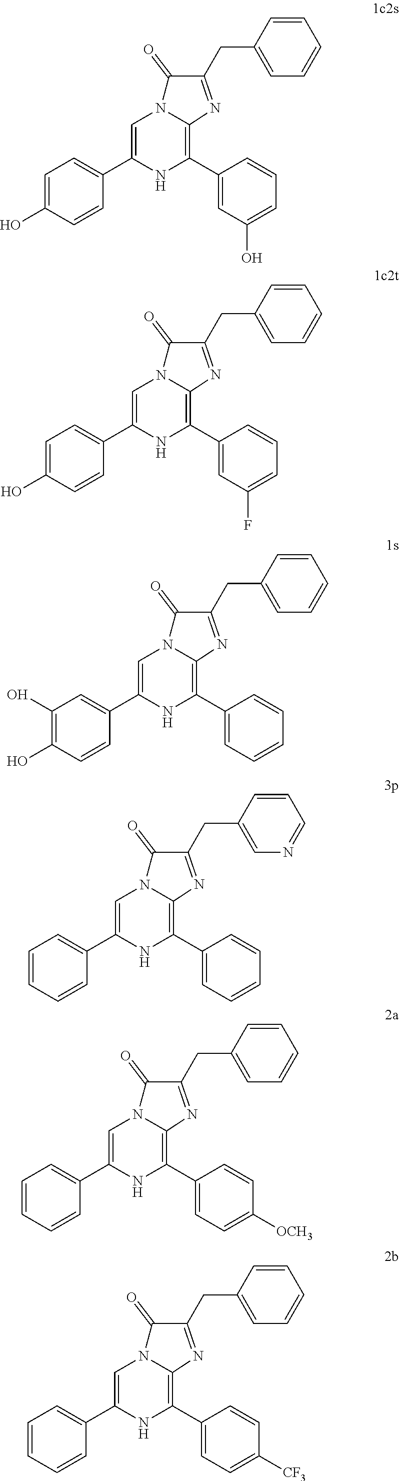

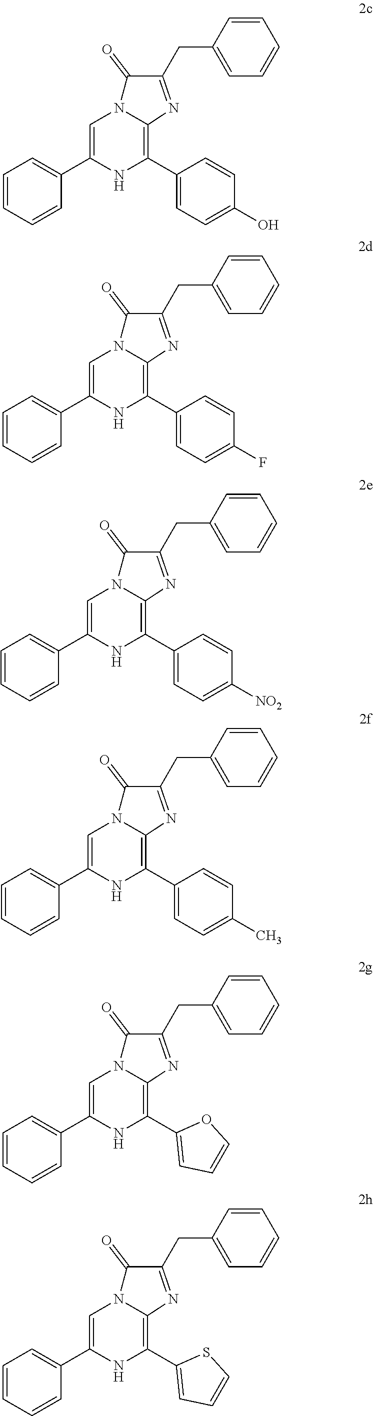

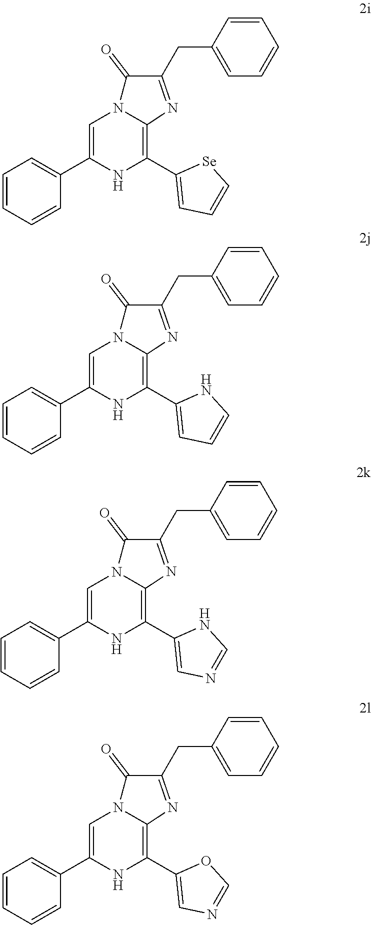

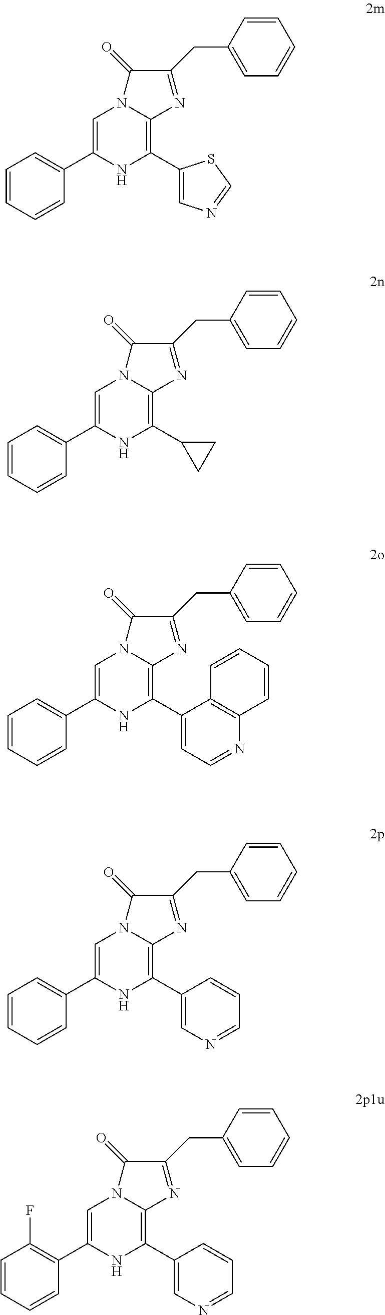 [SMILES: O=c1c(Cc2ccccc2)nc2c(-c3cncs3)[nH]c(-c3ccccc3)cn1-2 ; O=c1c(Cc2ccccc2)nc2c(C3CC3)[nH]c(-c3ccccc3)cn1-2 ; O=c1c(Cc2ccccc2)nc2c(-c3ccnc4ccccc34)[nH]c(-c3ccccc3)cn1-2 ; O=c1c(Cc2ccccc2)nc2c(-c3cccnc3)[nH]c(-c3ccccc3)cn1-2 ; O=c1c(Cc2ccccc2)nc2c(-c3cccnc3)[nH]c(-c3ccccc3F)cn1-2]

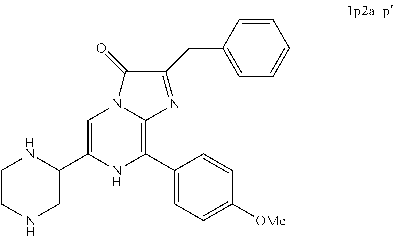 [SMILES: COc1ccc(-c2[nH]c(C3CNCCN3)cn3c(=O)c(Cc4ccccc4)nc2-3)cc1]

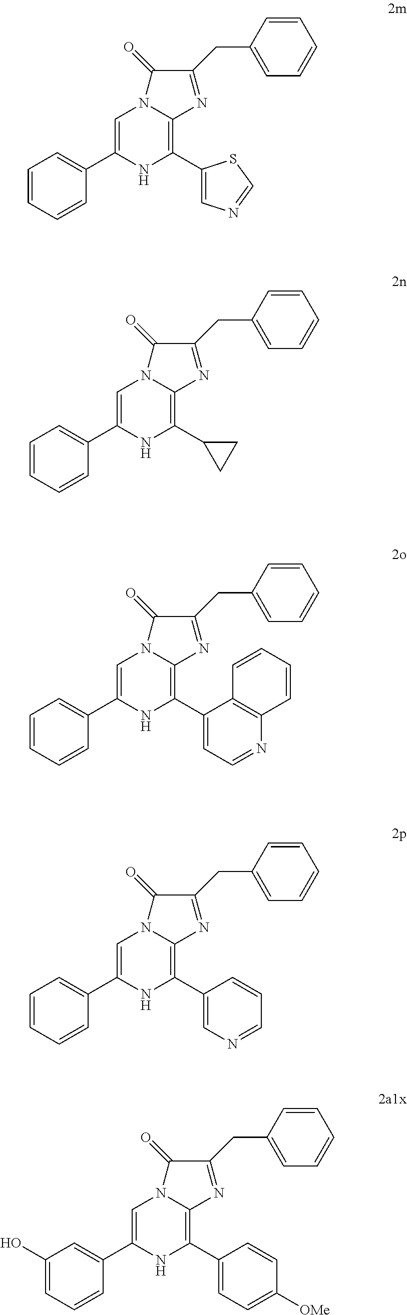 [SMILES: O=c1c(Cc2ccccc2)nc2c(-c3cncs3)[nH]c(-c3ccccc3)cn1-2 ; O=c1c(Cc2ccccc2)nc2c(C3CC3)[nH]c(-c3ccccc3)cn1-2 ; O=c1c(Cc2ccccc2)nc2c(-c3ccnc4ccccc34)[nH]c(-c3ccccc3)cn1-2 ; O=c1c(Cc2ccccc2)nc2c(-c3cccnc3)[nH]c(-c3ccccc3)cn1-2 ; COc1ccc(-c2[nH]c(-c3cccc(O)c3)cn3c(=O)c(Cc4ccccc4)nc2-3)cc1]

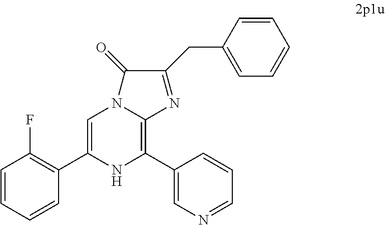 [SMILES: O=c1c(Cc2ccccc2)nc2c(-c3cccnc3)[nH]c(-c3ccccc3F)cn1-2]

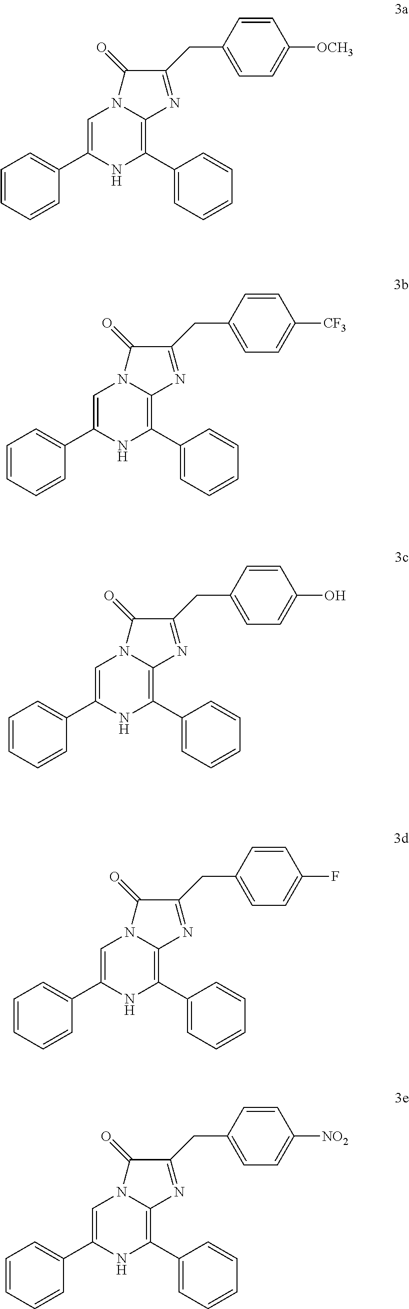 [SMILES: COc1ccc(Cc2nc3c(-c4ccccc4)[nH]c(-c4ccccc4)cn-3c2=O)cc1 ; O=c1c(Cc2ccc(C(F)(F)F)cc2)nc2c(-c3ccccc3)[nH]c(-c3ccccc3)cn1-2 ; O=c1c(Cc2ccc(O)cc2)nc2c(-c3ccccc3)[nH]c(-c3ccccc3)cn1-2 ; O=c1c(Cc2ccc(F)cc2)nc2c(-c3ccccc3)[nH]c(-c3ccccc3)cn1-2 ; O=c1c(Cc2ccc([N+](=O)[O-])cc2)nc2c(-c3ccccc3)[nH]c(-c3ccccc3)cn1-2]

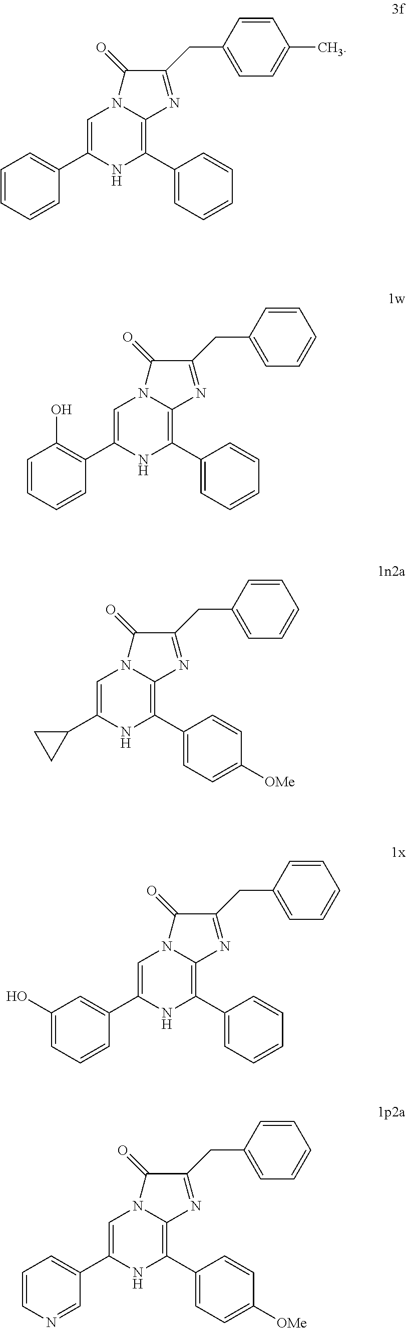 [SMILES: Cc1ccc(Cc2nc3c(-c4ccccc4)[nH]c(-c4ccccc4)cn-3c2=O)cc1 ; O=c1c(Cc2ccccc2)nc2c(-c3ccccc3)[nH]c(-c3ccccc3O)cn1-2 ; COc1ccc(-c2[nH]c(C3CC3)cn3c(=O)c(Cc4ccccc4)nc2-3)cc1 ; O=c1c(Cc2ccccc2)nc2c(-c3ccccc3)[nH]c(-c3cccc(O)c3)cn1-2 ; COc1ccc(-c2[nH]c(-c3cccnc3)cn3c(=O)c(Cc4ccccc4)nc2-3)cc1]

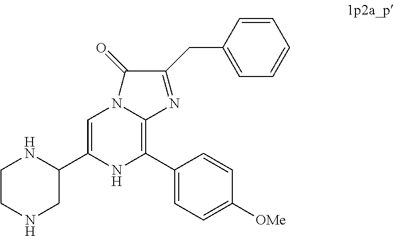 [SMILES: COc1ccc(-c2[nH]c(C3CNCCN3)cn3c(=O)c(Cc4ccccc4)nc2-3)cc1]

195. A method for detecting luminescence in a sample, the method comprising
contacting a sample with a luciferin analog, wherein the luciferin analog is a compound of formula (I), or a stereoisomer, a tautomer or a salt thereof, a compound of formula (Ia), a compound of formula (Ib), a compound of formula (Ic), a compound of formula (Id), a compound of formula (Ie), or a compound set forth in claim 194; and
detecting luminescence.

196. The method according to claim 195, wherein the sample comprises a luciferase, optionally wherein the luciferase is a protein of any one of claims 1-85, 94-113 or the polypeptide of any one of claims 86-93.

197. The method according to claim 195 or 196, wherein the sample contains live cells.

198. A method for detecting luminescence in a transgenic animal, the method comprising
administering a luciferin analog to a transgenic animal, wherein the luciferin analog is a compound of formula (I), or a stereoisomer, a tautomer or a salt thereof, a compound of formula (Ia), a compound of formula (Ib), a compound of formula (Ic), a compound of formula (Id), a compound of formula (Ie), or a compound set forth in claim 194; and
detecting luminescence;
wherein the transgenic animal expresses a luciferase, optionally wherein the luciferase is a protein of any one of claims 1-85, 94-113 or the polypeptide of any one of claims 86-93.

199. A method for assaying luciferase activity using the protein, polypeptide component, fusion protein, nucleic acid, expression vector, host cell, and/or kit of any preceding claim.

200. The method of claim 199, where the method comprises performing a luminescent reporting assay, diagnostic assay, cellular localization of a target of interest, cellular imaging, gene editing, live animal imaging, cancer labeling, CART-cells reporting, secreted assay, gene delivery, and/or tissue engineering.

201. A solution for measuring luciferase activity of a protein, the solution comprising 1 mM-1000 mM imidazole.

202. The solution of claim 201, wherein the pH of the solution is pH6-pH9, e.g., pH7-pH9 or pH7.5-8.5.

203. The solution of claim 201 or 202, further comprising phosphate-buffered saline.

204. The solution of any one of claims 201-203, comprising 5 mM-500 mM imidazole, 10 mM-1000 mM imidazole, 50 mM-1000 mM imidazole, 10 mM-500 mM imidazole, 50 mM-500 mM imidazole, 75 mM-250 mM imidazole, 75 mM-150 mM imidazole, 10 mM-250 mM imidazole, 10 mM-200 mM imidazole, or 5 mM-300 mM imidazole.

205. The solution of any one of claims 201-204, further comprising a stabilizing agent.

206. The solution of claim 205, wherein the stabilizing agent is ascorbic acid, glycine, and/or propylene glycol.

207. The solution of claim 205, wherein the stabilizing agent is glycine, optionally, wherein the solution comprises 200 mM-500 mM glycine or 200 mM-400 mM glycine.

208. The solution of claim 206 or claim 207, wherein the stabilizing agent is propylene glycol, optionally, wherein the solution comprises 0.1%-10% propylene glycol, 0.1%-0.3% propylene glycol, 0.3%-0.5% propylene glycol, 0.5%-0.8% propylene glycol, 0.3%-0.8% propylene glycol, 0.8%-1% propylene glycol, 1%-2% propylene glycol, 2%-3% propylene glycol, or 3%-5% propylene glycol.

209. The solution of any one of claims 201-208, further comprising a luciferin substrate or a kit comprising the solution of any one of claims 201-208 and a luciferin substrate.

210. The solution of claim 209 or the kit of claim 209, wherein the luciferin substrate is DTZ, a compound of formula (I), a compound of formula (Ia), a compound of formula (Ib), a compound of formula (Ic), a compound of formula (Id), a compound of formula (Ie), or a compound of claim 194.

211. A kit comprising: a compound of formula (I), a compound of formula (Ia), a compound of formula (Ib), a compound of formula (Ic), a compound of formula (Id), a compound of formula (Ie), or a compound of claim 194, optionally further comprising a luciferase and/or an assay buffer, further optionally wherein the luciferase is a multipartite protein comprising two self-complementing components or three self-complementing components and/or the assay buffer is the solution of any one of claims 201-210.

212. A kit comprising: a nucleic acid comprising a nucleotide sequence encoding the protein of any one of claims 1-82, 112-113, 117-136, 141-142, the first polypeptide component of any one of claims 83-85, or the second polypeptide component of any one of claims 83-85 and optionally an assay buffer.

213. The kit of claim 212, wherein the nucleic acid is the nucleic acid of claim 114, claim 143, or is the expression vector of claim 144.

214. The kit of claim 212, wherein the nucleic acid is present in an expression vector.

215. The kit of claim 212, wherein the nucleic acid is the first nucleic acid or the second nucleic acid of any one of claims 146-149, optionally wherein the first nucleic acid or the second nucleic acid is in an expression vector.

216. The kit of claim 212, wherein the kit comprises a first nucleic acid and a second nucleic acid of any of one claims 146-149, optionally wherein the first nucleic acid is in a first expression vector and the second nucleic acid is in a second expression vector.

217. The kit of any one of claims 212-216, comprising a compound of any one of claims 153-194.

218. The kit of claim 217, wherein the compound is compound 1c.

219. The kit of any one of claims 212-218, comprising an assay buffer.

220. The kit of claim 219, wherein the assay buffer comprises phosphate buffered saline.

221. The kit of claim 219 or claim 220, wherein the assay buffer comprises imidazole.

222. The kit of claim 221, wherein the assay buffer comprises 5 mM-500 mM imidazole, 10 mM-1000 mM imidazole, 50 mM-1000 mM imidazole, 10 mM-500 mM imidazole, 50 mM-500 mM imidazole, 75 mM-250 mM imidazole, 75 mM-150 mM imidazole, 10 mM-250 mM imidazole, or 5 mM-300 mM imidazole.

223. The kit of any one of claims 219-222, wherein the assay buffer comprises a stabilizing agent.

224. The kit of claim 223, wherein the stabilizing agent is ascorbic acid, glycine, and/or propylene glycol.

225. The kit of claim 223 or 224, wherein the stabilizing agent is glycine, optionally, wherein the solution comprises 200 mM-500 mM glycine.

226. The kit of any one of claims 223-225, wherein the stabilizing agent is propylene glycol, optionally, wherein the assay buffer comprises 0.1%-10% propylene glycol, 0.1%-0.3% propylene glycol, 0.3%-0.5% propylene glycol, 0.5%-0.8% propylene glycol, 0.8%-1% propylene glycol, 1%-2% propylene glycol, 2%-3% propylene glycol, or 3%-5% propylene glycol.

227. The kit of any one of claims 212-226, wherein the nucleic acid is in a lyophilized form, the kit of any one of claims 217-218, wherein the compound is in a lyophilized form, and/or the kit of any one of claims 219-226, wherein the assay buffer is in a lyophilized form.
